(19) United States
(12) Patent Application Publication
(10) Pub. No.: US 2025/0129160 A1
(43) Pub. Date: Apr. 24, 2025

(54) T CELL ENGAGER MOLECULES AND USES THEREOF

(71) Applicants: AMGEN RESEARCH (MUNICH) GmbH, Munich (DE); AMGEN INC., Thousand Oaks, CA (US)

(72) Inventors: Johannes BROZY, Munich (DE); Christoph DAHLHOFF, Geretsried (DE); Tobias RAUM, Eurasburg (DE); Jochen S. PENDZIALEK, Gauting (DE); Lisa WINKEL, Munich (DE); Marcus MUENZ, Lenggries (DE); Nathan William PIERCE, Mountain View, CA (US); Agnieszka KIELCZEWSKA, Vancouver (CA); Wentao CHEN, Thousand Oaks, CA (US); Darren L. BATES, Thousand Oaks, CA (US); Claudia BLUEMEL, Munich (DE); Jonas Karl-Josef HONER, Munich (DE)

(73) Assignees: AMGEN RESEARCH (MUNICH) GmbH, Munich (DE); AMGEN INC., Thousand Oaks, CA (US)

(21) Appl. No.: 18/566,228

(22) PCT Filed: Jun. 2, 2022
(86) PCT No.: PCT/US2022/032006

§ 371 Date: Dec. 1, 2023

Related U.S. Application Data
(60) Provisional application No. 63/236,547, filed on Aug. 24, 2021.
(60) Provisional application No. 63/197,265, filed on Jun. 4, 2021.

(51) Int. Cl. C07K 16/28

(52) U.S. Cl. CPC C07K 16/2809; C07K 16/2818; C07K 16/2827; C07K 2317/31; C07K 2317/33; C07K 2317/522; C07K 2317/55; C07K 2317/622; C07K 2317/66; C07K 2317/92

(57) ABSTRACT

The present invention provides single chain T cell engager (TCE) molecules having an scFab that binds a target antigen and an scFv that binds CD3, and TCE molecules that bind CCR8 and CD3. Methods of treating cancer are also provided.

### CROSS REFERENCE TO RELATED APPLICATIONS

This application claims the benefit of U.S. Provisional Application No. 63/197,265, filed Jun. 4, 2021, and U.S. Provisional Application No. 63/236,547, filed Aug. 24, 2021, each of which is incorporated by reference herein in its entirety.

### FIELD OF THE INVENTION

The present invention relates to the field of oncology, in particular, to bispecific T cell engager (TCE) molecules and treatment of cancer patients with said molecules.

### BACKGROUND OF THE INVENTION

The redirection of T cell activity against tumor cells by means of bispecific molecules independent of T cell receptor specificity is an evolving approach in immunooncology (Frankel S R, Baeuerle P A. Targeting T cells to tumor cells using bispecific antibodies. Curr Opin Chem Biol 2013; 17:385-92). Such new protein-based pharmaceuticals typically can simultaneously bind to two different types of antigen. They are known in several structural formats, and current applications have been explored for cancer immunotherapy and drug delivery (Fan, Gaowei; Wang, Zujian; Hao, Mingju; Li, Jinming (2015). “Bispecific antibodies and their applications”. Journal of Hematology & Oncology. 8:130).

Bispecific molecules useful in immunooncology can be antigen-binding polypeptides such as antibodies, e.g. IgG-like, i.e. full-length bispecific antibodies, or non-IgG-like bispecific antibodies, which are not full-length antibody constructs. Full length bispecific antibodies typically retain the traditional monoclonal antibody (mAb) structure of two Fab arms and one Fc region, except the two Fab sites bind different antigens. Non-full-length bispecific antibodies can lack an Fc region entirely. These include chemically linked Fabs, consisting of only the Fab regions, and various types of bivalent and trivalent single-chain variable fragments (scFvs). There are also fusion proteins mimicking the variable domains of two antibodies. An example of such a format is the bispecific T-cell engager (BiTER) (Yang, Fa; Wen, Weihong; Qin, Weijun (2016). “Bispecific Antibodies as a Development Platform for New Concepts and Treatment Strategies”. International Journal of Molecular Sciences. 18 (1): 48).

BiTE molecules are recombinant protein constructs made from two flexibly linked antibody derived binding domains. One binding domain of BiTE is specific for a selected tumor-associated surface antigen on target cells; the second binding domain is specific for CD3, a subunit of the T cell receptor complex on T cells. By their particular design, BiTE molecules are uniquely suited to transiently connect T cells with target cells and, at the same time, potently activate the inherent cytolytic potential of T cells against target cells.

There exists a need for bispecific molecules, specifically T cell engager (“TCE”) molecules, that bind a target antigen and CD3, and that demonstrate increased lysis of target cells and have desirable manufacturing properties such as increased aggregation temperatures and steeper HIC elution peak slopes. The present invention provides single-chain TCE molecules having an scFab that binds a target antigen (e.g. tumor antigen) and an scFv that binds CD3. Some TCE molecules further have an scFc, connected by a linker to the scFv, to extend the molecule half-life. The TCE molecules of the present invention demonstrate improved lysis of target cells and improved properties related to manufacturing.

The present invention also provides CCR8 TCE molecules that bind CCR8 and CD3. The C-C chemokine receptor type 8 (CCR8) is a member of the beta chemokine receptor family and is a seven transmembrane G-protein-coupled receptor with a 35 amino acid extracellular N-terminus. The ligand for CCR8 is CCL1, and CCL1-induced CCR8 signaling occurs via G-coupled proteins. CCR8 is expressed with much higher prevalence and at higher levels on the surface of cancer-resident Tregs compared to circulating or normal tissue Tregs and conventional T effector (Teff) cells. Treg cell infiltration in solid tumors is associated with poor clinical outcome, and Tregs suppress the anti-cancer immune response through inhibition of Teff cell cytotoxicity.

CCR8 TCE molecules of the present invention are thought to induce redirected T cell lysis of tumor-resident CCR8+ Tregs while sparing normal tissue Tregs that have little to no CCR8 expression. CCR8 TCE molecules of the present invention are thought to have an improved safety profile compared to other Treg-depleting therapeutic candidates targeting other markers that do not specifically deplete cancer-resident Tregs.

CCR8 TCE molecules of the present invention are single chain molecules and have either an (i) scFv that binds CCR8 and an scFv that binds CD3, wherein the two scFvs are connected by a linker; or (ii) an scFab that binds CCR8 and an scFv that binds CD3, wherein the scFab and scFv are connected by a linker. Some TCE molecules further have a scFc, connected by a linker to the scFv that binds CD3, to extend the half-life of the molecule. The CCR8 TCE molecules of the present invention demonstrate pM range cytotoxicity and bind both cynomolgus monkey and human CCR8. Interestingly, CCR8 TCE molecules were discovered that bind a unique epitope on CCR8 and do not block ligand binding to CCR8. Binding to this unique epitope is thought to contribute to high affinity and bioactivity of the TCE molecule. Binding to this unique epitope may also contribute to an acceptable pharmacokinetic profile.

### SUMMARY OF THE INVENTION

The present invention provides a T cell engager (TCE) molecule, which may be referred to as an scFab-containing TCE molecule, comprising (i) an scFab that binds a tumor antigen, wherein the scFab comprises a first heavy chain variable region (scFab VH), a CHI domain, a first light chain variable region (scFab VL), and a Ck or CA domain, and (ii) an scFv that binds CD3, comprising a second VL and a second VH, wherein the TCE molecule is a single chain. In some embodiments, the scFab comprises a C-terminus portion that is connected by a linker to an N-terminal portion of the scFv. In some embodiment the TCE molecule further comprises an scFc. In some embodiments, the scFc comprises an N-terminus portion that is connected by a linker to the C-terminal portion of the scFv. In a particular embodiment, the scFv binds human CD3. In some embodiments, the tumor antigen is CCR8.

In some embodiments, the scFab of a TCE molecule of the present invention has an orientation in the following order, from N-terminus to C-terminus, VH, CH1, VL, and either Cκ or Cλ. In other embodiments, the scFab has an orientation in the following order, from N-terminus to C-terminus, VL, either Cκ or Cλ, VH, and CH1. In some embodiments, the scFab comprises a linker that connects the CH1 and VL, wherein the linker is (G4S)6, (G4S)7, (G4S)8, (G4Q)6, (G4Q)7, or (G4Q)8. In some embodiments, the scFab comprises a linker that connects Cκ or Cλ and VH, wherein the linker is (G4S)6, (G4S)7, (G4S)8, (G4Q)6, (G4Q)7, or (G4Q)8. In some embodiments, the scFab contains a natural cysteine clamp between the heavy and light chain constant domains. In some embodiments, the TCE molecule comprises an engineered cysteine clamp in the scFab between residue 44 in the VH domain and residue 100 in the VL domain (Kabat numbering). In some embodiments, the scFab contains a natural cysteine clamp between the heavy and light chain constant domains and an engineered cysteine clamp between residue 44 in the VH domain and residue 100 in the VL domain. In some embodiments, the TCE molecule CH1, Cκ and/or Cλ domains are IgG, IgM, IgA, IgD, or IgE. In a particular embodiment, the domains are IgG. In a more particular embodiment, the domains are IgG1. In some embodiments, the domains are human. In a particular embodiment, the domains are human IgG1.

The present invention provides a single-chain TCE molecule having the following orientation, from N-terminus to C-terminus: scFab (VH, CH1, linker, VL, either Cκ or Cλ), linker, scFv (VH, linker, VL). In an embodiment, the TCE molecule further comprises a scFc, and has the following orientation: scFab (VH, CHI, linker, VL, either Cκ or Cλ), linker, scFv (VH, linker, VL), linker, Fc1 (hinge, CH2, CH3), linker, Fc2 (hinge, CH2, CH3).

The present invention provides a single-chain TCE molecule having the following orientation, from N-terminus to C-terminus: scFab (VL, either Cκ or C2, linker, VH, CH1), linker, scFv (VH, linker, VL). In an embodiment, the TCE molecule further comprises a scFc, and has the following orientation: scFab (VL, either Cκ or Cλ, linker, VH, CH1), linker, scFv (VH, linker, VL), linker, Fc1 (hinge, CH2, CH3), linker, Fc2 (hinge, CH2, CH3).

The present invention provides a single-chain TCE molecule having the following orientation: scFv that binds CCR8 (VH, linker, VL), linker, scFv that binds CD3 (VH, linker, VL). In an embodiment, the TCE molecule further comprises a scFc, and has the following orientation: scFv that binds CCR8 (VH, linker, VL), linker, scFv that binds CD3 (VH, linker, VL)-Linker-Fc1 (hinge, CH2, CH3), linker, Fc2 (hinge, CH2, CH3).

The present invention also provides a TCE molecule having the following orientation from N-terminus to C-terminus: scFv that binds CCR8 (VH, linker, VL)-Linker-scFv that binds CD3 (VH, linker, VL)-Linker-Fc1 (CH2-CH3)-Linker-Fc2 (CH2-CH3). In an embodiment, the TCE molecule binds CCR8 and CD3. The present invention provides a TCE molecule having the following orientation from N-terminus to C-terminus: scFv that binds CCR8 (VL-Linker-VH)-Linker-scFv that binds CD3 (VH-Linker-VL)-Linker-Fc1 (CH2-CH3)-Linker-Fc2 (CH2-CH3). In an embodiment, the TCE molecule binds CCR8 and CD3.

The present invention provides a single-chain TCE molecule having a scFab-scFv-scFv-scFc format. In some embodiments, the TCE molecule comprises the following orientation: VH-CH1-Linker-VL-Cκ/Cλ-Linker-VH-Linker-VL-Linker-VH-Linker-VL-Linker-Fc1-Linker-Fc2. In some embodiments, the TCE molecule comprises the following orientation: VL-Cκ/Cλ-Linker-VH-CH1-Linker-VH-Linker-VL-Linker-VH-Linker-VL-Linker-Fc1-Linker-Fc2. In some embodiments, the TCE molecule comprises the following orientation: VL-Cκ/Cλ-Linker-VH-CH1-Linker-VL-Linker-VH-Linker-VH-Linker-VL-Linker-Fc1-Linker-Fc2. In some embodiments, the TCE molecule comprises the following orientation: VH-CH1-Linker-VL-Cκ/C2-Linker-VL-Linker-VH-Linker-VH-Linker-VL-Linker-Fc1-Linker-Fc2. In some embodiments, the TCE molecule comprises Cκ. In some embodiments, the TCE molecule comprises Cλ.

The present invention also provides a single-chain TCE molecule having an scFab-scFab-scFv-scFc format. In some embodiments, the TCE molecule comprises the following orientation: VH-CH1-Linker-VL-Cκ/Cλ-Linker-VH-CH1-Linker-VL-Cκ/Cλ-Linker-VH-Linker-VL-Linker-Fc1-Linker-Fc2. In some embodiments, the TCE molecule comprises the following orientation: VL-Cκ/C2-Linker-VH-CH1-Linker-VH-CH1-Linker-VL-Cκ/Cλ-Linker-VH-Linker-VL-Linker-Fc1-Linker-Fc2. In some embodiments, the TCE molecule comprises the following orientation: VH-CH1-Linker-VL-Cκ/Cλ-Linker-VL-Cκ/Cλ-Linker-VH-CH1-Linker-VH-Linker-VL-Linker-Fc1-Linker-Fc2. In some embodiments, the TCE molecule comprises the following orientation: VL-Cκ/Cλ-Linker-VH-CH1-Linker-VL-Cκ/Cλ-Linker-VH-CH1-Linker-VH-Linker-VL-Linker-Fc1-Linker-Fc2. In some embodiments, the TCE molecule comprises Cκ. In some embodiments, the TCE molecule comprises Cλ. In some embodiments, the TCE molecule comprises Cκ and Cλ.

In an embodiment, the scFab VH and CHI of an scFab-containing TCE molecule of the present invention comprise an amino acid sequence given by SEQ ID NO: 12, SEQ ID NO: 28, SEQ ID NO: 44, SEQ ID NO: 60, SEQ ID NO: 76, SEQ ID NO: 92, SEQ ID NO: 108, or SEQ ID NO: 124. In an embodiment, the TCE molecule of the present invention comprises a Cκ. In a particular embodiment, the scFab VL and Cκ of an scFab-containing TCE molecule of the present invention comprise an amino acid sequence given by SEQ ID NO: 13, SEQ ID NO: 29, SEQ ID NO: 45, SEQ ID NO: 61, SEQ ID NO: 77, SEQ ID NO: 93, SEQ ID NO: 109, or SEQ ID NO: 125. In another particular embodiment, the TCE molecule comprises an amino acid sequence given by SEQ ID NO: 14, SEQ ID NO: 30, SEQ ID NO: 46, SEQ ID NO: 62, SEQ ID NO: 78, SEQ ID NO: 94, SEQ ID NO: 110, or SEQ ID NO: 126. In another particular embodiment, the TCE molecule comprises an amino acid sequence given by SEQ ID NO: 15, SEQ ID NO: 31, SEQ ID NO: 47, SEQ ID NO: 63, SEQ ID NO: 79, SEQ ID NO: 95, SEQ ID NO: 111, or SEQ ID NO: 127. In another particular embodiment, the TCE molecule comprises an amino acid sequence given by SEQ ID NO: 16, SEQ ID NO: 32, SEQ ID NO: 48, SEQ ID NO: 64, SEQ ID NO: 80, SEQ ID NO: 96, SEQ ID NO: 112, or SEQ ID NO: 128.

In another embodiment, the scFab VH and CHI or scFab VL and Cκ comprise a sequence of amino acids that is at least 70%, 75%, 80%, 85%, 90%, 91%, 92%, 93%, 94%, 95%, 96%, 97%, 98%, 99% or 100% identical to the sequence of a scFab VH and CHI or scFab VL and Cκ sequence listed herein. In another embodiment, the TCE molecule comprises a sequence of amino acids that is at least 70%, 75%, 80%, 85%, 90%, 91%, 92%, 93%, 94%, 95%, 96%, 97%, 98%, 99% or 100% identical to the sequence of a TCE molecule sequence listed herein.

The present invention also provides a TCE molecule comprising (i) a first scFv that binds CCR8, wherein the first scFv comprises a first VH region (CCR8 scFv VH) and a first VL region (CCR8 scFv VL), and (ii) a second scFv that binds CD3, wherein the second scFv comprises a second VH region and a second VL region. A molecule having this structure and that binds CCR8 and CD3 may be referred to as a CCR8 TCE molecule. In a preferred embodiment, the CCR8 TCE molecule is a single chain.

In an embodiment, the CCR8 TCE molecule scFv VH comprises an amino acid sequence given by SEQ ID NO: 7, SEQ ID NO: 23, SEQ ID NO: 39, SEQ ID NO: 55, SEQ ID NO: 71, SEQ ID NO: 87, SEQ ID NO: 103, or SEQ ID NO: 119, and wherein the CCR8 scFv VL comprises an amino acid sequence given by SEQ ID NO: 8, SEQ ID NO: 24, SEQ ID NO: 40, SEQ ID NO: 56, SEQ ID NO: 72, SEQ ID NO:88, SEQ ID NO: 104, or SEQ ID NO: 120. In another embodiment, the first scFv comprises an amino acid sequence given by SEQ ID NO: 9, 25, 41, 57, 73, 89, 105, or 121. In another embodiment, the TCE molecule comprises an amino acid sequence given by SEQ ID NO: 10, SEQ ID NO: 26, SEQ ID NO: 42, SEQ ID NO: 58, SEQ ID NO: 74, SEQ ID NO: 90, SEQ ID NO: 106, or SEQ ID NO: 122. In another embodiment, the TCE molecule further comprises an scFc, wherein the TCE molecule comprises an amino acid sequence given by SEQ ID NO: 11, SEQ ID NO: 27, SEQ ID NO: 59, SEQ ID NO: 75, SEQ ID NO: 91, SEQ ID NO: 107, or SEQ ID NO: 123. In another embodiment, the CCR8 scFv VH comprises a sequence of amino acids that is at least 70%, 75%, 80%, 85%, 90%, 91%, 92%, 93%, 94%, 95%, 96%, 97%, 98%, 99% or 100% identical to CCR8 scFv VH sequences listed herein. In another embodiment, the CCR8 scFv VL comprises a sequence of amino acids that is at least 70%, 75%, 80%, 85%, 90%, 91%, 92%, 93%, 94%, 95%, 96%, 97%, 98%, 99% or 100% identical to the CCR8 scFv VL sequences listed herein.

In another embodiment, the first scFv (that binds CCR8) comprises a sequence of amino acids that is at least 70%, 75%, 80%, 85%, 90%, 91%, 92%, 93%, 94%, 95%, 96%, 97%, 98%, 99% or 100% identical to the first scFv sequences listed herein.

In another embodiment, a CCR8 TCE molecule of the present invention comprises a sequence of amino acids that is at least 70%, 75%, 80%, 85%, 90%, 91%, 92%, 93%, 94%, 95%, 96%, 97%, 98%, 99% or 100% identical to CCR8 TCE molecule sequences listed herein.

In some embodiments, the first VH (scFab VH and/or CCR8 scFv VH) of a TCE molecule of the present invention comprises HCDR1, HCDR2, HCDR3, and the first VL (scFab VL and/or CCR8 scFv VL) comprises LCDR1, LCDR2, and LCDR3, and wherein:
a) HCDR1 comprises an amino acid sequence given by SEQ ID NO: 1, SEQ ID NO: 17, SEQ ID NO: 33, SEQ ID NO: 49, SEQ ID NO: 65, SEQ ID NO: 81, SEQ ID NO: 97, or SEQ ID NO: 113;
b) HCDR2 comprises an amino acid sequence given by SEQ ID NO: 2, SEQ ID NO: 18, SEQ ID NO: 34, SEQ ID NO: 50, SEQ ID NO: 66, or SEQ ID NO: 82;
c) HCDR3 comprises an amino acid sequence given by SEQ ID NO: 3, SEQ ID NO: 19, SEQ ID NO: 35, SEQ ID NO: 51, SEQ ID NO: 67, or SEQ ID NO: 83;
d) LCDR1 comprises an amino acid sequence given by SEQ ID NO: 4, SEQ ID NO: 20, SEQ ID NO: 36, SEQ ID NO: 52, SEQ ID NO: 68, or SEQ ID NO: 84;
e) LCDR2 comprises an amino acid sequence given by SEQ ID NO:5, SEQ ID NO: 21, SEQ ID NO: 37, SEQ ID NO: 53, SEQ ID NO: 69, or SEQ ID NO: 85, and
f) LCDR3 comprises an amino acid sequence given by SEQ ID NO: 6, SEQ ID NO: 22, SEQ ID NO: 38, SEQ ID NO: 54, SEQ ID NO: 70, or SEQ ID NO: 86.

In a particular embodiment, HCDR1 comprises an amino acid sequence given by SEQ ID NO: 1, HCDR2 comprises an amino acid sequence given by SEQ ID NO: 2, HCDR3 comprises an amino acid sequence given by SEQ ID NO: 3, LCDR1 comprises an amino acid sequence given by SEQ ID NO: 4, LCDR2 comprises an amino acid sequence given by SEQ ID NO: 5, and LCDR3 comprises an amino acid sequence given by SEQ ID NO: 6.

In another particular embodiment, HCDR1 comprises an amino acid sequence given by SEQ ID NO: 17, HCDR2 comprises an amino acid sequence given by SEQ ID NO: 18, HCDR3 comprises an amino acid sequence given by SEQ ID NO: 19, LCDR1 comprises an amino acid sequence given by SEQ ID NO: 20, LCDR2 comprises an amino acid sequence given by SEQ ID NO: 21, and LCDR3 comprises an amino acid sequence given by SEQ ID NO: 22.

In another particular embodiment, HCDR1 comprises an amino acid sequence given by SEQ ID NO: 33, HCDR2 comprises an amino acid sequence given by SEQ ID NO: 34, HCDR3 comprises an amino acid sequence given by SEQ ID NO: 35, LCDR1 comprises an amino acid sequence given by SEQ ID NO: 36, LCDR2 comprises an amino acid sequence given by SEQ ID NO: 37, and LCDR3 comprises an amino acid sequence given by SEQ ID NO: 38.

In yet another particular embodiment, HCDR1 comprises an amino acid sequence given by SEQ ID NO: 49, HCDR2 comprises an amino acid sequence given by SEQ ID NO: 50, HCDR3 comprises an amino acid sequence given by SEQ ID NO: 51, LCDR1 comprises an amino acid sequence given by SEQ ID NO: 52, LCDR2 comprises an amino acid sequence given by SEQ ID NO: 53, and LCDR3 comprises an amino acid sequence given by SEQ ID NO: 54.

In another particular embodiment, HCDR1 comprises an amino acid sequence given by SEQ ID NO: 65, HCDR2 comprises an amino acid sequence given by SEQ ID NO: 66, HCDR3 comprises an amino acid sequence given by SEQ ID NO: 67, LCDR1 comprises an amino acid sequence given by SEQ ID NO: 68, LCDR2 comprises an amino acid sequence given by SEQ ID NO: 69, and LCDR3 comprises an amino acid sequence given by SEQ ID NO: 70.

In another particular embodiment, HCDR1 comprises an amino acid sequence given by SEQ ID NO: 81, HCDR2 comprises an amino acid sequence given by SEQ ID NO: 82, HCDR3 comprises an amino acid sequence given by SEQ ID NO: 83, LCDR1 comprises an amino acid sequence given by SEQ ID NO: 84, LCDR2 comprises an amino acid sequence given by SEQ ID NO: 85, and LCDR3 comprises an amino acid sequence given by SEQ ID NO: 86.

In yet another particular embodiment, HCDR1 comprises an amino acid sequence given by SEQ ID NO: 97, HCDR2 comprises an amino acid sequence given by SEQ ID NO: 98, HCDR3 comprises an amino acid sequence given by SEQ ID NO: 99, LCDR1 comprises an amino acid sequence given by SEQ ID NO: 100, LCDR2 comprises an amino acid sequence given by SEQ ID NO: 101, and LCDR3 comprises an amino acid sequence given by SEQ ID NO: 102.

In another particular embodiment, HCDR1 comprises an amino acid sequence given by SEQ ID NO: 113, HCDR2 comprises an amino acid sequence given by SEQ ID NO: 114, HCDR3 comprises an amino acid sequence given by SEQ ID NO: 115, LCDR1 comprises an amino acid sequence given by SEQ ID NO: 116 or SEQ ID NO: 336 (KSSQSVLYSSNNXINYLA, wherein X1 is K or R), LCDR2 comprises an amino acid sequence given by SEQ ID NO: 117, and LCDR3 comprises an amino acid sequence given by SEQ ID NO: 118.

The present invention provides a TCE molecule comprising an orientation, from N-terminus to C-terminus, of an scFv that binds CCR8 (VH, linker, VL), linker, scFv that binds CD3 (VH, linker, VL), wherein the scFv that binds CCR8 comprises CDR1, HCDR2, HCDR3, LCDR1, LCDR2, and LCDR3 comprising amino acid residues given by SEQ ID NO: 217, SEQ ID NO: 218, SEQ ID NO: 219, SEQ ID NO: 220, SEQ ID NO: 221, and SEQ ID NO: 222, respectively. In an embodiment, the scFv that binds CCR8 comprises a VH and VL given by SEQ ID NO: 223 and SEQ ID NO: 224, respectively. In a particular embodiment, the scFv that binds CCR8 comprises amino acid residues given by SEQ ID NO: 225. In an embodiment, the TCE molecule comprises G4S linkers. In an embodiment, the TCE molecule comprises G4Q linkers. In an embodiment, the CD3-binding scFv is I2E. In another embodiment, the CD3-binding scFv is I2C. In another embodiment, the TCE molecule comprises the amino sequence given by SEQ ID NO: 226. In a further embodiment, the TCE molecule comprises the amino acid sequence given by SEQ ID NO: 227. In some such embodiments, the TCE molecule is TCE 1.1. In a preferred embodiment, the TCE molecule is a single chain. In some embodiments, the TCE molecule may have an orientation such that the VL is N-terminal to the VH.

The present invention provides a TCE molecule comprising an orientation, from N-terminus to C-terminus, of an scFv that binds CCR8 (VH, linker, VL), linker, scFv that binds CD3 (VH, linker, VL)-Linker-Fc1 (hinge, CH2, CH3), linker, Fc2 (hinge, CH2, CH3), wherein the scFv that binds CCR8 comprises CDR1, HCDR2, HCDR3, LCDR1, LCDR2, and LCDR3 comprising amino acid residues given by SEQ ID NO: 228, SEQ ID NO: 229, SEQ ID NO: 230, SEQ ID NO: 231, SEQ ID NO: 232, and SEQ ID NO: 233, respectively. In an embodiment, the scFv that binds CCR8 comprises a VH and VL given by SEQ ID NO: 234 and SEQ ID NO: 235, respectively. In a particular embodiment, the an scFv that binds CCR8 comprises amino acid residues given by SEQ ID NO: 236. In an embodiment, the TCE molecule comprises G4S linkers. In an embodiment, the TCE molecule comprises G4Q linkers. In an embodiment, the CD3-binding scFv is I2E. In another embodiment, the CD3-binding scFv is I2C. In another embodiment, the TCE molecule comprises the amino sequence given by SEQ ID NO: 237. In a further embodiment, the TCE molecule comprises the amino acid sequence given by SEQ ID NO: 238. In some such embodiments, the TCE molecule is TCE 1.2. In a preferred embodiment, the TCE molecule is a single chain. In some embodiments, the TCE molecule may have an orientation such that the VL is N-terminal to the VH.

The present invention provides a TCE molecule comprising an orientation, from N-terminus to C-terminus, of a scFab that binds CCR8 (VH, CHI, linker, VL, either Cκ or Cλ), linker, an scFv that binds CD3 (VH, linker, VL), wherein the scFab that binds CCR8 comprises CDR1, HCDR2, HCDR3, LCDR1, LCDR2, and LCDR3 comprising amino acid residues given by SEQ ID NO: 239, SEQ ID NO: 240, SEQ ID NO: 241, SEQ ID NO: 242, SEQ ID NO: 243, and SEQ ID NO: 244, respectively. In an embodiment, the scFab comprises a VH and VL given by SEQ ID NO: 245 and SEQ ID NO: 246, respectively. In a particular embodiment, the scFab comprises amino acid residues given by SEQ ID NO: 247. In an embodiment, the TCE molecule comprises G4S linkers. In an embodiment, the TCE molecule comprises G4Q linkers. In an embodiment, the CD3-binding scFv is I2E. In another embodiment, the CD3-binding scFv is I2C. In another embodiment, the TCE molecule comprises the amino sequence given by SEQ ID NO: 248. In a further embodiment, the TCE molecule comprises the amino acid sequence given by SEQ ID NO: 249. In some such embodiments, the TCE molecule is TCE 1.3. In a preferred embodiment, the TCE molecule is a single chain. In some embodiments, the TCE molecule may have an orientation such that the VL is N-terminal to the VH.

The present invention provides a TCE molecule comprising an orientation, from N-terminus to C-terminus, of scFab that binds CCR8 (VH, CH1, linker, VL, either Cκ or Cλ), linker, scFv that binds CD3 (VH, linker, VL), linker, Fc1 (hinge, CH2, CH3), linker, Fc2 (hinge, CH2, CH3), wherein the scFab that binds CCR8 comprises CDR1, HCDR2, HCDR3, LCDR1, LCDR2, and LCDR3 comprising amino acid residues given by SEQ ID NO: 250, SEQ ID NO: 251, SEQ ID NO: 252, SEQ ID NO: 253, SEQ ID NO: 254, and SEQ ID NO: 255, respectively. In an embodiment, the scFab comprises a VH and VL given by SEQ ID NO: 256 and SEQ ID NO: 257, respectively. In a particular embodiment, the scFab comprises amino acid residues given by SEQ ID NO: 258. In an embodiment, the TCE molecule comprises G4S linkers. In an embodiment, the TCE molecule comprises G4Q linkers. In an embodiment, the CD3-binding scFv is I2E. In another embodiment, the CD3-binding scFv is I2C. In another embodiment, the TCE molecule comprises the amino sequence given by SEQ ID NO: 259. In a further embodiment, the TCE molecule comprises the amino acid sequence given by SEQ ID NO: 260. In some such embodiments, the TCE molecule is TCE 1.4. In a preferred embodiment, the TCE molecule is a single chain. In some embodiments, the TCE molecule may have an orientation such that the VL is N-terminal to the VH.

The present invention further provides a TCE molecule that binds to human CCR8, which comprises an HCDR1 amino acid sequence of SEQ ID NO: 787; an HCDR2 amino acid sequence of SEQ ID NO: 788; an HCDR3 amino acid sequence of SEQ ID NO: 789; an LCDR1 amino acid sequence of SEQ ID NO: 790; an LCDR2 amino acid sequence of SEQ ID NO: 791; and an LCDR3 amino acid sequence of SEQ ID NO: 792. In some embodiments, the TCE molecule comprises a VH amino acid sequence of SEQ ID NO: 965 and a VL amino acid sequence of SEQ ID NO: 966.

The present invention further provides a TCE molecule that binds to human CCR8, which comprises an HCDR1 amino acid sequence of SEQ ID NO: 787; an HCDR2 amino acid sequence of SEQ ID NO: 788; an HCDR3 amino acid sequence of SEQ ID NO: 789; an LCDR1 amino acid sequence of SEQ ID NO: 336, wherein X1 is K or R; an LCDR2 amino acid sequence of SEQ ID NO: 791; and an LCDR3 amino acid sequence of SEQ ID NO: 792. In some embodiments, the TCE molecule comprises a VH amino acid sequence of SEQ ID NO: 965 and a VL amino acid sequence of SEQ ID NO: 342, wherein X1 is K or R, X2 is H or Q, and/or X3 is S or P.

The present invention further provides a TCE molecule that binds to human CCR8, which comprises an HCDR1 amino acid sequence of SEQ ID NO: 805, an HCDR2 amino acid sequence of SEQ ID NO: 806, an HCDR3 amino acid sequence of SEQ ID NO: 807, an LCDR1 amino acid sequence of SEQ ID NO: 808, an LCDR2 amino acid sequence of SEQ ID NO: 809, and an LCDR3 amino acid sequence of SEQ ID NO: 810.

The present invention further provides a TCE molecule that binds to human CCR8, which comprises: (a) an HCDR1 amino acid sequence of X₁X₂GX₄H, (SEQ ID NO: 1181), wherein (i) X₁ is N, S, D, G, T, or R, (ii) X₂ is C, N, Y, S, or F, and (iii) X₄ is M or F; (b) an HCDR2 amino acid sequence of SEQ ID NOs: 596, 602, 608, 614, 620, 626, 632, 638, 644, 650, 656, 662, 668, 674, 680, 686, 692, 698, 704, 710, 716, 722, 728, 734, 740, 746, 752, 758, 764, 770, 776, 782, 788, 794, 800, 806, 815, 821, 827, 833, 839, 845, 851, 857, 863, 869, 875, 881, 887, or 893, or a variant thereof that comprises 1-4 amino acid substitutions or is at least 90% identical to any one of the foregoing HCDR2 amino acid sequences; (c) an HCDR3 amino acid sequence of SEQ ID NOs: 597, 603, 609, 615, 621, 627, 633, 639, 645, 651, 657, 663, 669, 675, 681, 687, 693, 699, 705, 711, 717, 723, 729, 735, 741, 747, 753, 759, 765, 771, 777, 783, 795, 801, 807, 816, 822, 828, 834, 840, 846, 852, 858, 864, 870, 876, 882, 888, or 894 or a variant thereof that comprises 1-4 amino acid substitutions or is at least 90% identical to any one of the foregoing HCDR3 amino acid sequences; (d) an LCDR1 amino acid sequence of SEQ ID NOs: 598, 604, 610, 616, 622, 628, 634, 640, 646, 652, 658, 664, 670, 676, 682, 688, 694, 700, 706, 712, 718, 724, 730, 736, 742, 748, 754, 760, 766, 772, 778, 784, 796, 802, 808, 811, 817, 823, 829, 835, 841, 847, 853, 859, 865, 871, 877, 883, or 889 or a variant thereof that comprises 1-4 amino acid substitutions or is at least 90% identical to any one of the foregoing LCDR1 amino acid sequences; (e) an LCDR2 amino acid sequence of RX₂X₃X₄RPS (SEQ ID NO: 1182), wherein (i) X₂ is A, N, D, S, or Q, (ii) X₃ is S, T, N, I, F, or A, and (iii) X₄ is N or V; and (f) an LCDR3 amino acid sequence of SEQ ID NOs: 600, 606, 612, 618, 624, 630, 636, 642, 648, 654, 660, 666, 672, 678, 684, 690, 696, 702, 708, 714, 720, 726, 732, 738, 744, 750, 756, 762, 768, 774, 780, 786, 798, 804, 810, 813, 819, 825, 831, 837, 843, 849, 855, 861, 867, 873, 879, 885, or 891 or a variant thereof that comprises 1-4 amino acid substitutions or is at least 90% identical to any one of the foregoing LCDR3 amino acid sequences. In some embodiments, the HCDR1 comprises an amino acid sequence of SEQ ID NOs: 595, 601, 607, 613, 619, 625, 631, 637, 643, 649, 655, 661, 667, 673, 679, 685, 691, 697, 703, 709, 715, 721, 727, 733, 739, 745, 751, 757, 763, 769, 775, 781, 793, 799, 805, 814, 820, 826, 832, 838, 844, 850, 856, 862, 868, 874, 880, 886, or 892. In some embodiments, the LCDR2 comprises an amino acid sequence of SEQ ID NOs: 599, 605, 611, 617, 623, 629, 635, 641, 647, 653, 659, 665, 671, 677, 683, 689, 695, 701, 707, 713, 719, 725, 731, 737, 743, 749, 755, 761, 767, 773, 779, 785, 797, 803, 809, 812, 818, 824, 830, 836, 842, 848, 854, 860, 866, 872, 878, 884, or 890. In some embodiments, the VH comprises an amino acid sequence of SEQ ID NOs: 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 967, 969, 971, 974, 976, 978, 980, 982, 984, 986, 988, 990, 992, 994, 996, 998, or 1000. In some embodiments, the VL comprises an amino acid sequence of SEQ ID NOs: 912, 914, 916, 918, 920, 922, 924, 926, 928, 930, 932, 934, 936, 938, 940, 942, 944, 946, 948, 950, 952, 954, 956, 958, 960, 962, 964, 968, 970, 972, 973, 975, 977, 979, 981, 983, 985, 987, 989, 991, 993, 995, 997, or 999.

In some embodiments, the TCE molecule comprises: (a) a VH comprising an amino acid sequence of SEQ ID NO: 967 and a VL comprising an amino acid sequence of SEQ ID NO: 968; (b) a VH comprising an amino acid sequence of SEQ ID NO: 969 and a VL comprising an amino acid sequence of SEQ ID NO: 970; (c) a VH comprising an amino acid sequence of SEQ ID NO: 971 and a VL comprising an amino acid sequence of SEQ ID NO: 972; (d) a VH comprising an amino acid sequence of SEQ ID NO: 974 and a VL comprising an amino acid sequence of SEQ ID NO: 973; (e) a VH comprising an amino acid sequence of SEQ ID NO: 976 and a VL comprising an amino acid sequence of SEQ ID NO: 975; (f) a VH comprising an amino acid sequence of SEQ ID NO: 978 and a VL comprising an amino acid sequence of SEQ ID NO: 977; (g) a VH comprising an amino acid sequence of SEQ ID NO: 980 and a VL comprising an amino acid sequence of SEQ ID NO: 979; (h) a VH comprising an amino acid sequence of SEQ ID NO: 982 and a VL comprising an amino acid sequence of SEQ ID NO: 981; (i) a VH comprising an amino acid sequence of SEQ ID NO: 984 and a VL comprising an amino acid sequence of SEQ ID NO: 983; (j) a VH comprising an amino acid sequence of SEQ ID NO: 986 and a VL comprising an amino acid sequence of SEQ ID NO: 985; (k) a VH comprising an amino acid sequence of SEQ ID NO: 988 and a VL comprising an amino acid sequence of SEQ ID NO: 987; (1) a VH comprising an amino acid sequence of SEQ ID NO: 990 and a VL comprising an amino acid sequence of SEQ ID NO: 989; (m) a VH comprising an amino acid sequence of SEQ ID NO: 992 and a VL comprising an amino acid sequence of SEQ ID NO: 991; (n) a VH comprising an amino acid sequence of SEQ ID NO: 994 and a VL comprising an amino acid sequence of SEQ ID NO: 993; (0) a VH comprising an amino acid sequence of SEQ ID NO: 996 and a VL comprising an amino acid sequence of SEQ ID NO: 995; (p) a VH comprising an amino acid sequence of SEQ ID NO: 998 and a VL comprising an amino acid sequence of SEQ ID NO: 997; or (q) a VH comprising an amino acid sequence of SEQ ID NO: 1000 and a VL comprising an amino acid sequence of SEQ ID NO: 999.

In some embodiments, the TCE molecule comprises a heavy chain variable region (VH) amino acid sequence of SEQ ID NO: 965, and a light chain variable region (VL) comprising the amino acid sequence: DIVMTQSPDSLAVSLGERATINCKSSQSVLYSSNNXINYLAWYX2QKPGQX3PKLLISWA STRESGVPDRFSGSGSGTDFTLTINSLQAEDVAVYYCQQYYSIPITFGGGTKVEIKR (SEQ ID NO: 342), wherein X₁ is K or R, X₂ is H or Q, and/or X₃ is S or P. In some embodiments, a TCE molecule of the present invention comprises a YTE motif in the Fc region, corresponding to M252Y/S254T/T256E in the constant heavy chain region of IgG1 or IgG4. The YTE extends the half-life of the molecule (see e.g. Booth et al., MAbs 2018 October; 10 (7): 1098-1110). In some embodiments, the TCE molecule of the present invention comprising YTE is a TCE molecule that binds CCR8 and CD3.

In some embodiments, a TCE molecule of the present invention comprises an I2E scFv. In some embodiments, a TCE molecule of the present invention comprises an I2C scFv. The amino acid sequences of I2E are given by SEQ ID NOs. 199 to 206. The amino acid sequences of I2C are given by SEQ ID NOs. 191 to 198.

The present invention provides additional TCE molecules described in Table 25. The amino acid sequences of these TCE molecules are given by SEQ ID NOs 261 to 589 in Table 25.

The present invention also provides a method of treating cancer in a patient comprising administering an effective amount of a TCE molecule of the present invention to the patient. In an embodiment, the cancer is a solid tumor. In a particular embodiment, the cancer is non-small cell lung cancer, gastric cancer, head and neck squamous cell carcinoma, hepatocellular carcinoma, triple-negative breast cancer, colorectal cancer, pancreatic cancer, or metastatic castrate-resistant prostate cancer. In some embodiments, the method further comprises administering to the patient a PD-1 antagonist antibody or a PD-L1 antagonist antibody. In some such embodiments, the PD-1 antagonist antibody or PD-L1 antagonist antibody is administered prior to, concurrently with, and/or after administration of the TCE molecule. In particular embodiments, the PD-1 antagonist antibody is pembrolizumab, nivolumab, cemiplimab, or antibody 20C1.009. In other particular embodiments, the PD-L1 antagonist antibody is atezolizumab, avelumab, or durvalumab. In some embodiments, the method further comprises administering to the patient a chemotherapeutic agent. In some such embodiments, the chemotherapeutic agent may be administered prior to, concurrently with, or after administration of the TCE molecule. In some embodiments, the method comprises administering to the patient a TCE molecule of the present invention and a chemotherapeutic agent. In some embodiments, the method comprises administering to the patient a TCE molecule of the present invention, a PD-1 or PD-L1 antagonist antibody, and a chemotherapeutic agent.

The present invention provides a TCE molecule of the present invention for use in therapy.

The present invention also provides a TCE molecule for use in treating cancer. In an embodiment, the cancer is a solid tumor. In a particular embodiment, the cancer is non-small cell lung cancer, gastric cancer, head and neck squamous cell carcinoma, hepatocellular carcinoma, triple-negative breast cancer, colorectal cancer, pancreatic cancer, or metastatic castrate-resistant prostate cancer. In a more particular embodiment, the cancer is non-small cell lung cancer, gastric cancer, head and neck squamous cell carcinoma, hepatocellular carcinoma, or triple-negative breast cancer. In some embodiments, the use further comprises administering to the patient a PD-1 antagonist antibody or PD-L1 antagonist antibody. In some such embodiments, the PD-1 antagonist antibody or PD-L1 antagonist antibody is administered prior to, concurrently with, and/or after administration of the TCE molecule. In particular embodiments, the PD-1 antagonist antibody is pembrolizumab, nivolumab, cemiplimab, or antibody 20C1.009. In other particular embodiments, the PD-L1 antagonist antibody is atezolizumab, avelumab, or durvalumab. In some embodiments, the use further comprises administering to the patient a chemotherapeutic agent. In some such embodiments, the chemotherapeutic agent may be administered prior to, concurrently with, or after administration of the TCE molecule. In some embodiments, the use comprises administering to the patient a TCE molecule of the present invention and a chemotherapeutic agent. In some embodiments, the use comprises administering to the patient a TCE molecule of the present invention, a PD-1 or PD-L1 antagonist antibody, and a chemotherapeutic agent.

The present invention provides the use of a TCE molecule of the present invention for the manufacture of a medicament for the treatment of cancer. In an embodiment, the cancer is a solid tumor. In a particular embodiment, the cancer is non-small cell lung cancer, gastric cancer, head and neck squamous cell carcinoma, hepatocellular carcinoma, triple-negative breast cancer, colorectal cancer, pancreatic cancer, or metastatic castrate-resistant prostate cancer. In a more particular embodiment, the cancer is non-small cell lung cancer, gastric cancer, head and neck squamous cell carcinoma, hepatocellular carcinoma, or triple-negative breast cancer.

The present invention also provides a pharmaceutical composition comprising a TCE molecule of the present invention and one or more pharmaceutically acceptable carriers, diluents, or excipients.

The present invention also provides a polynucleotide that encodes an amino acid sequence of a TCE molecule of the present invention. The term “encoding” or “encodes” refers to a polynucleotide sequence encoding one or more amino acids. The term does not require a start or stop codon. The present invention encompasses nucleic acid molecules encoding anti-CCR8 TCE polypeptide sequences.

In an embodiment, the TCE molecule of the present invention is encoded by a polynucleotide sequence given by SEQ ID NO: 590. In a particular embodiment, the TCE molecule encoded by the polynucleotide sequence given by SEQ ID NO: 590 comprises the amino acid sequence given by SEQ ID NO: 227.

In an embodiment, the TCE molecule of the present invention is encoded by a polynucleotide sequence given by SEQ ID NO: 592. In a particular embodiment, the TCE molecule encoded by the polynucleotide sequence given by SEQ ID NO: 592 comprises the amino acid sequence given by SEQ ID NO: 249.

In an embodiment, the TCE molecule of the present invention is encoded by a polynucleotide sequence given by SEQ ID NO: 593. In a particular embodiment, the TCE molecule encoded by the polynucleotide sequence given by SEQ ID NO: 593 comprises the amino acid sequence given by SEQ ID NO: 260.

In an embodiment, the TCE molecule of the present invention is encoded by a polynucleotide sequence given by SEQ ID NO: 591. In a particular embodiment, the TCE molecule encoded by the polynucleotide sequence given by SEQ ID NO: 591 comprises the amino acid sequence given by SEQ ID NO: 238.

The present invention also provides a DNA molecule comprising a polynucleotide that encodes an amino acid sequence of a TCE molecule of the present invention. In an embodiment, the TCE molecule of the present invention is encoded by a polynucleotide sequence given by SEQ ID NO: 590. In a particular embodiment, the TCE molecule encoded by the polynucleotide sequence given by SEQ ID NO: 590 comprises the amino acid sequence given by SEQ ID NO: 227. In another embodiment, the TCE molecule of the present invention is encoded by a polynucleotide sequence given by SEQ ID NO: 592. In a particular embodiment, the TCE molecule encoded by the polynucleotide sequence given by SEQ ID NO: 592 comprises the amino acid sequence given by SEQ ID NO: 249. In another embodiment, the TCE molecule of the present invention is encoded by a polynucleotide sequence given by SEQ ID NO: 593. In a particular embodiment, the TCE molecule encoded by the polynucleotide sequence given by SEQ ID NO: 593 comprises the amino acid sequence given by SEQ ID NO: 260. In another embodiment, the TCE molecule of the present invention is encoded by a polynucleotide sequence given by SEQ ID NO: 591. In a particular embodiment, the TCE molecule encoded by the polynucleotide sequence given by SEQ ID NO: 591 comprises the amino acid sequence given by SEQ ID NO: 238.

The present invention further provides a mammalian cell transformed with a DNA molecule of the present invention, wherein the transformed mammalian cell is capable of expressing a TCE molecule of the present invention.

The present invention also provides a process for producing a TCE molecule of the present invention, wherein the process comprises cultivating a mammalian cell under conditions such that the TCE molecule is expressed and recovering the expressed TCE molecule. The present invention also provides a mammalian cell transformed with a DNA molecule of the present invention, wherein the transformed mammalian cell is capable of expressing a TCE molecule of the present invention. The present invention also provides a TCE molecule obtainable by the process.

In another embodiment, the present invention provides a CCR8 TCE molecule that binds human CCR8 at an epitope wherein the epitope comprises at least one residue of SEQ ID NO: 134. In an embodiment, the epitope comprises at least two residues of SEQ ID NO: 134. In an embodiment, the epitope comprises at least three residues of SEQ ID NO: 134. In an embodiment, the epitope comprises at least four residues of SEQ ID NO: 134. In an embodiment, the epitope comprises at least five residues of SEQ ID NO: 134. In an embodiment, the epitope comprises six or more residues of SEQ ID NO: 134. In an embodiment, the epitope comprises seven or more residues of SEQ ID NO: 134. In an embodiment, the epitope comprises eight or more residues of SEQ ID NO: 134. In an embodiment, the epitope comprises nine or more residues of SEQ ID NO: 134. In an embodiment, the epitope comprises ten or more residues of SEQ ID NO: 134. In an embodiment, the epitope comprises eleven or more residues of SEQ ID NO: 134. In an embodiment, the epitope comprises twelve residues of SEQ ID NO: 134. In a particular embodiment, the epitope comprises the threonine residue at position 4 of SEQ ID NO: 134. The term “epitope” as used herein refers to sites of an antigen that are in contact with (e.g. binds) the molecule. The epitope may be determined by a method known to a person of ordinary skill, including flow cytometry of bound TCE molecule to peptides, hydrogen-deuterium exchange, alanine scanning, and/or x-ray crystallography. In an embodiment, the epitope is determined by epitope binning. In an embodiment, the epitope is determined by TCE molecule binding to CCR8 peptide-nanobody complexes. In an embodiment, the epitope is determined by screening TCE molecule binding to CCR8 by phage display. In an embodiment, the epitope is determined by determining binding to a CCR8 peptide expressed in human cells, wherein the peptide comprises an amino acid sequence given by SEQ ID NO: 134 or amino acid residues 1-12 of SEQ ID NO: 133. In some embodiments, the epitope is determined by anti-CCR8 TCE molecule binding to the T4R mutation in cynomolgus monkey CCR8. In an embodiment, binding to the T4R mutation is determined in a cell based affinity assay, wherein TCE molecule binding to cells expressing cynomolgus monkey cells CCR8 containing a T4R mutation is compared to TCE molecule binding to cells expressing wild-type cynomolgus monkey CCR8 (comprising a threonine at position four). In some embodiments, an anti-CCR8 TCE molecule binds threonine at position four if it shows reduced binding to CCR8 comprising a T4R mutation. In particular embodiments, an anti-CCR8 TCE molecule binds threonine at position four if it shows no detectable binding to CCR8 comprising a T4R mutation. In some embodiments, wild-type cynomolgus monkey CCR8 comprises an amino acid sequence given by SEQ ID NO: 129. In some embodiments, cynomolgus monkey CCR8 comprising a T4R mutation comprises an amino acid sequence given by SEQ ID NO: 130.

The present invention provides a method of treating cancer in a patient comprising administering to the patient an effective amount of a CCR8 TCE molecule that binds human CCR8 at an epitope wherein the epitope comprises at least one residue of SEQ ID NO: 134. In an embodiment, the epitope comprises at least two residues of SEQ ID NO: 134. In an embodiment, the epitope comprises at least three residues of SEQ ID NO: 134. In an embodiment, the epitope comprises at least four residues of SEQ ID NO: 134. In an embodiment, the epitope comprises at least five residues of SEQ ID NO: 134. In an embodiment, the epitope comprises six or more residues of SEQ ID NO: 134. In an embodiment, the epitope comprises seven or more residues of SEQ ID NO: 134. In an embodiment, the epitope comprises eight or more residues of SEQ ID NO: 134. In an embodiment, the epitope comprises nine or more residues of SEQ ID NO: 134. In an embodiment, the epitope comprises ten or more residues of SEQ ID NO: 134. In an embodiment, the epitope comprises eleven or more residues of SEQ ID NO: 134. In an embodiment, the epitope comprises twelve residues of SEQ ID NO: 134. In an embodiment, the epitope comprises a threonine residue at position 4 of SEQ ID NO: 134. In an embodiment, the epitope is determined by epitope binning. In an embodiment, the epitope is determined by TCE molecule binding to CCR8 peptide-nanobody complexes. In an embodiment, the epitope is determined by screening TCE molecule binding to CCR8 by phage display. In an embodiment, the epitope is determined by determining binding to a CCR8 peptide expressed in human cells, wherein the peptide comprises an amino acid sequence given by SEQ ID NO: 134 or amino acid residues 1-12 of SEQ ID NO: 133. In some embodiments, the epitope is determined by anti-CCR8 TCE molecule binding to the T4R mutation in cynomolgus monkey CCR8. In an embodiment, binding to the T4R mutation is determined in a cell based affinity assay, wherein TCE molecule binding to cells expressing cynomolgus monkey cells CCR8 containing a T4R mutation is compared to TCE molecule binding to cells expressing wild-type cynomolgus monkey CCR8 (comprising a threonine at position four). In some embodiments, an anti-CCR8 TCE molecule binds threonine at position four if it shows reduced binding to CCR8 comprising a T4R mutation. In particular embodiments, an anti-CCR8 TCE molecule binds threonine at position four if it shows no detectable binding to CCR8 comprising a T4R mutation. In some embodiments, wild-type cynomolgus monkey CCR8 comprises an amino acid sequence given by SEQ ID NO: 129. In some embodiments, cynomolgus monkey CCR8 comprising a T4R mutation comprises an amino acid sequence given by SEQ ID NO: 130.

In another embodiment, the present invention provides a CCR8 TCE molecule that binds human CCR8 at an epitope wherein the epitope consists of one residue of SEQ ID NO: 134. In an embodiment, the epitope consists of two residues of SEQ ID NO: 134. In an embodiment, the epitope consists of three residues of SEQ ID NO: 134. In an embodiment, the epitope consists of four residues of SEQ ID NO: 134. In an embodiment, the epitope consists of five residues of SEQ ID NO: 134. In an embodiment, the epitope consists of six residues of SEQ ID NO: 134. In an embodiment, the epitope consists of seven residues of SEQ ID NO: 134. In an embodiment, the epitope consists of eight residues of SEQ ID NO: 134. In an embodiment, the epitope consists of nine residues of SEQ ID NO: 134. In an embodiment, the epitope consists of ten residues of SEQ ID NO: 134. In an embodiment, the epitope consists of eleven residues of SEQ ID NO: 134. In an embodiment, the epitope consists of twelve residues of SEQ ID NO: 134. In an embodiment, the epitope consists of a threonine residue at position 4 of SEQ ID NO: 134.

The present invention provides a method of treating cancer in a patient comprising administering to the patient an effective amount of a CCR8 TCE molecule that binds human CCR8 at an epitope wherein the epitope consists of one residue of SEQ ID NO: 134. In an embodiment, the epitope consists of two residues of SEQ ID NO: 134. In an embodiment, the epitope consists of three residues of SEQ ID NO: 134. In an embodiment, the epitope consists of four residues of SEQ ID NO: 134. In an embodiment, the epitope consists of five residues of SEQ ID NO: 134. In an embodiment, the epitope consists of six residues of SEQ ID NO: 134. In an embodiment, the epitope consists of seven residues of SEQ ID NO: 134. In an embodiment, the epitope consists of eight residues of SEQ ID NO: 134. In an embodiment, the epitope consists of nine residues of SEQ ID NO: 134. In an embodiment, the epitope consists of ten residues of SEQ ID NO: 134. In an embodiment, the epitope consists of eleven residues of SEQ ID NO: 134. In an embodiment, the epitope consists of twelve residues of SEQ ID NO: 134. In an embodiment, the epitope consists of a threonine residue at position 4 of SEQ ID NO: 134.

In another embodiment, the present invention provides a CCR8 TCE molecule that binds human CCR8 at an epitope wherein the epitope comprises at least one residue of amino acid residues 1-12 OF SEQ ID NO: 133. In an embodiment, the epitope comprises at least two residues of amino acid residues 1-12 OF SEQ ID NO: 133. In an embodiment, the epitope comprises at least three residues of amino acid residues 1-12 OF SEQ ID NO: 133. In an embodiment, the epitope comprises at least four residues of amino acid residues 1-12 OF SEQ ID NO: 133. In an embodiment, the epitope comprises at least five residues of amino acid residues 1-12 OF SEQ ID NO: 133. In an embodiment, the epitope comprises six or more residues of amino acid residues 1-12 OF SEQ ID NO: 133. In an embodiment, the epitope comprises seven or more residues of amino acid residues 1-12 OF SEQ ID NO: 133. In an embodiment, the epitope comprises eight or more residues of amino acid residues 1-12 OF SEQ ID NO: 133. In an embodiment, the epitope comprises nine or more residues of amino acid residues 1-12 of SEQ ID NO: 133. In an embodiment, the epitope comprises ten or more residues amino acid residues 1-12 of SEQ ID NO: 133. In an embodiment, the epitope comprises eleven or more residues of amino acid residues 1-12 OF SEQ ID NO: 133. In an embodiment, the epitope comprises twelve residues of amino acid residues 1-12 OF SEQ ID NO: 133. In a particular embodiment, the epitope comprises the threonine residue at position 4 of amino acid residues 1-12 OF SEQ ID NO: 133. The term “epitope” as used herein refers to sites of an antigen that are in contact with (e.g. binds) the molecule. The epitope may be determined by a method known to a person of ordinary skill, including flow cytometry of bound TCE molecule to peptides, hydrogen-deuterium exchange, alanine scanning, and/or x-ray crystallography. In an embodiment, the epitope is determined by epitope binning. In an embodiment, the epitope is determined by TCE molecule binding to CCR8 peptide-nanobody complexes. In an embodiment, the epitope is determined by screening TCE molecule binding to CCR8 by phage display. In an embodiment, the epitope is determined by determining binding to a CCR8 peptide expressed in human cells, wherein the peptide comprises an amino acid sequence given by SEQ ID NO: 134 or amino acid residues 1-12 of SEQ ID NO: 133. In some embodiments, the epitope is determined by anti-CCR8 TCE molecule binding to the T4R mutation in cynomolgus monkey CCR8. In an embodiment, binding to the T4R mutation is determined in a cell based affinity assay, wherein TCE molecule binding to cells expressing cynomolgus monkey cells CCR8 containing a T4R mutation is compared to TCE molecule binding to cells expressing wild-type cynomolgus monkey CCR8 (comprising a threonine at position four). In some embodiments, an anti-CCR8 TCE molecule binds threonine at position four if it shows reduced binding to CCR8 comprising a T4R mutation. In particular embodiments, an anti-CCR8 TCE molecule binds threonine at position four if it shows no detectable binding to CCR8 comprising a T4R mutation. In some embodiments, wild-type cynomolgus monkey CCR8 comprises an amino acid sequence given by SEQ ID NO: 129. In some embodiments, cynomolgus monkey CCR8 comprising a T4R mutation comprises an amino acid sequence given by SEQ ID NO: 130.

The present invention provides a method of treating cancer in a patient comprising administering to the patient an effective amount of a CCR8 TCE molecule that binds human CCR8 at an epitope wherein the epitope comprises at least one residue of amino acid residues 1-12 OF SEQ ID NO: 133. In an embodiment, the epitope comprises at least two residues of amino acid residues 1-12 OF SEQ ID NO: 133. In an embodiment, the epitope comprises at least three residues of amino acid residues 1-12 OF SEQ ID NO: 133. In an embodiment, the epitope comprises at least four residues of amino acid residues 1-12 OF SEQ ID NO: 133. In an embodiment, the epitope comprises at least five residues of amino acid residues 1-12 OF SEQ ID NO: 133. In an embodiment, the epitope comprises six or more residues of amino acid residues 1-12 OF SEQ ID NO: 133. In an embodiment, the epitope comprises seven or more residues of amino acid residues 1-12 OF SEQ ID NO: 133. In an embodiment, the epitope comprises eight or more residues of amino acid residues 1-12 OF SEQ ID NO: 133. In an embodiment, the epitope comprises nine or more residues of amino acid residues 1-12 of SEQ ID NO: 133. In an embodiment, the epitope comprises ten or more residues amino acid residues 1-12 of SEQ ID NO: 133. In an embodiment, the epitope comprises eleven or more residues of amino acid residues 1-12 OF SEQ ID NO: 133. In an embodiment, the epitope comprises twelve residues of amino acid residues 1-12 OF SEQ ID NO: 133. In an embodiment, the epitope comprises a threonine residue at position 4 of amino acid residues 1-12 OF SEQ ID NO: 133. In an embodiment, the epitope is determined by epitope binning. In an embodiment, the epitope is determined by TCE molecule binding to CCR8 peptide-nanobody complexes. In an embodiment, the epitope is determined by screening TCE molecule binding to CCR8 by phage display. In an embodiment, the epitope is determined by determining binding to a CCR8 peptide expressed in human cells, wherein the peptide comprises an amino acid sequence given by SEQ ID NO: 134 or amino acid residues 1-12 of SEQ ID NO: 133. In some embodiments, the epitope is determined by anti-CCR8 TCE molecule binding to the T4R mutation in cynomolgus monkey CCR8. In an embodiment, binding to the T4R mutation is determined in a cell based affinity assay, wherein TCE molecule binding to cells expressing cynomolgus monkey cells CCR8 containing a T4R mutation is compared to TCE molecule binding to cells expressing wild-type cynomolgus monkey CCR8 (comprising a threonine at position four). In some embodiments, an anti-CCR8 TCE molecule binds threonine at position four if it shows reduced binding to CCR8 comprising a T4R mutation. In particular embodiments, an anti-CCR8 TCE molecule binds threonine at position four if it shows no detectable binding to CCR8 comprising a T4R mutation. In some embodiments, wild-type cynomolgus monkey CCR8 comprises an amino acid sequence given by SEQ ID NO: 129. In some embodiments, cynomolgus monkey CCR8 comprising a T4R mutation comprises an amino acid sequence given by SEQ ID NO: 130.

In another embodiment, the present invention provides a CCR8 TCE molecule that binds human CCR8 at an epitope wherein the epitope consists of at least one residue of amino acid residues 1-12 OF SEQ ID NO: 133. In an embodiment, the epitope consists of two residues of amino acid residues 1-12 OF SEQ ID NO: 133. In an embodiment, the epitope consists of three residues of amino acid residues 1-12 OF SEQ ID NO: 133. In an embodiment, the epitope consists of four residues of amino acid residues 1-12 OF SEQ ID NO: 133. In an embodiment, the epitope consists of five residues of amino acid residues 1-12 OF SEQ ID NO: 133. In an embodiment, the epitope consists of six residues of amino acid residues 1-12 OF SEQ ID NO: 133. In an embodiment, the epitope consists of seven residues of amino acid residues 1-12 OF SEQ ID NO: 133. In an embodiment, the epitope consists of eight residues of amino acid residues 1-12 OF SEQ ID NO: 133. In an embodiment, the epitope consists of nine residues of amino acid residues 1-12 OF SEQ ID NO: 133. In an embodiment, the epitope consists of ten residues of amino acid residues 1-12 OF SEQ ID NO: 133. In an embodiment, the epitope consists of eleven residues of amino acid residues 1-12 OF SEQ ID NO: 133. In an embodiment, the epitope consists of twelve residues of amino acid residues 1-12 OF SEQ ID NO: 133. In an embodiment, the epitope consists of a threonine residue at position 4 of amino acid residues 1-12 OF SEQ ID NO: 133.

The present invention provides a method of treating cancer in a patient comprising administering to the patient an effective amount of a CCR8 TCE molecule that binds human CCR8 at an epitope wherein the epitope consists of one residue of amino acid residues 1-12 OF SEQ ID NO: 133. In an embodiment, the epitope consists of two residues of amino acid residues 1-12 OF SEQ ID NO: 133. In an embodiment, the epitope consists of three residues of amino acid residues 1-12 OF SEQ ID NO: 133. In an embodiment, the epitope consists of four residues of amino acid residues 1-12 OF SEQ ID NO: 133. In an embodiment, the epitope consists of five residues of amino acid residues 1-12 OF SEQ ID NO: 133. In an embodiment, the epitope consists of six residues of amino acid residues 1-12 OF SEQ ID NO: 133. In an embodiment, the epitope consists of seven residues of amino acid residues 1-12 OF SEQ ID NO: 133. In an embodiment, the epitope consists of eight residues of amino acid residues 1-12 OF SEQ ID NO: 133. In an embodiment, the epitope consists of nine residues of amino acid residues 1-12 OF SEQ ID NO: 133. In an embodiment, the epitope consists of ten residues of amino acid residues 1-12 OF SEQ ID NO: 133. In an embodiment, the epitope consists of eleven residues of amino acid residues 1-12 OF SEQ ID NO: 133. In an embodiment, the epitope consists of twelve residues of amino acid residues 1-12 OF SEQ ID NO: 133. In an embodiment, the epitope consists of a threonine residue at position 4 of amino acid residues 1-12 OF SEQ ID NO: 133.

In an embodiment, the epitope is determined by epitope binning. In an embodiment, the epitope is determined by TCE molecule binding to CCR8 peptide-nanobody complexes. In an embodiment, the epitope is determined by screening TCE molecule binding to CCR8 by phage display. In an embodiment, the epitope is determined by determining binding to a CCR8 peptide expressed in human cells, wherein the peptide comprises an amino acid sequence given by SEQ ID NO: 134 or amino acid residues 1-12 of SEQ ID NO: 133. In some embodiments, the epitope is determined by anti-CCR8 TCE molecule binding to the T4R mutation in cynomolgus monkey CCR8. In an embodiment, binding to the T4R mutation is determined in a cell based affinity assay, wherein TCE molecule binding to cells expressing cynomolgus monkey cells CCR8 containing a T4R mutation is compared to TCE molecule binding to cells expressing wild-type cynomolgus monkey CCR8 (comprising a threonine at position four). In some embodiments, an anti-CCR8 TCE molecule binds threonine at position four if it shows reduced binding to CCR8 comprising a T4R mutation. In particular embodiments, an anti-CCR8 TCE molecule binds threonine at position four if it shows no detectable binding to CCR8 comprising a T4R mutation. In some embodiments, wild-type cynomolgus monkey CCR8 comprises an amino acid sequence given by SEQ ID NO: 129. In some embodiments, cynomolgus monkey CCR8 comprising a T4R mutation comprises an amino acid sequence given by SEQ ID NO: 130.

In some embodiments, the present invention provides a molecule that competes for binding CCR8 with a CCR8 TCE molecule of the present invention. Such molecule that competes for binding may be, for example, a TCE molecule, an antibody, antibody fragment, or polypeptide. In some embodiments, the present invention provides a molecule that binds the same epitope as a CCR8 TCE molecule of the present invention.

In some embodiments, a TCE molecule of the present invention can be administered concurrently with, before, or after a variety of drugs and treatments widely employed in cancer treatment such as, for example, chemotherapeutic agents, non-chemotherapeutic agents (e.g. anti-PD-1 or anti-PD-L1 inhibitors, such as antagonist antibodies), anti-neoplastic agents, and/or radiation. For example, administration can occur before, during, and/or after any of the treatments described herein. Examples of chemotherapeutic agents are discussed herein and include, but are not limited to, cisplatin, taxol, etoposide, mitoxantrone (Novantrone®), actinomycin D, cycloheximide, camptothecin (or water soluble derivatives thereof), methotrexate, mitomycin (e.g., mitomycin C), dacarbazine (DTIC), anti-neoplastic antibiotics such as adriamycin (doxorubicin) and daunomycin, and all the chemotherapeutic agents mentioned herein.

In some embodiments, a TCE molecule of the present invention may be administered concurrently with, before, or after a PD-1 antagonist antibody or a PD-L1 antagonist antibody. The term “PD-1 antagonist antibody” refers to an antibody that specifically binds to PD-1 and decreases, blocks, inhibits, abrogates or interferes with signal transduction resulting from the interaction of PD-1 and one or more of its ligands, such as PD-L1 and PD-L2. In some embodiments, a PD-1 antagonist antibody inhibits the binding of PD-1 to PD-L1 and/or PD-L2. The term “PD-L1 antagonist antibody” refers to an antibody that specifically binds to PD-L1 and decreases, blocks, inhibits, abrogates or interferes with signal transduction resulting from the interaction of PD-L1 with the PD-1 receptor. In some embodiments, a PD-L1 antagonist antibody inhibits the binding of PD-L1 to PD-1. In some embodiments, the PD-1 antagonist is any one of Antibody 20C1.006 (SEQ ID NOs: 179-188), Antibody 20C1.009 (SEQ ID NOS: 139-148, or 139-147 and 212), Antibody 20A2.3 (SEQ ID NOs: 149-158), Antibody 20D4.6 (SEQ ID NOs: 159-168), or Antibody 20D4.17 (SEQ ID NOs: 169-178). In one embodiment, the PD-1 antagonist antibody is pembrolizumab. In another embodiment, the PD-1 antagonist antibody is nivolumab. In yet another embodiment, the PD-1 antagonist antibody is cemiplimab. In a particular embodiment, the PD-1 antagonist antibody is antibody 20C1.009, for which the amino acid sequences of the CDRs, variable regions, and full light and heavy chains are provided in SEQ ID NOs: 139-148 and 212. 20C1.009 is also known as AMG 404 and is also known as zeluvalimab. In exemplary aspects, an anti-PD-1 antibody such as 20C1.009 comprises a HC comprising a C-terminal lysine, as in SEQ ID NO: 148. In alternative aspects, the antibody comprises a HC without the C-terminal lysine, as in SEQ ID NO: 212.

In some embodiments, the 1185-1200

### BRIEF DESCRIPTION OF THE DRAWINGS

FIG. 1. Depicted are the domains and domain order of TCE molecules of the present invention. Exemplary TCE molecules comprise the following domain order from N-terminus to C-terminus: VH-Linker-VL-Linker-VH-Linker-VL-Linker-Fc1-Linker-Fc2 (left; “CCR8-CD3 TCE”). Other exemplary TCE molecules of the present invention comprise the following domain order from N-terminus to C-terminus: VH-CH1-Linker-VL-Ck/Cλ-Linker-VH-Linker-VL-Linker-Fc1-Linker-Fc2 (right; “scFab TCE”). Abbreviations: scFab=single chain Fab (VH-CH1-Linker-VL-Ck/Cλ); scFv=single chain Fv (VH-Linker-VL); scFc-single chain Fc. Depicted formats may comprise scFab or scFv in either orientation, from N-terminus to C-terminus: VH-VL, VL-VH, VH-CH1—VL-Ck/Cλ, or VL-Ck/Cλ-VH-CHI, including linker. For simplicity, VH-VL and VH-CH1-VL-Ck/Cλ orientations are depicted. Depicted formats may comprise G4S linkers or G4Q linkers. For simplicity, G4S linkers are depicted.

FIG. 2. Depicted are the domains and domain order of multitargeting BiTE HLE formats of the present invention. Multitargeting BiTE HLE molecules of the scFv-scFv-scFv-scFc format comprises the following domain order from N- to C-terminus: VH-Linker-VL-Linker-VH-Linker-VL-Linker-Fc1-Linker-Fc2, whereas the scFab-scFv-scFv-scFc format comprises VH-CH1-Linker-VL-Cκ/Cλ-Linker-VH-Linker-VL-Linker-VH-Linker-VL-Linker-Fc1-Linker-Fc2. The scFab-scFab-scFv-scFc format comprises VH-CH1-Linker-VL-Cκ/Cλ-Linker-VH-CH1-Linker-VL-Cκ/Cλ-Linker-VH-Linker-VL-Linker-Fc1-Linker-Fc2. Depicted BiTE formats can comprise scFab or scFv in either orientation, HL or LH; for simplicity only HL orientations are depicted. Cκ/Cλ-either Cκ or Cλ. Depicted BiTE formats may comprise G4S linkers or G4Q linkers. For simplicity, G4S linkers are depicted.

### DETAILED DESCRIPTION OF THE INVENTION

The present disclosure provides single chain TCE molecules comprising an scFab that binds a target antigen and an scFv that binds CD3. The present disclosure also provides TCE molecules comprising an scFv that binds CCR8 and an scFv that binds CD3. Methods of treating cancer are also provided, as well as methods of making said TCE molecules.

A “single-chain variable fragment” (“scFv”) is a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., Science 242:423-26 (1988) and Huston et al., 1988, Proc. Natl. Acad. Sci. USA 85:5879-83 (1988)). When in the context of other additional moieties (e.g., an Fc region), the scFv can be arranged VH-linker-VL (anti-CD3 scFv), or VL-linker-VH, for example. An anti-target scFv is an scFv that binds an antigen, such as a tumor antigen. An anti-target scFv may bind CCR8. An anti-CD3 scFv binds CD3. Examples of anti-CD3 scFvs include I2E and I2C, given by amino acid sequences 199-206 and 191-198, respectively.

A “single-chain antigen-binding fragment” (“scFab”) is a fusion protein in which a VH and CHI are joined via a linker to a VL and Cκ or Cλ to form a continuous protein chain wherein the linker is long enough to allow the protein chain to fold back on itself and form a monovalent antigen binding site independent of the orientation. The linker may be, for example, a (G4S)6, (G4S)7, or (G4S)8 linker. A G4S linker is a linker made of amino acids GGGGS (SEQ ID NO: 189), from N-terminus to C-terminus, and may be repeated multiple times. A (G4S)4 linker, for example, means a linker comprising the following amino acids, from N-terminus to C-terminus: GGGGSGGGGSGGGGSGGGGS (SEQ ID NO: 190). Alternatively, the linker may be, for example, a (G4Q)6, (G4Q)7, or (G4Q)8 linker. A G4Q linker is a linker made of amino acids GGGGQ (SEQ ID NO: 207), from N-terminus to C-terminus, and may be repeated multiple times. A (G4Q)4 linker, for example, means a linker comprising the following amino acids, from N-terminus to C-terminus: GGGGQGGGGQGGGGQGGGGQ (SEQ ID NO: 208). The CCR8 TCEs of the present invention comprise G4Q linkers.

The scFab, scFv, and/or scFc may also have a cysteine clamp. A “cysteine clamp” involves the introduction of a cysteine into a polypeptide domain at a specific location, typically through replacing an existing amino acid at the specific location, so that when in proximity with another polypeptide domain, also having a cysteine introduced at a specific location, a disulfide bond (a “cysteine clamp”) may be formed between the two domains. In certain embodiments, an scFc comprises at least one cysteine clamp that results in a disulfide bond across both CH2 domains. In a further specific embodiment, an scFc comprises at least two cysteine clamps that results in a disulfide bond across both CH2 domains. In other embodiments, a binding construct's VH and VL domains may comprise the cysteine clamp(s) to result in disulfide bond formation between the VH and VL domains. These cysteine clamps will stabilize the VH and VL domains in an antigen-binding configuration.

A cysteine clamp may be naturally occurring, or it may be a result of a molecule engineered to contain cysteines. For example, a scFab may have a natural cysteine clamp between the heavy and light chain constant domains. An scFab may also have a natural cysteine clamp between the heavy and light chain constant domains and an engineered cysteine clamp between cysteines at residue 44 of the heavy chain variable region and residue 100 of the light chain variable region. In addition, an anti-target scFv may also contain a cysteine clamp between cysteines at residue 44 of the heavy chain variable region and residue 100 of the light chain variable region, whereas an anti-CD3 scFv does not contain an engineered cysteine clamp. An scFc may contain hinge cysteine clamps, natural CH2/CH3 cysteine clamps, and/or an engineered CH2 cysteine clamp (intrachain).

The VH and VL contain CDRs, which are interspersed with regions that are more conserved, termed framework regions (“FR”). Each variable region is composed of 3 CDRs and 4 FRs, arranged from amino-terminus to carboxy-terminus in the following order: FR1, CDR1, FR2, CDR2, FR3, CDR3, FR4. The 3 CDRs of the VL are referred to as “LCDR1, LCDR2, and LCDR3,” and the 3 CDRs of the VH are referred to as “HCDR1, HCDR2, and HCDR3.” The CDRs contain most of the residues which form specific interactions with the antigen. That is, the CDRs contain most of the residues that are in contact with the antigen's residues. Assignment of amino acids to CDR domains within the VL and HL regions of the TCE molecules of the present invention is based on the well-known Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)). It is understand that other numbering conventions may also be used, such as Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), and/or North (North et al., “A New Clustering of Antibody CDR Loop Conformations”, Journal of Molecular Biology, 406, 228-256 (2011)).

In the most general sense, a T cell engager (“TCE”) molecule as described herein comprises a single chain polypeptide that can bind to two different antigens. A “TCE molecule” may be used interchangeably with a “BiTE molecule”. A BiTE molecule can comprise an scFv or scFab, as long as it is bispecific, meaning that it binds two targets (target antigen and CD3) at the same time. A TCE molecule is an antigen-binding molecule. A TCE molecule of the present invention may comprise an scFab that binds a target (e.g. tumor or target antigen) and an scFv that binds CD3. Such molecule may have the orientation, from N-terminus to C-terminus: scFab (VH, CH1, linker, VL, either Cκ or Cλ), linker, scFv (VH, linker, VL). Such molecules may alternatively have the orientation, from N-terminus to C-terminus: scFab (VL, either Cκ or Cλ, linker, VH, CH1), linker, scFv (VH, linker, VL). In some embodiments, the scFab binds CCR8. In particular embodiments, the TCE molecule comprises a Cκ.

A TCE molecule of the present invention may also be comprised of an scFv that binds CCR8 and an scFv that binds CD3. Such TCE molecule may have the following orientation, from N-terminus to C-terminus: scFv that binds CCR8 (VH, linker, VL), linker, scFv that binds CD3 (VH, linker, VL).

A TCE molecule of the present invention may also have a half-life extending (HLE) moiety. An HLE moiety may extend the in vivo half-life of the TCE molecules of the present invention. Nonlimiting examples of half-life extending moieties include an Fc polypeptide, a single-chain Fc polypeptide (scFc), albumin, an albumin fragment, a moiety that binds to albumin or to the neonatal Fc receptor (FcRn), a derivative of fibronectin that has been engineered to bind albumin or a fragment thereof, a peptide, a single domain protein fragment, or other polypeptide that can increase serum half-life. In other embodiments, a half-life-extending moiety can be a non-polypeptide molecule such as, for example, polyethylene glycol (PEG). In some embodiments, the HLE is a single-chain Fc (“scFc”).

A scFc is a fusion protein in which a CH2 and CH3 (Fc1) are joined via a linker to another CH2 and CH3 (Fc2) to form a continuous protein chain wherein the linker is long enough to allow the protein chain to fold back on itself. In some embodiments, the scFc comprises cysteine clamps. An scFc may also comprise an Ig-Fc hinge region, or part of an Ig-Fc hinge region. The hinge is amino terminal to the CH2 domain, and the scFc may have the following orientation: (Fc1: hinge, CH2, CH3), linker, (Fc2: hinge, CH2, CH3). It is envisaged that the hinge region promotes dimerization. Such Fc polypeptide molecules can be obtained by papain digestion of an immunoglobulin region (resulting in a dimer of two Fc polypeptide), for example and not limitation. In an embodiment, the polypeptide sequence of an Fc monomer is substantially similar to an Fc polypeptide sequence of: an IgG1 Fc region, an IgG2 Fc region, an IgG3 Fc region, an IgG4 Fc region, an IgM Fc region, an IgA Fc region, an IgD Fc region and an IgE Fc region. (See, e.g., Padlan, Molecular Immunology, 31 (3), 169-217 (1993)).

A TCE molecule of the present invention having an HLE moiety (e.g. scFc) may have the following orientation: scFab (VH, CH1, linker, VL, Ck), linker, scFv (VH, linker, VL), linker, scFc (hinge, CH2, CH3, linker, hinge, CH2, CH3). A TCE molecule of the present invention having an HLE moiety may also be in the following orientation: scFab (VL, either Cκ or Cλ, linker, VH, CH1), linker, scFv (VH, linker, VL). A TCE molecule of the present invention having an HLE moiety may also be in the following orientation: scFv that binds CCR8 (VH, linker, VL), linker, scFv that binds CD3 (VH, linker, VL), scFc (hinge, CH2, CH3, linker, hinge, CH2, CH3). An scFc may also be referred to as Fc1 (hinge, CH2, CH3), linker, Fc2 (hinge, CH2, CH3, herein.

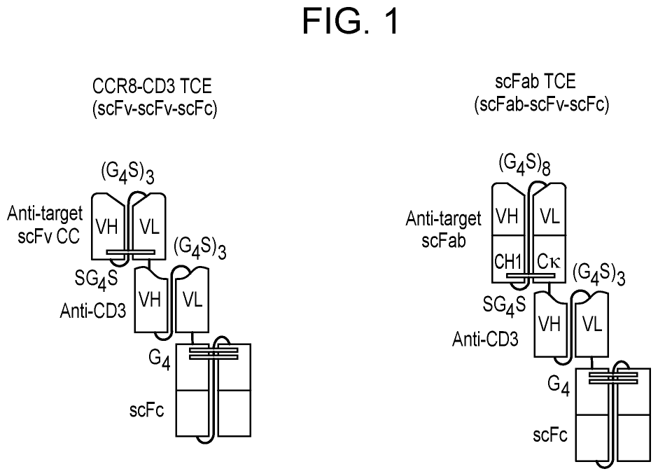

FIG. 1 depicts examples of the structures of TCE molecules of the present invention.

It will be appreciated that a TCE molecule of the present invention may have at least one amino acid substitution, providing that the TCE molecule retains the same or better desired binding specificity (e.g., binding to CCR8 and/or CD3). Therefore, modifications to the TCE molecule structures are encompassed within the scope of the invention. Such modifications may include amino acid substitutions, which may be conservative or non-conservative that do not destroy the desired binding capability of a binding construct. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

A TCE molecule of the present invention may comprise a fragment of an amino acid sequence described herein.

A TCE molecule of the present invention can bind a target antigen (e.g. antigen expressed on a tumor cell) and CD3 expressed on T cells. A target antigen can be a human protein or a protein from another species, such as mouse, rat, rabbit, and/or cynomolgus monkey. A target antigen may be any protein expressed on tumor cells, in the case for treating cancer. Nonlimiting examples of target antigens include CCR8, claudin-6, and MAGE-B2.

In another embodiment, the present invention provides vectors comprising a nucleic acid encoding a polypeptide of the invention or a portion thereof. Examples of vectors include, but are not limited to, plasmids, viral vectors, non-episomal mammalian vectors and expression vectors, for example, recombinant expression vectors.

The recombinant expression vectors of the invention can comprise a nucleic acid of the invention in a form suitable for expression of the nucleic acid in a host cell. The recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

In another embodiment, the present invention provides host cells into which a recombinant expression vector of the invention has been introduced. A host cell can be any prokaryotic cell or eukaryotic cell. Prokaryotic host cells include gram negative or gram positive organisms, for example E. coli or bacilli. Higher eukaryotic cells include insect cells, yeast cells, and established cell lines of mammalian origin. Examples of suitable mammalian host cell lines include Chinese hamster ovary (CHO) cells or their derivatives such as Veggie CHO and related cell lines which grow in serum-free media (see Rasmussen et al., 1998, Cytotechnology 28:31) or CHO strain DXB-11, which is deficient in DHFR (see Urlaub et al., 1980, Proc. Natl. Acad. Sci. USA 77:4216-20). Additional CHO cell lines include CHO-K1 (ATCC #CCL-61), EM9 (ATCC #CRL-1861), and UV20 (ATCC #CRL-1862). Additional host cells include the COS-7 line of monkey kidney cells (ATCC CRL 1651) (see Gluzman et al., 1981, Cell 23:175), L cells, C127 cells, 3T3 cells (ATCC CCL 163), AM-1/D cells (described in U.S. Pat. No. 6,210,924), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Appropriate cloning and expression vectors for use with bacterial, fungal, yeast, and mammalian cellular hosts are described by Pouwels et al. (Cloning Vectors: A Laboratory Manual, Elsevier, New York, 1985).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. The leader sequence may comprise an amino acid sequence given by SEQ ID NO: 213 (MDMRVPAQLL GLLLLWLRGA RC) which is encoded by SEQ ID NO: 214 (atggacatga gagtgcctgc acagctgctg ggcctgctgc tgctgtggct gagaggegcc agatgc). The leader sequence may comprise an amino acid sequence given by SEQ ID NO: 215 (MAWALLLLTL LTQGTGSWA) which is encoded by SEQ ID NO: 216 (atggcctggg ctctgctgct cctcaccctc ctcactcagg gcacagggtc ctgggcc). The leader polynucleotide sequence may comprise a polynucleotide sequence given by SEQ ID NO: 594 (ATGGACATGAGAGTGCCTGCACAGCTGCTGGGCCTGCTGCTGCTGTGGCTGAGA GGCGCCAGATG)

Vector DNA can be introduced into prokaryotic or eukaryotic cells via conventional transformation or transfection techniques. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Additional selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

A polynucleotide encoding an amino acid sequence of a TCE molecule of the present invention can be any length as appropriate for the desired use or function, and can comprise one or more additional sequences, for example, regulatory sequences, and/or be part of a larger nucleic acid, for example, a vector. The skilled artisan will appreciate that, due to the degeneracy of the genetic code, each of the polypeptide sequences disclosed herein is encoded by a large number of other nucleic acid sequences. Mutations can also be introduced into a nucleic acid without significantly altering the biological activity of a polypeptide that it encodes. For example, one can make nucleotide substitutions leading to amino acid substitutions at non-essential amino acid residues.

Transformed cells can be cultured under conditions that promote expression of the polypeptide, and the polypeptide recovered by conventional protein purification procedures. Polypeptides contemplated for use herein include substantially homogeneous recombinant mammalian polypeptides substantially free of contaminating endogenous materials. Cells containing the nucleic acid encoding the TCE molecules of the present invention also include hybridomas.

In some embodiments, a vector comprising a nucleic acid molecule as described herein is provided. In some embodiments, the invention comprises a host cell comprising a nucleic acid molecule as described herein. In some embodiments, a nucleic acid molecule encoding a TCE molecule as described herein is provided. In some embodiments, a pharmaceutical composition comprising at least one TCE molecule described herein is provided.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the TCE molecules included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

The TCR-CD3 complex is a heteromultimer comprising a heterodimer comprising TCRα and TCRβ or TCRγ and TCRδ plus various CD3 chains from among the CD3 zeta (CD35) chain, CD3 epsilon (CD3ϵ) chain, CD3 gamma (CD3γ) chain, and CD3 delta (CD3δ) chain.

The CD3 receptor complex is a protein complex and is composed of four chains. In mammals, the complex contains a CD3γ (gamma) chain, a CD38 (delta) chain, and two CD3ε (epsilon) chains. These chains associate with the T cell receptor (TCR) and the so-called ((zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3E gene which resides on chromosome 11. The most preferred epitope of CD3 epsilon is comprised within amino acid residues 1-27 of the human CD3 epsilon extracellular domain. It is envisaged that the TCE molecules according to the present invention typically and advantageously show less unspecific T cell activation, which is not desired in specific immunotherapy. This translates to a reduced risk of side effects.

In some embodiments the effector cell protein can be the human CD3 epsilon (CD3ϵ) chain, which can be part of a multimeric protein. Alternatively, the effector cell protein can be human and/or cynomolgus monkey TCRα, TCRβ, TCRδ, TCRγ, CD3 beta (CD3B) chain, CD3 gamma (CD3γ) chain, CD3 delta (CD38) chain, or CD3 zeta (CD35) chain.

Moreover, in some embodiments, a TCE molecule can also bind to a CD3ϵ chain from a non-human species, such as mouse, rat, rabbit, new world monkey, and/or old world monkey species. Such species include, without limitation, the following mammalian species: Mus musculus; Rattus; Rattus norvegicus; the cynomolgus monkey, Macaca fascicularis; the hamadryas baboon, Papio hamadryas; the Guinea baboon, Papio; the olive baboon, Papio anubis; the yellow baboon, Papio cynocephalus; the Chacma baboon, Papio ursinus; Callithrix jacchus; Saguinus oedipus; and Saimiri sciureus. Having a therapeutic molecule that has comparable activity in humans and species commonly used for preclinical testing, such as mice and monkeys, can simplify, accelerate, and ultimately provide improved outcomes in drug development. In the long and expensive process of bringing a drug to market, such advantages can be critical.

As used interchangeably herein, “treatment” and/or “treating” and/or “treat” are intended to refer to all processes wherein there may be a slowing, interrupting, arresting, controlling, stopping, or reversing of the progression of the disorders described herein, but does not necessarily indicate a total elimination of all disorder symptoms. Treatment includes administration of a TCE molecule of the present invention for treatment of a disease or condition in a human that would benefit from activity of a TCE molecule of the present invention and includes: (a) inhibiting further progression of the disease; and (b) relieving the disease, i.e., causing regression of the disease or disorder or alleviating symptoms or complications thereof.

Suitable PD-L1 antagonist antibodies for use in combination with a TCE molecule of the present invention include, but are not limited to, atezolizumab, avelumab, or durvalumab. Examples of PD-1 antagonist antibodies suitable for use in the methods of the invention include, but are not limited to pembrolizumab, nivolumab, cemiplimab, pidilizumab, spartalizumab, camrelizumab, sintilimab, tislelizumab, toripalimab, dostarlimab, Antibody 20C1.006 (SEQ ID NOs: 72-81), Antibody 20C1.009 (SEQ ID NOs: 32-41 or SEQ ID NOs: 32-40 and SEQ ID NO: 212), Antibody 20A2.003 (SEQ ID NOs: 42-51), Antibody 20D4.006 (SEQ ID NOs: 52-61), or Antibody 20D4.17 (SEQ ID NOs: 62-71), and any of the PD-1 antagonist antibodies described in WO 2019/140196.

Therapeutically effective doses of a TCE molecule can be administered. The amount of TCE molecule that constitutes a therapeutically dose may vary with the indication treated, the weight of the patient, the calculated skin surface area of the patient. Dosing of a TCE molecule can be adjusted to achieve the desired effects. In many cases, repeated dosing may be required. Dosages and the frequency of administration may vary according to such factors as the route of administration, the particular TCE molecule employed, the nature and severity of the disease to be treated, whether the condition is acute or chronic, and the size and general condition of the subject.

As used herein, an “effective amount” means the amount of a TCE molecule of the present invention or pharmaceutical composition comprising such TCE molecule that will elicit the biological or medical response of or desired therapeutic effect on a tissue, system, animal, mammal, or human that is being sought by the researcher, medical doctor, or other clinician. An effective amount of the TCE molecule may vary according to factors such as the disease state, age, sex, and weight of the individual, and the ability of the TCE molecule to elicit a desired response in the individual. An effective amount is also one in which any toxic or detrimental effect of the TCE molecule is outweighed by the therapeutically beneficial effects. Such benefit includes improving signs or symptoms of cancer. An effective amount can be readily determined by one skilled in the art, by the use of known techniques, and by observing results obtained under analogous circumstances. An effective amount of a TCE molecule of the present invention may be administered in a single dose or in multiple doses. In determining the effective amount for a patient, a number of factors are considered by the attending medical practitioner, including, but not limited to: the patient's size (e.g., weight or mass), body surface area, age, and general health; the specific disease or disorder involved; the degree of, or involvement, or the severity of the disease or disorder; the response of the individual patient; the particular compound administered; the mode of administration; the bioavailability characteristics of the preparation administered; the dose regimen selected; the use of concomitant medication; and other relevant circumstances known to medical practitioners.

A TCE molecule, or a pharmaceutical composition containing such a molecule, can be administered by any feasible method. Protein therapeutics will ordinarily be administered by a parenteral route, for example by injection, since oral administration, in the absence of some special formulation or circumstance, would lead to hydrolysis of the protein in the acid environment of the stomach. Subcutaneous, intramuscular, intravenous, intraarterial, intralesional, or peritoneal bolus injection are possible routes of administration. A TCE molecule can also be administered via infusion, for example intravenous or subcutaneous infusion.

TCE molecules can be administered in the form of a composition comprising one or more additional components such as a physiologically acceptable carrier, excipient or diluent. Optionally, the composition additionally comprises one or more physiologically active agents. In various particular embodiments, the composition comprises one, two, three, four, five, or six physiologically active agents in addition to one or more TCE molecules.

### EXAMPLES

### Example: TCE Molecule Affinities

### Claudin-6 TCE Molecule Affinities

Claudin-6 T cell engager (“TCE”) molecules are examined for affinities to human Claudin-6. TCE molecules are represented below in Table 1 by unique identifiers. For example, the TCE molecule “CL6 3C1 HL CC×12C×scFc” refers to a TCE molecule having, from N-terminus to C-terminus, an scFv with an engineered cysteine clamp (“CC”; clamp between VH44 and VL 100 (Kabat numbering)) that binds Claudin-6 (“CL6”) and has the VH N-terminal to the VL, an I2C scFv that targets CD3 (VH N-terminal to the VL), and an scFc. The TCE molecule “CL6 3C1 HL scFab×I2C×scFc” refers to a TCE molecule having an scFab that binds Claudin-6 having the VH N-terminal to the VL, an I2C scFv that targets CD3 (VH N-terminal to the VL), and an scFc. An “x” represents a linker. The CDR sequences for both CL6 3C1 molecules are identical. FIG. 1 depicts a generic structure for each molecule. The human Claudin 6 sequence is given by UniProt entry P56747 and includes variants and isoforms thereof.

Cell-based affinity of TCE molecules is determined by nonlinear regression (one site-specific binding) analysis. CHO cells transfected with human Claudin-6 were incubated with decreasing concentrations of TCE molecules (up to 50 nM, step 1:1, 10 steps) for 16 h at 4° C. Bound TCE molecules are detected with Alexa Fluor 488-conjugated AffiniPure Fab Fragment Goat Anti-Human IgG (H+L). Fixed cells are detected with FACS flow and signals are detected by fluorescence cytometry. Respective equilibrium dissociation constant (Kd) values are calculated with the one site specific binding evaluation tool of the GraphPad Prism software. Mean Kd values and standard deviation are calculated with Microsoft Excel. Mean Kd values are calculated from three independent experiments.

According to procedures essentially as described above, the following affinities were obtained.

TABLE 1
Cell-based affinities of Claudin-6
TCE molecules to human Claudin-6.

| | Mean |
|---|---|
| TCE molecule | Kd [nM] |
| CL6 3C1 HL CC scFv × I2C × scFc | 7.77 ± 1.49 |
| CL6 3C1 HL scFab × I2C × scFc | 2.48 ± 0.24 |
| CL6 3C1-01 HL (scFv) CC × I2C × | 2.09 ± 0.76 |
| scFc | |
| CL6 3C1-01 HL scFab × I2C × scFc | 1.19 ± 0.09 |
| CL6 3C1-03 HL CC scFv × I2C × scFc | 7.42 ± 2.11 |
| CL6 3C1-03 HL scFab × I2C × scFc | 4.56 ± 1.42 |
| CL6 3C1-04 HL CC(scFv) × I2C × scFc | 24.82 ± 10.3 |
| CL6 3C1-04 HL scFab × I2C × scFc | 5.73 ± 2.38 |
| CL6 3C1-04.1 HL CC scFv × I2C × | 12.94 ± 4.23 |
| scFc | |
| CL6 3C1-04.1 HL scFab × I2C × scFc | 3.55 ± 0.91 |
| CL6 3C1-05 HL CC scFv × I2C × scFc | 24.39 ± 11.42 |
| CL6 3C1-05 HL scFab × I2C × scFc | 3.03 ± 0.73 |
| CL6 3C1-05.1 HL CC scFv × I2C × | 2.70 ± 0.66 |
| scFc | |
| CL6 3C1-05.1 HL scFab × I2C × scFc | 1.12 ± 0.49 |
| CL6 3C1-06 HL CC scFv × I2C × scFc | 17.61 ± 7.46 |
| CL6 3C1-06 HL scFab × I2C × scFc | 4.84 ± 2.17 |
| CL6 3D4-01.1 LH CC scFv × I2C × | 5.83 ± 1.36 |
| scFc | |
| CL6 3D4-01.1 LH scFab × I2C × scFc | 2.51 ± 1.05 |
| CL6 3D4-02 LH CC scFv × I2C × scFc | 4.23 ± 1.02 |
| CL6 3D4-02 LH scFab × I2C × scFc | 1.41 ± 0.42 |
| CL6 3D4-03.1 LH CC scFv × I2C × | 11.83 ± 3.14 |
| scFc | |
| CL6 3D4-03.1 LH scFab × I2C × scFc | 2.23 ± 0.96 |
| CL6 3D4-04 LH CC scFv × I2C × scFc | 17.00 ± 8.23 |
| CL6 3D4-04 LH scFab × I2C × scFc | 6.99 ± 2.04 |
| CL6 3D4-04.G2 LH CC scFv × I2C × | 11.42 ± 3.08 |
| scFc | |
| CL6 3D4-04.G2 LH scFab × I2C × scFc | 27.04 ± 2.52 |
| CL6 3C1-02 HL CC scFv × I2C × scFc | 5.45 ± 0.81 |
| CL6 3C1-02 HL scFab × I2C × scFc | 2.62 ± 0.30 |

As shown in Table 1, these data demonstrate that TCE molecules having an scFab target binder show higher affinity for human Claudin-6 compared to TCE molecules having an scFv target binder.

### Mage-B2 TCE Molecule Affinities

Similarly, cell-based affinity of MAGE-B2 TCE molecules is determined by nonlinear regression (one site-specific binding) analysis. HLA-A*02:01 expressing T2 cells exogenously loaded with human MAGE-B2 peptide are incubated with decreasing concentrations of TCE molecules (up to 400 nM, 1:2 dilutions, 11 steps) for 16 hours at 4° C. Bound TCE molecules are detected with Alexa Fluor 488-conjugated AffiniPure Fab Fragment Goat Anti-Human IgG (H+L). Fixed cells are stained with DRAQ5, Far-Red Fluorescent Live-Cell Permeant DNA Dye, and signals are detected by fluorescence cytometry. Respective equilibrium dissociation constant (Kd) values are calculated with the one site specific binding evaluation tool of the GraphPad Prism software. Mean Kd values and standard deviations are calculated with Microsoft Excel. Mean Kd values are calculated from three independent experiments.

Following procedures essentially as described above, the following data were obtained.

TABLE 2
Cell-based affinities of MAGE-B2 TCE molecules.

| Construct | Mean Kd [nM] |
|---|---|
| MA 03-D8 AS CC scFv × I2C × scFc | 0.54 ± 0.10 |
| MA 03-D8 AS scFab × I2C × scFc | 0.42 ± 0.11 |
| MA 09-E2 CC scFv × I2C × scFc | 0.70 ± 0.19 |
| MA 09-E2 scFab × I2C × scFc | 0.39 ± 0.03 |
| MA 09-F12 CC scFv × I2C × scFc | 0.53 ± 0.18 |
| MA 09-F12 scFab × I2C × scFc | 0.46 ± 0.13 |
| MA 09-G10 CC scFv × I2C × scFc | 0.83 ± 0.30 |
| MA 09-G10 scFab × I2C × scFc | 0.52 ± 0.10 |
| MA 09-H10 CC scFv × I2C × scFc | 0.62 ± 0.13 |
| MA 09-H10 scFab × I2C × scFc | 0.49 ± 0.18 |
| MA 09-H7 CC scFv × I2C × scFc | 0.79 ± 0.37 |
| MA 09-H7 scFab × I2C × scFc | 0.50 ± 0.11 |
| MA 10-D3 CC scFv × I2C × scFc | 1.10 ± 0.23 |
| MA 10-D3 scFab × I2C × scFc | 0.54 ± 0.10 |
| MA 10-D6 CC scFv × I2C × scFc | 0.67 ± 0.12 |
| MA 10-D6 scFab × I2C × scFc | 0.50 ± 0.10 |
| MA 10-G10 AS CC scFv × I2C × scFc | 1.75 ± 0.52 |
| MA 10-G10 AS scFab × I2C × scFc | 0.65 ± 0.10 |
| MA 98-C7 CC scFv × I2C × scFc | 0.72 ± 0.22 |
| MA 98-C7 scFab × I2C × scFc | 0.45 ± 0.08 |
| MA 03-E11 AS CC scFv × I2C × scFc | 4.21 ± 0.46 |
| MA 03-E11 AS scFab × I2C × scFc | 1.25 ± 0.15 |
| MA 86-A4-N-F5 CC scFv × I2C × scFc | 51.80 ± 12.18 |
| MA 86-A4-N-F5 scFab × I2C × scFc | 65.87 ± 15.79 |
| MA 88-B3-F9 CC scFv × I2C × scFc | 74.06 ± 36.22 |
| MA 88-B3-F9 scFab × I2C × scFc | 83.67 ± 8.70 |
| MA SG-F28 CC scFv × I2C × scFc | 76.77 ± 18.34 |
| MA SG-F28 scFab × I2C × scFc | 62.81 ± 8.08 |

As shown in Table 2, these data demonstrate that TCE molecules having an scFab target binder show higher affinity for human MAGE-B2 compared to TCE molecules having an scFv target binder.

### Example: FACS-Based Cytotoxicity Assay with Unstimulated Human PBMC

Human peripheral blood mononuclear cells (PBMC) are prepared by Ficoll density gradient centrifugation from enriched lymphocyte preparations (buffy coats), a side product of blood banks collecting blood for transfusions. Buffy coats are supplied by a local blood bank and PBMC are prepared on the day after blood collection. After Ficoll density centrifugation and extensive washes with Dulbecco's PBS (Gibco), remaining erythrocytes are removed from PBMC via incubation with erythrocyte lysis buffer (155 mM NH4Cl, 10 mM KHCO3, 100 μM EDTA). Remaining lymphocytes mainly encompass B and T lymphocytes, NK cells, and monocytes. PBMC are kept in culture at 37° C./5% CO2 in RPMI medium (Gibco) with 10% FCS (Gibco).

Human T cells are isolated from PBMC using human Pan T cell isolation kit (Miltenyi Biotec, #130-096-535) according to the manufacturer's protocol. T cells are isolated using LS Columns (Milteny Biotec, #130-042-401). T cells are cultured in RPMI complete medium (RPMI1640; Biochrom AG, #FG1215) supplemented with 10% FBS (Bio West, #S1810), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C.

For the analysis of cell lysis in flow cytometry assays, the fluorescent membrane dye DiOC18 (DiO) (Thermo Fisher, #V22886) is used to label target antigen positive cells (Claudin-6 stable transfected CHO cells or DAN-G stable transfected MAGE-B2 cells) as target cells and distinguish them from effector cells. Briefly, cells are harvested, washed once with PBS and adjusted to 10^{e}6 cells/mL in PBS containing 2% (v/v) FBS and the membrane dye DiO (5 μL/10^{e}6 cells). After incubation for 3 minutes at 37° C., cells are washed twice in complete RPMI medium and the cell number is adjusted to 1.25×10^{e}5 cells/mL. The vitality of cells is determined using Nucleocounter NC-250 (Chemometec) and Solution18 Dye containing Acridine Orange and DAPI (Chemometec).

To quantify the lysis of target antigen positive cell lines in the presence of serial dilutions of TCE molecules, equal volumes of DiO-labeled target cells and effector cells (i.e., PBMC w/o CD14+ cells) are mixed, resulting in an E:T cell ratio of 10:1. 160 μl of this suspension is transferred to each well of a 96-well plate. Forty μL of serial dilutions of the corresponding TCE molecule, a negative control, or RPMI complete medium as an additional negative control are added. The TCE molecule-mediated cytotoxic reaction proceeds for 48 hours in a 7% CO2 humidified incubator. Cells are transferred to a new 96-well plate and loss of target cell membrane integrity is monitored by adding propidium iodide (PI) at a final concentration of 1 μg/mL. PI is a membrane impermeable dye that normally is excluded from viable cells, whereas dead cells take it up and become identifiable by fluorescent emission.

Samples are measured by flow cytometry on an iQue Plus (Intellicyt, now Sartorius) instrument and analyzed by Forecyt software (Intellicyt). Target cells are identified as DiO-positive cells. PI-negative target cells are classified as living target cells. Percentage of cytotoxicity is calculated as dead target cells/target cells×100. Using GraphPad Prism 7.04 software (Graph Pad Software, San Diego), the percentage of cytotoxicity is plotted against the corresponding TCE molecule concentrations. Dose response curves are analyzed with the four parametric logistic regression models for evaluation of sigmoid dose response curves with fixed hill slope and EC₅₀ values are calculated.

Following procedures essentially as described above, the following data were obtained.

TABLE 3
Claudin-6 TCE molecule mediated cytotoxicity.

| | EC₅₀ |
|---|---|
| TCE Molecule | [pM] |
| CL6 3C1 HL CC scFv × I2C × scFc | 2.35 |
| CL6 3C1 HL scFab × I2C × scFc | 0.13 |
| CL6 3C1-01 HL CC scFv × I2C × scFc | 1.86 |
| CL6 3C1-01 HL scFab × I2C × scFc | 0.11 |
| CL6 3C1-03 HL CC scFv × I2C × scFc | 12.80 |
| CL6 3C1-03 HL scFab × I2C × scFc | 0.45 |
| CL6 3C1-04 HL CC scFv × I2C × scFc | 33.7 |
| CL6 3C1-04 HL scFab × I2C × scFc | 0.67 |
| CL6 3C1-04.1 HL CC scFv × I2C × | 15.43 |
| scFc | |
| CL6 3C1-04.1 HL scFab × I2C × scFc | 0.38 |
| CL6 3C1-05 HL CC scFv × I2C × scFc | 18.75 |
| CL6 3C1-05 HL scFab × I2C × scFc | 0.42 |
| CL6 3C1-05.1 HL CC scFv × I2C × | 5.27 |
| scFc | |
| CL6 3C1-05.1 HL scFab × I2C × scFc | 0.11 |
| CL6 3C1-06 HL CC scFv × I2C × scFc | 36.7 |
| CL6 3C1-06 HL scFab × I2C × scFc | 0.58 |
| CL6 3D4-01.1 LH CC scFv × I2C × | 25.9 |
| scFc | |
| CL6 3D4-01.1 LH scFab × I2C × scFc | 1.41 |
| CL6 3D4-02 LH CC scFv × I2C × scFc | 4.69 |
| CL6 3D4-02 LH scFab × I2C × scFc | 0.52 |
| CL6 3D4-03.1 LH CC scFv × I2C × | 23.3 |
| scFc | |
| CL6 3D4-03.1 LH scFab × I2C × scFc | 1.03 |
| CL6 3D4-04 LH CC scFv × I2C × scFc | 37.2 |
| CL6 3D4-04 LH scFab × I2C × scFc | 3.15 |
| CL6 3D4-04.G2 LH CC scFv × I2C × | 48.5 |
| scFc | |
| CL6 3D4-04.G2 LH scFab × I2C × scFc | 11.99 |

These data demonstrate that the Claudin-6 TCE molecules having an scFab target binder demonstrated improved potency of Claudin-6 transfected CHO cells compared to TCE molecules having an scFv target binder.

Data demonstrating MAGE-B2 TCE molecule cytotoxicity is shown in Table 4.

TABLE 4
MAGE-B2 TCE molecule mediated cytotoxicity.

| | EC₅₀ |
|---|---|
| TCE Molecule | [pM] |
| MA 03-D8 AS scFab × I2C × scFc | 4.8 |
| MA 03-D8 AS CC scFv × I2C × scFc | 15.2 |
| MA 09-E2 scFab × I2C × scFc | 21.7 |
| MA 09-E2 CC scFv × I2C × scFc | 68.7 |
| MA 09-F12 scFab × I2C × scFc | 48.8 |
| MA 09-F12 CC scFv × I2C × scFc | 83.5 |
| MA 09-G10 scFab × I2C × scFc | 52.8 |
| MA 09-G10 CC scFv × I2C × scFc | 90.8 |
| MA 09-H10 scFab × I2C × scFc | 40.5 |
| MA 09-H10 CC scFv × I2C × scFc | 103.4 |
| MA 09-H7 scFab × I2C × scFc | 74.6 |
| MA 09-H7 CC scFv × I2C × scFc | 227.2 |
| MA 10-B5 scFab × I2C × scFc | 11.7 |
| MA 10-B5 CC scFv × I2C × scFc | 33.3 |
| MA 10-D3 scFab × I2C × scFc | 60.8 |
| MA 10-D3 CC scFv × I2C × scFc | 196.9 |
| MA 10-D6 scFab × I2C × scFc | 27.7 |
| MA 10-D6 CC scFv × I2C × scFc | 45.0 |
| MA 10-G10 AS scFab × I2C × scFc | 32.9 |
| MA 10-G10 AS CC scFv × I2C × scFc | 141.0 |
| MA 98-C7 scFab × I2C × scFc | 9.9 |
| MA 98-C7 CC scFv × I2C × scFc | 70.5 |
| MA 03-E11 AS scFab × I2C × scFc | 60.6 |
| MA 03-E11 AS CC scFv × I2C × scFc | 212.4 |

These data demonstrate that the MAGE-B2 TCE molecules having an scFab target binder demonstrated increased lysis of DAN-G cells compared to TCE molecules having an scFv target binder.

In a similar experiment, a scFab-containing TCE molecule having a disulfide-bridge (cysteine clamp (“CC”); cysteines at Kabat residues VH44/VL100; CL6 3C1-02scFabCC×I2C×scFc) is tested for cytotoxic activity. These data (shown in Table 5) demonstrate that the scFab-containing TCE molecule having a disulfide bridge showed similar activity compared to the scFab-containing TCE molecule without this disulfide bridge. Both scFab-containing TCE molecules demonstrated increased cytotoxicity compared to TCE molecules having an scFv target binder.

TABLE 5
CL 3C1-02-based BiTE molecules.

| | EC50 |
|---|---|
| CLDN6 BiTE molecules | [pM] |
| CL6 3C1-02 CC scFv × I2C × | 89 |
| scFc | |
| CL6 3C1-02 scFab × I2C × scFc | 4.2 |
| CL6 3C1-02 scFab CC × I2C × | 4.3 |
| scFc | |

Data from TCE molecules binding a target antigen and CD3 is shown below in Table 6. The TCE molecules either contained a (G4S)8 linker in a scFab (VH-CHI-(G4S)8 linker-VL-Ck)-linker-aCd3scFv (VH-linker-VL)-linker-scFc (Fc-linker-Fc) orientation (top row), a (G4S)6 linker (second row), a disulfide-bridge stabilized (Kabat VH44/VL100) scFv target binding moiety (third row), or a (G4S)8 linker in a scFab (VH-CHI-linker-VL-Ck)-linker-aCd3scFv (VH-linker-VL)-linker-scFc (Fc-linker-Fc) orientation (bottom row).

TABLE 6
TCE molecule mediated cytotoxicity.

| | EC50 |
|---|---|
| TCE Molecule | [pM] |
| HL scFab(G4S)8 × I2C × scFc | 3.9 |
| HL scFab(G4S)6 × I2C × scFc | 7.5 |
| HL CC scFv × I2C × scFc | 40 |
| LH scFab(G4S)8 × I2C × scFc | 60 |

These data demonstrate that the scFab-containing TCE molecule having an scFab (G4S)8 linker showed the highest cytotoxic activity compared to the other molecules tested. The scFab-containing TCE molecule with the VL-Ck-linker-VH-CH1-aCD3-scFc orientation demonstrated the least activity, which could be due to the orientation difference as compared to scFab-containing TCE molecule having the orientation VH-CH1-VL-Ck-aCD3-scFc.

### Example: Application of scFab Moiety in TCE Formats

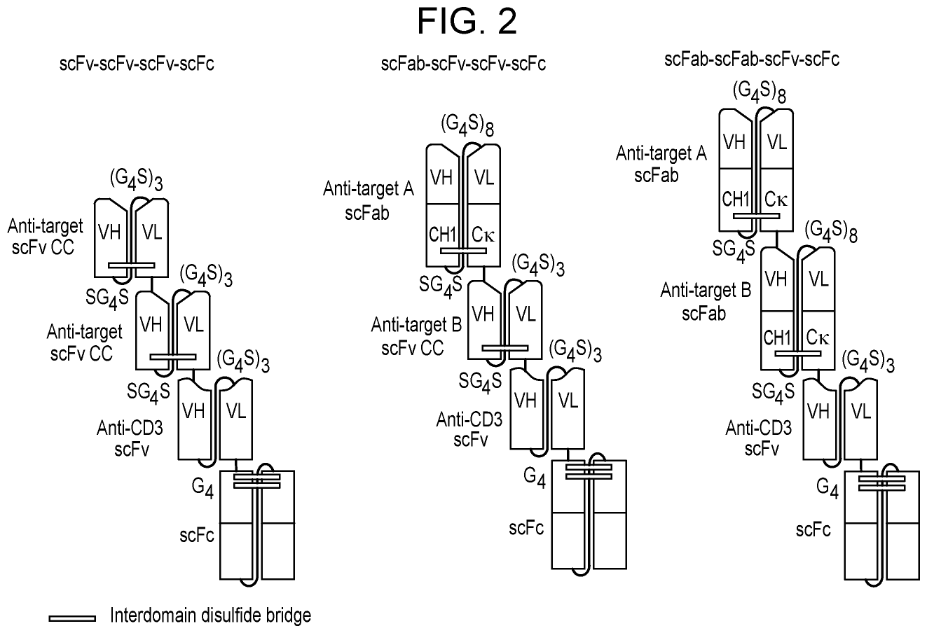

Multitargeting TCE molecules are tested for cytotoxicity. As shown in the structures in FIG. 2, tested TCE molecules have either two anti-target scFvs (CD22 11-C3 CC scFv×CD20 29-F5 CC scFv×12C×scFc and CD20 99-E5 CC scFv×CD22 28-B7N655 CC scFv×I2C×scFc), an anti-target scFab and an anti-target scFv (CD20 99-E5 scFab×CD22 28-B7N655 CC scFv×I2C×scFc), or two anti-target scFabs (CD22 11-C3 scFab×CD20 29-F5 scFab×I2C×scFc). Tested TCE molecules have an anti-CD3 scFv (“I2C”) and an scFc. 11-C3, 29-F5, 99-E5, and 28-B7N655 refer to target binders, such that 11-C3 scFab will have the same CDRs as 11-C3 scFv, for example. Cytotoxicity of Raji cells (double positive for CD20 and CD22) is essentially determined as described above, and the following data were obtained.

TABLE 7
TCE molecule mediated cytotoxicity (EC50).

| | | CHO | CHO |
|---|---|---|---|
| TCE molecule | Raji | CD20 | CD22 |
| CD22 11-C3 scFab × CD20 | 872 | 14.5 | 17.0 |
| 29-F5 scFab × I2C × scFc | | | |
| CD22 11-C3 CC scFv × CD20 | 2308 | 64.7 | 16.4 |
| 29-F5 CC scFv × I2C × scFc | | | |
| CD20 99-E5 scFab × CD22 | 28.8 | 20.9 | |
| 28-B7N655 CC scFv × I2C × scFc | | | |
| CD20 99-E5 CC scFv × CD22 | 35.4 | 52.4 | |
| 28-B7N655 CC scFv × I2C × scFc | | | |

As shown in Table 7, TCE molecules having scFabs as both target binders (first row) have improved potency against single positive target cells (CD20 transfected CHO) and on double-positive Raji cells compared to TCE molecules having scFvs as both target binders (second row). In addition, TCE molecules having a CD20-binding scFab (third row) demonstrate improved potency for CD20 transfected CHO cells and double-positive Raji cells compared to TCE molecules having a CD20-binding scFv (fourth row).

### Example: Evaluation of TCE Molecules Protein Surface Hydrophobicity

To measure protein surface hydrophobicity, isolated and formulated TCE molecule monomer adjusted to a defined protein concentration is transferred into autosampler fitting sample vials and measured on a FPLC system. A Hydrophobic Interaction Chromatography (HIC) column is equilibrated with formulation buffer and a defined volume of protein solution applied at a constant formulation buffer flow. Detection is done by OD280 nm optical absorption.

Elution behavior is determined by peak shape respectively mathematically calculation of declining signal peak slope. Steeper slope/higher slope values indicate less hydrophobic interaction of the protein surface compared to constructs with more flat elution behavior and lower slope value.

Following procedures essentially as described above, the following data were obtained. “I2C” refers to an scFv that binds CD3. “CC” refers to an scFv containing an engineered cysteine clamp between a cysteine at position 44 (VH) and a cysteine at position 100 (VL) (Kabat).

TABLE 8
HIC elution behavior of MAGE-B2 TCE molecules.

| | HIC Elution |
|---|---|
| TCE molecule | Slope |
| MA 03-D8 AS CC scFv × I2C × scFc | 49.69 |
| MA 03-D8 AS scFab × I2C × scFc | 50.4 |
| MA 03-E11 AS CC scFv × I2C × scFc | 25.35 |
| MA 03-E11 AS scFab × I2C × scFc | 45.93 |
| MA 09-E2 CC scFv × I2C × scFc | 21.55 |
| MA 09-E2 scFab × I2C × scFc | 39.58 |
| MA 09-F12 CC scFv × I2C × scFc | 25.39 |
| MA 09-F12 scFab × I2C × scFc | 35.03 |
| MA 09-G10 CC scFv × I2C × scFc | 37.3 |
| MA 09-G10 scFab × I2C × scFc | 40.26 |
| MA 09-H10 CC scFv × I2C × scFc | 29.02 |
| MA 09-H10 scFab × I2C × scFc | 40.21 |
| MA 09-H7 CC scFv × I2C × scFc | 35.56 |
| MA 09-H7 scFab × I2C × scFc | 43.47 |
| MA 10-D3 CC scFv × I2C × scFc | 16.23 |
| MA 10-D3 scFab × I2C × scFc | 35.17 |
| MA 10-D6 CC scFv × I2C × scFc | 7.4 |
| MA 10-D6 scFab × I2C × scFc | 27.65 |
| MA 10-G10 AS CC scFv × I2C × scFc | 51.54 |
| MA 10-G10 AS scFab × I2C × scFc | 43.73 |
| MA 86-A4-N-F5 CC scFv × I2C × scFc | 35.66 |
| MA 86-A4-N-F5 scFab × I2C × scFc | 60.06 |
| MA 88-B3-F9 CC scFv × I2C × scFc | 51.05 |
| MA 88-B3-F9 scFab × I2C × scFc | 54.89 |
| MA 98-C7 CC scFv × I2C × scFc | 44.38 |
| MA 98-C7 scFab × I2C × scFc | 52.04 |
| MA SG-F28 CC scFv × I2C × scFc | 19.77 |
| MA SG-F28 scFab × I2C × scFc | 30.47 |
| MA 10-B5 CC scFv × I2C × scFc | 16.81 |
| MA 10-B5 scFab × I2C × scFc | 42.19 |

TABLE 9
HIC elution behavior of CD20/CD22 TCE molecules.

| | HIC Elution |
|---|---|
| TCE Molecule | Slope |
| CD22 11-C3 scFab × CD20 29-F5 | 46.27 |
| scFab × I2C × scFc | |
| CD22 11-C3 CC scFv × CD20_29-F5_CC | 17.49 |
| scFv × I2C ×scFc | |
| CD20 99-E5scFab × CD22 28-B7 N655 | 17.4 |
| CC scFv × I2C × scFc | |
| CD20 99-E5 CC scFv × CD22 28-B7 N655 | 10.99 |
| CC scFv × I2C × scFc | |

TABLE 10
HIC elution behavior of Claudin-6 TCE molecules.

| | HIC Elution |
|---|---|
| TCE Molecule | Slope |
| CL6 3C1 HL CC scFv × I2C × scFc | 48.1 |
| CL6 3C1 HL scFab × I2C × scFc | 51.85 |
| CL6 3C1-01 HL CC scFv × I2C × scFc | 41.54 |
| CL6 3C1-01 HL scFab × I2C × scFc | 44.32 |
| CL6 3C1-03 HL CC scFv × I2C × scFc | 52.18 |
| CL6 3C1-03 HL scFab × I2C × scFc | 46.19 |
| CL6 3C1-04 HL CC scFv × I2C × scFc | 44.08 |
| CL6 3C1-04 HL scFab × I2C × scFc | 51.49 |
| CL6 3C1-04.1 HL CC scFv × I2C × scFc | 36.65 |
| CL6 3C1-04.1 HL scFab × I2C × scFc | 35.92 |
| CL6 3C1-05 HL CC scFv × I2C × scFc | 37.31 |
| CL6 3C1-05 HL scFab × I2C × scFc | 45.42 |
| CL6 3C1-05.1 HL CC scFv × I2C × scFc | 33.43 |
| CL6 3C1-05.1 HL scFab × I2C × scFc | 36.13 |
| CL6 3C1-06 HL CC scFv × I2C × scFc | 42.98 |
| CL6 3C1-06 HL scFab × I2C × scFc | 44.7 |
| CL6 3D4-01.1 LH CC scFv × I2C × scFc | 16.52 |
| CL6 3D4-01.1 LH scFab × I2C × scFc | 15.83 |
| CL6 3D4-02 LH CC scFv × I2C × scFc | 10.1 |
| CL6 3D4-02 LH scFab × I2C × scFc | 16.36 |
| CL6 3D4-03.1 LH CC scFv × I2C × scFc | 16.01 |
| CL6 3D4-03.1 LH scFab × I2C × scFc | 18.23 |
| CL6 3D4-04 LH CC scFv × I2C × scFc | 13.58 |
| CL6 3D4-04 LH scFab × I2C × scFc | 18.23 |
| CL6 3D4-04.G2 LH CC scFv × I2C × scFc | 15.43 |
| CL6 3D4-04.G2 LH scFab × I2C × scFc | 27.12 |
| CL6 3C1-02 CC scFv × I2C × scFc | 46.25 |
| CL6 3C1-02 scFab × I2C × scFc | 45.79 |

These data demonstrate the degree of interaction of the applied construct with the hydrophobic column matrix surface. In most cases, peak slopes for the scFab comprising TCE molecules are steeper and have higher mathematically values compared to the other tested TCE molecules. Constructs having a higher surface hydrophobicity will have a stronger interaction with the matrix compared to constructs with less surface hydrophobicity and will therefore elute over a bigger elution volume with less curve steepness.

### Example: Evaluation of TCE Molecules Aggregation Temperature

Rising temperatures may destabilize protein constructs, which will expose structures originally buried by protein folding. These structures can be sticky and can get in contact with other constructs resulting in aggregation and therefore a larger hydrodynamic radius. Molecules having higher aggregations temperature are more stable compared to molecules having lower aggregation temperatures.

To determine aggregation temperature of scFab-containing TCE molecules, isolated and formulated TCE molecule monomer adjusted to a defined protein concentration is pipetted in duplicates into a 96-well plate and covered with paraffin oil. The 96-well plate is transferred to a dynamic light scattering DLS reader capable of heating the plate at a defined rate in a defined temperature range. Measurement is performed from 40° C. to 70° C. at a defined rate of temperature increase. Detection is done by dynamic light scattering determining the hydrodynamic radius of the constructs over the temperature ramp. Temperature at the beginning of increase of hydrodynamic radius is defined as aggregation temperature. “I2C” refers to an scFv that binds CD3. “CC” refers to an scFv containing an engineered cysteine clamp (cysteine clamp between a cysteine at position 44 (VH) and a cysteine at position 100 (VL) (Kabat)) that binds a target (i.e. MAGE-B2 in Table 11).

TABLE 11
DLS aggregation temperature of MAGE-B2 (“MA”) TCE molecules.

| | Mean Aggregation |
|---|---|
| TCE molecule | Temperature [° C.] |
| MA 03-D8 AS CC scFv × I2C × scFc | 53.57 |
| MA 03-D8 AS scFab × I2C × scFc | 57.86 |
| MA 03-E11 AS CC scFv × I2C × scFc | 54.51 |
| MA 03-E11 AS scFab× I2C × scFc | 57.96 |
| MA 09-E2 CC scFv × I2C × scFc | 52.09 |
| MA 09-E2 scFab × I2C × scFc | 57.83 |
| MA 09-F12 CC scFv × I2C × scFc | 53.45 |
| MA 09-F12 scFab × I2C × scFc | 58.44 |
| MA 09-G10 CC scFv × I2C × scFc | 49.84 |
| MA 09-G10 scFab × I2C × scFc | 58.56 |
| MA 09-H10 CC scFv × I2C × scFc | 55.14 |
| MA 09-H10 scFab × I2C × scFc | 55.42 |
| MA 09-H7 CC scFv × I2C × scFc | 53.11 |
| MA 09-H7 scFab × I2C × scFc | 58.64 |
| MA 10-D3 CC scFv × I2C × scFc | 52.67 |
| MA 10-D3 scFab × I2C × scFc | 58.01 |
| MA 10-D6 CC scFv × I2C × scFc | 52.19 |
| MA 10-D6 scFab × I2C × scFc | 58.23 |
| MA 10-G10 AS CC scFv × I2C × scFc | 54.11 |
| MA 10-G10 AS scFab × I2C × scFc | 57.79 |
| MA 86-A4-N-F5 CC scFv × I2C × scFc | 52.54 |
| MA 86-A4-N-F5 scFab × I2C × scFc | 58.47 |
| MA 88-B3-F9 CC scFv × I2C × scFc | 56.44 |
| MA 88-B3-F9 scFab × I2C × scFc | 57.7 |
| MA 98-C7 CC × I2C scFv × scFc | 56.96 |
| MA 98-C7 scFab × I2C × scFc | 57.17 |
| MA SG-F28 CC scFv × I2C × scFc | 53.36 |
| MA SG-F28 scFab × I2C × scFc | 58.48 |
| MA 10-B5 CC scFv × I2C × scFc | 52.6 |
| MA 10-B5 scFab × I2C × scFc | 58.22 |

TABLE 12
DLS aggregation temperature of CD20 and CD22
TCE molecules.

| | Mean |
|---|---|
| | Aggregation |
| | Temperature |
| TCE molecule | [° C.] |
| CD22 11-C3 scFab × CD20 29-F5 | 60.40 |
| scFab × I2C × scFc | |
| CD22 11-C3 CC scFv × CD20 29-F5 | 54.66 |
| CC scFv × I2C × scFc | |
| CD20 99-E5 scFab × CD22 28-B7 N655 | 56.24 |
| CC scFv × I2C × scFc | |
| CD20 99-E5 CC scFv CD22 28-B7 N655 | 56.15 |
| CC scFv × I2C × scFc | |

TABLE 13
DLS aggregation temperature of Claudin-6 TCE molecules.

| | | Mean Aggregation |
|---|---|---|
| | | Temperature |
| | TCE molecule (×I2C × scFc) | [° C.] |
| CL6 3C1 | HL CC scFv | 50.50 |
| | HL scFab | 55.98 |
| | 01 HL CC scFv | 54.64 |
| | 01 HL scFab | 56.77 |
| | 03 HL CC scFv | 51.72 |
| | 03 HL scFab | 56.45 |
| | 04 HL CC scFv | 49.84 |
| | 04 HL scFab | 56.48 |
| | 04.1 HL CC scFv | 50.73 |
| | 04.1 HL scFab | 56.37 |
| | 05 HL CC scFv | 49.33 |
| | 05 HL scFab | 56.29 |
| | 05.1 HL CC scFv | 51.77 |
| | 05.1 HL scFab | 55.94 |
| | 06 HL CC scFv | 50.03 |
| | 06 HL scFab | 55.94 |
| CL6 3D4 | 01.1 LH CC scFv | 51.61 |
| | 01.1 LH scFab | 56.62 |
| | 02 LH CC scFv | 52.76 |
| | 02 LH scFab | 55.91 |
| | 03.1 LH CC scFv | 49.60 |
| | 03.1 LH scFab | 56.54 |
| | 04 LH CC scFv | 53.38 |
| | 04 LH scFab | 57.25 |
| | 04.G2 LH CC scFv | 54.11 |
| | 04.G2 LH scFab | 56.29 |
| CL6 3C1 | 02 CC scFv | 54.79 |
| | 02 scFab | 57.26 |

These data demonstrate that scFab TCE molecules show higher mean aggregation temperatures compared to the other tested TCE molecules (having an scFv target binder) and are therefore more stable than TCE molecules with an scFv target binder.

### Example: Cell-based CCR8 Binding Competition Assay

Effects of biochemical competition with CCR8 ligand CCLI on CCR8-binding properties of CCR8-binding TCE molecules and scFab-containing CCR8-binding TCE molecules are assessed by flow cytometry, based on an engineered variant of the human T lymphocyte cell line HuT 78 expressing native CCR8 but not expressing human CD3 epsilon chain. The cell line bore a defined knockout in the CD3E gene.

Fifty thousand cells are incubated with 200 nM recombinant human CCL1 (Abcam, cat. no. ab9854) (diluted in PBS/2% FCS) or with PBS/2% FCS in 50 μl for 55 min at 4° C. Purified CCR8-binding TCE and scFab-containing CCR8-binding TCE molecules, diluted in PBS/2% FCS, are added to a final concentration of 100 nM in a total volume of 100 μl, followed by incubation at 4° C. for 45 minutes. For a negative control, PBS/2% FCS is added instead of purified CCR8-binding TCE or scFab-containing CCR8-binding TCE molecules. After three washes, bound molecules are detected for 45 minutes at 4° C. with R-Phycoerythrin-conjugated, anti-human IgG, Fc gamma fragment-specific antibody (Jackson, cat. no. 109-116-098), diluted 1:50 in PBS/2% FCS. After three washes, samples are measured on a FACSCanto II instrument (Becton Dickinson). All conditions are performed in three replicates.

Data are analyzed by FlowJo software (FlowJo/Becton Dickinson), generating median values for PE signals (median PE). From the three replicates per condition, average median PE values (with standard deviations) as well as ratios of average median PE in CCL1-treated setting over average median PE in CCL1-untreated setting were calculated using Excel software (Microsoft).

According to procedures essentially as described above, the following data were obtained.

Table 14 shows averages (with standard deviations) of median values of PE-signals, and ratios of average median PE of CCL1-treated condition over CCL1-untreated condition. Identifiers of CCR8-binding TCE molecules and scFab-containing CCR8-binding TCE molecules are indicated in the left column.

“I2E” represents an scFv that binds CD3. TCE1 TCE molecule (either scFab-12E-scFc or scFv CC×12E×scFc) amino acid sequences are given by SEQ ID NOs: 113-128. TCE8 TCE molecule (either scFab-12E-scFc or scFv CC×12E×scFc) amino acid sequences are given by SEQ ID NOs: 97-112. TCE2 TCE molecule (either scFab-12E-scFc or scFv CC×12E×scFc) amino acid sequences are given by SEQ ID NOs: 49-64.

TABLE 14
CCR8 TCE molecule binding in the presence or absence of CCL1.

| | Average | Average | Ratio |
|---|---|---|---|
| | Median PE, | Median PE, | Average Median PE |
| | CCL1-treated | CCL1-untreated | (+CCL1)/Average |
| TCE molecule | (+CCL1) | (−CCL1) | (−CCL1) |
| TCE1 scFab × I2E × scFc | 27047.33 ± 231.37 | 27174.00 ± 376.72 | 1.00 |
| TCE8 scFab × I2E × scFc | 8799.00 ± 197.89 | 18919.67 ± 250.31 | 0.47 |
| TCE2 scFab × I2E × scFc | 1554.33 ± 15.17 | 2424.33 ± 108.19 | 0.64 |
| TCE1 scFv CC × I2E × scFc | 23129.0 ± 189.7 | 23195.67 ± 368.24 | 1.00 |
| TCE8 scFv CC × I2E × scFc | 7346.00 ± 195.16 | 15940.33 ± 108.19 | 0.46 |
| Negative control | 416.67 ± 58.93 | 374.67 ± 2.49 | 1.11 |

These data demonstrate that the presence of CCLI did not affect binding of the CCR8-binding TCE molecule TCE1 or scFab-containing CCR8-binding TCE molecule TCE1. In contrast, CCL1 did block binding of TCE8 and TCE2 TCE molecules.

### Example: Evaluation of CCR8-Binding TCE Molecule and scFab-Containing CCR8-Binding TCE Molecule Surface Hydrophobicity

To determine surface hydrophobicity of scFab-containing CCR8 binding TCE molecules, isolated and formulated CCR8-binding TCE molecule and scFab-containing CCR8-binding TCE molecule monomer adjusted to a defined protein concentration are transferred into autosampler fitting sample vials and measured on a FPLC system. A Hydrophobic Interaction Chromatography (HIC) column is equilibrated with formulation buffer and a defined volume of protein solution applied at a constant formulation buffer flow. Detection is done by OD280 nm optical absorption. Elution behavior is determined by peak shape respectively mathematically calculation of declining signal peak slope. Steeper slope/higher slope values indicate less hydrophobic interaction of the protein surface compared to constructs with more flat elution behavior and lower slope value. Following procedures essentially as described above, the following data were obtained.

TCE4 TCE molecule (either scFab-12E-scFc or scFv CC×12E×scFc) amino acid sequences are given by SEQ ID NOs: 17-32. TCE1 TCE molecule (either scFab-12E-scFc or scFv CC×I2E×scFc) amino acid sequences are given by SEQ ID NOs: 113-128. TCE8 TCE molecule (either scFab-12E-scFc or scFv CC×12E×scFc) amino acid sequences are given by SEQ ID NOs: 97-112. TCE2 TCE molecule (either scFab-12E-scFc or scFv CC×12E×scFc) amino acid sequences are given by SEQ ID NOs: 49-64. TCE7 TCE molecule (either scFab-12E-scFc or scFv CC×12E×scFc) amino acid sequences are given by SEQ ID NOs: 81-96. TCE5 TCE molecule (either scFab-12E-scFc or scFv CC×12E×scFc) amino acid sequences are given by SEQ ID NOs: 33-48. TCE6 TCE molecule (either scFab-12E-scFc or scFv CC×12E×scFc) amino acid sequences are given by SEQ ID NOs: 65-80. “CC” denotes an engineered cysteine clamp between cysteines at residue 44 of the heavy chain variable region and residue 100 of the light chain variable region of the anti-target scFv.

TABLE 15
HIC elution slopes of CCR8-binding TCE molecules and
scFab-containing CCR8-binding TCE molecules.

| Construct | HIC Elution Slope |
|---|---|
| TCE4 scFab × I2E × scFc | 50.29 |
| TCE4 scFv CC × I2E × scFc | 8.95 |
| TCE1 scFab × I2E × scFc | 50.52 |
| TCE1 scFv CC × I2E × scFc | 34.61 |
| TCE3 scFab × I2E × scFc | 44.97 |
| TCE3 scFv CC × I2E × scFc | 2.50 |
| TCE8 scFab × I2E × scFc | 31.71 |
| TCE8 scFv CC × I2E × scFc | 11.61 |
| TCE2 scFab × I2E × scFc | 53.70 |
| TCE7 scFab × I2E × scFc | 28.59 |
| TCE7 scFv CC × I2E × scFc | 18.44 |
| TCE5 scFab × I2E × scFc | 11.65 |
| TCE5 scFv CC × I2E × scFc | 9.87 |
| TCE6 scFab × I2E × scFc | 8.37 |
| TCE6 scFv CC × I2E × scFc | 8.66 |

These data demonstrate HIC elution peak slopes for the scFab-containing CCR8-binding TCE molecules are steeper and have higher mathematically values compared to the CCR8-binding TCE molecules having an scFv that binds CCR8.

### Example: CCR8 TCE Molecule Affinity

Cell-based affinity of CCR8-binding TCE molecules and scFab-containing CCR8-binding TCE molecules is determined by nonlinear regression (one site-specific binding) analysis. CHO cells expressing human CCR8 (SEQ ID NO: 131), cynomolgus monkey CCR8 (SEQ ID NO: 129) or cynomolgus CCR8 (T4R; SEQ ID NO: 130) are incubated with decreasing concentrations of CCR8 bispecific constructs (50-3200 nM, step 1:2, 11 steps) for 16 hours at 4° C. Bound CCR8-binding TCE molecules and scFab-containing CCR8-binding TCE molecules are detected with Alexa Fluor 488-conjugated AffiniPure Fab Fragment Goat Anti-Human IgG (H+L). Fixed cells are stained with DRAQ5, Far-Red Fluorescent Live-Cell Permeant DNA Dye and signals are detected by fluorescence cytometry. Respective equilibrium dissociation constant (Kd) values are calculated with the one site specific binding evaluation tool of the GraphPad Prism software. Mean Kd values and affinity gaps are calculated with Microsoft Excel. Mean Kd values are calculated from two or three independent experiments. The affinity gaps are determined by dividing the cyno Kd by the human Kd. Following procedures essentially as described above, the following data were obtained.

TABLE 16
Cell-based affinities of CCR8-binding TCE molecules and scFab-containing
CCR8-binding TCE molecules.

| | Cell based | Cell based | Affinity | | Affinity gap |
|---|---|---|---|---|---|
| | affinity hu | affinity cyno | gap | Cell based affinity cy | Kdcy |
| | CCR8 Mean | CCR8 Mean Kd | Kdcy/Kdhu | CCR8 (T4R) Mean | (T4R)/Kdhu |
| TCE Molecule | Kd [nM] | [nM] | CCR8 | Kd [nM] | CCR8 |
| TCE4 scFab × I2E × scFc | 11.41 ± 5.34 | 456.40 ± 383.53 | 40.00 | 1263.70 ± 1582.93 | 110.75 |
| TCE4 scFv CC × I2E × scFc | 33.24 ± 7.95 | 795.73 ± 901.90 | 23.94 | 267.90 ± 369.48 | 8.06 |
| TCE1 scFab × I2E × scFc | 0.88 ± 0.080 | 1.58 ± 0.38 | 1.80 | 138.50 ± 66.19 | 157.39 |
| TCE1 scFv CC × I2E × scFc | 0.96 ± 0.26 | 2.00 ± 0.55 | 2.08 | 186.21 ± 258.79 | 193.97 |
| TCE3 scFab × I2E × scFc | 12.84 ± 4.89 | 271.11 ± 219.01 | 21.11 | 5.96E+17 ± 1.03E+18 | 4.64E+16 |
| TCE3 scFv CC × I2E × scFc | 6.86 ± 2.47 | 704.62 ± 740.47 | 102.71 | 142.08 ± 110.91 | 20.71 |
| TCE8 scFab × I2E × scFc | 0.59 ± 0.18 | 442.57 ± 361.67 | 750.12 | 790.07 ± 399.5 | 1339.10 |
| TCE8 scFv CC × I2E × scFc | 2.20 ± 0.91 | 200.83 ± 157.75 | 91.29 | 305.07 ± 243.03 | 138.67 |
| TCE7 scFab × I2E × scFc | 0.88 ± 0.59 | 307.98 ± 238.88 | 349.98 | 706.15 ± 266.51 | 802.44 |
| TCE7 scFv CC × I2E × scFc | 1.13 ± 0.48 | 780.06 ± 831.30 | 690.32 | 322.55 ± 49.71 | 285.44 |
| TCE5 scFab × I2E × scFc | 0.83 ± 0.49 | 234.80 ± 94.47 | 282.89 | 197.45 ± 4.17 | 237.89 |
| TCE5 scFv CC × I2E × scFc | 0.63 ± 0.15 | 342.43 ± 253.83 | 543.54 | 275.10 ± 30.55 | 436.67 |
| TCE6 scFab × I2E × scFc | 0.88 ± 0.20 | 305.84 ± 241.93 | 347.55 | 310.10 ± 184.98 | 352.39 |
| TCE6 scFv CC × I2E × scFc | 0.61 ± 0.060 | 731.77 ± 530.08 | 1199.62 | 374.45 ± 188.59 | 613.85 |
| TCE2 scFab × I2E × scFc | 21.95 ± 1.76 | 551.74 ± 446.63 | 25.14 | 711.83 ± 710.91 | 32.43 |

These cell-based affinity measurements demonstrate that TCE molecules such as TCE1, with or without an scFab, have high affinity for both human CCR8 and cynomolgus monkey CCR8 without the T4R mutation. The affinity of the TCE1 TCE molecules was reduced against cynomolgus monkey cells having the T4R mutation.

In addition, the TCE molecule CCR8 TCE2 was not able to be produced in a sufficient amount. However, the TCE molecule having an scFab moiety (CCR8 TCE2 scFab) was able to be produced, demonstrating that the scFab moiety provides an advantage for molecule production.

### Example: Evaluation of CCR8-Binding TCE Molecule and scFab-Containing CCR8-Binding TCE Molecule Aggregation Temperature

To measure aggregation temperature, isolated and formulated CCR8-binding TCE molecule and scFab-containing CCR8-binding TCE molecule monomer adjusted to a defined protein concentration are pipetted in duplicates into a 96-well plate and overlaid with paraffin oil. The 96-well plate is transferred to a dynamic light scattering DLS reader capable of heating the plate at a defined rate in a given temperature range. Measurement is performed from 40° C. to 70° C. at a defined rate of temperature increase. Detection is done by dynamic light scattering determining the hydrodynamic radius of the constructs over the temperature ramp. The temperature at begin of increase of hydrodynamic radius is defined as aggregation temperature. Following procedures essentially as described above, the following data were obtained.

TABLE 17
DLS aggregation temperature of CCR8 TCE molecules.

| | Aggregation temperature |
|---|---|
| TCE Molecule | [° C.] |
| TCE4 scFab × I2E × scFc | 60.18 |
| TCE4 scFv CC × I2E × scFc | 56.83 |
| TCE1 scFab × I2E × scFc | 58.20 |
| TCE1 scFv CC × I2E × scFc | 53.60 |
| TCE3 scFab × I2E × scFc | 55.90 |
| TCE3 scFv CC × I2E × scFc | 48.01 |
| TCE8 scFab × I2E × scFc | 58.44 |
| TCE8 scFv CC × I2E × scFc | 53.82 |
| TCE7 scFab × I2E × scFc | 60.33 |
| TCE7 scFv CC × I2E × scFc | 49.92 |
| TCE5 scFab × I2E × scFc | 60.29 |
| TCE5 scFv CC × I2E × scFc | 46.25 |
| TCE6 scFab × I2E × scFc | 59.89 |
| TCE6 scFv CC × I2E × scFc | 53.27 |
| TCE2 scFab × I2E × scFc | 54.79 |

According to procedures essentially as described above, scFab-containing CCR8-binding TCE molecules demonstrated higher aggregation temperatures compared to the CCR8-binding TCE molecules having an scFv that binds CCR8. As described above, the TCE molecule CCR8 TCE2 was not able to be produced in a sufficient amount, whereas the TCE molecule having an scFab moiety (CCR8 TCE2 scFab) was able to be produced.

### Example: CCR8 Molecule Cytotoxicity Assay with Unstimulated Human PBMC

Human peripheral blood mononuclear cells (PBMC) are prepared by Ficoll density gradient centrifugation from enriched lymphocyte preparations (buffy coats), a side product of blood banks collecting blood for transfusions. Buffy coats are supplied by a local blood bank and PBMC are prepared on the same day of blood collection. After Ficoll density centrifugation and extensive washes with Dulbecco's PBS (Gibco), remaining erythrocytes are removed from PBMC via incubation with erythrocyte lysis buffer (155 mM NH₄Cl, 10 mM KHCO₃, 100 μM EDTA). Platelets are removed via the supernatant upon centrifugation of PBMC at 100×g. Remaining lymphocytes mainly encompass B and T lymphocytes, NK cells and monocytes. PBMC are kept in culture at 37° C./5% CO₂ in RPMI medium (Gibco) with 10% FCS (Gibco).

For depletion of CD14+ cells, human CD14 MicroBeads (Milteny Biotec, MACS, #130-050-201) are used. For depletion of NK cells, human CD56 MicroBeads (MACS, #130-050-401) are used. PBMC are counted and centrifuged for 10 minutes at room temperature with 300×g. The supernatant is discarded, and the cell pellet resuspended in MACS isolation buffer [80 μL/10⁷ cells; PBS (Invitrogen, #20012-043), 0.5% (v/v) FBS (Gibco, #10270-106), 2 mM EDTA (Sigma-Aldrich, #E-6511)]. CD14 MicroBeads and CD56 MicroBeads (20 μL/10⁷ cells) are added and incubated for 15 minutes at 4 to 8° C. The cells are washed with MACS isolation buffer (1-2 mL/10⁷ cells). After centrifugation (see above), supernatant is discarded, and cells are resuspended in MACS isolation buffer (500 μL/108 cells). CD14/CD56 negative cells are isolated using LS Columns (Miltenyi Biotec, #130-042-401). PBMC without CD14+/CD56+ cells are cultured in RPMI complete medium i.e. RPMI1640 (Biochrom AG, #FG1215) supplemented with 10% FBS (Biochrom AG, #S0115), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C.

For the analysis of cell lysis in flow cytometry assays, the fluorescent membrane dye DiOC₁₈ (DiO) (Molecular Probes, #V22886) is used to label human CCR8- or macaque CCR8-transfected CHO cells as target cells and distinguish them from effector cells. Briefly, cells are harvested, washed once with PBS and adjusted to 106 cell/mL in PBS containing 2% (v/v) FBS and the membrane dye DiO (5 μL/10⁶ cells). After incubation for 3 minutes at 37° C., cells are washed twice in complete RPMI medium and the cell number adjusted to 1.25×10⁵ cells/mL. The vitality of cells is determined using the NC-250 cell counter (Chemometec)

To quantify the lysis of cyno or human CCR8-transfected CHO cells in the presence of serial dilutions of CCR8-binding TCE molecule or scFab-containing CCR8-binding TCE molecule, equal volumes of DiO-labeled target cells and effector cells (i.e., PBMC w/o CD14⁺ cells) are mixed, resulting in an E:T cell ratio of 10:1. 80 μl of this suspension is transferred to each well of a 96-well plate. 20 μL of serial dilutions of the CCR8-binding TCE molecule or scFab-containing CCR8-binding TCE molecule and a negative control (a CD3-based TCE molecule recognizing an irrelevant target antigen) or RPMI complete medium as an additional negative control are added. The TCE molecule or scFab-containing TCE molecule-mediated cytotoxic reaction proceeded for 48 hours in a 7% CO₂ humidified incubator. Cells are transferred to a new 96-well plate and loss of target cell membrane integrity is monitored by adding propidium iodide (PI) at a final concentration of 1 μg/mL. PI is a membrane impermeable dye that normally is excluded from viable cells, whereas dead cells take it up and become identifiable by fluorescent emission.

Samples are measured by flow cytometry on an iQue Plus instrument and analyzed by Forecyt software (both from Intellicyt). Target cells are identified as DiO-positive cells. PI-negative target cells are classified as living target cells. Percentage of cytotoxicity is calculated as number of dead targets cells/number of target cells×100. Using GraphPad Prism 5 software (Graph Pad Software, San Diego), the percentage of cytotoxicity is plotted against the corresponding TCE molecule or scFab-containing TCE molecule concentrations. Dose response curves are analyzed with the four parametric logistic regression models for evaluation of sigmoid dose response curves with fixed hill slope and EC₅₀ values are calculated.

Following procedures essentially as described above, data from a 48-hour FACS-based cytotoxicity assay of scFab-containing CCR8-binding TCE molecule with human CCR8 transfected CHO cells (clone #A2) and human CCR8 isoform A27G (SEQ ID NO: 132) transfected CHO cells as target cells and unstimulated human PBMC (CD14−/CD56−) as effector cells (E:T ratio 10:1) is shown below in Table 18.

TABLE 18
48-hour FACS based cytotoxicity assay of
scFab-containing CCR8-binding TCE molecules.

| | #A2 EC₅₀ | A27G EC₅₀ |
|---|---|---|
| TCE Molecule | [pM] | [pM] |
| CCR8 TCE1 scFab × | 14.39 | 15.68 |
| I2E × scFc | | |
| CCR8 TCE8 scFab × | 7.26 | 5.93 |
| I2E × scFc | | |
| CCR8 TCE2 scFab × | 290 | 342 |
| I2E × scFc | | |

These data demonstrate that binder TCE1 and TCE8 show comparable bioactivity in a one-to two-digit pM range.

Data from a 48-hour FACS-based cytotoxicity assay of bispecific constructs with Chinese cynomolgus CCR8 (SEQ ID NO: 129) transfected CHO cells (clone #G4) and the mauritian cyno CCR8 isoform T4R (SEQ ID NO: 130) transfected CHO cells as target cells and unstimulated human PBMC (CD14−/CD56−) as effector cells (E:T ratio 10:1) is shown below in Table 19.

TABLE 19
48-hour FACS based cytotoxicity assay of CCR8
scFab-containing CCR8-binding TCE molecules.

| | #G4 EC₅₀ | T4R EC₅₀ |
|---|---|---|
| TCE Molecule | [pM] | [pM] |
| CCR8 TCE1 | 3.91 | X |
| scFab × I2E × scFc | | |
| CCR8 TCE8 | X | X |
| scFab × I2E × scFc | | |
| CCR8 TCE2 | X | X |
| scFab × I2E × scFc | | |

These data demonstrate that only the tested TCE1 TCE molecule (having a scFab targeting CCR8) shows pM bioactivity on the chinese cynomolgus monkey CCR8 transfected CHO cell line. The other tested molecules demonstrated no activity (as depicted by “X” in the table).

Data from a 48-hour FACS-based cytotoxicity assay of CCR8-binding TCE molecule and scFab-containing CCR8-binding TCE molecule with HUT-78 (CD3+) as target cells and unstimulated human PBMC (CD14−/CD56−) as effector cells (E:T ratio 10:1) is shown below in Table 20. EC₅₀ values are determined by the four parametric logistic regression models for evaluation of sigmoid dose response curves with fixed hill slope.

TABLE 20
48-hour FACS based cytotoxicity assay of CCR8-binding TCE
molecules and scFab-containing CCR8-binding TCE molecules.

| TCE Molecule | EC₅₀ [pM] |
|---|---|
| CCR8 TCE4 scFab × I2E × scFc | 1246 |
| CCR8 TCE4 scFv × I2E × scFc | 265 |
| CCR8 TCE1 scFab × I2E × scFc | 46.9 |
| CCR8 TCE1 scFv × I2E × scFc | 58.2 |
| CCR8 TCE3 scFab × I2E × scFc | 478 |
| CCR8 TCE3 scFv × I2E × scFc | 28112 |
| CCR8 TCE8 scFab × I2E × scFc | 416 |
| CCR8 TCE8 scFv × I2E × scFc | 1565 |
| CCR8 TCE5 scFab × I2E × scFc | 506 |
| CCR8 TCE5 scFv × I2E × scFc | 2695 |
| CCR8 TCE7 scFab × I2E × scFc | 413 |
| CCR8 TCE7 scFv × I2E × scFc | 3269 |
| CCR8 TCE6 scFab × I2E × scFc | 576 |
| CCR8 TCE6 scFv × I2E × scFc | 354 |
| CCR8 TCE2 scFab × I2E × scFc | 3510 |

These data demonstrate that binder TCE1 shows high bioactivity on the endogenous cell line HUT-78 (CD3+).

### Example: Luciferase-Based Cytotoxicity Assay With Unstimulated Human PBMC

Isolation of effector cells and depletion of CD14⁺ and CD56⁺ cells are performed as described above. Target cells (described below) are harvested, spun down, and adjusted to 1.2×10⁵ cells/mL in complete RPMI medium. The vitality of cells is determined using Nucleocounter NC-250 (Chemometec) and Solution18 Dye containing Acridine Orange and DAPI (Chemometec).

To quantify the lysis of target cells in the presence of serial dilutions of CCR8-binding TCE molecules or scFab-containing CCR8-binding TCE molecules, equal volumes of luciferase-positive target cells and effector cells (i.e., PBMC w/o CD14⁺; CD56⁺ cells) are mixed, resulting in an E:T cell ratio of 10:1. 42 μL of this suspension is transferred to each well of a 384-well plate. 8 μL of serial dilutions of the corresponding CCR8-binding TCE molecule or scFab-containing CCR8-binding TCE molecule and a negative control (a CD3-based TCE molecule recognizing an irrelevant target antigen) or RPMI complete medium as an additional negative control are added. The TCE molecule cytotoxic reaction proceeds for 48 hours in a 5% CO₂ humidified incubator. Then, 25 μL substrate (Steady-Glo® Reagent, Promega) is transferred to the 384-well plate. Only living luciferase-positive cells react to the substrate and create a luminescence signal. Samples are measured with a SPARK microplate reader (TECAN) and analyzed by Spark Control Magellan software (TECAN). Percentage of cytotoxicity was calculated as (1-RLU_{Sample}/RLU_{Negative-Control})×100. RLU mean relative light unites. “Negative-Control” means cells without TCE molecule.

Using GraphPad Prism 7.04 software (Graph Pad Software, San Diego), the percentage of cytotoxicity is plotted against the corresponding multi-specific TCE molecule concentrations. Dose response curves are analyzed with the four parametric logistic regression models for evaluation of sigmoid dose response curves with fixed hill slope and EC₅₀ values are calculated.

Following procedures essentially as described above, the following data were obtained. Data shown are of CCR8 scFab-containing CCR8-binding TCE molecules against the human CCR8 positive HUT-78 CD3ε-ko cell line (parental cells and clones shown below) or the human CCR8 negative (ko) HUT-78 (CD3ε+) cell line (monoplex clone 2E3; negative control) as target cells, and unstimulated human PBMC (CD14−/CD56−) as effector cells (E:T ratio 10:1).

TABLE 21
48-hour Luciferase-based cytotoxicity assay of CCR8
scFab-containing CCR8-binding TCE molecules.

| | | | | | CCR8 |
|---|---|---|---|---|---|
| | CCR8 positive HUT-78 | | | | negative |
| | Parental | | | | HUT-78 |
| | cells | Monoplex | Monoplex | Monoplex | (CD3ε+) |
| | CCR8+ | clone 2B3 | clone 2E9 | clone 1F9 | Negative |
| | CD3e+ | CD3e− | CD3e− | CD3e− | control |
| Construct | EC₅₀ | | | | |
| TCE1 scFab × | 6.6 | 4.8 | 2.4 | 3.6 | X |
| I2E × scFc | | | | | |
| TCE8 scFab × | 93.1 | 50.7 | 32.6 | 40.1 | X |
| I2E × scFc | | | | | |
| TCE2 scFab × | 4279.4 | 943.3 | 1036.8 | 998.4 | X |
| I2E × scFc | | | | | |

These data demonstrate that the TCE1 scFab-containing TCE molecule shows a superior bioactivity on the human CCR8 positive HUT-78 (CD3ε−) cell lines compared to TCE8 and TCE2.

### Example: Bispecific Binding and Interspecies Cross-Reactivity

For confirmation of binding to human CCR8 and CD3 and to cyno CCR8 and CD3, TCE1 scFab-containing CCR8-binding TCE molecule, control TCE molecule (CD3-based TCE molecule recognizing an irrelevant target antigen), or anti-CCR8 antibodies clone L263G8 (BioLegend) and 433H (BD) are tested by flow cytometry using CHO cells transfected with human CCR8 and or macaque CCR8, the human CCR8 and CD3 positive human cell line HUT-78, the human CCR8 positive and CD3 negative HUT-78 cell line, CD3-expressing human T cell leukemia cell line HPB-all (DSMZ, Braunschweig, ACC483), and the cynomolgus CD3-expressing T cell line LnPx 4119.

Following procedures essentially as described above, the following data were obtained. A “-” indicates that no signal was detected. Data represent mean BL2 (channel in which the signal was detected) of the sample/mean BL2 of the secondary antibody control.

TABLE 22
Binding of TCE molecules to human and
cynomolgus monkey CCR8 and CD3.

| | CCR8 | | Anti | Anti |
|---|---|---|---|---|
| | TCE1 | Control | CCR8 mAb | CCR8 |
| | scFab × | TCE | (Clone | mAb |
| Cell line | I2E × scFc | molecule | L263G8) | (433H) |
| hu CCR8+ CHO | 42 | 1 | 40 | — |
| Cy CCR8+ CHO | 196 | 1 | — | 136 |
| Cy CCR8 (T4R)+ CHO | 1 | 1 | — | 106 |
| Parental CHO | 1 | 1 | — | — |
| HUT-78 (CCR8+/CD3+) | 62 | 72 | 9 | — |
| HUT-78 (CCR8+/CD3−) | 30 | 1 | 6 | — |
| HPB-all | 298 | 398 | — | — |
| LnPx4119 | 33 | 75 | — | — |

These data demonstrate that the scFab-containing TCE1 TCE molecule bound human CCR8 and cynomolgus monkey CCR8 in human or cyno expressing CHO cells but did not bind the cynomolgus monkey CCR8 having a T4R mutation. These data also demonstrate that TCE1 binds CCR8 expressed on a naturally-expressing CCR8 cell line (HUT-78 CCR8+/CD3e−).

### Example: Epitope Clustering of CCR8 TCE Molecules

The extracellular domain of human CCR8 comprises three loops and a N-terminal peptide of 35 amino acids. For epitope mapping, the N-terminal peptide of human CCR8 (designated P_1-35 (SEQ ID NO: 133)) is divided into three consecutive segments (designated P_1-12 (SEQ ID NO: 134), P_13-24 (SEQ ID NO: 135), P_25-35 (SEQ ID NO: 136)). To cover the adjacent N- or C-terminal regions of the consecutive segments two additional overlapping fragments (designated P_7-18 (SEQ ID NO: 137 and P_19-30 (SEQ ID NO: 138)) are made. At the C-terminal end of the full-length N-terminal peptide and all truncated N-terminal peptides of human CCR8 described above a V5 tag is fused via a G4S-linker. Following the V5 tag, chicken albumin is fused via a further G4S-linker followed by a FLAG tag, BAP (biotin acceptor protein) for in vivo biotinylation, and H3G, each fused via a SG-linker. All constructs described above are cloned into a pEFDHFR vector and transiently transfected into HEK 293 cells.

HEK 293 cells (1×10E8) are resuspended in 100 ml FreeStyle expression medium (Gibco 12338-018) and transfected with 4 ml OptiMEM (Gibco 31985-047), 100 μl 293fectin (Invitrogen 12347-019) and 50 μg DNA encoding either the full-length or truncated N-terminal CCR8 constructs according to the manufacturers protocol. Cells are grown in FreeStyle expression medium for 72 hours at 130 rpm in a humidified incubator with 8% CO₂. Cells are centrifuged at 1,500 rpm for 10 minutes and the supernatant is harvested. 10 ml of the supernatant of each of the transfected cells or 9 ml of HEK 293 cells as negative control are 20× concentrated with Amicon Ultra-15 tubes (UFC901008) to 500 μL. For each of the full-length and truncated N-terminal CCR8 constructs as well as HEK 293 negative control, 18×1086 washed streptavidin-beads (Streptavidin Microspheres, 6 μm; Polysciences 24172-1) are resuspended in 500 μL of the concentrated supernatant and incubated slowly shaking for one hour. Beads coupled with the respective antigen or negative control are washed and stored at 4° C. overnight.

To verify expression and binding of the full-length and truncated N-terminal CCR8 constructs to streptavidin-beads, 2×10F5 beads per staining are incubated with 5 μg/mL of an anti-FLAG antibody (clone M2, Sigma F3165/F1804) and 5 μg/mL of an anti-V5 antibody (clone SV5-Pk1; AbD Serotec, MCA 1360) and a 1:100 dilution of PE-labeled anti mouse Fcy secondary antibody (Jackson 115-116-071). Antigen-bound beads are incubated with three different anti-human CCR8 antibodies. Binding of two of the anti-human CCR8 antibodies (clone L263G8; BioLegend, 360602 and clone 433H; BD 747578; 5 μg/ml each) is detected with a 1:100 dilution of a PE-labeled anti mouse Fcy secondary antibody (Jackson 115-116-071). Binding of anti-human CCR8 antibody (polyclonal; Abcam, ab140796) is detected with a 1:50 dilution of PE-labeled anti goat Fcy secondary antibody (Jackson 109-116-098).

To evaluate binding of CCR8-binding TCE molecules and scFab-containing CCR8-binding TCE molecules to the full-length and truncated N-terminal CCR8 constructs bound to streptavidin-beads, beads are incubated with 5 μg/mL of the respective TCE molecule. Binding of these CCR8-binding TCE molecules and scFab-containing CCR8-binding TCE molecules is detected using 2 μg/ml of an anti-Histidin-antibody (clone AD1.1.10; AbD Serotec MCA 1396) and a 1:100 dilution of a PE-labeled anti mouse Fcy secondary antibody (Jackson 115-116-071). All antibodies, CCR8-binding TCE molecules, and scFab-containing CCR8-binding TCE molecules are diluted in PBS with 2% FBS and all incubations are performed at 4° C. for 45 minutes (primary antibodies) or for 30 minutes (secondary antibodies). Washes are performed using PBS with 2% FBS and the final suspension buffer prior to FACS analysis is also PBS with 2% FBS. Antibody and TCE binding is detected using an Intellicyte IQue. Changes in mean fluorescence are analyzed with an Intellicyte IQue and FlowJo. Binding to the various full-length and truncated N-terminal CCR8 constructs is reflected as a positive signal detected by flow cytometry.

Following procedures essentially as described above, expression and binding of the full-length and the various truncated N-terminal CCR8 constructs to streptavidin-beads could be verified by flow cytometry.

TABLE 23a
Flow Cytometry Binding Analysis of CCR8 Antibodies to Full-length
or Truncated N-terminal Peptides of Human CCR8.

| | Peptide or controls | | | | | | | |
|---|---|---|---|---|---|---|---|---|
| | Beads | HEK | | | | | | |
| | (control) | (control) | 1-35 | 1-12 | 7-18 | 13-24 | 19-30 | 25-35 |
| | Median Fluorescence of Sample/Median | | | | | | | |
| Sample | Fluorescence of Negative Control | | | | | | | |
| PBS | 0.9 | 0.9 | 0.9 | 0.9 | 0.9 | 0.9 | 0.9 | 1.9 |
| Flag | 0.9 | 0.9 | 522.0 | 596.0 | 588.7 | 628.1 | 589.5 | 582.0 |
| V5 Tag | 0.9 | 0.9 | 952.6 | 1091.5 | 1085.9 | 1303.2 | 1016.2 | 1094.2 |
| anti-CCR8 | 1.2 | 0.9 | 751.2 | 0.9 | 0.9 | 284.8 | 0.9 | 0.9 |
| (clone | | | | | | | | |
| L263G8) | | | | | | | | |
| anti-CCR8 | 1.8 | 0.9 | 290.2 | 0.9 | 0.9 | 300.3 | 0.9 | 0.9 |
| (clone | | | | | | | | |
| BV510) | | | | | | | | |
| anti-CCR8 | 0.9 | 1.0 | 259.7 | 0.9 | 222.6 | 385.6 | 0.9 | 0.9 |
| (polyclonal) | | | | | | | | |

These data demonstrate that anti-human CCR8 antibodies bound the full-length N-terminal peptide of human CCR8 P_1-35 indicating they recognized the N-terminal peptide of human CCR8. None of the antibodies showed binding to either streptavidin-beads alone or to the HEK 293 control. The anti-human CCR8 antibodies (clone L263G8 and clone 433H) showed the same binding pattern while the polyclonal anti-human CCR8 antibody showed additional binding to the overlapping fragment P_7-18.

As shown in Table 23, when binding to the truncated N-terminal peptides of CCR8 was evaluated, CCR8-binding TCE molecules and scFab-containing CCR8-binding TCE molecules showed two different binding patterns. For example, TCE4, TCE3 and TCE8 each bound to the truncated N-terminal peptide P_13-24. However, TCE1 bound to the truncated N-terminal peptide P_1-12.

TABLE 23b
Flow Cytometry Binding Analysis of CCR8 Antibodies and
scFab-containing CCR8-binding TCE molecules to
Full-length or Truncated N-terminal Peptides of Human CCR8.

| | Peptide or controls | | | | | | | |
|---|---|---|---|---|---|---|---|---|
| | Beads | HEK | | | | | | |
| | (control) | (control) | 1-35 | 1-12 | 7-18 | 13-24 | 19-30 | 25-35 |
| | Median Fluorescence of Sample/Median | | | | | | | |
| Construct | Fluorescence of Negative Control | | | | | | | |
| PBS | 1.0 | 1.0 | 1.0 | 1.0 | 1.0 | 1.0 | 1.0 | 1.0 |
| CCR8 TCE1 | 1.2 | 1.1 | 470.8 | 413.5 | 1.0 | 1.1 | 1.1 | 1.1 |
| scFab x 12E x | | | | | | | | |
| scFc | | | | | | | | |
| CCR8 TCE1 | 1.1 | 1.0 | 381.8 | 306.2 | 1.0 | 1.1 | 1.1 | 1.0 |
| scFv x I2E x | | | | | | | | |
| scFc | | | | | | | | |
| CCR8 TCE4 | 1.0 | 1.0 | 1167.5 | 1.0 | 1.0 | 995.6 | 1.1 | 1.1 |
| scFab x I2E x | | | | | | | | |
| scFc | | | | | | | | |
| CCR8 TCE4 | 1.0 | 1.0 | 842.6 | 1.0 | 1.0 | 745.5 | 1.1 | 1.1 |
| scFv x 12E x | | | | | | | | |
| scFc | | | | | | | | |
| CCR8 TCE3 | 1.0 | 1.0 | 1281.6 | 1.0 | 1.0 | 1090.0 | 1.1 | 1.1 |
| scFab x I2E x | | | | | | | | |
| scFc | | | | | | | | |
| CCR8 TCE3 | 1.0 | 1.0 | 1265.9 | 1.1 | 1.0 | 1041.4 | 1.1 | 1.1 |
| scFv x I2E x | | | | | | | | |
| scFc | | | | | | | | |
| CCR8 TCE8 | 1.1 | 1.0 | 1298.3 | 1.0 | 1.0 | 435.6 | 1.1 | 1.1 |
| scFab x I2E x | | | | | | | | |
| scFc | | | | | | | | |
| CCR8 TCE8 | 1.0 | 1.0 | 1081.1 | 1.0 | 1.0 | 381.4 | 1.1 | 1.0 |
| scFv x 12E x | | | | | | | | |
| scFc | | | | | | | | |
| CCR8 TCE5 | 1.1 | 1.0 | 1370.6 | 1.0 | 1.0 | 500.4 | 1.1 | 1.1 |
| scFab x 12E x | | | | | | | | |
| scFc | | | | | | | | |
| CCR8 TCE5 | 1.1 | 1.0 | 1402.0 | 1.0 | 1.0 | 525.6 | 1.1 | 1.2 |
| scFv x 12E x | | | | | | | | |
| scFc | | | | | | | | |
| CCR8 TCE6 | 1.1 | 1.0 | 1289.8 | 1.0 | 1.0 | 441.6 | 1.0 | 1.1 |
| scFab x I2E x | | | | | | | | |
| scFc | | | | | | | | |
| CCR8 TCE6 | 1.5 | 1.1 | 1360.9 | 1.0 | 1.0 | 445.1 | 1.1 | 1.1 |
| scFv x 12E x | | | | | | | | |
| scFc | | | | | | | | |
| CCR8 TCE7 | 1.0 | 1.0 | 1578.8 | 1.0 | 1.0 | 536.0 | 1.1 | 1.1 |
| scFab x 12E x scFc | | | | | | | | |
| CCR8 TCE7 | 1.1 | 1.0 | 1409.5 | 1.0 | 1.0 | 539.5 | 1.1 | 1.1 |
| scFv x I2E x | | | | | | | | |
| scFc | | | | | | | | |
| CCR8 TCE2 | 1.0 | 1.0 | 814.0 | 1.0 | 1.0 | 432.5 | 1.1 | 1.0 |
| scFab x 12E x scFc | | | | | | | | |
| Negative | 1.0 | 1.0 | 1.0 | 1.0 | 1.0 | 1.0 | 1.0 | 1.1 |
| control | | | | | | | | |

These data demonstrate that CCR8-binding TCE molecules and scFab-containing CCR8-binding TCE molecules bound to the full-length N-terminal CCR8 peptide P_1-35. All molecules except TCE1 bound to the truncated N-terminal CCR8 peptide P_13-24. Interestingly, TCE1 binds to the truncated N-terminal CCR8 peptide P_1-12, suggesting TCE1 binds a unique epitope on CCR8, which is thought to contribute to the high affinity and bioactivity of TCE1.

Additional scFvs representing a new sequence family that binds in the 1-12 amino acid epitope cluster have been generated and screened by phage display.

SEQUENCES
TCE3 CCR8 scFv and scFab HCDR1
(SEQ ID NO: 1)
SYVMH
TCE3 CCR8 scFv and scFab HCDR2
(SEQ ID NO: 2)
VISYDGSSQYYTDSVKG
TCE3 CCR8 scFv and scFab HCDR3
(SEQ ID NO: 3)
GRLATAILFDY
TCE3 CCR8 scFv and scFab LCDR1
(SEQ ID NO: 4)
KSSQSLLYSDGKTYLF
TCE3 CCR8 scFv and scFab LCDR2
(SEQ ID NO: 5)
EVSNRFS
TCE3 CCR8 scFv and scFab LCDR3
(SEQ ID NO: 6)
MQSIKLPLT
TCE3 CCR8 scFv VH
(SEQ ID NO: 7)
QVQLVESGGGVVQPGRSLRLSCEASGFTFSSYVMHWVRQAPGKCLEWVSVISYDGSSQYYTDS
VKGRFTISRDNSKNTLNLQMNSLRAEDTAVYYCVRGRLATAILFDYWGQGTLVTVSS
TCE3 CCR8 scFv VL
(SEQ ID NO: 8)
EILMTQTPLSLSVTPGQPASISCKSSQSLLYSDGKTYLFWYLQRPGQPPQLLIYEVSNRFSGV
PDRFSGSGSGTDFTLKISRVEAEDVGIYYCMQSIKLPLTFGCGTKVEIK
TCE3 CCR8 scFv
(SEQ ID NO: 9)
QVQLVESGGGVVQPGRSLRLSCEASGFTFSSYVMHWVRQAPGKCLEWVSVISYDGSSQYYTDS
VKGRFTISRDNSKNTLNLQMNSLRAEDTAVYYCVRGRLATAILFDYWGQGTLVTVSSGGGGQG
GGGQGGGGQEILMTQTPLSLSVTPGQPASISCKSSQSLLYSDGKTYLFWYLQRPGQPPQLLIY
EVSNRFSGVPDRFSGSGSGTDFTLKISRVEAEDVGIYYCMQSIKLPLTFGCGTKVEIK
TCE3 scFv (CCR8) x scFv (CD3) TCE
(SEQ ID NO: 10)
QVQLVESGGGVVQPGRSLRLSCEASGFTFSSYVMHWVRQAPGKCLEWVSVISYDGSSQYYTDS
VKGRFTISRDNSKNTLNLQMNSLRAEDTAVYYCVRGRLATAILFDYWGQGTLVTVSSGGGGQG
GGGQGGGGQEILMTQTPLSLSVTPGQPASISCKSSQSLLYSDGKTYLFWYLQRPGQPPQLLIY
EVSNRFSGVPDRFSGSGSGTDFTLKISRVEAEDVGIYYCMQSIKLPLTFGCGTKVEIKSGGGG
QEVQLVESGGGLVQPGGSLKLSCAASGFTFNKYAINWVRQAPGKGLEWVARIRSKYNNYATYY
ADAVKDRFTISRDDSKNTVYLQMNNLKTEDTAVYYCARAGNFGSSYISYWAYWGQGTLVTVSS
GGGGQGGGGQGGGGQQTVVTQEPSLTVSPGGTVTITCGSSTGAVTSGNYPNWVQKKPGQAPRG
LIGGTKFLAPGTPARFSGSLSGGKAALTLSGVQPEDEAEYYCVLWYSNRWVFGSGTKLTVL
TCE3 scFv (CCR8) x scFv (CD3) x scFc
(SEQ ID NO: 11)
QVQLVESGGGVVQPGRSLRLSCEASGFTFSSYVMHWVRQAPGKCLEWVSVISYDGSSQYYTDS
VKGRFTISRDNSKNTLNLQMNSLRAEDTAVYYCVRGRLATAILFDYWGQGTLVTVSSGGGGQG
GGGQGGGGQEILMTQTPLSLSVTPGQPASISCKSSQSLLYSDGKTYLFWYLQRPGQPPQLLIY
EVSNRFSGVPDRFSGSGSGTDFTLKISRVEAEDVGIYYCMQSIKLPLTFGCGTKVEIKSGGGG
QEVQLVESGGGLVQPGGSLKLSCAASGFTFNKYAINWVRQAPGKGLEWVARIRSKYNNYATYY
ADAVKDRFTISRDDSKNTVYLQMNNLKTEDTAVYYCARAGNFGSSYISYWAYWGQGTLVTVSS
GGGGQGGGGQGGGGQQTVVTQEPSLTVSPGGTVTITCGSSTGAVTSGNYPNWVQKKPGQAPRG
LIGGTKFLAPGTPARFSGSLSGGKAALTLSGVQPEDEAEYYCVLWYSNRWVFGSGTKLTVLGG
GGCPPCPAPELLGGPSVFLFPPKPKDTLMISRTPEVTCVVVDVSHEEPEVKFNWYVDGVEVHN
AKTKPCEEQYGSTYRCVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGKGGGGQGGGGQGGGGQGGGGQGGGGQGGG
GQCPPCPAPELLGGPSVFLFPPKPKDTLMISRTPEVTCVVVDVSHEEPEVKFNWYVDGVEVHN
AKTKPCEEQYGSTYRCVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
TCE3 CCR8 scFab VH and CH1
(SEQ ID NO: 12)
QVQLVESGGGVVQPGRSLRLSCEASGFTFSSYVMHWVRQAPGKGLEWVSVISYDGSSQYYTDS
VKGRFTISRDNSKNTLNLQMNSLRAEDTAVYYCVRGRLATAILFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSC
TCE3 CCR8 scFab VL and Ck
(SEQ ID NO: 13)
EILMTQTPLSLSVTPGQPASISCKSSQSLLYSDGKTYLFWYLQRPGQPPQLLIYEVSNRFSGV
PDRFSGSGSGTDFTLKISRVEAEDVGIYYCMQSIKLPLTFGGGTKVEIKRTVAAPSVFIFPPS
DEQLKSGTASVVCLLNNFYPREAKVQWKVDNALQSGNSQESVTEQDSKDSTYSLSSTLTLSKA
DYEKHKVYACEVTHQGLSSPVTKSFNRGEC
TCE3 CCR8 scFab
(SEQ ID NO: 14)
QVQLVESGGGVVQPGRSLRLSCEASGFTFSSYVMHWVRQAPGKGLEWVSVISYDGSSQYYTDS
VKGRFTISRDNSKNTLNLQMNSLRAEDTAVYYCVRGRLATAILFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCGGGGQGGGGQGGGGQGGGGQGGGGQGGGG
QGGGGQGGGGQEILMTQTPLSLSVTPGQPASISCKSSQSLLYSDGKTYLFWYLQRPGQPPQLL
IYEVSNRFSGVPDRFSGSGSGTDFTLKISRVEAEDVGIYYCMQSIKLPLTFGGGTKVEIKRTV
AAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
TCE3 scFab (CCR8) x scFv (CD3) TCE
(SEQ ID NO: 15)
QVQLVESGGGVVQPGRSLRLSCEASGFTFSSYVMHWVRQAPGKGLEWVSVISYDGSSQYYTDS
VKGRFTISRDNSKNTLNLQMNSLRAEDTAVYYCVRGRLATAILFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCGGGGQGGGGQGGGGQGGGGQGGGGQGGGG
QGGGGQGGGGQEILMTQTPLSLSVTPGQPASISCKSSQSLLYSDGKTYLFWYLQRPGQPPQLL
IYEVSNRFSGVPDRFSGSGSGTDFTLKISRVEAEDVGIYYCMQSIKLPLTFGGGTKVEIKRTV
AAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECSGGGGQEVQLVESGGGLVQPGG
SLKLSCAASGFTFNKYAINWVRQAPGKGLEWVARIRSKYNNYATYYADAVKDRFTISRDDSKN
TVYLQMNNLKTEDTAVYYCARAGNFGSSYISYWAYWGQGTLVTVSSGGGGQGGGGQGGGGQQT
VVTQEPSLTVSPGGTVTITCGSSTGAVTSGNYPNWVQKKPGQAPRGLIGGTKFLAPGTPARFS
GSLSGGKAALTLSGVQPEDEAEYYCVLWYSNRWVFGSGTKLTVL
TCE3 scFab (CCR8) x scFv (CD3) x scFc
(SEQ ID NO: 16)
QVQLVESGGGVVQPGRSLRLSCEASGFTFSSYVMHWVRQAPGKGLEWVSVISYDGSSQYYTDS
VKGRFTISRDNSKNTLNLQMNSLRAEDTAVYYCVRGRLATAILFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCGGGGQGGGGQGGGGQGGGGQGGGGQGGGG
QGGGGQGGGGQEILMTQTPLSLSVTPGQPASISCKSSQSLLYSDGKTYLFWYLQRPGQPPQLL
IYEVSNRFSGVPDRFSGSGSGTDFTLKISRVEAEDVGIYYCMQSIKLPLTFGGGTKVEIKRTV
AAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECSGGGGQEVQLVESGGGLVQPGG
SLKLSCAASGFTFNKYAINWVRQAPGKGLEWVARIRSKYNNYATYYADAVKDRFTISRDDSKN
TVYLQMNNLKTEDTAVYYCARAGNFGSSYISYWAYWGQGTLVTVSSGGGGQGGGGQGGGGQQT
VVTQEPSLTVSPGGTVTITCGSSTGAVTSGNYPNWVQKKPGQAPRGLIGGTKFLAPGTPARFS
GSLSGGKAALTLSGVQPEDEAEYYCVLWYSNRWVFGSGTKLTVLGGGGCPPCPAPELLGGPSV
FLFPPKPKDTLMISRTPEVTCVVVDVSHEEPEVKFNWYVDGVEVHNAKTKPCEEQYGSTYRCV
SVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMH
EALHNHYTQKSLSLSPGKGGGGQGGGGQGGGGQGGGGQGGGGQGGGGQCPPCPAPELLGGPSV
FLFPPKPKDTLMISRTPEVTCVVVDVSHEEPEVKFNWYVDGVEVHNAKTKPCEEQYGSTYRCV
SVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMH
EALHNHYTQKSLSLSPGK
TCE4 CCR8 scFv and scFab HCDR1
(SEQ ID NO: 17)
SYGMH
TCE4 CCR8 scFv and scFab HCDR2
(SEQ ID NO: 18)
VISYDGSNKYYADSVKG
TCE4 CCR8 scFv and scFab HCDR3
(SEQ ID NO: 19)
GRYFDWFLFDY
TCE4 CCR8 scFv and scFab LCDR1
(SEQ ID NO: 20)
KSSQSLLHSDGKTYLF
TCE4 CCR8 scFv and scFab LCDR2
(SEQ ID NO: 21)
EVSNRFS
TCE4 CCR8 scFv and scFab LCDR3
(SEQ ID NO: 22)
MQSLRLPLT
TCE4 CCR8 scFv VH (CCR8)
(SEQ ID NO: 23)
QVQLVESGGGVVQPGRSLRLSCAASGFTFSSYGMHWVRQAPGKCLEWVAVISYDGSNKYYADS
VKGRFTISRDNSKNTLYLQMNSLRAEDTAVYYCARGRYFDWFLFDYWGQGTLVTVSS
TCE4 CCR8 scFv VL (CCR8)
(SEQ ID NO: 24)
DTVMTQTPLSLSVTPGQPASISCKSSQSLLHSDGKTYLFWYLQKPGQPPQLLISEVSNRFSGV
PDRFSGSGSGTDFTLKISRVEAEDVGFYYCMQSLRLPLTFGCGTKVEIK
TCE4 CCR8 scFv
(SEQ ID NO: 25)
QVQLVESGGGVVQPGRSLRLSCAASGFTFSSYGMHWVRQAPGKCLEWVAVISYDGSNKYYADS
VKGRFTISRDNSKNTLYLQMNSLRAEDTAVYYCARGRYFDWFLFDYWGQGTLVTVSSGGGGQG
GGGQGGGGQDTVMTQTPLSLSVTPGQPASISCKSSQSLLHSDGKTYLFWYLQKPGQPPQLLIS
EVSNRFSGVPDRFSGSGSGTDFTLKISRVEAEDVGFYYCMQSLRLPLTFGCGTKVEIK
TCE4 scFv (CCR8) x scFv (CD3) TCE
(SEQ ID NO: 26)
QVQLVESGGGVVQPGRSLRLSCAASGFTFSSYGMHWVRQAPGKCLEWVAVISYDGSNKYYADS
VKGRFTISRDNSKNTLYLQMNSLRAEDTAVYYCARGRYFDWFLFDYWGQGTLVTVSSGGGGQG
GGGQGGGGQDTVMTQTPLSLSVTPGQPASISCKSSQSLLHSDGKTYLFWYLQKPGQPPQLLIS
EVSNRFSGVPDRFSGSGSGTDFTLKISRVEAEDVGFYYCMQSLRLPLTFGCGTKVEIKSGGGG
QEVQLVESGGGLVQPGGSLKLSCAASGFTFNKYAINWVRQAPGKGLEWVARIRSKYNNYATYY
ADAVKDRFTISRDDSKNTVYLQMNNLKTEDTAVYYCARAGNFGSSYISYWAYWGQGTLVTVSS
GGGGQGGGGQGGGGQQTVVTQEPSLTVSPGGTVTITCGSSTGAVTSGNYPNWVQKKPGQAPRG
LIGGTKFLAPGTPARFSGSLSGGKAALTLSGVQPEDEAEYYCVLWYSNRWVFGSGTKLTVL
TCE4 CCR8 scFv (CCR8) x scFv (CD3) TCE x scFc
(SEQ ID NO: 27)
QVQLVESGGGVVQPGRSLRLSCAASGFTFSSYGMHWVRQAPGKCLEWVAVISYDGSNKYYADS
VKGRFTISRDNSKNTLYLQMNSLRAEDTAVYYCARGRYFDWFLFDYWGQGTLVTVSSGGGGQG
GGGQGGGGQDTVMTQTPLSLSVTPGQPASISCKSSQSLLHSDGKTYLFWYLQKPGQPPQLLIS
EVSNRFSGVPDRFSGSGSGTDFTLKISRVEAEDVGFYYCMQSLRLPLTFGCGTKVEIKSGGGG
QEVQLVESGGGLVQPGGSLKLSCAASGFTFNKYAINWVRQAPGKGLEWVARIRSKYNNYATYY
ADAVKDRFTISRDDSKNTVYLQMNNLKTEDTAVYYCARAGNFGSSYISYWAYWGQGTLVTVSS
GGGGQGGGGQGGGGQQTVVTQEPSLTVSPGGTVTITCGSSTGAVTSGNYPNWVQKKPGQAPRG
LIGGTKFLAPGTPARFSGSLSGGKAALTLSGVQPEDEAEYYCVLWYSNRWVFGSGTKLTVLGG
GGCPPCPAPELLGGPSVFLFPPKPKDTLMISRTPEVTCVVVDVSHEEPEVKFNWYVDGVEVHN
AKTKPCEEQYGSTYRCVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGKGGGGQGGGGQGGGGQGGGGQGGGGQGGG
GQCPPCPAPELLGGPSVFLFPPKPKDTLMISRTPEVTCVVVDVSHEEPEVKFNWYVDGVEVHN
AKTKPCEEQYGSTYRCVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
TCE4 CCR8 scFab VH and CH1
(SEQ ID NO: 28)
QVQLVESGGGVVQPGRSLRLSCAASGFTFSSYGMHWVRQAPGKGLEWVAVISYDGSNKYYADS
VKGRFTISRDNSKNTLYLQMNSLRAEDTAVYYCARGRYFDWFLFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSC
TCE4 CCR8 scFab VL and Ck
(SEQ ID NO: 29)
DTVMTQTPLSLSVTPGQPASISCKSSQSLLHSDGKTYLFWYLQKPGQPPQLLISEVSNRFSGV
PDRFSGSGSGTDFTLKISRVEAEDVGFYYCMQSLRLPLTFGGGTKVEIKRTVAAPSVFIFPPS
DEQLKSGTASVVCLLNNFYPREAKVQWKVDNALQSGNSQESVTEQDSKDSTYSLSSTLTLSKA
DYEKHKVYACEVTHQGLSSPVTKSFNRGEC
TCE4 CCR8 scFab
(SEQ ID NO: 30)
QVQLVESGGGVVQPGRSLRLSCAASGFTFSSYGMHWVRQAPGKGLEWVAVISYDGSNKYYADS
VKGRFTISRDNSKNTLYLQMNSLRAEDTAVYYCARGRYFDWFLFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCGGGGQGGGGQGGGGQGGGGQGGGGQGGGG
QGGGGQGGGGQDTVMTQTPLSLSVTPGQPASISCKSSQSLLHSDGKTYLFWYLQKPGQPPQLL
ISEVSNRFSGVPDRFSGSGSGTDFTLKISRVEAEDVGFYYCMQSLRLPLTFGGGTKVEIKRTV
AAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
TCE4 scFab (CCR8) x scFv (CD3) TCE
(SEQ ID NO: 31)
QVQLVESGGGVVQPGRSLRLSCAASGFTFSSYGMHWVRQAPGKGLEWVAVISYDGSNKYYADS
VKGRFTISRDNSKNTLYLQMNSLRAEDTAVYYCARGRYFDWFLFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCGGGGQGGGGQGGGGQGGGGQGGGGQGGGG
QGGGGQGGGGQDTVMTQTPLSLSVTPGQPASISCKSSQSLLHSDGKTYLFWYLQKPGQPPQLL
ISEVSNRFSGVPDRFSGSGSGTDFTLKISRVEAEDVGFYYCMQSLRLPLTFGGGTKVEIKRTV
AAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECSGGGGQEVQLVESGGGLVQPGG
SLKLSCAASGFTFNKYAINWVRQAPGKGLEWVARIRSKYNNYATYYADAVKDRFTISRDDSKN
TVYLQMNNLKTEDTAVYYCARAGNFGSSYISYWAYWGQGTLVTVSSGGGGQGGGGQGGGGQQT
VVTQEPSLTVSPGGTVTITCGSSTGAVTSGNYPNWVQKKPGQAPRGLIGGTKFLAPGTPARFS
GSLSGGKAALTLSGVQPEDEAEYYCVLWYSNRWVFGSGTKLTVL
TCE4 CCR8 scFab (CCR8) x scFv (CD3) x scFc
(SEQ ID NO: 32)
QVQLVESGGGVVQPGRSLRLSCAASGFTFSSYGMHWVRQAPGKGLEWVAVISYDGSNKYYADS
VKGRFTISRDNSKNTLYLQMNSLRAEDTAVYYCARGRYFDWFLFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCGGGGQGGGGQGGGGQGGGGQGGGGQGGGG
QGGGGQGGGGQDTVMTQTPLSLSVTPGQPASISCKSSQSLLHSDGKTYLFWYLQKPGQPPQLL
ISEVSNRFSGVPDRFSGSGSGTDFTLKISRVEAEDVGFYYCMQSLRLPLTFGGGTKVEIKRTV
AAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECSGGGGQEVQLVESGGGLVQPGG
SLKLSCAASGFTFNKYAINWVRQAPGKGLEWVARIRSKYNNYATYYADAVKDRFTISRDDSKN
TVYLQMNNLKTEDTAVYYCARAGNFGSSYISYWAYWGQGTLVTVSSGGGGQGGGGQGGGGQQT
VVTQEPSLTVSPGGTVTITCGSSTGAVTSGNYPNWVQKKPGQAPRGLIGGTKFLAPGTPARFS
GSLSGGKAALTLSGVQPEDEAEYYCVLWYSNRWVFGSGTKLTVLGGGGCPPCPAPELLGGPSV
FLFPPKPKDTLMISRTPEVTCVVVDVSHEEPEVKFNWYVDGVEVHNAKTKPCEEQYGSTYRCV
SVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMH
EALHNHYTQKSLSLSPGKGGGGQGGGGQGGGGQGGGGQGGGGQGGGGQCPPCPAPELLGGPSV
FLFPPKPKDTLMISRTPEVTCVVVDVSHEEPEVKFNWYVDGVEVHNAKTKPCEEQYGSTYRCV
SVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMH
EALHNHYTQKSLSLSPGK
TCE5 CCR8 scFv and scFab HCDR1
(SEQ ID NO: 33)
NAWMS
TCE5 CCR8 scFv and scFab HCDR2
(SEQ ID NO: 34)
RIKRKTDGGTTDYAAPVKG
TCE5 CCR8 scFv and scFab HCDR3
(SEQ ID NO: 35)
VTLVRGVIFDY
TCE5 CCR8 scFv and scFab LCDR1
(SEQ ID NO: 36)
RVSQSVSSSQLA
TCE5 CCR8 scFv and scFab LCDR2
(SEQ ID NO: 37)
GASSRAT
TCE5 CCR8 scFv and scFab LCDR3
(SEQ ID NO: 38)
QQYGNSRT
TCE5 CCR8 scFv VH (CCR8)
(SEQ ID NO: 39)
EVQLVESGGGLVKPGGSLRLACAASGFIFSNAWMSWVRQAPGKCLEWVGRIKRKTDGGTTDYA
APVKGRFTISRDDSKNTLYLLMNSLKIEDTAVYYCTVVTLVRGVIFDYWGQGTLVTVSS
TCE5 CCR8 scFv VL (CCR8)
(SEQ ID NO: 40)
EIVLTQFPGTLSLSPGESATLSCRVSQSVSSSQLAWYQQKPGQAPRLLIYGASSRATGIPDRF
SGSGSGTDFTLIISRLEPEDFAVYYCQQYGNSRTFGCGTKVEIK
TCE5 CCR8 scFv
(SEQ ID NO: 41)
EVQLVESGGGLVKPGGSLRLACAASGFIFSNAWMSWVRQAPGKCLEWVGRIKRKTDGGTTDYA
APVKGRFTISRDDSKNTLYLLMNSLKIEDTAVYYCTVVTLVRGVIFDYWGQGTLVTVSSGGGG
QGGGGQGGGGQEIVLTQFPGTLSLSPGESATLSCRVSQSVSSSQLAWYQQKPGQAPRLLIYGA
SSRATGIPDRFSGSGSGTDFTLIISRLEPEDFAVYYCQQYGNSRTFGCGTKVEIK
TCE5 scFv (CCR8) x scFv (CD3) TCE
(SEQ ID NO: 42)
EVQLVESGGGLVKPGGSLRLACAASGFIFSNAWMSWVRQAPGKCLEWVGRIKRKTDGGTTDYA
APVKGRFTISRDDSKNTLYLLMNSLKIEDTAVYYCTVVTLVRGVIFDYWGQGTLVTVSSGGGG
QGGGGQGGGGQEIVLTQFPGTLSLSPGESATLSCRVSQSVSSSQLAWYQQKPGQAPRLLIYGA
SSRATGIPDRFSGSGSGTDFTLIISRLEPEDFAVYYCQQYGNSRTFGCGTKVEIKSGGGGQEV
QLVESGGGLVQPGGSLKLSCAASGFTFNKYAINWVRQAPGKGLEWVARIRSKYNNYATYYADA
VKDRFTISRDDSKNTVYLQMNNLKTEDTAVYYCARAGNFGSSYISYWAYWGQGTLVTVSSGGG
GQGGGGQGGGGQQTVVTQEPSLTVSPGGTVTITCGSSTGAVTSGNYPNWVQKKPGQAPRGLIG
GTKFLAPGTPARFSGSLSGGKAALTLSGVQPEDEAEYYCVLWYSNRWVFGSGTKLTVL
TCE5 scFv (CCR8) x scFv (CD3) x scFc
(SEQ ID NO: 43)
EVQLVESGGGLVKPGGSLRLACAASGFIFSNAWMSWVRQAPGKCLEWVGRIKRKTDGGTTDYA
APVKGRFTISRDDSKNTLYLLMNSLKIEDTAVYYCTVVTLVRGVIFDYWGQGTLVTVSSGGGG
QGGGGQGGGGQEIVLTQFPGTLSLSPGESATLSCRVSQSVSSSQLAWYQQKPGQAPRLLIYGA
SSRATGIPDRFSGSGSGTDFTLIISRLEPEDFAVYYCQQYGNSRTFGCGTKVEIKSGGGGQEV
QLVESGGGLVQPGGSLKLSCAASGFTFNKYAINWVRQAPGKGLEWVARIRSKYNNYATYYADA
VKDRFTISRDDSKNTVYLQMNNLKTEDTAVYYCARAGNFGSSYISYWAYWGQGTLVTVSSGGG
GQGGGGQGGGGQQTVVTQEPSLTVSPGGTVTITCGSSTGAVTSGNYPNWVQKKPGQAPRGLIG
GTKFLAPGTPARFSGSLSGGKAALTLSGVQPEDEAEYYCVLWYSNRWVFGSGTKLTVLGGGGC
PPCPAPELLGGPSVFLFPPKPKDTLMISRTPEVTCVVVDVSHEEPEVKFNWYVDGVEVHNAKT
KPCEEQYGSTYRCVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGKGGGGQGGGGQGGGGQGGGGQGGGGQGGGGQC
PPCPAPELLGGPSVFLFPPKPKDTLMISRTPEVTCVVVDVSHEEPEVKFNWYVDGVEVHNAKT
KPCEEQYGSTYRCVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
TCE5 CCR8 scFab VH and CH1
(SEQ ID NO: 44)
EVQLVESGGGLVKPGGSLRLACAASGFIFSNAWMSWVRQAPGKGLEWVGRIKRKTDGGTTDYA
APVKGRFTISRDDSKNTLYLLMNSLKIEDTAVYYCTVVTLVRGVIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSC
TCE5 CCR8 scFab VL and Ck
(SEQ ID NO: 45)
EIVLTQFPGTLSLSPGESATLSCRVSQSVSSSQLAWYQQKPGQAPRLLIYGASSRATGIPDRF
SGSGSGTDFTLIISRLEPEDFAVYYCQQYGNSRTFGQGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
TCE5 CCR8 scFab
(SEQ ID NO: 46)
EVQLVESGGGLVKPGGSLRLACAASGFIFSNAWMSWVRQAPGKGLEWVGRIKRKTDGGTTDYA
APVKGRFTISRDDSKNTLYLLMNSLKIEDTAVYYCTVVTLVRGVIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQFPGTLSLSPGESATLSCRVSQSVSSSQLAWYQQKPGQAPRLLIY
GASSRATGIPDRFSGSGSGTDFTLIISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
TCE5 scFab (CCR8) x scFv (CD3)
(SEQ ID NO: 47)
EVQLVESGGGLVKPGGSLRLACAASGFIFSNAWMSWVRQAPGKGLEWVGRIKRKTDGGTTDYA
APVKGRFTISRDDSKNTLYLLMNSLKIEDTAVYYCTVVTLVRGVIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQFPGTLSLSPGESATLSCRVSQSVSSSQLAWYQQKPGQAPRLLIY
GASSRATGIPDRFSGSGSGTDFTLIISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECSGGGGQEVQLVESGGGLVQPGGSLK
LSCAASGFTFNKYAINWVRQAPGKGLEWVARIRSKYNNYATYYADAVKDRFTISRDDSKNTVY
LQMNNLKTEDTAVYYCARAGNFGSSYISYWAYWGQGTLVTVSSGGGGQGGGGQGGGGQQTVVT
QEPSLTVSPGGTVTITCGSSTGAVTSGNYPNWVQKKPGQAPRGLIGGTKFLAPGTPARFSGSL
SGGKAALTLSGVQPEDEAEYYCVLWYSNRWVFGSGTKLTVL
TCE5 scFab (CCR8) x scFv (CD3) x scFc
(SEQ ID NO: 48)
EVQLVESGGGLVKPGGSLRLACAASGFIFSNAWMSWVRQAPGKGLEWVGRIKRKTDGGTTDYA
APVKGRFTISRDDSKNTLYLLMNSLKIEDTAVYYCTVVTLVRGVIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQFPGTLSLSPGESATLSCRVSQSVSSSQLAWYQQKPGQAPRLLIY
GASSRATGIPDRFSGSGSGTDFTLIISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECSGGGGQEVQLVESGGGLVQPGGSLK
LSCAASGFTFNKYAINWVRQAPGKGLEWVARIRSKYNNYATYYADAVKDRFTISRDDSKNTVY
LQMNNLKTEDTAVYYCARAGNFGSSYISYWAYWGQGTLVTVSSGGGGQGGGGQGGGGQQTVVT
QEPSLTVSPGGTVTITCGSSTGAVTSGNYPNWVQKKPGQAPRGLIGGTKFLAPGTPARFSGSL
SGGKAALTLSGVQPEDEAEYYCVLWYSNRWVFGSGTKLTVLGGGGCPPCPAPELLGGPSVFLF
PPKPKDTLMISRTPEVTCVVVDVSHEEPEVKFNWYVDGVEVHNAKTKPCEEQYGSTYRCVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGKGGGGQGGGGQGGGGQGGGGQGGGGQGGGGQCPPCPAPELLGGPSVFLF
PPKPKDTLMISRTPEVTCVVVDVSHEEPEVKFNWYVDGVEVHNAKTKPCEEQYGSTYRCVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGK
TCE2 CCR8 scFv and scFab HCDR1
(SEQ ID NO: 49)
NYGMH
TCE2 CCR8 scFv and scFab HCDR2
(SEQ ID NO: 50)
VISYDGSNKFYADSVKG
TCE2 CCR8 scFv and scFab HCDR3
(SEQ ID NO: 51)
AGGIGRFDY
TCE2 CCR8 scFv and scFab LCDR1
(SEQ ID NO: 52)
KYSQSLLHSDGKTYLF
TCE2 CCR8 scFv and scFab LCDR2
(SEQ ID NO: 53)
EVSNRFS
TCE2 CCR8 scFv and scFab LCDR3
(SEQ ID NO: 54)
MQTLKLPLT
TCE2 CCR8 scFv VH
(SEQ ID NO: 55)
QVQLVESGGGVVQPGRSLRLSCAASGFTFSNYGMHWVRQAPGKCLEWVAVISYDGSNKFYADS
VKGRFTISRDNSKKTLYLQMSSLRVEDTAVYYCARAGGIGRFDYWGQGTLVTVSS
TCE2 CCR8 scFv VL
(SEQ ID NO: 56)
DFVMTQTPLSLSVTPGQPASISCKYSQSLLHSDGKTYLFWYLQKPGQPPHLLIYEVSNRFSGV
PDRFSGSGSGTDFTLKISRVEAEDVGLYYCMQTLKLPLTFGCGTKVEIN
TCE2 CCR8 scFv
(SEQ ID NO: 57)
QVQLVESGGGVVQPGRSLRLSCAASGFTFSNYGMHWVRQAPGKCLEWVAVISYDGSNKFYADS
VKGRFTISRDNSKKTLYLQMSSLRVEDTAVYYCARAGGIGRFDYWGQGTLVTVSSGGGGQGGG
GQGGGGQDFVMTQTPLSLSVTPGQPASISCKYSQSLLHSDGKTYLFWYLQKPGQPPHLLIYEV
SNRFSGVPDRFSGSGSGTDFTLKISRVEAEDVGLYYCMQTLKLPLTFGCGTKVEIN
TCE2 scFv (CCR8) x scFv (CD3)
(SEQ ID NO: 58)
QVQLVESGGGVVQPGRSLRLSCAASGFTFSNYGMHWVRQAPGKCLEWVAVISYDGSNKFYADS
VKGRFTISRDNSKKTLYLQMSSLRVEDTAVYYCARAGGIGRFDYWGQGTLVTVSSGGGGQGGG
GQGGGGQDFVMTQTPLSLSVTPGQPASISCKYSQSLLHSDGKTYLFWYLQKPGQPPHLLIYEV
SNRFSGVPDRFSGSGSGTDFTLKISRVEAEDVGLYYCMQTLKLPLTFGCGTKVEINSGGGGQE
VQLVESGGGLVQPGGSLKLSCAASGFTFNKYAINWVRQAPGKGLEWVARIRSKYNNYATYYAD
AVKDRFTISRDDSKNTVYLQMNNLKTEDTAVYYCARAGNFGSSYISYWAYWGQGTLVTVSSGG
GGQGGGGQGGGGQQTVVTQEPSLTVSPGGTVTITCGSSTGAVTSGNYPNWVQKKPGQAPRGLI
GGTKFLAPGTPARFSGSLSGGKAALTLSGVQPEDEAEYYCVLWYSNRWVFGSGTKLTVL
TCE2 scFv (CCR8) x scFv (CD3) x scFc
(SEQ ID NO: 59)
QVQLVESGGGVVQPGRSLRLSCAASGFTFSNYGMHWVRQAPGKCLEWVAVISYDGSNKFYADS
VKGRFTISRDNSKKTLYLQMSSLRVEDTAVYYCARAGGIGRFDYWGQGTLVTVSSGGGGQGGG
GQGGGGQDFVMTQTPLSLSVTPGQPASISCKYSQSLLHSDGKTYLFWYLQKPGQPPHLLIYEV
SNRFSGVPDRFSGSGSGTDFTLKISRVEAEDVGLYYCMQTLKLPLTFGCGTKVEINSGGGGQE
VQLVESGGGLVQPGGSLKLSCAASGFTFNKYAINWVRQAPGKGLEWVARIRSKYNNYATYYAD
AVKDRFTISRDDSKNTVYLQMNNLKTEDTAVYYCARAGNFGSSYISYWAYWGQGTLVTVSSGG
GGQGGGGQGGGGQQTVVTQEPSLTVSPGGTVTITCGSSTGAVTSGNYPNWVQKKPGQAPRGLI
GGTKFLAPGTPARFSGSLSGGKAALTLSGVQPEDEAEYYCVLWYSNRWVFGSGTKLTVLGGGG
CPPCPAPELLGGPSVFLFPPKPKDTLMISRTPEVTCVVVDVSHEEPEVKFNWYVDGVEVHNAK
TKPCEEQYGSTYRCVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGKGGGGQGGGGQGGGGQGGGGQGGGGQGGGGQ
CPPCPAPELLGGPSVFLFPPKPKDTLMISRTPEVTCVVVDVSHEEPEVKFNWYVDGVEVHNAK
TKPCEEQYGSTYRCVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
TCE2 CCR8 scFab VH and CH1
(SEQ ID NO: 60)
QVQLVESGGGVVQPGRSLRLSCAASGFTFSNYGMHWVRQAPGKGLEWVAVISYDGSNKFYADS
VKGRFTISRDNSKKTLYLQMSSLRVEDTAVYYCARAGGIGRFDYWGQGTLVTVSSASTKGPSV
FPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSC
TCE2 scFab CCR8 VL and Ck
(SEQ ID NO: 61)
DFVMTQTPLSLSVTPGQPASISCKYSQSLLHSDGKTYLFWYLQKPGQPPHLLIYEVSNRFSGV
PDRFSGSGSGTDFTLKISRVEAEDVGLYYCMQTLKLPLTFGGGTKVEINRTVAAPSVFIFPPS
DEQLKSGTASVVCLLNNFYPREAKVQWKVDNALQSGNSQESVTEQDSKDSTYSLSSTLTLSKA
DYEKHKVYACEVTHQGLSSPVTKSFNRGEC
TCE2 CCR8 scFab
(SEQ ID NO: 62)
QVQLVESGGGVVQPGRSLRLSCAASGFTFSNYGMHWVRQAPGKGLEWVAVISYDGSNKFYADS
VKGRFTISRDNSKKTLYLQMSSLRVEDTAVYYCARAGGIGRFDYWGQGTLVTVSSASTKGPSV
FPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCGGGGQGGGGQGGGGQGGGGQGGGGQGGGGQG
GGGQGGGGQDFVMTQTPLSLSVTPGQPASISCKYSQSLLHSDGKTYLFWYLQKPGQPPHLLIY
EVSNRFSGVPDRFSGSGSGTDFTLKISRVEAEDVGLYYCMQTLKLPLTFGGGTKVEINRTVAA
PSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
TCE2 scFab (CCR8) x scFv (CD3)
(SEQ ID NO: 63)
QVQLVESGGGVVQPGRSLRLSCAASGFTFSNYGMHWVRQAPGKGLEWVAVISYDGSNKFYADS
VKGRFTISRDNSKKTLYLQMSSLRVEDTAVYYCARAGGIGRFDYWGQGTLVTVSSASTKGPSV
FPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCGGGGQGGGGQGGGGQGGGGQGGGGQGGGGQG
GGGQGGGGQDFVMTQTPLSLSVTPGQPASISCKYSQSLLHSDGKTYLFWYLQKPGQPPHLLIY
EVSNRFSGVPDRFSGSGSGTDFTLKISRVEAEDVGLYYCMQTLKLPLTFGGGTKVEINRTVAA
PSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGECSGGGGQEVQLVESGGGLVQPGGSL
KLSCAASGFTFNKYAINWVRQAPGKGLEWVARIRSKYNNYATYYADAVKDRFTISRDDSKNTV
YLQMNNLKTEDTAVYYCARAGNFGSSYISYWAYWGQGTLVTVSSGGGGQGGGGQGGGGQQTVV
TQEPSLTVSPGGTVTITCGSSTGAVTSGNYPNWVQKKPGQAPRGLIGGTKFLAPGTPARFSGS
LSGGKAALTLSGVQPEDEAEYYCVLWYSNRWVFGSGTKLTVL
TCE2 scFab (CCR8) x scFv (CD3) x scFc
(SEQ ID NO: 64)
QVQLVESGGGVVQPGRSLRLSCAASGFTFSNYGMHWVRQAPGKGLEWVAVISYDGSNKFYADS
VKGRFTISRDNSKKTLYLQMSSLRVEDTAVYYCARAGGIGRFDYWGQGTLVTVSSASTKGPSV
FPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCGGGGQGGGGQGGGGQGGGGQGGGGQGGGGQG
GGGQGGGGQDFVMTQTPLSLSVTPGQPASISCKYSQSLLHSDGKTYLFWYLQKPGQPPHLLIY
EVSNRFSGVPDRFSGSGSGTDFTLKISRVEAEDVGLYYCMQTLKLPLTFGGGTKVEINRTVAA
PSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGECSGGGGQEVQLVESGGGLVQPGGSL
KLSCAASGFTFNKYAINWVRQAPGKGLEWVARIRSKYNNYATYYADAVKDRFTISRDDSKNTV
YLQMNNLKTEDTAVYYCARAGNFGSSYISYWAYWGQGTLVTVSSGGGGQGGGGQGGGGQQTVV
TQEPSLTVSPGGTVTITCGSSTGAVTSGNYPNWVQKKPGQAPRGLIGGTKFLAPGTPARFSGS
LSGGKAALTLSGVQPEDEAEYYCVLWYSNRWVFGSGTKLTVLGGGGCPPCPAPELLGGPSVFL
FPPKPKDTLMISRTPEVTCVVVDVSHEEPEVKFNWYVDGVEVHNAKTKPCEEQYGSTYRCVSV
LTVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEA
LHNHYTQKSLSLSPGKGGGGQGGGGQGGGGQGGGGQGGGGQGGGGQCPPCPAPELLGGPSVFL
FPPKPKDTLMISRTPEVTCVVVDVSHEEPEVKFNWYVDGVEVHNAKTKPCEEQYGSTYRCVSV
LTVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEA
LHNHYTQKSLSLSPGK
TCE6 CCR8 scFv and scFab HCDR1
(SEQ ID NO: 65)
NAWMS
TCE6 CCR8 scFv and scFab HCDR2
(SEQ ID NO: 66)
RIKRKTDGGTTDYAAPVKG
TCE6 CCR8 scFv and scFab HCDR3
(SEQ ID NO: 67)
VTLVRGIIFDY
TCE6 CCR8 scFv and scFab LCDR1
(SEQ ID NO: 68)
RVSQSVSSSQLA
TCE6 CCR8 scFv and scFab LCDR2
(SEQ ID NO: 69)
GASSRAT
TCE6 CCR8 scFv and scFab LCDR3
(SEQ ID NO: 70)
QQYGNSRT
TCE6 CCR8 scFv VH
(SEQ ID NO: 71)
EVQLVESGGGLVKPGGSLRLSCAASGFIFSNAWMSWVRQAPGKCLEWVGRIKRKTDGGTTDYA
APVKGRFTISRDDSKNTLYLLMNSLKIEDTAVYYCTVVTLVRGIIFDYWGQGTLVTVSS
TCE6 CCR8 scFv VL
(SEQ ID NO: 72)
EIVLTQSPGTLSLSPGESATLSCRVSQSVSSSQLAWYQQKPGQAPRLLIYGASSRATGIPDRF
SGSGSGTDFTLTISRLEPEDFAVYYCQQYGNSRTFGCGTKVEIK
TCE6 CCR8 scFv
(SEQ ID NO: 73)
EVQLVESGGGLVKPGGSLRLSCAASGFIFSNAWMSWVRQAPGKCLEWVGRIKRKTDGGTTDYA
APVKGRFTISRDDSKNTLYLLMNSLKIEDTAVYYCTVVTLVRGIIFDYWGQGTLVTVSSGGGG
QGGGGQGGGGQEIVLTQSPGTLSLSPGESATLSCRVSQSVSSSQLAWYQQKPGQAPRLLIYGA
SSRATGIPDRFSGSGSGTDFTLTISRLEPEDFAVYYCQQYGNSRTFGCGTKVEIK
TCE6 scFv (CCR8) x scFv (CD3)
(SEQ ID NO: 74)
EVQLVESGGGLVKPGGSLRLSCAASGFIFSNAWMSWVRQAPGKCLEWVGRIKRKTDGGTTDYA
APVKGRFTISRDDSKNTLYLLMNSLKIEDTAVYYCTVVTLVRGIIFDYWGQGTLVTVSSGGGG
QGGGGQGGGGQEIVLTQSPGTLSLSPGESATLSCRVSQSVSSSQLAWYQQKPGQAPRLLIYGA
SSRATGIPDRFSGSGSGTDFTLTISRLEPEDFAVYYCQQYGNSRTFGCGTKVEIKSGGGGQEV
QLVESGGGLVQPGGSLKLSCAASGFTFNKYAINWVRQAPGKGLEWVARIRSKYNNYATYYADA
VKDRFTISRDDSKNTVYLQMNNLKTEDTAVYYCARAGNFGSSYISYWAYWGQGTLVTVSSGGG
GQGGGGQGGGGQQTVVTQEPSLTVSPGGTVTITCGSSTGAVTSGNYPNWVQKKPGQAPRGLIG
GTKFLAPGTPARFSGSLSGGKAALTLSGVQPEDEAEYYCVLWYSNRWVFGSGTKLTVL
TCE6 scFv (CCR8) x scFv (CD3) x scFc
(SEQ ID NO: 75)
EVQLVESGGGLVKPGGSLRLSCAASGFIFSNAWMSWVRQAPGKCLEWVGRIKRKTDGGTTDYA
APVKGRFTISRDDSKNTLYLLMNSLKIEDTAVYYCTVVTLVRGIIFDYWGQGTLVTVSSGGGG
QGGGGQGGGGQEIVLTQSPGTLSLSPGESATLSCRVSQSVSSSQLAWYQQKPGQAPRLLIYGA
SSRATGIPDRFSGSGSGTDFTLTISRLEPEDFAVYYCQQYGNSRTFGCGTKVEIKSGGGGQEV
QLVESGGGLVQPGGSLKLSCAASGFTFNKYAINWVRQAPGKGLEWVARIRSKYNNYATYYADA
VKDRFTISRDDSKNTVYLQMNNLKTEDTAVYYCARAGNFGSSYISYWAYWGQGTLVTVSSGGG
GQGGGGQGGGGQQTVVTQEPSLTVSPGGTVTITCGSSTGAVTSGNYPNWVQKKPGQAPRGLIG
GTKFLAPGTPARFSGSLSGGKAALTLSGVQPEDEAEYYCVLWYSNRWVFGSGTKLTVLGGGGC
PPCPAPELLGGPSVFLFPPKPKDTLMISRTPEVTCVVVDVSHEEPEVKFNWYVDGVEVHNAKT
KPCEEQYGSTYRCVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGKGGGGQGGGGQGGGGQGGGGQGGGGQGGGGQC
PPCPAPELLGGPSVFLFPPKPKDTLMISRTPEVTCVVVDVSHEEPEVKFNWYVDGVEVHNAKT
KPCEEQYGSTYRCVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
TCE6 CCR8 scFab VH and CH1
(SEQ ID NO: 76)
EVQLVESGGGLVKPGGSLRLSCAASGFIFSNAWMSWVRQAPGKGLEWVGRIKRKTDGGTTDYA
APVKGRFTISRDDSKNTLYLLMNSLKIEDTAVYYCTVVTLVRGIIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSC
TCE6 CCR8 scFab VL and Ck
(SEQ ID NO: 77)
EIVLTQSPGTLSLSPGESATLSCRVSQSVSSSQLAWYQQKPGQAPRLLIYGASSRATGIPDRF
SGSGSGTDFTLTISRLEPEDFAVYYCQQYGNSRTFGQGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
TCE6 CCR8 scFab
(SEQ ID NO: 78)
EVQLVESGGGLVKPGGSLRLSCAASGFIFSNAWMSWVRQAPGKGLEWVGRIKRKTDGGTTDYA
APVKGRFTISRDDSKNTLYLLMNSLKIEDTAVYYCTVVTLVRGIIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGESATLSCRVSQSVSSSQLAWYQQKPGQAPRLLIY
GASSRATGIPDRFSGSGSGTDFTLTISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
TCE6 scFab (CCR8) x scFv (CD3)
(SEQ ID NO: 79)
EVQLVESGGGLVKPGGSLRLSCAASGFIFSNAWMSWVRQAPGKGLEWVGRIKRKTDGGTTDYA
APVKGRFTISRDDSKNTLYLLMNSLKIEDTAVYYCTVVTLVRGIIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGESATLSCRVSQSVSSSQLAWYQQKPGQAPRLLIY
GASSRATGIPDRFSGSGSGTDFTLTISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECSGGGGQEVQLVESGGGLVQPGGSLK
LSCAASGFTFNKYAINWVRQAPGKGLEWVARIRSKYNNYATYYADAVKDRFTISRDDSKNTVY
LQMNNLKTEDTAVYYCARAGNFGSSYISYWAYWGQGTLVTVSSGGGGQGGGGQGGGGQQTVVT
QEPSLTVSPGGTVTITCGSSTGAVTSGNYPNWVQKKPGQAPRGLIGGTKFLAPGTPARFSGSL
SGGKAALTLSGVQPEDEAEYYCVLWYSNRWVFGSGTKLTVL
TCE6 scFab (CCR8) x scFv (CD3) x scFc
(SEQ ID NO: 80)
EVQLVESGGGLVKPGGSLRLSCAASGFIFSNAWMSWVRQAPGKGLEWVGRIKRKTDGGTTDYA
APVKGRFTISRDDSKNTLYLLMNSLKIEDTAVYYCTVVTLVRGIIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGESATLSCRVSQSVSSSQLAWYQQKPGQAPRLLIY
GASSRATGIPDRFSGSGSGTDFTLTISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECSGGGGQEVQLVESGGGLVQPGGSLK
LSCAASGFTFNKYAINWVRQAPGKGLEWVARIRSKYNNYATYYADAVKDRFTISRDDSKNTVY
LQMNNLKTEDTAVYYCARAGNFGSSYISYWAYWGQGTLVTVSSGGGGQGGGGQGGGGQQTVVT
QEPSLTVSPGGTVTITCGSSTGAVTSGNYPNWVQKKPGQAPRGLIGGTKFLAPGTPARFSGSL
SGGKAALTLSGVQPEDEAEYYCVLWYSNRWVFGSGTKLTVLGGGGCPPCPAPELLGGPSVFLF
PPKPKDTLMISRTPEVTCVVVDVSHEEPEVKFNWYVDGVEVHNAKTKPCEEQYGSTYRCVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGKGGGGQGGGGQGGGGQGGGGQGGGGQGGGGQCPPCPAPELLGGPSVFLF
PPKPKDTLMISRTPEVTCVVVDVSHEEPEVKFNWYVDGVEVHNAKTKPCEEQYGSTYRCVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGK
TCE7 CCR8 scFv and scFab HCDR1
(SEQ ID NO: 81)
NAWMS
TCE7 CCR8 scFv and scFab HCDR2
(SEQ ID NO: 82)
RIKRKTDGGTTDYAAPVKG
TCE7 CCR8 scFv and scFab HCDR3
(SEQ ID NO: 83)
VTLVRGVIFDY
TCE7 CCR8 scFv and scFab LCDR1
(SEQ ID NO: 84)
RASQSVSSSQLA
TCE7 CCR8 scFv and scFab LCDR2
(SEQ ID NO: 85)
GASSRAT
TCE7 CCR8 scFv and scFab LCDR3
(SEQ ID NO: 86)
QQYGNSRT
TCE7 CCR8 scFv VH
(SEQ ID NO: 87)
EVQLVESGGDLVKPGGSLRLSCAASGFIFSNAWMSWVRQAPGKCLEWVGRIKRKTDGGTTDYA
APVKGRFTISRDDSKNTLYLLMNSLKIEDTAVYYCTVVTLVRGVIFDYWGQGTLVTVSS
TCE7 CCR8 scFv VL
(SEQ ID NO: 88)
EIVLTQSPGTLSLSPGESATLSCRASQSVSSSQLAWYQQKPGQTPRLLIYGASSRATGIPDRF
SGSGSGTDFTLTISRLEPEDFAVYYCQQYGNSRTFGCGTKVEIK
TCE7 CCR8 scFv
(SEQ ID NO: 89)
EVQLVESGGDLVKPGGSLRLSCAASGFIFSNAWMSWVRQAPGKCLEWVGRIKRKTDGGTTDYA
APVKGRFTISRDDSKNTLYLLMNSLKIEDTAVYYCTVVTLVRGVIFDYWGQGTLVTVSSGGGG
QGGGGQGGGGQEIVLTQSPGTLSLSPGESATLSCRASQSVSSSQLAWYQQKPGQTPRLLIYGA
SSRATGIPDRFSGSGSGTDFTLTISRLEPEDFAVYYCQQYGNSRTFGCGTKVEIK
TCE7 scFv (CCR8) x scFv (CD3)
(SEQ ID NO: 90)
EVQLVESGGDLVKPGGSLRLSCAASGFIFSNAWMSWVRQAPGKCLEWVGRIKRKTDGGTTDYA
APVKGRFTISRDDSKNTLYLLMNSLKIEDTAVYYCTVVTLVRGVIFDYWGQGTLVTVSSGGGG
QGGGGQGGGGQEIVLTQSPGTLSLSPGESATLSCRASQSVSSSQLAWYQQKPGQTPRLLIYGA
SSRATGIPDRFSGSGSGTDFTLTISRLEPEDFAVYYCQQYGNSRTFGCGTKVEIKSGGGGQEV
QLVESGGGLVQPGGSLKLSCAASGFTFNKYAINWVRQAPGKGLEWVARIRSKYNNYATYYADA
VKDRFTISRDDSKNTVYLQMNNLKTEDTAVYYCARAGNFGSSYISYWAYWGQGTLVTVSSGGG
GQGGGGQGGGGQQTVVTQEPSLTVSPGGTVTITCGSSTGAVTSGNYPNWVQKKPGQAPRGLIG
GTKFLAPGTPARFSGSLSGGKAALTLSGVQPEDEAEYYCVLWYSNRWVFGSGTKLTVL
TCE7 scFv (CCR8) x scFv (CD3) x scFc
(SEQ ID NO: 91)
EVQLVESGGDLVKPGGSLRLSCAASGFIFSNAWMSWVRQAPGKCLEWVGRIKRKTDGGTTDYA
APVKGRFTISRDDSKNTLYLLMNSLKIEDTAVYYCTVVTLVRGVIFDYWGQGTLVTVSSGGGG
QGGGGQGGGGQEIVLTQSPGTLSLSPGESATLSCRASQSVSSSQLAWYQQKPGQTPRLLIYGA
SSRATGIPDRFSGSGSGTDFTLTISRLEPEDFAVYYCQQYGNSRTFGCGTKVEIKSGGGGQEV
QLVESGGGLVQPGGSLKLSCAASGFTFNKYAINWVRQAPGKGLEWVARIRSKYNNYATYYADA
VKDRFTISRDDSKNTVYLQMNNLKTEDTAVYYCARAGNFGSSYISYWAYWGQGTLVTVSSGGG
GQGGGGQGGGGQQTVVTQEPSLTVSPGGTVTITCGSSTGAVTSGNYPNWVQKKPGQAPRGLIG
GTKFLAPGTPARFSGSLSGGKAALTLSGVQPEDEAEYYCVLWYSNRWVFGSGTKLTVLGGGGC
PPCPAPELLGGPSVFLFPPKPKDTLMISRTPEVTCVVVDVSHEEPEVKFNWYVDGVEVHNAKT
KPCEEQYGSTYRCVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGKGGGGQGGGGQGGGGQGGGGQGGGGQGGGGQC
PPCPAPELLGGPSVFLFPPKPKDTLMISRTPEVTCVVVDVSHEEPEVKFNWYVDGVEVHNAKT
KPCEEQYGSTYRCVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
TCE7 CCR8 scFab VH and CH1
(SEQ ID NO: 92)
EVQLVESGGDLVKPGGSLRLSCAASGFIFSNAWMSWVRQAPGKGLEWVGRIKRKTDGGTTDYA
APVKGRFTISRDDSKNTLYLLMNSLKIEDTAVYYCTVVTLVRGVIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSC
TCE7 CCR8 scFab VL and Ck
(SEQ ID NO: 93)
EIVLTQSPGTLSLSPGESATLSCRASQSVSSSQLAWYQQKPGQTPRLLIYGASSRATGIPDRF
SGSGSGTDFTLTISRLEPEDFAVYYCQQYGNSRTFGQGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
TCE7 CCR8 scFab
(SEQ ID NO: 94)
EVQLVESGGDLVKPGGSLRLSCAASGFIFSNAWMSWVRQAPGKGLEWVGRIKRKTDGGTTDYA
APVKGRFTISRDDSKNTLYLLMNSLKIEDTAVYYCTVVTLVRGVIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGESATLSCRASQSVSSSQLAWYQQKPGQTPRLLIY
GASSRATGIPDRFSGSGSGTDFTLTISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
TCE7 scFab (CCR8) x scFv (CD3)
(SEQ ID NO: 95)
EVQLVESGGDLVKPGGSLRLSCAASGFIFSNAWMSWVRQAPGKGLEWVGRIKRKTDGGTTDYA
APVKGRFTISRDDSKNTLYLLMNSLKIEDTAVYYCTVVTLVRGVIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGESATLSCRASQSVSSSQLAWYQQKPGQTPRLLIY
GASSRATGIPDRFSGSGSGTDFTLTISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECSGGGGQEVQLVESGGGLVQPGGSLK
LSCAASGFTFNKYAINWVRQAPGKGLEWVARIRSKYNNYATYYADAVKDRFTISRDDSKNTVY
LQMNNLKTEDTAVYYCARAGNFGSSYISYWAYWGQGTLVTVSSGGGGQGGGGQGGGGQQTVVT
QEPSLTVSPGGTVTITCGSSTGAVTSGNYPNWVQKKPGQAPRGLIGGTKFLAPGTPARFSGSL
SGGKAALTLSGVQPEDEAEYYCVLWYSNRWVFGSGTKLTVL
TCE7 scFab (CCR8) x scFv (CD3) x scFc
(SEQ ID NO: 96)
EVQLVESGGDLVKPGGSLRLSCAASGFIFSNAWMSWVRQAPGKGLEWVGRIKRKTDGGTTDYA
APVKGRFTISRDDSKNTLYLLMNSLKIEDTAVYYCTVVTLVRGVIFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGESATLSCRASQSVSSSQLAWYQQKPGQTPRLLIY
GASSRATGIPDRFSGSGSGTDFTLTISRLEPEDFAVYYCQQYGNSRTFGQGTKVEIKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECSGGGGQEVQLVESGGGLVQPGGSLK
LSCAASGFTFNKYAINWVRQAPGKGLEWVARIRSKYNNYATYYADAVKDRFTISRDDSKNTVY
LQMNNLKTEDTAVYYCARAGNFGSSYISYWAYWGQGTLVTVSSGGGGQGGGGQGGGGQQTVVT
QEPSLTVSPGGTVTITCGSSTGAVTSGNYPNWVQKKPGQAPRGLIGGTKFLAPGTPARFSGSL
SGGKAALTLSGVQPEDEAEYYCVLWYSNRWVFGSGTKLTVLGGGGCPPCPAPELLGGPSVFLF
PPKPKDTLMISRTPEVTCVVVDVSHEEPEVKFNWYVDGVEVHNAKTKPCEEQYGSTYRCVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGKGGGGQGGGGQGGGGQGGGGQGGGGQGGGGQCPPCPAPELLGGPSVFLF
PPKPKDTLMISRTPEVTCVVVDVSHEEPEVKFNWYVDGVEVHNAKTKPCEEQYGSTYRCVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGK
TCE8 CCR8 scFv and scFab HCDR1
(SEQ ID NO: 97)
NAWMS
TCE8 CCR8 scFv and scFab HCDR2
(SEQ ID NO: 98)
RIKRRTDGGTTDYAAPVKD
TCE8 CCR8 scFv and scFab HCDR3
(SEQ ID NO: 99)
VTMVRGVIADY
TCE8 CCR8 scFv and scFab LCDR1
(SEQ ID NO: 100)
RASQSVSSGSLA
TCE8 CCR8 scFv and scFab LCDR2
(SEQ ID NO: 101)
GASSRAT
TCE8 CCR8 scFv and scFab LCDR3
(SEQ ID NO: 102)
QQYGSSRT
TCE8 CCR8 scFv VH
(SEQ ID NO: 103)
EVQLVESGGGLVKPGGSLRLSCAASGFIFSNAWMSWVRQAPGKCLEWVARIKRRTDGGTTDYA
APVKDRFTISRDDSKNTLFLQMNSLKTEDTAVYYCTTVTMVRGVIADYWGQGTLVTVSS
TCE8 CCR8 scFv VL
(SEQ ID NO: 104)
EIVLTQSPGTLSLSPGERATLSCRASQSVSSGSLAWYQQKLGQAPRLLIYGASSRATGIPDRF
SGSGSGTDFTLTISSLEPEDFAVYYCQQYGSSRTFGCGTKVELK
TCE8 CCR8 scFv
(SEQ ID NO: 105)
EVQLVESGGGLVKPGGSLRLSCAASGFIFSNAWMSWVRQAPGKCLEWVARIKRRTDGGTTDYA
APVKDRFTISRDDSKNTLFLQMNSLKTEDTAVYYCTTVTMVRGVIADYWGQGTLVTVSSGGGG
QGGGGQGGGGQEIVLTQSPGTLSLSPGERATLSCRASQSVSSGSLAWYQQKLGQAPRLLIYGA
SSRATGIPDRFSGSGSGTDFTLTISSLEPEDFAVYYCQQYGSSRTFGCGTKVELK
TCE8 scFv (CCR8) x scFv (CD3)
(SEQ ID NO: 106)
EVQLVESGGGLVKPGGSLRLSCAASGFIFSNAWMSWVRQAPGKCLEWVARIKRRTDGGTTDYA
APVKDRFTISRDDSKNTLFLQMNSLKTEDTAVYYCTTVTMVRGVIADYWGQGTLVTVSSGGGG
QGGGGQGGGGQEIVLTQSPGTLSLSPGERATLSCRASQSVSSGSLAWYQQKLGQAPRLLIYGA
SSRATGIPDRFSGSGSGTDFTLTISSLEPEDFAVYYCQQYGSSRTFGCGTKVELKSGGGGQEV
QLVESGGGLVQPGGSLKLSCAASGFTFNKYAINWVRQAPGKGLEWVARIRSKYNNYATYYADA
VKDRFTISRDDSKNTVYLQMNNLKTEDTAVYYCARAGNFGSSYISYWAYWGQGTLVTVSSGGG
GQGGGGQGGGGQQTVVTQEPSLTVSPGGTVTITCGSSTGAVTSGNYPNWVQKKPGQAPRGLIG
GTKFLAPGTPARFSGSLSGGKAALTLSGVQPEDEAEYYCVLWYSNRWVFGSGTKLTVL
TCE8 scFv (CCR8) x scFv (CD3) x scFc
(SEQ ID NO: 107)
EVQLVESGGGLVKPGGSLRLSCAASGFIFSNAWMSWVRQAPGKCLEWVARIKRRTDGGTTDYA
APVKDRFTISRDDSKNTLFLQMNSLKTEDTAVYYCTTVTMVRGVIADYWGQGTLVTVSSGGGG
QGGGGQGGGGQEIVLTQSPGTLSLSPGERATLSCRASQSVSSGSLAWYQQKLGQAPRLLIYGA
SSRATGIPDRFSGSGSGTDFTLTISSLEPEDFAVYYCQQYGSSRTFGCGTKVELKSGGGGQEV
QLVESGGGLVQPGGSLKLSCAASGFTFNKYAINWVRQAPGKGLEWVARIRSKYNNYATYYADA
VKDRFTISRDDSKNTVYLQMNNLKTEDTAVYYCARAGNFGSSYISYWAYWGQGTLVTVSSGGG
GQGGGGQGGGGQQTVVTQEPSLTVSPGGTVTITCGSSTGAVTSGNYPNWVQKKPGQAPRGLIG
GTKFLAPGTPARFSGSLSGGKAALTLSGVQPEDEAEYYCVLWYSNRWVFGSGTKLTVLGGGGC
PPCPAPELLGGPSVFLFPPKPKDTLMISRTPEVTCVVVDVSHEEPEVKFNWYVDGVEVHNAKT
KPCEEQYGSTYRCVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGKGGGGQGGGGQGGGGQGGGGQGGGGQGGGGQC
PPCPAPELLGGPSVFLFPPKPKDTLMISRTPEVTCVVVDVSHEEPEVKFNWYVDGVEVHNAKT
KPCEEQYGSTYRCVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
TCE8 CCR8 scFab VH and CH1
(SEQ ID NO: 108)
EVQLVESGGGLVKPGGSLRLSCAASGFIFSNAWMSWVRQAPGKGLEWVARIKRRTDGGTTDYA
APVKDRFTISRDDSKNTLFLQMNSLKTEDTAVYYCTTVTMVRGVIADYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSC
TCE8 CCR8 scFab VL and CK
(SEQ ID NO: 109)
EIVLTQSPGTLSLSPGERATLSCRASQSVSSGSLAWYQQKLGQAPRLLIYGASSRATGIPDRF
SGSGSGTDFTLTISSLEPEDFAVYYCQQYGSSRTFGQGTKVELKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
TCE8 CCR8 scFab
(SEQ ID NO: 110)
EVQLVESGGGLVKPGGSLRLSCAASGFIFSNAWMSWVRQAPGKGLEWVARIKRRTDGGTTDYA
APVKDRFTISRDDSKNTLFLQMNSLKTEDTAVYYCTTVTMVRGVIADYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGERATLSCRASQSVSSGSLAWYQQKLGQAPRLLIY
GASSRATGIPDRFSGSGSGTDFTLTISSLEPEDFAVYYCQQYGSSRTFGQGTKVELKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
TCE8 scFab (CCR8) x scFv (CD3)
(SEQ ID NO: 111)
EVQLVESGGGLVKPGGSLRLSCAASGFIFSNAWMSWVRQAPGKGLEWVARIKRRTDGGTTDYA
APVKDRFTISRDDSKNTLFLQMNSLKTEDTAVYYCTTVTMVRGVIADYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGERATLSCRASQSVSSGSLAWYQQKLGQAPRLLIY
GASSRATGIPDRFSGSGSGTDFTLTISSLEPEDFAVYYCQQYGSSRTFGQGTKVELKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECSGGGGQEVQLVESGGGLVQPGGSLK
LSCAASGFTFNKYAINWVRQAPGKGLEWVARIRSKYNNYATYYADAVKDRFTISRDDSKNTVY
LQMNNLKTEDTAVYYCARAGNFGSSYISYWAYWGQGTLVTVSSGGGGQGGGGQGGGGQQTVVT
QEPSLTVSPGGTVTITCGSSTGAVTSGNYPNWVQKKPGQAPRGLIGGTKFLAPGTPARFSGSL
SGGKAALTLSGVQPEDEAEYYCVLWYSNRWVFGSGTKLTVL
TCE8 scFab (CCR8) x scFv (CD3) x scFc
(SEQ ID NO: 112)
EVQLVESGGGLVKPGGSLRLSCAASGFIFSNAWMSWVRQAPGKGLEWVARIKRRTDGGTTDYA
APVKDRFTISRDDSKNTLFLQMNSLKTEDTAVYYCTTVTMVRGVIADYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCGGGGQGGGGQGGGGQGGGGQGGGGQGG
GGQGGGGQGGGGQEIVLTQSPGTLSLSPGERATLSCRASQSVSSGSLAWYQQKLGQAPRLLIY
GASSRATGIPDRFSGSGSGTDFTLTISSLEPEDFAVYYCQQYGSSRTFGQGTKVELKRTVAAP
SVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECSGGGGQEVQLVESGGGLVQPGGSLK
LSCAASGFTFNKYAINWVRQAPGKGLEWVARIRSKYNNYATYYADAVKDRFTISRDDSKNTVY
LQMNNLKTEDTAVYYCARAGNFGSSYISYWAYWGQGTLVTVSSGGGGQGGGGQGGGGQQTVVT
QEPSLTVSPGGTVTITCGSSTGAVTSGNYPNWVQKKPGQAPRGLIGGTKFLAPGTPARFSGSL
SGGKAALTLSGVQPEDEAEYYCVLWYSNRWVFGSGTKLTVLGGGGCPPCPAPELLGGPSVFLF
PPKPKDTLMISRTPEVTCVVVDVSHEEPEVKFNWYVDGVEVHNAKTKPCEEQYGSTYRCVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGKGGGGQGGGGQGGGGQGGGGQGGGGQGGGGQCPPCPAPELLGGPSVFLF
PPKPKDTLMISRTPEVTCVVVDVSHEEPEVKFNWYVDGVEVHNAKTKPCEEQYGSTYRCVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGK
TCE1 CCR8 scFv and scFab HCDR1
(SEQ ID NO: 113)
NARMG
TCE1 CCR8 scFv and scFab HCDR2
(SEQ ID NO: 114)
RIKSKTEGGTRDYAAPVKG
TCE1 CCR8 scFv and scFab HCDR3
(SEQ ID NO: 115)
YSGV
TCE1 CCR8 scFv and scFab LCDR1
(SEQ ID NO: 116)
KSSQSVLYSSNNKNYLA
TCE1 CCR8 scFv and scFab LCDR2
(SEQ ID NO: 117)
WASTRES
TCE1 CCR8 scFv and scFab LCDR3
(SEQ ID NO: 118)
QQYYSIPIT
TCE1 CCR8 scFv VH
(SEQ ID NO: 119)
EVQLVESGGGLVKPGGSLRLSCAASGFTFSNARMGWVRQAPGKCLEWVGRIKSKTEGGTRDYA
APVKGRFTISRDDSKNTLYLQMNSLKTEDTAVYYCTSYSGVWGQGTMVTVSS
TCE1 CCR8 scFv VL
(SEQ ID NO: 120)
EIVMTQSPDSLAVSLGERATINCKSSQSVLYSSNNKNYLAWYHQKPGQSPKLLISWASTRESG
VPDRFSGSGSGTDFTLTINSLQAEDVAVYYCQQYYSIPITFGCGTKVEIK
TCE1 CCR8 scFv
(SEQ ID NO: 121)
EVQLVESGGGLVKPGGSLRLSCAASGFTFSNARMGWVRQAPGKCLEWVGRIKSKTEGGTRDYA
APVKGRFTISRDDSKNTLYLQMNSLKTEDTAVYYCTSYSGVWGQGTMVTVSSGGGGQGGGGQG
GGGQEIVMTQSPDSLAVSLGERATINCKSSQSVLYSSNNKNYLAWYHQKPGQSPKLLISWAST
RESGVPDRFSGSGSGTDFTLTINSLQAEDVAVYYCQQYYSIPITFGCGTKVEIK
TCE1 scFv (CCR8) x scFv (CD3)
(SEQ ID NO: 122)
EVQLVESGGGLVKPGGSLRLSCAASGFTFSNARMGWVRQAPGKCLEWVGRIKSKTEGGTRDYA
APVKGRFTISRDDSKNTLYLQMNSLKTEDTAVYYCTSYSGVWGQGTMVTVSSGGGGQGGGGQG
GGGQEIVMTQSPDSLAVSLGERATINCKSSQSVLYSSNNKNYLAWYHQKPGQSPKLLISWAST
RESGVPDRFSGSGSGTDFTLTINSLQAEDVAVYYCQQYYSIPITFGCGTKVEIKSGGGGQEVQ
LVESGGGLVQPGGSLKLSCAASGFTFNKYAINWVRQAPGKGLEWVARIRSKYNNYATYYADAV
KDRFTISRDDSKNTVYLQMNNLKTEDTAVYYCARAGNFGSSYISYWAYWGQGTLVTVSSGGGG
QGGGGQGGGGQQTVVTQEPSLTVSPGGTVTITCGSSTGAVTSGNYPNWVQKKPGQAPRGLIGG
TKFLAPGTPARFSGSLSGGKAALTLSGVQPEDEAEYYCVLWYSNRWVFGSGTKLTVL
TCE1 scFv (CCR8) x scFv (CD3) x scFc
(SEQ ID NO: 123)
EVQLVESGGGLVKPGGSLRLSCAASGFTFSNARMGWVRQAPGKCLEWVGRIKSKTEGGTRDYA
APVKGRFTISRDDSKNTLYLQMNSLKTEDTAVYYCTSYSGVWGQGTMVTVSSGGGGQGGGGQG
GGGQEIVMTQSPDSLAVSLGERATINCKSSQSVLYSSNNKNYLAWYHQKPGQSPKLLISWAST
RESGVPDRFSGSGSGTDFTLTINSLQAEDVAVYYCQQYYSIPITFGCGTKVEIKSGGGGQEVQ
LVESGGGLVQPGGSLKLSCAASGFTFNKYAINWVRQAPGKGLEWVARIRSKYNNYATYYADAV
KDRFTISRDDSKNTVYLQMNNLKTEDTAVYYCARAGNFGSSYISYWAYWGQGTLVTVSSGGGG
QGGGGQGGGGQQTVVTQEPSLTVSPGGTVTITCGSSTGAVTSGNYPNWVQKKPGQAPRGLIGG
TKFLAPGTPARFSGSLSGGKAALTLSGVQPEDEAEYYCVLWYSNRWVFGSGTKLTVLGGGGCP
PCPAPELLGGPSVFLFPPKPKDTLMISRTPEVTCVVVDVSHEEPEVKFNWYVDGVEVHNAKTK
PCEEQYGSTYRCVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSR
WQQGNVFSCSVMHEALHNHYTQKSLSLSPGKGGGGQGGGGQGGGGQGGGGQGGGGQGGGGQCP
PCPAPELLGGPSVFLFPPKPKDTLMISRTPEVTCVVVDVSHEEPEVKFNWYVDGVEVHNAKTK
PCEEQYGSTYRCVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
TCE1 CCR8 scFab VH and CH1
(SEQ ID NO: 124)
EVQLVESGGGLVKPGGSLRLSCAASGFTFSNARMGWVRQAPGKGLEWVGRIKSKTEGGTRDYA
APVKGRFTISRDDSKNTLYLQMNSLKTEDTAVYYCTSYSGVWGQGTMVTVSSASTKGPSVFPL
APSSKSTSGGTAALGCLVKDYFPEPVTVSWNSGALTSGVHTFPAVLQSSGLYSLSSVVTVPSS
SLGTQTYICNVNHKPSNTKVDKKVEPKSC
TCE1 CCR8 scFab VL and Ck
(SEQ ID NO: 125)
EIVMTQSPDSLAVSLGERATINCKSSQSVLYSSNNKNYLAWYHQKPGQSPKLLISWASTRESG
VPDRFSGSGSGTDFTLTINSLQAEDVAVYYCQQYYSIPITFGGGTKVEIKRTVAAPSVFIFPP
SDEQLKSGTASVVCLLNNFYPREAKVQWKVDNALQSGNSQESVTEQDSKDSTYSLSSTLTLSK
ADYEKHKVYACEVTHQGLSSPVTKSFNRGEC
TCE1 CCR8 scFab
(SEQ ID NO: 126)
EVQLVESGGGLVKPGGSLRLSCAASGFTFSNARMGWVRQAPGKGLEWVGRIKSKTEGGTRDYA
APVKGRFTISRDDSKNTLYLQMNSLKTEDTAVYYCTSYSGVWGQGTMVTVSSASTKGPSVFPL
APSSKSTSGGTAALGCLVKDYFPEPVTVSWNSGALTSGVHTFPAVLQSSGLYSLSSVVTVPSS
SLGTQTYICNVNHKPSNTKVDKKVEPKSCGGGGQGGGGQGGGGQGGGGQGGGGQGGGGQGGGG
QGGGGQEIVMTQSPDSLAVSLGERATINCKSSQSVLYSSNNKNYLAWYHQKPGQSPKLLISWA
STRESGVPDRFSGSGSGTDFTLTINSLQAEDVAVYYCQQYYSIPITFGGGTKVEIKRTVAAPS
VFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
TCE1 scFab (CCR8) x scFv (CD3)
(SEQ ID NO: 127)
EVQLVESGGGLVKPGGSLRLSCAASGFTFSNARMGWVRQAPGKGLEWVGRIKSKTEGGTRDYA
APVKGRFTISRDDSKNTLYLQMNSLKTEDTAVYYCTSYSGVWGQGTMVTVSSASTKGPSVFPL
APSSKSTSGGTAALGCLVKDYFPEPVTVSWNSGALTSGVHTFPAVLQSSGLYSLSSVVTVPSS
SLGTQTYICNVNHKPSNTKVDKKVEPKSCGGGGQGGGGQGGGGQGGGGQGGGGQGGGGQGGGG
QGGGGQEIVMTQSPDSLAVSLGERATINCKSSQSVLYSSNNKNYLAWYHQKPGQSPKLLISWA
STRESGVPDRFSGSGSGTDFTLTINSLQAEDVAVYYCQQYYSIPITFGGGTKVEIKRTVAAPS
VFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGECSGGGGQEVQLVESGGGLVQPGGSLKL
SCAASGFTFNKYAINWVRQAPGKGLEWVARIRSKYNNYATYYADAVKDRFTISRDDSKNTVYL
QMNNLKTEDTAVYYCARAGNFGSSYISYWAYWGQGTLVTVSSGGGGQGGGGQGGGGQQTVVTQ
EPSLTVSPGGTVTITCGSSTGAVTSGNYPNWVQKKPGQAPRGLIGGTKFLAPGTPARFSGSLS
GGKAALTLSGVQPEDEAEYYCVLWYSNRWVFGSGTKLTVL
TCE1 scFab (CCR8) x scFv (CD3) x scFc
(SEQ ID NO: 128)
EVQLVESGGGLVKPGGSLRLSCAASGFTFSNARMGWVRQAPGKGLEWVGRIKSKTEGGTRDYA
APVKGRFTISRDDSKNTLYLQMNSLKTEDTAVYYCTSYSGVWGQGTMVTVSSASTKGPSVFPL
APSSKSTSGGTAALGCLVKDYFPEPVTVSWNSGALTSGVHTFPAVLQSSGLYSLSSVVTVPSS
SLGTQTYICNVNHKPSNTKVDKKVEPKSCGGGGQGGGGQGGGGQGGGGQGGGGQGGGGQGGGG
QGGGGQEIVMTQSPDSLAVSLGERATINCKSSQSVLYSSNNKNYLAWYHQKPGQSPKLLISWA
STRESGVPDRFSGSGSGTDFTLTINSLQAEDVAVYYCQQYYSIPITFGGGTKVEIKRTVAAPS
VFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGECSGGGGQEVQLVESGGGLVQPGGSLKL
SCAASGFTFNKYAINWVRQAPGKGLEWVARIRSKYNNYATYYADAVKDRFTISRDDSKNTVYL
QMNNLKTEDTAVYYCARAGNFGSSYISYWAYWGQGTLVTVSSGGGGQGGGGQGGGGQQTVVTQ
EPSLTVSPGGTVTITCGSSTGAVTSGNYPNWVQKKPGQAPRGLIGGTKFLAPGTPARFSGSLS
GGKAALTLSGVQPEDEAEYYCVLWYSNRWVFGSGTKLTVLGGGGCPPCPAPELLGGPSVFLFP
PKPKDTLMISRTPEVTCVVVDVSHEEPEVKFNWYVDGVEVHNAKTKPCEEQYGSTYRCVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALH
NHYTQKSLSLSPGKGGGGQGGGGQGGGGQGGGGQGGGGQGGGGQCPPCPAPELLGGPSVFLFP
PKPKDTLMISRTPEVTCVVVDVSHEEPEVKFNWYVDGVEVHNAKTKPCEEQYGSTYRCVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALH
NHYTQKSLSLSPGK
Cynomolgus monkey chinese origin CCR8
(SEQ ID NO: 129)
MDYTLDPSMTTMTDYYYPDSLSSPCDGELIQRNDKLLLAVFYCLLFVFSLLGNSLVILVLVVC
KKLRNITDIYLLNLALSDLLFVFSFPFQTYYQLDQWVFGTVMCKVVSGFYYIGFYSSMFFITL
MSVDRYLAVVHAVYAIKVRTIRMGTTLSLVVWLTAIMATIPLLVFYQVASEDGVLQCYSFYNQ
QTLKWKIFTNFEMNILGLLIPFTIFMFCYIKILHQLKRCQNHNKTKAIRLVLIVVIASLLFWV
PFNVVLFLTSLHSMHILDGCSISQQLNYATHVTEIISFTHCCVNPVIYAFVGEKFKKHLSEIF
QKSCSHIFIYLGRQMPRESCEKSSSCQQHSFRSSSIDYIL
Cynomolgus monkey Mauritian origin T4R CCR8
(SEQ ID NO: 130)
MDYRLDPSMTTMTDYYYPDSLSSPCDGELIQRNDKLLLAVFYCLLFVFSLLGNSLVILVLVVC
KKLRNITDIYLLNLALSDLLFVFSFPFQTYYQLDQWVFGTVMCKVVSGFYYIGFYSSMFFITL
MSVDRYLAVVHAVYAIKVRTIRMGTTLSLVVWLTAIMATIPLLVFYQVASEDGVLQCYSFYNQ
QTLKWKIFTNFEMNILGLLIPFTIFMFCYIKILHQLKRCQNHNKTKAIRLVLIVVIASLLFWV
PFNVVLFLTSLHSMHILDGCSISQQLNYATHVTEIISFTHCCVNPVIYAFVGEKFKKHLSEIF
QKSCSHIFIYLGRQMPRESCEKSSSCQQHSFRSSSIDYIL
Human CCR8
(SEQ ID NO: 131)
MDYTLDLSVTTVTDYYYPDIFSSPCDAELIQTNGKLLLAVFYCLLFVFSLLGNSLVILVLVVC
KKLRSITDVYLLNLALSDLLFVFSFPFQTYYLLDQWVFGTVMCKVVSGFYYIGFYSSMFFITL
MSVDRYLAVVHAVYALKVRTIRMGTTLCLAVWLTAIMATIPLLVFYQVASEDGVLQCYSFYNQ
QTLKWKIFTNFKMNILGLLIPFTIFMFCYIKILHQLKRCQNHNKTKAIRLVLIVVIASLLFWV
PFNVVLFLTSLHSMHILDGCSISQQLTYATHVTEIISFTHCCVNPVIYAFVGEKFKKHLSEIF
QKSCSQIFNYLGRQMPRESCEKSSSCQQHSSRSSSVDYIL
Human A27G CCR8
(SEQ ID NO: 132)
MDYTLDLSVTTVTDYYYPDIFSSPCDGELIQTNGKLLLAVFYCLLFVFSLLGNSLVILVLVVC
KKLRSITDVYLLNLALSDLLFVFSFPFQTYYLLDQWVFGTVMCKVVSGFYYIGFYSSMFFITL
MSVDRYLAVVHAVYALKVRTIRMGTTLCLAVWLTAIMATIPLLVFYQVASEDGVLQCYSFYNQ
QTLKWKIFTNFKMNILGLLIPFTIFMFCYIKILHQLKRCQNHNKTKAIRLVLIVVIASLLFWV
PFNVVLFLTSLHSMHILDGCSISQQLTYATHVTEIISFTHCCVNPVIYAFVGEKFKKHLSEIF
QKSCSQIFNYLGRQMPRESCEKSSSCQQHSSRSSSVDYIL
CCR8 P_1-35 peptide
(SEQ ID NO: 133)
MDYTLDLSVTTVTDYYYPDIFSSPCDAELIQTNGK
CCR8 P_1-12 peptide
(SEQ ID NO: 134)
MDYTLDLSVTTV
CCR8 P_13-24 peptide
(SEQ ID NO: 135)
TDYYYPDIFSSP
CCR8 P_25-35 peptide
(SEQ ID NO: 136)
CDAELIQTNGK
CCR8 P_7-18 peptide
(SEQ ID NO: 137)
LSVTTVTDYYYP
CCR8 P_19-30 peptide
(SEQ ID NO: 138)
DIFSSPCDAELI
Antibody 20C1.009 LCDR1
(SEQ ID NO: 139)
RASQGISNWLA
Antibody 20C1.009 LCDR2
(SEQ ID NO: 140)
AASSLQS
Antibody 20C1.009 LCDR3
(SEQ ID NO: 141)
QQAESFPHT
Antibody 20C1.009 HCDR1
(SEQ ID NO: 142)
SYDMS
Antibody 20C1.009 HCDR2
(SEQ ID NO: 143)
LISGGGSQTYYAESVKG
Antibody 20C1.009 HCDR3
(SEQ ID NO: 144)
PSGHYFYAMDV
Antibody 20C1.009 VL
(SEQ ID NO: 145)
DIQMTQSPSSVSASVGDRVTITCRASQGISNWLAWYQQKPGKAPKLLIFAASSLQSGVPSRFS
GSGSGTDFTLTISSLQPEDFATYYCQQAESFPHTFGGGTKVEIK
Antibody 20C1.009 VH
(SEQ ID NO: 146)
EVQLLESGGGLVQPGGSLRLSCAASGFTFSSYDMSWVRQAPGKGLEWVSLISGGGSQTYYAES
VKGRFTISRDNSKNTLYLQMNSLRAEDTAVYFCASPSGHYFYAMDVWGQGTTVTVSS
Antibody 20C1.009 LC
(SEQ ID NO: 147)
DIQMTQSPSSVSASVGDRVTITCRASQGISNWLAWYQQKPGKAPKLLIFAASSLQSGVPSRFS
GSGSGTDFTLTISSLQPEDFATYYCQQAESFPHTFGGGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
Antibody 20C1.009 HC
(SEQ ID NO: 148)
EVQLLESGGGLVQPGGSLRLSCAASGFTFSSYDMSWVRQAPGKGLEWVSLISGGGSQTYYAES
VKGRFTISRDNSKNTLYLQMNSLRAEDTAVYFCASPSGHYFYAMDVWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPCEEQYGSTYRCVSVLTVLHQD
WLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQ
KSLSLSPGK
Antibody 20A2.3 LCDR1
(SEQ ID NO: 149)
SGDKLGDKYAS
Antibody 20A2.3 LCDR2
(SEQ ID NO: 150)
QDRKRPS
Antibody 20A2.3 LCDR3
(SEQ ID NO: 151)
QAFESSTEV
Antibody 20A2.3 HCDR1
(SEQ ID NO: 152)
NYGMH
Antibody 20A2.3 HCDR2
(SEQ ID NO: 153)
LIWYDASKKYYAESVKG
Antibody 20A2.3 HCDR3
(SEQ ID NO: 154)
DPSSLTGSTGYYGMDV
Antibody 20A2.3 VL
(SEQ ID NO: 155)
SYELTQPPSVSVSPGQTASITCSGDKLGDKYASWYQQKPGQSPVLVIYQDRKRPSGIPERFSG
SNSGNTATLTISGTQAMDEADYYCQAFESSTEVFGGGTKLTVL
Antibody 20A2.3 VH
(SEQ ID NO: 156)
QVQLVESGGGVVQPGRSLRLSCAASGFTFSNYGMHWVRQAPGKGLEWVALIWYDASKKYYAES
VKGRFTISRDNSKNTLYLQMNSLRAEDTAAYYCARDPSSLTGSTGYYGMDVWGQGTTVTVSS
Antibody 20A2.3 LC
(SEQ ID NO: 157)
SYELTQPPSVSVSPGQTASITCSGDKLGDKYASWYQQKPGQSPVLVIYQDRKRPSGIPERFSG
SNSGNTATLTISGTQAMDEADYYCQAFESSTEVFGGGTKLTVLGQPKAAPSVTLFPPSSEELQ
ANKATLVCLISDFYPGAVTVAWKADSSPVKAGVETTTPSKQSNNKYAASSYLSLTPEQWKSHR
SYSCQVTHEGSTVEKTVAPTECS
Antibody 20A2.3 HC
(SEQ ID NO: 158)
QVQLVESGGGVVQPGRSLRLSCAASGFTFSNYGMHWVRQAPGKGLEWVALIWYDASKKYYAES
VKGRFTISRDNSKNTLYLQMNSLRAEDTAAYYCARDPSSLTGSTGYYGMDVWGQGTTVTVSSA
STKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPCEEQYGSTYRCVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALH
NHYTQKSLSLSPGK
Antibody 20D4.6 LCDR1
(SEQ ID NO: 159)
SGDALPKKYAY
Antibody 20D4.6 LCDR2
(SEQ ID NO: 160)
EDAKRPS
Antibody 20D4.6 LCDR3
(SEQ ID NO: 161)
YSTDASGNHRV
Antibody 20D4.6 HCDR1
(SEQ ID NO: 162)
DYSMS
Antibody 20D4.6 HCDR2
(SEQ ID NO: 163)
GINWNGGRTRYADAVKG
Antibody 20D4.6 HCDR3
(SEQ ID NO: 164)
EFNNFESNWFDP
Antibody 20D4.6 VL
(SEQ ID NO: 165)
SYELTQPPSVSVSPGQTARITCSGDALPKKYAYWYQQKPGQAPVLVISEDAKRPSGIPERFSG
SSSGTMATLTISGAQVEDEADYYCYSTDASGNHRVFGGGTKLTVL
Antibody 20D4.6 VH
(SEQ ID NO: 166)
EVQLVESGGSVVRPGGSLRLSCAASGFTVDDYSMSWVRQVPGKGLEWVSGINWNGGRTRYADA
VKGRFTISRDSAKNSLYLQMNSLRAEDTALYYCAREFNNFESNWFDPWGQGTLVTVSS
Antibody 20D4.6 LC
(SEQ ID NO: 167)
SYELTQPPSVSVSPGQTARITCSGDALPKKYAYWYQQKPGQAPVLVISEDAKRPSGIPERFSG
SSSGTMATLTISGAQVEDEADYYCYSTDASGNHRVFGGGTKLTVLGQPKAAPSVTLFPPSSEE
LQANKATLVCLISDFYPGAVTVAWKADSSPVKAGVETTTPSKQSNNKYAASSYLSLTPEQWKS
HRSYSCQVTHEGSTVEKTVAPTECS
Antibody 20D4.6 HC
(SEQ ID NO: 168)
EVQLVESGGSVVRPGGSLRLSCAASGFTVDDYSMSWVRQVPGKGLEWVSGINWNGGRTRYADA
VKGRFTISRDSAKNSLYLQMNSLRAEDTALYYCAREFNNFESNWFDPWGQGTLVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPCEEQYGSTYRCVSVLTVLHQ
DWLNGKEYKCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHEALHNHYT
QKSLSLSPGK
Antibody 20D4.17 LCDR1
(SEQ ID NO: 169)
SGDALPKKYAY
Antibody 20D4.17 LCDR2
(SEQ ID NO: 170)
EDAKRPS
Antibody 20D4.17 LCDR3
(SEQ ID NO: 171)
YSTDASGNHRV
Antibody 20D4.17 HCDR1
(SEQ ID NO: 172)
DYSMS
Antibody 20D4.17 HCDR2
(SEQ ID NO: 173)
GINWNAGRTRYADAVKG
Antibody 20D4.17 HCDR3
(SEQ ID NO: 174)
EFNNFESNWFDP
Antibody 20D4.17 VL
(SEQ ID NO: 175)
SYELTQPPSVSVSPGQTARITCSGDALPKKYAYWYQQKPGQAPVLVISEDAKRPSGIPERFSG
SSSGTMATLTISGAQVEDEADYYCYSTDASGNHRVFGGGTKLTVL
Antibody 20D4.17 VH
(SEQ ID NO: 176)
EVQLVESGGSVVRPGGSLRLSCAASGFTVDDYSMSWVRQVPGKGLEWVSGINWNAGRTRYADA
VKGRFTISRDSAKNSLYLQMNSLRAEDTALYYCAREFNNFESNWFDPWGQGTLVTVSS
Antibody 20D4.17 LC
(SEQ ID NO: 177)
SYELTQPPSVSVSPGQTARITCSGDALPKKYAYWYQQKPGQAPVLVISEDAKRPSGIPERFSG
SSSGTMATLTISGAQVEDEADYYCYSTDASGNHRVFGGGTKLTVLGQPKAAPSVTLFPPSSEE
LQANKATLVCLISDFYPGAVTVAWKADSSPVKAGVETTTPSKQSNNKYAASSYLSLTPEQWKS
HRSYSCQVTHEGSTVEKTVAPTECS
Antibody 20D4.17 HC
(SEQ ID NO: 178)
EVQLVESGGSVVRPGGSLRLSCAASGFTVDDYSMSWVRQVPGKGLEWVSGINWNAGRTRYADA
VKGRFTISRDSAKNSLYLQMNSLRAEDTALYYCAREFNNFESNWFDPWGQGTLVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPCEEQYGSTYRCVSVLTVLHQ
DWLNGKEYKCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHEALHNHYT
QKSLSLSPGK
Antibody 20C1.006 LCDR1
(SEQ ID NO: 179)
RASQGISNWLA
Antibody 20C1.006 LCDR2
(SEQ ID NO: 180)
AASSLQS
Antibody 20C1.006 LCDR3
(SEQ ID NO: 181)
QQAESFPHT
Antibody 20C1.006 HCDR1
(SEQ ID NO: 182)
SYDMS
Antibody 20C1.006 HCDR2
(SEQ ID NO: 183)
LISGGGSNTYYAESVKG
Antibody 20C1.006 HCDR3
(SEQ ID NO: 184)
PSGHYFYAMDV
Antibody 20C1.006 VL
(SEQ ID NO: 185)
DIQMTQSPSSVSASVGDRVTITCRASQGISNWLAWYQQKPGKAPKLLIFAASSLQSGVPSRFS
GSGSGTDFTLTISSLQPEDFATYYCQQAESFPHTFGGGTKVEIK
Antibody 20C1.006 VH
(SEQ ID NO: 186)
EVQLLESGGGLVQPGGSLRLSCAASGFTFSSYDMSWVRQAPGKGLEWVSLISGGGSNTYYAES
VKGRFTISRDNSKNTLYLQMNSLRAEDTAVYFCASPSGHYFYAMDVWGQGTTVTVSS
Antibody 20C1.006 LC
(SEQ ID NO: 187)
DIQMTQSPSSVSASVGDRVTITCRASQGISNWLAWYQQKPGKAPKLLIFAASSLQSGVPSRFS
GSGSGTDFTLTISSLQPEDFATYYCQQAESFPHTFGGGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
Antibody 20C1.006 HC
(SEQ ID NO: 188)
EVQLLESGGGLVQPGGSLRLSCAASGFTFSSYDMSWVRQAPGKGLEWVSLISGGGSNTYYAES
VKGRFTISRDNSKNTLYLQMNSLRAEDTAVYFCASPSGHYFYAMDVWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPCEEQYGSTYRCVSVLTVLHQD
WLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQ
KSLSLSPGK
G4S linker
(SEQ ID NO: 189)
GGGGS
(G4S)4 linker
(SEQ ID NO: 190)
GGGGSGGGGSGGGGSGGGGS
I2C - HCDR1
(SEQ ID NO: 191)
KYAMN
I2C - HCDR2
(SEQ ID NO: 192)
RIRSKYNNYATYYADSVKD
I2C - HCDR3
(SEQ ID NO: 193)
HGNFGNSYISYWAY
I2C - LCDR1
(SEQ ID NO: 194)
GSSTGAVTSGNYPN
I2C - LCDR2
(SEQ ID NO: 195)
GTKFLAP
I2C - LCDR3
(SEQ ID NO: 196)
VLWYSNRWV
I2C - VH
(SEQ ID NO: 197)
EVQLVESGGGLVQPGGSLKLSCAASGFTFNKYAMNWVRQAPGKGLEWVARIRSKYNNYATYYA
DSVKDRFTISRDDSKNTAYLQMNNLKTEDTAVYYCVRHGNFGNSYISYWAYWGQGTLVTVSS
I2C - VL
(SEQ ID NO: 198)
QTVVTQEPSLTVSPGGTVTLTCGSSTGAVTSGNYPNWVQQKPGQAPRGLIGGTKFLAPGTPAR
FSGSLLGGKAALTLSGVQPEDEAEYYCVLWYSNRWVFGGGTKLTVL
I2E - HCDR1
(SEQ ID NO: 199)
KYAIN
I2E - HCDR2
(SEQ ID NO: 200)
RIRSKYNNYATYYADAVKD
I2E - HCDR3
(SEQ ID NO: 201)
AGNFGSSYISYWAY
I2E - LCDR1
(SEQ ID NO: 202)
GSSTGAVTSGNYPN
I2E - LCDR2
(SEQ ID NO: 203)
GTKFLAP
I2E - LCDR3
(SEQ ID NO: 204)
VLWYSNRWV
I2E - VH
(SEQ ID NO: 205)
EVQLVESGGGLVQPGGSLKLSCAASGFTFNKYAINWVRQAPGKGLEWVARIRSKYNNYATYYA
DAVKDRFTISRDDSKNTVYLQMNNLKTEDTAVYYCARAGNFGSSYISYWAYWGQGTLVTVSS
I2E - VL
(SEQ ID NO: 206)
QTVVTQEPSLTVSPGGTVTITCGSSTGAVTSGNYPNWVQKKPGQAPRGLIGGTKFLAPGTPAR
FSGSLSGGKAALTLSGVQPEDEAEYYCVLWYSNRWVFGSGTKLTVL
G4Q Linker
(SEQ ID NO: 207)
GGGGQ
(G4Q)4 Linker
(SEQ ID NO: 208)
GGGGQGGGGQGGGGQGGGGQ
mCCR8
(SEQ ID NO: 209)
MDYTMEPNVTMTDYYPDFFTAPCDAEFLLRGSMLYLAILYCVLFVLGLLGNSLVILVLVGCKK
LRSITDIYLLNLAASDLLFVLSIPFQTHNLLDQWVFGTAMCKVVSGLYYIGFFSSMFFITLMS
VDRYLAIVHAVYAIKVRTASVGTALSLTVWLAAVTATIPLMVFYQVASEDGMLQCFQFYEEQS
LRWKLFTHFEINALGLLLPFAILLFCYVRILQQLRGCLNHNRTRAIKLVLTVVIVSLLFWVPF
NVALFLTSLHDLHILDGCATRQRLALAIHVTEVISFTHCCVNPVIYAFIGEKFKKHLMDVFQK
SCSHIFLYLGRQMPVGALERQLSSNQRSSHSSTLDDIL
Rat CCR8
(SEQ ID NO: 210)
MDYTLEPNVTMTDYYPDFFTTPCDTELLLRGGTLYLAVLYCILFVLGLLGNSLVILVLVACKK
LRSITDVYLLNLAASDLLFVLSIPFQTHNLLDQWVFGTVMCKVVSGLYYIGFFSSMLFITLMS
VDRYLAVVHPVHAIKVRTARVGTALSLAVWLAAIAATVPLMVFYQVSSEDGMLQCFQLYDEQS
LRWKLFTHFEVNALGLLLPFAILLFCYVRILQQLRGCLNHNRTRAIKLVLTIVVVSLLFWVPF
NVVLFLTSLHDMHILEGCATRQRLALATHVTEVISFMHCCVNPVIYAFIGEKFKKHLVDVFQK
SCSHIFLYVGRQMPVGALERQLSSNQRSSHSSTLDYIL
hCCR4
(SEQ ID NO: 211)
MNPTDIADTTLDESIYSNYYLYESIPKPCTKEGIKAFGELFLPPLYSLVFVFGLLGNSVVVLV
LFKYKRLRSMTDVYLLNLAISDLLFVFSLPFWGYYAADQWVFGLGLCKMISWMYLVGFYSGIF
FVMLMSIDRYLAIVHAVFSLRARTLTYGVITSLATWSVAVFASLPGFLFSTCYTERNHTYCKT
KYSLNSTTWKVLSSLEINILGLVIPLGIMLFCYSMIIRTLQHCKNEKKNKAVKMIFAVVVLFL
GFWTPYNIVLFLETLVELEVLQDCTFERYLDYAIQATETLAFVHCCLNPIIYFFLGEKFRKYI
LQLFKTCRGLFVLCQYCGLLQIYSADTPSSSYTQSTMDHDLHDAL
Antibody 20C1.009 HC without C-terminal lysine
(SEQ ID NO: 212)
EVQLLESGGGLVQPGGSLRLSCAASGFTFSSYDMSWVRQAPGKGLEWVSLISGGGSQTYYAES
VKGRFTISRDNSKNTLYLQMNSLRAEDTAVYFCASPSGHYFYAMDVWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPCEEQYGSTYRCVSVLTVLHQD
WLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQ
KSLSLSPG
Leader sequence
(SEQ ID NO: 213)
MDMRVPAQLL GLLLLWLRGA RC
DNA encoding leader sequence of SEQ ID NO: 213
(SEQ ID NO: 214)
atggacatga gagtgcctgc acagctgctg ggcctgctgc tgctgtggct
gagaggcgcc agatgc.
Leader sequence
(SEQ ID NO: 215)
MAWALLLLTL LTQGTGSWA
DNA encoding leader sequence of SEQ ID NO: 215
(SEQ ID NO: 216)
atggcctggg ctctgctgct cctcaccctc ctcactcagg gcacagggtc
ctgggcc

TABLE 24
TCE molecule sequences.

| | | |
|---|---|---|
| SEQ ID | | |
| NO: | Designation | Sequence |

CCR8 TCE 1.1 (CC EI GQ x I2E GQ x scFcmod delGK)

| | | |
|---|---|---|
| 217 | HCDR1 | NARMG |
| 218 | HCDR2 | RIKSKTEGGTRDYAAPVKG |
| 219 | HCDR3 | YSGV |
| 220 | LCDR1 | KSSQSVLYSSNNKNYLA |
| 221 | LCDR2 | WASTRES |
| 222 | LCDR3 | QQYYSIPIT |
| 223 | VH | EVQLVESGGGLVKPGGSLRLSCAASGFTFSNARMGWVRQA |
| | | PGKCLEWVGRIKSKTEGGTRDYAAPVKGRFTISRDDSKNT |
| | | LYLQMNSLKTEDTAVYYCTSYSGVWGQGTMVTVSS |
| 224 | VL | EIVMTQSPDSLAVSLGERATINCKSSQSVLYSSNNKNYLA |
| | | WYHQKPGQSPKLLISWASTRESGVPDRFSGSGSGTDFTLT |
| | | INSLQAEDVAVYYCQQYYSIPITFGCGTKVEIK |
| 225 | SCFV | EVQLVESGGGLVKPGGSLRLSCAASGFTFSNARMGWVRQA |
| | | PGKCLEWVGRIKSKTEGGTRDYAAPVKGRFTISRDDSKNT |
| | | LYLQMNSLKTEDTAVYYCTSYSGVWGQGTMVTVSSGGGGQ |
| | | GGGGQGGGGQEIVMTQSPDSLAVSLGERATINCKSSQSVL |
| | | YSSNNKNYLAWYHQKPGQSPKLLISWASTRESGVPDRFSG |
| | | SGSGTDFTLTINSLQAEDVAVYYCQQYYSIPITFGCGTKV |
| | | EIK |
| 226 | TCE 1.1 | EVQLVESGGGLVKPGGSLRLSCAASGFTFSNARMGWVRQA |
| | | PGKCLEWVGRIKSKTEGGTRDYAAPVKGRFTISRDDSKNT |
| | | LYLQMNSLKTEDTAVYYCTSYSGVWGQGTMVTVSSGGGGQ |
| | | GGGGQGGGGQEIVMTQSPDSLAVSLGERATINCKSSQSVL |
| | | YSSNNKNYLAWYHQKPGQSPKLLISWASTRESGVPDRFSG |
| | | SGSGTDFTLTINSLQAEDVAVYYCQQYYSIPITFGCGTKV |
| | | EIKSGGGGQEVQLVESGGGLVQPGGSLKLSCAASGFTFNK |
| | | YAINWVRQAPGKGLEWVARIRSKYNNYATYYADAVKDRFT |
| | | ISRDDSKNTVYLQMNNLKTEDTAVYYCARAGNFGSSYISY |
| | | WAYWGQGTLVTVSSGGGGQGGGGQGGGGQQTVVTQEPSLT |
| | | VSPGGTVTITCGSSTGAVTSGNYPNWVQKKPGQAPRGLIG |
| | | GTKFLAPGTPARFSGSLSGGKAALTLSGVQPEDEAEYYCV |
| | | LWYSNRWVFGSGTKLTVL |
| 227 | TCE 1.1 HLE | EVQLVESGGGLVKPGGSLRLSCAASGFTFSNARMGWVRQA |
| | | PGKCLEWVGRIKSKTEGGTRDYAAPVKGRFTISRDDSKNT |
| | | LYLQMNSLKTEDTAVYYCTSYSGVWGQGTMVTVSSGGGGQ |
| | | GGGGQGGGGQEIVMTQSPDSLAVSLGERATINCKSSQSVL |
| | | YSSNNKNYLAWYHQKPGQSPKLLISWASTRESGVPDRFSG |
| | | SGSGTDFTLTINSLQAEDVAVYYCQQYYSIPITFGCGTKV |
| | | EIKSGGGGQEVQLVESGGGLVQPGGSLKLSCAASGFTFNK |
| | | YAINWVRQAPGKGLEWVARIRSKYNNYATYYADAVKDRFT |
| | | ISRDDSKNTVYLQMNNLKTEDTAVYYCARAGNFGSSYISY |
| | | WAYWGQGTLVTVSSGGGGQGGGGQGGGGQQTVVTQEPSLT |
| | | VSPGGTVTITCGSSTGAVTSGNYPNWVQKKPGQAPRGLIG |
| | | GTKFLAPGTPARFSGSLSGGKAALTLSGVQPEDEAEYYCV |
| | | LWYSNRWVFGSGTKLTVLGGGGCPPCPAPELLGGPSVFLF |
| | | PPKPKDTLMISRTPEVTCVVVDVSHEEPEVKFNWYVDGVE |
| | | VHNAKTKPCEEQYGSTYRCVSVLTVLHQDWLNGKEYKCKV |
| | | SNKALPAPIEKTISKAKGQPREPQVYTLPPSREEMTKNQV |
| | | SLTCLVKGFYPSDIAVEWESNGQPENNYKTTPPVLDSDGS |
| | | FFLYSKLTVDKSRWQQGNVFSCSVMHEALHNHYTQKSLSL |
| | | SPGGGGQGGGGQGGGGQGGGGQGGGGQGGGGQCPPCPAPE |
| | | LLGGPSVFLFPPKPKDTLMISRTPEVTCVVVDVSHEEPEV |
| | | KFNWYVDGVEVHNAKTKPCEEQYGSTYRCVSVLTVLHQDW |
| | | LNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVMHEALH |
| | | NHYTQKSLSLSPGK |

CCR8 TCE 1.2 (CC EI GQ x I2E GQ x scFcmod delGK_YTE)

| | | |
|---|---|---|
| 228 | HCDR1 | NARMG |
| 229 | HCDR2 | RIKSKTEGGTRDYAAPVKG |
| 230 | HCDR3 | YSGV |
| 231 | LCDR1 | KSSQSVLYSSNNKNYLA |
| 232 | LCDR2 | WASTRES |
| 233 | LCDR3 | QQYYSIPIT |
| 234 | VH | EVQLVESGGGLVKPGGSLRLSCAASGFTFSNARMGWVRQA |
| | | PGKCLEWVGRIKSKTEGGTRDYAAPVKGRFTISRDDSKNT |
| | | LYLQMNSLKTEDTAVYYCTSYSGVWGQGTMVTVSS |
| 235 | VL | EIVMTQSPDSLAVSLGERATINCKSSQSVLYSSNNKNYLA |
| | | WYHQKPGQSPKLLISWASTRESGVPDRFSGSGSGTDFTLT |
| | | INSLQAEDVAVYYCQQYYSIPITFGCGTKVEIK |
| 236 | SCFV | EVQLVESGGGLVKPGGSLRLSCAASGFTFSNARMGWVRQA |
| | | PGKCLEWVGRIKSKTEGGTRDYAAPVKGRFTISRDDSKNT |
| | | LYLQMNSLKTEDTAVYYCTSYSGVWGQGTMVTVSSGGGGQ |
| | | GGGGQGGGGQEIVMTQSPDSLAVSLGERATINCKSSQSVL |
| | | YSSNNKNYLAWYHQKPGQSPKLLISWASTRESGVPDRFSG |
| | | SGSGTDFTLTINSLQAEDVAVYYCQQYYSIPITFGCGTKV |
| | | EIK |
| 237 | TCE 1.2 | EVQLVESGGGLVKPGGSLRLSCAASGFTFSNARMGWVRQA |
| | | PGKCLEWVGRIKSKTEGGTRDYAAPVKGRFTISRDDSKNT |
| | | LYLQMNSLKTEDTAVYYCTSYSGVWGQGTMVTVSSGGGGQ |
| | | GGGGQGGGGQEIVMTQSPDSLAVSLGERATINCKSSQSVL |
| | | YSSNNKNYLAWYHQKPGQSPKLLISWASTRESGVPDRFSG |
| | | SGSGTDFTLTINSLQAEDVAVYYCQQYYSIPITFGCGTKV |
| | | EIKSGGGGQEVQLVESGGGLVQPGGSLKLSCAASGFTFNK |
| | | YAINWVRQAPGKGLEWVARIRSKYNNYATYYADAVKDRFT |
| | | ISRDDSKNTVYLQMNNLKTEDTAVYYCARAGNFGSSYISY |
| | | WAYWGQGTLVTVSSGGGGQGGGGQGGGGQQTVVTQEPSLT |
| | | VSPGGTVTITCGSSTGAVTSGNYPNWVQKKPGQAPRGLIG |
| | | GTKFLAPGTPARFSGSLSGGKAALTLSGVQPEDEAEYYCV |
| | | LWYSNRWVFGSGTKLTVL |
| 238 | TCE 1.2 HLE | EVQLVESGGGLVKPGGSLRLSCAASGFTFSNARMGWVRQA |
| | | PGKCLEWVGRIKSKTEGGTRDYAAPVKGRFTISRDDSKNT |
| | | LYLQMNSLKTEDTAVYYCTSYSGVWGQGTMVTVSSGGGGQ |
| | | GGGGQGGGGQEIVMTQSPDSLAVSLGERATINCKSSQSVL |
| | | YSSNNKNYLAWYHQKPGQSPKLLISWASTRESGVPDRFSG |
| | | SGSGTDFTLTINSLQAEDVAVYYCQQYYSIPITFGCGTKV |
| | | EIKSGGGGQEVQLVESGGGLVQPGGSLKLSCAASGFTFNK |
| | | YAINWVRQAPGKGLEWVARIRSKYNNYATYYADAVKDRFT |
| | | ISRDDSKNTVYLQMNNLKTEDTAVYYCARAGNFGSSYISY |
| | | WAYWGQGTLVTVSSGGGGQGGGGQGGGGQQTVVTQEPSLT |
| | | VSPGGTVTITCGSSTGAVTSGNYPNWVQKKPGQAPRGLIG |
| | | GTKFLAPGTPARFSGSLSGGKAALTLSGVQPEDEAEYYCV |
| | | LWYSNRWVFGSGTKLTVLGGGGCPPCPAPELLGGPSVFLF |
| | | PPKPKDTLYITREPEVTCVVVDVSHEEPEVKFNWYVDGVE |
| | | VHNAKTKPCEEQYGSTYRCVSVLTVLHQDWLNGKEYKCKV |
| | | SNKALPAPIEKTISKAKGQPREPQVYTLPPSREEMTKNQV |
| | | SLTCLVKGFYPSDIAVEWESNGQPENNYKTTPPVLDSDGS |
| | | FFLYSKLTVDKSRWQQGNVFSCSVMHEALHNHYTQKSLSL |
| | | SPGGGGQGGGGQGGGGQGGGGQGGGGQGGGGQCPPCPAPE |
| | | LLGGPSVFLFPPKPKDTLYITREPEVTCVVVDVSHEEPEV |
| | | KFNWYVDGVEVHNAKTKPCEEQYGSTYRCVSVLTVLHQDW |
| | | LNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVMHEALH |
| | | NHYTQKSLSLSPGK |

CCR8 TCE 1.3 (EI scFab x I2E GQ x scFcmod delGK_YTE)

| | | |
|---|---|---|
| 239 | HCDR1 | NARMG |
| 240 | HCDR2 | RIKSKTEGGTRDYAAPVKG |
| 241 | HCDR3 | YSGV |
| 242 | LCDR1 | KSSQSVLYSSNNKNYLA |
| 243 | LCDR2 | WASTRES |
| 244 | LCDR3 | QQYYSIPIT |
| 245 | VH-CH1 | EVQLVESGGGLVKPGGSLRLSCAASGFTFSNARMGWVRQA |
| | | PGKGLEWVGRIKSKTEGGTRDYAAPVKGRFTISRDDSKNT |
| | | LYLQMNSLKTEDTAVYYCTSYSGVWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSC |
| 246 | VL-Ck | EIVMTQSPDSLAVSLGERATINCKSSQSVLYSSNNKNYLA |
| | | WYHQKPGQSPKLLISWASTRESGVPDRFSGSGSGTDFTLT |
| | | INSLQAEDVAVYYCQQYYSIPITFGGGTKVEIKRTVAAPS |
| | | VFIFPPSDEQLKSGTASVVCLLNNFYPREAKVQWKVDNAL |
| | | QSGNSQESVTEQDSKDSTYSLSSTLTLSKADYEKHKVYAC |
| | | EVTHQGLSSPVTKSFNRGEC |
| 247 | SCFAB | EVQLVESGGGLVKPGGSLRLSCAASGFTFSNARMGWVRQA |
| | | PGKGLEWVGRIKSKTEGGTRDYAAPVKGRFTISRDDSKNT |
| | | LYLQMNSLKTEDTAVYYCTSYSGVWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCGGGGQGGGGQGGGGQGGGGQGG |
| | | GGQGGGGQGGGGQGGGGQEIVMTQSPDSLAVSLGERATIN |
| | | CKSSQSVLYSSNNKNYLAWYHQKPGQSPKLLISWASTRES |
| | | GVPDRFSGSGSGTDFTLTINSLQAEDVAVYYCQQYYSIPI |
| | | TFGGGTKVEIKRTVAAPSVFIFPPSDEQLKSGTASVVCLL |
| | | NNFYPREAKVQWKVDNALQSGNSQESVTEQDSKDSTYSLS |
| | | STLTLSKADYEKHKVYACEVTHQGLSSPVTKSFNRGEC |
| 248 | TCE 1.3 | EVQLVESGGGLVKPGGSLRLSCAASGFTFSNARMGWVRQA |
| | | PGKGLEWVGRIKSKTEGGTRDYAAPVKGRFTISRDDSKNT |
| | | LYLQMNSLKTEDTAVYYCTSYSGVWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCGGGGQGGGGQGGGGQGGGGQGG |
| | | GGQGGGGQGGGGQGGGGQEIVMTQSPDSLAVSLGERATIN |
| | | CKSSQSVLYSSNNKNYLAWYHQKPGQSPKLLISWASTRES |
| | | GVPDRFSGSGSGTDFTLTINSLQAEDVAVYYCQQYYSIPI |
| | | TFGGGTKVEIKRTVAAPSVFIFPPSDEQLKSGTASVVCLL |
| | | NNFYPREAKVQWKVDNALQSGNSQESVTEQDSKDSTYSLS |
| | | STLTLSKADYEKHKVYACEVTHQGLSSPVTKSFNRGECSG |
| | | GGGQEVQLVESGGGLVQPGGSLKLSCAASGFTFNKYAINW |
| | | VRQAPGKGLEWVARIRSKYNNYATYYADAVKDRFTISRDD |
| | | SKNTVYLQMNNLKTEDTAVYYCARAGNFGSSYISYWAYWG |
| | | QGTLVTVSSGGGGQGGGGQGGGGQQTVVTQEPSLTVSPGG |
| | | TVTITCGSSTGAVTSGNYPNWVQKKPGQAPRGLIGGTKFL |
| | | APGTPARFSGSLSGGKAALTLSGVQPEDEAEYYCVLWYSN |
| | | RWVFGSGTKLTVL |
| 249 | TCE 1.3 HLE | EVQLVESGGGLVKPGGSLRLSCAASGFTFSNARMGWVRQA |
| | | PGKGLEWVGRIKSKTEGGTRDYAAPVKGRFTISRDDSKNT |
| | | LYLQMNSLKTEDTAVYYCTSYSGVWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCGGGGQGGGGQGGGGQGGGGQGG |
| | | GGQGGGGQGGGGQGGGGQEIVMTQSPDSLAVSLGERATIN |
| | | CKSSQSVLYSSNNKNYLAWYHQKPGQSPKLLISWASTRES |
| | | GVPDRFSGSGSGTDFTLTINSLQAEDVAVYYCQQYYSIPI |
| | | TFGGGTKVEIKRTVAAPSVFIFPPSDEQLKSGTASVVCLL |
| | | NNFYPREAKVQWKVDNALQSGNSQESVTEQDSKDSTYSLS |
| | | STLTLSKADYEKHKVYACEVTHQGLSSPVTKSFNRGECSG |
| | | GGGQEVQLVESGGGLVQPGGSLKLSCAASGFTFNKYAINW |
| | | VRQAPGKGLEWVARIRSKYNNYATYYADAVKDRFTISRDD |
| | | SKNTVYLQMNNLKTEDTAVYYCARAGNFGSSYISYWAYWG |
| | | QGTLVTVSSGGGGQGGGGQGGGGQQTVVTQEPSLTVSPGG |
| | | TVTITCGSSTGAVTSGNYPNWVQKKPGQAPRGLIGGTKFL |
| | | APGTPARFSGSLSGGKAALTLSGVQPEDEAEYYCVLWYSN |
| | | RWVFGSGTKLTVLGGGGCPPCPAPELLGGPSVFLFPPKPK |
| | | DTLYITREPEVTCVVVDVSHEEPEVKFNWYVDGVEVHNAK |
| | | TKPCEEQYGSTYRCVSVLTVLHQDWLNGKEYKCKVSNKAL |
| | | PAPIEKTISKAKGQPREPQVYTLPPSREEMTKNQVSLTCL |
| | | VKGFYPSDIAVEWESNGQPENNYKTTPPVLDSDGSFFLYS |
| | | KLTVDKSRWQQGNVFSCSVMHEALHNHYTQKSLSLSPGGG |
| | | GQGGGGQGGGGQGGGGQGGGGQGGGGQCPPCPAPELLGGP |
| | | SVFLFPPKPKDTLYITREPEVTCVVVDVSHEEPEVKFNWY |
| | | VDGVEVHNAKTKPCEEQYGSTYRCVSVLTVLHQDWLNGKE |
| | | YKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYTQ |
| | | KSLSLSPGK |

CCR8 TCE 1.4 (EI scFab x I2E GQ x scFcmod delGK)

| | | |
|---|---|---|
| 250 | HCDR1 | NARMG |
| 251 | HCDR2 | RIKSKTEGGTRDYAAPVKG |
| 252 | HCDR3 | YSGV |
| 253 | LCDR1 | KSSQSVLYSSNNKNYLA |
| 254 | LCDR2 | WASTRES |
| 255 | LCDR3 | QQYYSIPIT |
| 256 | VH-CH1 | EVQLVESGGGLVKPGGSLRLSCAASGFTFSNARMGWVRQA |
| | | PGKGLEWVGRIKSKTEGGTRDYAAPVKGRFTISRDDSKNT |
| | | LYLQMNSLKTEDTAVYYCTSYSGVWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSC |
| 257 | VL-Ck | EIVMTQSPDSLAVSLGERATINCKSSQSVLYSSNNKNYLA |
| | | WYHQKPGQSPKLLISWASTRESGVPDRFSGSGSGTDFTLT |
| | | INSLQAEDVAVYYCQQYYSIPITFGGGTKVEIKRTVAAPS |
| | | VFIFPPSDEQLKSGTASVVCLLNNFYPREAKVQWKVDNAL |
| | | QSGNSQESVTEQDSKDSTYSLSSTLTLSKADYEKHKVYAC |
| | | EVTHQGLSSPVTKSFNRGEC |
| 258 | SCFAB | EVQLVESGGGLVKPGGSLRLSCAASGFTFSNARMGWVRQA |
| | | PGKGLEWVGRIKSKTEGGTRDYAAPVKGRFTISRDDSKNT |
| | | LYLQMNSLKTEDTAVYYCTSYSGVWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCGGGGQGGGGQGGGGQGGGGQGG |
| | | GGQGGGGQGGGGQGGGGQEIVMTQSPDSLAVSLGERATIN |
| | | CKSSQSVLYSSNNKNYLAWYHQKPGQSPKLLISWASTRES |
| | | GVPDRFSGSGSGTDFTLTINSLQAEDVAVYYCQQYYSIPI |
| | | TFGGGTKVEIKRTVAAPSVFIFPPSDEQLKSGTASVVCLL |
| | | NNFYPREAKVQWKVDNALQSGNSQESVTEQDSKDSTYSLS |
| | | STLTLSKADYEKHKVYACEVTHQGLSSPVTKSFNRGEC |
| 259 | TCE 1.4 | EVQLVESGGGLVKPGGSLRLSCAASGFTFSNARMGWVRQA |
| | | PGKGLEWVGRIKSKTEGGTRDYAAPVKGRFTISRDDSKNT |
| | | LYLQMNSLKTEDTAVYYCTSYSGVWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCGGGGQGGGGQGGGGQGGGGQGG |
| | | GGQGGGGQGGGGQGGGGQEIVMTQSPDSLAVSLGERATIN |
| | | CKSSQSVLYSSNNKNYLAWYHQKPGQSPKLLISWASTRES |
| | | GVPDRFSGSGSGTDFTLTINSLQAEDVAVYYCQQYYSIPI |
| | | TFGGGTKVEIKRTVAAPSVFIFPPSDEQLKSGTASVVCLL |
| | | NNFYPREAKVQWKVDNALQSGNSQESVTEQDSKDSTYSLS |
| | | STLTLSKADYEKHKVYACEVTHQGLSSPVTKSFNRGECSG |
| | | GGGQEVQLVESGGGLVQPGGSLKLSCAASGFTFNKYAINW |
| | | VRQAPGKGLEWVARIRSKYNNYATYYADAVKDRFTISRDD |
| | | SKNTVYLQMNNLKTEDTAVYYCARAGNFGSSYISYWAYWG |
| | | QGTLVTVSSGGGGQGGGGQGGGGQQTVVTQEPSLTVSPGG |
| | | TVTITCGSSTGAVTSGNYPNWVQKKPGQAPRGLIGGTKFL |
| | | APGTPARFSGSLSGGKAALTLSGVQPEDEAEYYCVLWYSN |
| | | RWVFGSGTKLTVL |
| 260 | TCE 1.4 HLE | EVQLVESGGGLVKPGGSLRLSCAASGFTFSNARMGWVRQA |
| | | PGKGLEWVGRIKSKTEGGTRDYAAPVKGRFTISRDDSKNT |
| | | LYLQMNSLKTEDTAVYYCTSYSGVWGQGTMVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCGGGGQGGGGQGGGGQGGGGQGG |
| | | GGQGGGGQGGGGQGGGGQEIVMTQSPDSLAVSLGERATIN |
| | | CKSSQSVLYSSNNKNYLAWYHQKPGQSPKLLISWASTRES |
| | | GVPDRFSGSGSGTDFTLTINSLQAEDVAVYYCQQYYSIPI |
| | | TFGGGTKVEIKRTVAAPSVFIFPPSDEQLKSGTASVVCLL |
| | | NNFYPREAKVQWKVDNALQSGNSQESVTEQDSKDSTYSLS |
| | | STLTLSKADYEKHKVYACEVTHQGLSSPVTKSFNRGECSG |
| | | GGGQEVQLVESGGGLVQPGGSLKLSCAASGFTFNKYAINW |
| | | VRQAPGKGLEWVARIRSKYNNYATYYADAVKDRFTISRDD |
| | | SKNTVYLQMNNLKTEDTAVYYCARAGNFGSSYISYWAYWG |
| | | QGTLVTVSSGGGGQGGGGQGGGGQQTVVTQEPSLTVSPGG |
| | | TVTITCGSSTGAVTSGNYPNWVQKKPGQAPRGLIGGTKFL |
| | | APGTPARFSGSLSGGKAALTLSGVQPEDEAEYYCVLWYSN |
| | | RWVFGSGTKLTVLGGGGCPPCPAPELLGGPSVFLFPPKPK |
| | | DTLMISRTPEVTCVVVDVSHEEPEVKFNWYVDGVEVHNAK |
| | | TKPCEEQYGSTYRCVSVLTVLHQDWLNGKEYKCKVSNKAL |
| | | PAPIEKTISKAKGQPREPQVYTLPPSREEMTKNQVSLTCL |
| | | VKGFYPSDIAVEWESNGQPENNYKTTPPVLDSDGSFFLYS |
| | | KLTVDKSRWQQGNVFSCSVMHEALHNHYTQKSLSLSPGGG |
| | | GQGGGGQGGGGQGGGGQGGGGQGGGGQCPPCPAPELLGGP |
| | | SVFLFPPKPKDTLMISRTPEVTCVVVDVSHEEPEVKFNWY |
| | | VDGVEVHNAKTKPCEEQYGSTYRCVSVLTVLHQDWLNGKE |
| | | YKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYTQ |
| | | KSLSLSPGK |

TABLE 25
MAGE-B2 (“MA”) and CD20 × CD22 TCE molecule sequences.
MA 03-D8 AS CC scFv × 12C × scFc

| | | |
|---|---|---|
| 261 | HCDR1 | SHAMS |
| 262 | HCDR2 | TISGSGGGTYYAASVKG |
| 263 | HCDR3 | GKGVHLGFDY |
| 264 | LCDR1 | GGNNIGSKSVH |
| 265 | LCDR2 | DDNDRPS |
| 266 | LCDR3 | QVWDYRTLDWV |
| 267 | VH | EVQLLESGGGLVQPGGSLRLSCAASGFTFSSHAMSWVRQAPG |
| | | KCLEWVSTISGSGGGTYYAASVKGRFTISRDNSKNTLYLQMN |
| | | SLRAEDTAVYYCATGKGVHLGFDYWGQGTLVTVSS |
| 268 | VL | SYVLTQPPSVSVAPGQTARITCGGNNIGSKSVHWYQQKPGQA |
| | | PVMVVYDDNDRPSGIPERFSGSNFGNTATLIISRVEAGDEADY |
| | | YCQVWDYRTLDWVFGCGTKLTVL |
| 269 | SCFV | EVQLLESGGGLVQPGGSLRLSCAASGFTFSSHAMSWVRQAPG |
| | | KCLEWVSTISGSGGGTYYAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNFGNTATLIISR |
| | | VEAGDEADYYCQVWDYRTLDWVFGCGTKLTVL |
| 270 | BISPECIFIC | EVQLLESGGGLVQPGGSLRLSCAASGFTFSSHAMSWVRQAPG |
| | | KCLEWVSTISGSGGGTYYAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNFGNTATLIISR |
| | | VEAGDEADYYCQVWDYRTLDWVFGCGTKLTVLSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| 271 | MOL. | EVQLLESGGGLVQPGGSLRLSCAASGFTFSSHAMSWVRQAPG |
| | BiTE HLE | KCLEWVSTISGSGGGTYYAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNFGNTATLIISR |
| | | VEAGDEADYYCQVWDYRTLDWVFGCGTKLTVLSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

MA 03-D8 AS scFab × 12C × scFc

| | | |
|---|---|---|
| 272 | VH-CH1 | EVQLLESGGGLVQPGGSLRLSCAASGFTFSSHAMSWVRQAPG |
| | | KGLEWVSTISGSGGGTYYAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSC |
| 273 | VL-CL | SYVLTQPPSVSVAPGQTARITCGGNNIGSKSVHWYQQKPGQA |
| | | PVMVVYDDNDRPSGIPERFSGSNFGNTATLIISRVEAGDEADY |
| | | YCQVWDYRTLDWVFGGGTKLTVLGQPKAAPSVTLFPPSSEEL |
| | | QANKATLVCLISDFYPGAVTVAWKADSSPVKAGVETTTPSKQ |
| | | SNNKYAASSYLSLTPEQWKSHKSYSCQVTHEGSTVEKTVAPT |
| | | ECS |
| 274 | scFab | EVQLLESGGGLVQPGGSLRLSCAASGFTFSSHAMSWVRQAPG |
| | | KGLEWVSTISGSGGGTYYAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNFGNTATLIIS |
| | | RVEAGDEADYYCQVWDYRTLDWVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECS |
| 275 | scFab- | EVQLLESGGGLVQPGGSLRLSCAASGFTFSSHAMSWVRQAPG |
| | BISPECIFIC | KGLEWVSTISGSGGGTYYAASVKGRFTISRDNSKNTLYLQMN |
| | MOL. | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNFGNTATLIIS |
| | | RVEAGDEADYYCQVWDYRTLDWVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECSSGGGGSEVQLVESGGGLVQPGGSLKLSC |
| | | AASGFTFNKYAMNWVRQAPGKGLEWVARIRSKYNNYATYY |
| | | ADSVKDRFTISRDDSKNTAYLQMNNLKTEDTAVYYCVRHGN |
| | | FGNSYISYWAYWGQGTLVTVSSGGGGSGGGGSGGGGSQTVV |
| | | TQEPSLTVSPGGTVTLTCGSSTGAVTSGNYPNWVQQKPGQAP |
| | | RGLIGGTKFLAPGTPARFSGSLLGGKAALTLSGVQPEDEAEYY |
| | | CVLWYSNRWVFGGGTKLTVL |
| 276 | scFab-BiTE | EVQLLESGGGLVQPGGSLRLSCAASGFTFSSHAMSWVRQAPG |
| | HLE | KGLEWVSTISGSGGGTYYAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNFGNTATLIIS |
| | | RVEAGDEADYYCQVWDYRTLDWVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECSSGGGGSEVQLVESGGGLVQPGGSLKLSC |
| | | AASGFTFNKYAMNWVRQAPGKGLEWVARIRSKYNNYATYY |
| | | ADSVKDRFTISRDDSKNTAYLQMNNLKTEDTAVYYCVRHGN |
| | | FGNSYISYWAYWGQGTLVTVSSGGGGSGGGGSGGGGSQTVV |
| | | TQEPSLTVSPGGTVTLTCGSSTGAVTSGNYPNWVQQKPGQAP |
| | | RGLIGGTKFLAPGTPARFSGSLLGGKAALTLSGVQPEDEAEYY |
| | | CVLWYSNRWVFGGGTKLTVLGGGGDKTHTCPPCPAPELLGG |
| | | PSVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKG |
| | | GGGSGGGGSGGGGSGGGGSGGGGSGGGGSDKTHTCPPCPAP |
| | | ELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

MA 98-C7 CC scFv × I2C × scFc

| | | |
|---|---|---|
| 277 | HCDR1 | SHAMS |
| 278 | HCDR2 | SISGSGGGTYYAASVKG |
| 279 | HCDR3 | GKGVHLGFDY |
| 280 | LCDR1 | GGNNIGSKSVH |
| 281 | LCDR2 | DDNDRPS |
| 282 | LCDR3 | QVWDYSPLRHV |
| 283 | VH | EVQLLESGGGLVQPGGSLRLSCAASGFTFSSHAMSWVRQAPG |
| | | KCLEWVSSISGSGGGTYYAASVKGRFTISRDNSKNTLYLQMN |
| | | SLRAEDTAVYYCATGKGVHLGFDYWGQGTLVTVSS |
| 284 | VL | SYVLTQPPSVSVAPGQTARITCGGNNIGSKSVHWYQQKPGQA |
| | | PVMVVYDDNDRPSGIPERFSGSNSGNTATLTISRVEAGDEADY |
| | | YCQVWDYSPLRHVFGCGTKLTVL |
| 285 | SCFV | EVQLLESGGGLVQPGGSLRLSCAASGFTFSSHAMSWVRQAPG |
| | | KCLEWVSSISGSGGGTYYAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYSPLRHVFGCGTKLTVL |
| 286 | BISPECIFIC | EVQLLESGGGLVQPGGSLRLSCAASGFTFSSHAMSWVRQAPG |
| | MOL. | KCLEWVSSISGSGGGTYYAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYSPLRHVFGCGTKLTVLSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQ |
| 287 | BiTE HLE | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | EVQLLESGGGLVQPGGSLRLSCAASGFTFSSHAMSWVRQAPG |
| | | KCLEWVSSISGSGGGTYYAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYSPLRHVFGCGTKLTVLSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

MA 98-C7 scFab × I2C × scFc

| | | |
|---|---|---|
| 288 | VH-CH1 | EVQLLESGGGLVQPGGSLRLSCAASGFTFSSHAMSWVRQAPG |
| | | KGLEWVSSISGSGGGTYYAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSC |
| 289 | VL-CL | SYVLTQPPSVSVAPGQTARITCGGNNIGSKSVHWYQQKPGQA |
| | | PVMVVYDDNDRPSGIPERFSGSNSGNTATLTISRVEAGDEADY |
| | | YCQVWDYSPLRHVFGGGTKLTVLGQPKAAPSVTLFPPSSEEL |
| | | QANKATLVCLISDFYPGAVTVAWKADSSPVKAGVETTTPSKQ |
| | | SNNKYAASSYLSLTPEQWKSHKSYSCQVTHEGSTVEKTVAPT |
| | | ECS |
| 290 | scFab | EVQLLESGGGLVQPGGSLRLSCAASGFTFSSHAMSWVRQAPG |
| | | KGLEWVSSISGSGGGTYYAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYSPLRHVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECS |
| 291 | scFab- | EVQLLESGGGLVQPGGSLRLSCAASGFTFSSHAMSWVRQAPG |
| | BISPECIFIC | KGLEWVSSISGSGGGTYYAASVKGRFTISRDNSKNTLYLQMN |
| | MOL. | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYSPLRHVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECSSGGGGSEVQLVESGGGLVQPGGSLKLSC |
| | | AASGFTFNKYAMNWVRQAPGKGLEWVARIRSKYNNYATYY |
| | | ADSVKDRFTISRDDSKNTAYLQMNNLKTEDTAVYYCVRHGN |
| | | FGNSYISYWAYWGQGTLVTVSSGGGGSGGGGSGGGGSQTVV |
| | | TQEPSLTVSPGGTVTLTCGSSTGAVTSGNYPNWVQQKPGQAP |
| | | RGLIGGTKFLAPGTPARFSGSLLGGKAALTLSGVQPEDEAEYY |
| | | CVLWYSNRWVFGGGTKLTVL |
| | | EVQLLESGGGLVQPGGSLRLSCAASGFTFSSHAMSWVRQAPG |
| | | KGLEWVSSISGSGGGTYYAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| 292 | scFab-BiTE | KVDKKVEPKSCGGGGSGGGGSGGGGSGGGGSGGGGSGGGG |
| | HLE | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYSPLRHVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECSSGGGGSEVQLVESGGGLVQPGGSLKLSC |
| | | AASGFTFNKYAMNWVRQAPGKGLEWVARIRSKYNNYATYY |
| | | ADSVKDRFTISRDDSKNTAYLQMNNLKTEDTAVYYCVRHGN |
| | | FGNSYISYWAYWGQGTLVTVSSGGGGSGGGGSGGGGSQTVV |
| | | TQEPSLTVSPGGTVTLTCGSSTGAVTSGNYPNWVQQKPGQAP |
| | | RGLIGGTKFLAPGTPARFSGSLLGGKAALTLSGVQPEDEAEYY |
| | | CVLWYSNRWVFGGGTKLTVLGGGGDKTHTCPPCPAPELLGG |
| | | PSVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKG |
| | | GGGSGGGGSGGGGSGGGGSGGGGSGGGGSDKTHTCPPCPAP |
| | | ELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

MA 09-E2 CC scFv × I2C × scFc

| | | |
|---|---|---|
| 293 | HCDR1 | NAWMS |
| 294 | HCDR2 | RIKSKTYGGTTDYAAPVKG |
| 295 | HCDR3 | PSYSGSYYNYFSVMDV |
| 296 | LCDR1 | RTSQSISSYLN |
| 297 | LCDR2 | AASSLQG |
| 298 | LCDR3 | QQSYSSPFT |
| 299 | VH | EVQLVESGGGLVQPGGSLRLSCAASGFTFSNAWMSWVRQAP |
| | | GKCLEWVGRIKSKTYGGTTDYAAPVKGRFTISRDDSKNTLFL |
| | | QMNSLKTEDTAVYYCTTPSYSGSYYNYFSVMDVWGQGTTVT |
| | | VSS |
| 300 | VL | DIQMTQSPSSLSASVGDRVTITCRTSQSISSYLNWYQQKPGRA |
| | | PKLLIFAASSLQGGVPSRFSGSGSGTDFTLTISSLQPEDFATY |
| | | YCQQSYSSPFTFGCGTKVEIK |
| 301 | SCFV | EVQLVESGGGLVQPGGSLRLSCAASGFTFSNAWMSWVRQAP |
| | | GKCLEWVGRIKSKTYGGTTDYAAPVKGRFTISRDDSKNTLFL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQSYSSPFTFGCGTKVEIK |
| 302 | BISPECIFIC | EVQLVESGGGLVQPGGSLRLSCAASGFTFSNAWMSWVRQAP |
| | MOL. | GKCLEWVGRIKSKTYGGTTDYAAPVKGRFTISRDDSKNTLFL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQSYSSPFTFGCGTKVEIKSGGGGS |
| | | EVQLVESGGGLVQPGGSLKLSCAASGFTFNKYAMNWVRQAP |
| | | GKGLEWVARIRSKYNNYATYYADSVKDRFTISRDDSKNTAYL |
| | | QMNNLKTEDTAVYYCVRHGNFGNSYISYWAYWGQGTLVTV |
| | | SSGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| 303 | BiTE HLE | EVQLVESGGGLVQPGGSLRLSCAASGFTFSNAWMSWVRQAP |
| | | GKCLEWVGRIKSKTYGGTTDYAAPVKGRFTISRDDSKNTLFL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQSYSSPFTFGCGTKVEIKSGGGGS |
| | | EVQLVESGGGLVQPGGSLKLSCAASGFTFNKYAMNWVRQAP |
| | | GKGLEWVARIRSKYNNYATYYADSVKDRFTISRDDSKNTAYL |
| | | QMNNLKTEDTAVYYCVRHGNFGNSYISYWAYWGQGTLVTV |
| | | SSGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

MA 09-E2 scFab × I2C × scFc

| | | |
|---|---|---|
| 304 | VH-CH1 | EVQLVESGGGLVQPGGSLRLSCAASGFTFSNAWMSWVRQAP |
| | | GKGLEWVGRIKSKTYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSC |
| 305 | VL-CL | DIQMTQSPSSLSASVGDRVTITCRTSQSISSYLNWYQQKPGRA |
| | | PKLLIFAASSLQGGVPSRFSGSGSGTDFTLTISSLQPEDFATYYC |
| | | QQSYSSPFTFGPGTKVEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| 306 | scFab | EVQLVESGGGLVQPGGSLRLSCAASGFTFSNAWMSWVRQAP |
| | | GKGLEWVGRIKSKTYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQSYSSPFTFGPGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGEC |
| 307 | scFab- | EVQLVESGGGLVQPGGSLRLSCAASGFTFSNAWMSWVRQAP |
| | BISPECIFIC | GKGLEWVGRIKSKTYGGTTDYAAPVKGRFTISRDDSKNTLFL |
| | MOL. | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQSYSSPFTFGPGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGECSGGGGSEVQLVESGGGLVQPGGS |
| | | LKLSCAASGFTFNKYAMNWVRQAPGKGLEWVARIRSKYNNY |
| | | ATYYADSVKDRFTISRDDSKNTAYLQMNNLKTEDTAVYYCV |
| | | RHGNFGNSYISYWAYWGQGTLVTVSSGGGGSGGGGSGGGGS |
| | | QTVVTQEPSLTVSPGGTVTLTCGSSTGAVTSGNYPNWVQQKP |
| | | GQAPRGLIGGTKFLAPGTPARFSGSLLGGKAALTLSGVQPEDE |
| | | AEYYCVLWYSNRWVFGGGTKLTVL |
| 308 | scFab-BiTE | EVQLVESGGGLVQPGGSLRLSCAASGFTFSNAWMSWVRQAP |
| | HLE | GKGLEWVGRIKSKTYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQSYSSPFTFGPGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGECSGGGGSEVQLVESGGGLVQPGGS |
| | | LKLSCAASGFTFNKYAMNWVRQAPGKGLEWVARIRSKYNNY |
| | | ATYYADSVKDRFTISRDDSKNTAYLQMNNLKTEDTAVYYCV |
| | | RHGNFGNSYISYWAYWGQGTLVTVSSGGGGSGGGGSGGGGS |
| | | QTVVTQEPSLTVSPGGTVTLTCGSSTGAVTSGNYPNWVQQKP |
| | | GQAPRGLIGGTKFLAPGTPARFSGSLLGGKAALTLSGVQPEDE |
| | | AEYYCVLWYSNRWVFGGGTKLTVLGGGGDKTHTCPPCPAPE |
| | | LLGGPSVFLFPPKPKDTLMISRTPEVTCVVVDVSHEDPEVKFN |
| | | WYVDGVEVHNAKTKPCEEQYGSTYRCVSVLTVLHQDWLNG |
| | | KEYKCKVSNKALPAPIEKTISKAKGQPREPQVYTLPPSREEMT |
| | | KNQVSLTCLVKGFYPSDIAVEWESNGQPENNYKTTPPVLDSD |
| | | GSFFLYSKLTVDKSRWQQGNVFSCSVMHEALHNHYTQKSLSL |
| | | SPGKGGGGSGGGGSGGGGSGGGGSGGGGSGGGGSDKTHTCP |
| | | PCPAPELLGGPSVFLFPPKPKDTLMISRTPEVTCVVVDVSHEDP |
| | | EVKFNWYVDGVEVHNAKTKPCEEQYGSTYRCVSVLTVLHQD |
| | | WLNGKEYKCKVSNKALPAPIEKTISKAKGQPREPQVYTLPPSR |
| | | EEMTKNQVSLTCLVKGFYPSDIAVEWESNGQPENNYKTTPPV |
| | | LDSDGSFFLYSKLTVDKSRWQQGNVFSCSVMHEALHNHYTQ |
| | | KSLSLSPGK |

MA 03-E11 AS CC scFv × I2C × scFc

| | | |
|---|---|---|
| 309 | HCDR1 | SHAMS |
| 310 | HCDR2 | AISGSGGGTYNAASVKG |
| 311 | HCDR3 | GKGVHLGFDY |
| 312 | LCDR1 | GGNNIGSKSVH |
| 313 | LCDR2 | DDNDRPS |
| 314 | LCDR3 | QVWDYYSNRAV |
| 315 | VH | EVQLLESGGGLVQPGGSLRLSCAASGFTFSSHAMSWVRQAPG |
| | | KCLEWVSAISGSGGGTYNAASVKGRFTISRDNSKNTLYLQMN |
| | | SLRAEDTAAYYCVTGKGVHLGFDYWGQGTLVTVSS |
| 316 | VL | SYVLTQPPSVSVAPGQTARITCGGNNIGSKSVHWYQQKPGQA |
| | | PVMVVYDDNDRPSGIPERFSGSNSGNTATLTISRVEAGDEADY |
| | | YCQVWDYYSNRAVFGCGTKLTVL |
| 317 | SCFV | EVQLLESGGGLVQPGGSLRLSCAASGFTFSSHAMSWVRQAPG |
| | | KCLEWVSAISGSGGGTYNAASVKGRFTISRDNSKNTLYLQMN |
| | | SLRAEDTAAYYCVTGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYYSNRAVFGCGTKLTVL |
| 318 | BISPECIFIC | EVQLLESGGGLVQPGGSLRLSCAASGFTFSSHAMSWVRQAPG |
| | | KCLEWVSAISGSGGGTYNAASVKGRFTISRDNSKNTLYLQMN |
| | | SLRAEDTAAYYCVTGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYYSNRAVFGCGTKLTVLSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| 319 | MOL. | EVQLLESGGGLVQPGGSLRLSCAASGFTFSSHAMSWVRQAPG |
| | BiTE HLE | KCLEWVSAISGSGGGTYNAASVKGRFTISRDNSKNTLYLQMN |
| | | SLRAEDTAAYYCVTGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYYSNRAVFGCGTKLTVLSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEV |
| | | TCVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

MA 03-E11 AS scFab × I2C × scFc

| | | |
|---|---|---|
| 320 | VH-CH1 | EVQLLESGGGLVQPGGSLRLSCAASGFTFSSHAMSWVRQAPG |
| | | KGLEWVSAISGSGGGTYNAASVKGRFTISRDNSKNTLYLQMN |
| | | SLRAEDTAAYYCVTGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSC |
| 321 | VL-CL | SYVLTQPPSVSVAPGQTARITCGGNNIGSKSVHWYQQKPGQA |
| | | PVMVVYDDNDRPSGIPERFSGSNSGNTATLTISRVEAGDEADY |
| | | YCQVWDYYSNRAVFGGGTKLTVLGQPKAAPSVTLFPPSSEEL |
| | | QANKATLVCLISDFYPGAVTVAWKADSSPVKAGVETTTPSKQ |
| | | SNNKYAASSYLSLTPEQWKSHKSYSCQVTHEGSTVEKTVAPT |
| | | ECS |
| 322 | scFab | EVQLLESGGGLVQPGGSLRLSCAASGFTFSSHAMSWVRQAPG |
| | | KGLEWVSAISGSGGGTYNAASVKGRFTISRDNSKNTLYLQMN |
| | | SLRAEDTAAYYCVTGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYYSNRAVFGGGTKLTVLGQPKAA |
| | | PSVTLFPPSSEELQANKATLVCLISDFYPGAVTVAWKADSSPV |
| | | KAGVETTTPSKQSNNKYAASSYLSLTPEQWKSHKSYSCQVTH |
| | | EGSTVEKTVAPTECS |
| 323 | scFab- | EVQLLESGGGLVQPGGSLRLSCAASGFTFSSHAMSWVRQAPG |
| | BISPECIFIC | KGLEWVSAISGSGGGTYNAASVKGRFTISRDNSKNTLYLQMN |
| | MOL. | SLRAEDTAAYYCVTGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYYSNRAVFGGGTKLTVLGQPKAA |
| | | PSVTLFPPSSEELQANKATLVCLISDFYPGAVTVAWKADSSPV |
| | | KAGVETTTPSKQSNNKYAASSYLSLTPEQWKSHKSYSCQVTH |
| | | EGSTVEKTVAPTECSSGGGGSEVQLVESGGGLVQPGGSLKLS |
| | | CAASGFTFNKYAMNWVRQAPGKGLEWVARIRSKYNNYATY |
| | | YADSVKDRFTISRDDSKNTAYLQMNNLKTEDTAVYYCVRHG |
| | | NFGNSYISYWAYWGQGTLVTVSSGGGGSGGGGSGGGGSQTV |
| | | VTQEPSLTVSPGGTVTLTCGSSTGAVTSGNYPNWVQQKPGQA |
| | | PRGLIGGTKFLAPGTPARFSGSLLGGKAALTLSGVQPEDEAEY |
| | | YCVLWYSNRWVFGGGTKLTVL |
| 324 | scFab-BiTE | EVQLLESGGGLVQPGGSLRLSCAASGFTFSSHAMSWVRQAPG |
| | HLE | KGLEWVSAISGSGGGTYNAASVKGRFTISRDNSKNTLYLQMN |
| | | SLRAEDTAAYYCVTGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYYSNRAVFGGGTKLTVLGQPKAA |
| | | PSVTLFPPSSEELQANKATLVCLISDFYPGAVTVAWKADSSPV |
| | | KAGVETTTPSKQSNNKYAASSYLSLTPEQWKSHKSYSCQVTH |
| | | EGSTVEKTVAPTECSSGGGGSEVQLVESGGGLVQPGGSLKLS |
| | | CAASGFTFNKYAMNWVRQAPGKGLEWVARIRSKYNNYATY |
| | | YADSVKDRFTISRDDSKNTAYLQMNNLKTEDTAVYYCVRHG |
| | | NFGNSYISYWAYWGQGTLVTVSSGGGGSGGGGSGGGGSQTV |
| | | VTQEPSLTVSPGGTVTLTCGSSTGAVTSGNYPNWVQQKPGQA |
| | | PRGLIGGTKFLAPGTPARFSGSLLGGKAALTLSGVQPEDEAE |
| | | YYCVLWYSNRWVFGGGTKLTVLGGGGDKTHTCPPCPAPELLG |
| | | GPSVFLFPPKPKDTLMISRTPEVTCVVVDVSHEDPEVKFNWY |
| | | VDGVEVHNAKTKPCEEQYGSTYRCVSVLTVLHQDWLNGKEYK |
| | | CKVSNKALPAPIEKTISKAKGQPREPQVYTLPPSREEMTKNQV |
| | | SLTCLVKGFYPSDIAVEWESNGQPENNYKTTPPVLDSDGSFF |
| | | LYSKLTVDKSRWQQGNVFSCSVMHEALHNHYTQKSLSLSPGK |
| | | GGGGSGGGGSGGGGSGGGGSGGGGSGGGGSDKTHTCPPCPAP |
| | | ELLGGPSVFLFPPKPKDTLMISRTPEVTCVVVDVSHEDPEVK |
| | | FNWYVDGVEVHNAKTKPCEEQYGSTYRCVSVLTVLHQDWLNG |
| | | KEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

MA 09-F12 CC scFv × I2C × scFc

| | | |
|---|---|---|
| 325 | HCDR1 | THKMGVD |
| 326 | HCDR2 | LIYWNDDKRYSPSLQS |
| 327 | HCDR3 | RRYNWNYENWFDP |
| 328 | LCDR1 | QASQDISNYLN |
| 329 | LCDR2 | AASSLQS |
| 330 | LCDR3 | QQSYYYPTL |
| 331 | VH | QITLKESGPALVKPTQTLTLTCTFSGFSFTTHKMGVDWIRQPP |
| | | GKCLEWLALIYWNDDKRYSPSLQSRLTITKDTSKNQVVLTMT |
| | | NMDPVDTATYYCAYRRYNWNYENWFDPWGQGTLVTVSS |
| 332 | VL | DIQMTQSPSSLSASVGDRVTITCQASQDISNYLNWYQQKPGKA |
| | | PKLLIYAASSLQSGVPSRFSGRGSGTDFTLTISSLQPEDFATY |
| | | YCQQSYYYPTLFGCGTKVEIK |
| 333 | SCFV | QITLKESGPALVKPTQTLTLTCTFSGFSFTTHKMGVDWIRQP |
| | | PGKCLEWLALIYWNDDKRYSPSLQSRLTITKDTSKNQVVLTM |
| | | TNMDPVDTATYYCAYRRYNWNYENWFDPWGQGTLVTVSSGGG |
| | | GSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYYYPTLFGCGTKVEIK |
| 334 | BISPECIFIC | QITLKESGPALVKPTQTLTLTCTFSGFSFTTHKMGVDWIRQP |
| | MOL. | PGKCLEWLALIYWNDDKRYSPSLQSRLTITKDTSKNQVVLTM |
| | | TNMDPVDTATYYCAYRRYNWNYENWFDPWGQGTLVTVSSGGG |
| | | GSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYYYPTLFGCGTKVEIKSGGGGSE |
| | | VQLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| 335 | BiTE HLE | QITLKESGPALVKPTQTLTLTCTFSGFSFTTHKMGVDWIRQPP |
| | | GKCLEWLALIYWNDDKRYSPSLQSRLTITKDTSKNQVVLTMT |
| | | NMDPVDTATYYCAYRRYNWNYENWFDPWGQGTLVTVSSGGG |
| | | GSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYYYPTLFGCGTKVEIKSGGGGSE |
| | | VQLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVSS |
| | | GGGGSGGGGSGGGGSQTVVTQEPSLTVSPGGTVTLTCGSST |
| | | GAVTSGNYPNWVQQKPGQAPRGLIGGTKFLAPGTPARFSGS |
| | | LLGGKAALTLSGVQPEDEAEYYCVLWYSNRWVFGGGTKLTV |
| | | LGGGGDKTHTCPPCPAPELLGGPSVFLFPPKPKDTLMISRT |
| | | PEVTCVVVDVSHEDPEVKFNWYVDGVEVHNAKTKPCEEQYGS |
| | | TYRCVSVLTVLHQDWLNGKEYKCKVSNKALPAPIEKTISKAK |
| | | GQPREPQVYTLPPSREEMTKNQVSLTCLVKGFYPSDIAVEWE |
| | | SNGQPENNYKTTPPVLDSDGSFFLYSKLTVDKSRWQQGNVFS |
| | | CSVMHEALHNHYTQKSLSLSPGKGGGGSGGGGSGGGGSGGGG |
| | | SGGGGSGGGGSDKTHTCPPCPAPELLGGPSVFLFPPKPKDTL |
| | | MISRTPEVTCVVVDVSHEDPEVKFNWYVDGVEVHNAKTKPCE |
| | | EQYGSTYRCVSVLTVLHQDWLNGKEYKCKVSNKALPAPIEKT |
| | | ISKAKGQPREPQVYTLPPSREEMTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |

CCR8 TCE

| | | |
|---|---|---|
| 336 | CCR8 TCE | KSSQSVLYSSNNX₁NYLA; X₁ is K or R |
| | LCDR1 | |
| | consensus | |

MA 09-F12 scFab × I2C × scFc

| | | |
|---|---|---|
| 337 | VH-CH1 | QITLKESGPALVKPTQTLTLTCTFSGFSFTTHKMGVDWIRQPP |
| | | GKALEWLALIYWNDDKRYSPSLQSRLTITKDTSKNQVVLTMT |
| | | NMDPVDTATYYCAYRRYNWNYENWFDPWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSC |
| 338 | VL-CL | DIQMTQSPSSLSASVGDRVTITCQASQDISNYLNWYQQKPGK |
| | | APKLLIYAASSLQSGVPSRFSGRGSGTDFTLTISSLQPEDFATY |
| | | YCQQSYYYPTLFGPGTKVEIKRTVAAPSVFIFPPSDEQLKSGTA |
| | | SVVCLLNNFYPREAKVQWKVDNALQSGNSQESVTEQDSKDS |
| | | TYSLSSTLTLSKADYEKHKVYACEVTHQGLSSPVTKSFNRGEC |
| 339 | scFab | QITLKESGPALVKPTQTLTLTCTFSGFSFTTHKMGVDWIRQPP |
| | | GKALEWLALIYWNDDKRYSPSLQSRLTITKDTSKNQVVLTMT |
| | | NMDPVDTATYYCAYRRYNWNYENWFDPWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYYYPTLFGPGTKVEIKRTVAAPSVF |
| | | IFPPSDEQLKSGTASVVCLLNNFYPREAKVQWKVDNALQSGN |
| | | SQESVTEQDSKDSTYSLSSTLTLSKADYEKHKVYACEVTHQG |
| | | LSSPVTKSFNRGEC |
| 340 | scFab- | QITLKESGPALVKPTQTLTLTCTFSGFSFTTHKMGVDWIRQPP |
| | BISPECIFIC | GKALEWLALIYWNDDKRYSPSLQSRLTITKDTSKNQVVLTMT |
| | MOL. | NMDPVDTATYYCAYRRYNWNYENWFDPWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYYYPTLFGPGTKVEIKRTVAAPSVF |
| | | IFPPSDEQLKSGTASVVCLLNNFYPREAKVQWKVDNALQSGN |
| | | SQESVTEQDSKDSTYSLSSTLTLSKADYEKHKVYACEVTHQG |
| | | LSSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVL |
| 341 | scFab-BiTE | QITLKESGPALVKPTQTLTLTCTFSGFSFTTHKMGVDWIRQPP |
| | HLE | GKALEWLALIYWNDDKRYSPSLQSRLTITKDTSKNQVVLTMT |
| | | NMDPVDTATYYCAYRRYNWNYENWFDPWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYYYPTLFGPGTKVEIKRTVAAPSVF |
| | | IFPPSDEQLKSGTASVVCLLNNFYPREAKVQWKVDNALQSGN |
| | | SQESVTEQDSKDSTYSLSSTLTLSKADYEKHKVYACEVTHQG |
| | | LSSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVLGGGGDKTHTCPPCPAPELLGGP |
| | | SVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKG |
| | | GGGSGGGGSGGGGSGGGGSGGGGSGGGGSDKTHTCPPCPAP |
| | | ELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

CCR8 TCE

| | | |
|---|---|---|
| 342 | VL | DIVMTQSPDSLAVSLGERATINCKSSQSVLYSSNNX₁NYLAWY |
| | consensus | X₂QKPGQX₃PKLLISWASTRESGVPDRFSGSGSGTDFTLTINSL |
| | | QAEDVAVYYCQQYYSIPITFGGGTKVEIKR, |
| | | wherein X₁ is K or |
| | | R, X₂ is H or Q, and/or X₃ is S or P |

MA 09-G10 CC scFv × I2C × scFc

| | | |
|---|---|---|
| 343 | HCDR1 | THKMGVD |
| 344 | HCDR2 | GIHIYDDKRYSPSLQS |
| 345 | HCDR3 | RRYNWNYENWFDP |
| 346 | LCDR1 | QASQDISNYFN |
| 347 | LCDR2 | AASSLQS |
| 348 | LCDR3 | QQSYITPFT |
| 349 | VH | QITLKESGPTLVKPTQTLTLTCTFSGFSFTTHKMGVDWIRQPPG |
| | | KCLEWLAGIHIYDDKRYSPSLQSRLTITKDTSKNQVVLTMTN |
| | | MDPVDTATYYCAYRRYNWNYENWFDPWGQGTLVTVSS |
| 350 | VL | DIQMTQSPSSLSASVGDRVTITCQASQDISNYFNWYQQKPGKA |
| | | PKLLIYAASSLQSGVPSRFSGSGSGTDFTLTISSLQPEDFATYYC |
| | | QQSYITPFTFGCGTKVEIK |
| 351 | SCFV | QITLKESGPTLVKPTQTLTLTCTFSGFSFTTHKMGVDWIRQPPG |
| | | KCLEWLAGIHIYDDKRYSPSLQSRLTITKDTSKNQVVLTMTN |
| | | MDPVDTATYYCAYRRYNWNYENWFDPWGQGTLVTVSSGGG |
| | | GSGGGGSGGGGSDIQMTQSPSSLSASVGDRVTITCQASQDISN |
| | | YFNWYQQKPGKAPKLLIYAASSLQSGVPSRFSGSGSGTDFTLT |
| | | ISSLQPEDFATYYCQQSYITPFTFGCGTKVEIK |
| 352 | BISPECIFIC | QITLKESGPTLVKPTQTLTLTCTFSGFSFTTHKMGVDWIRQPPG |
| | MOL. | KCLEWLAGIHIYDDKRYSPSLQSRLTITKDTSKNQVVLTMTN |
| | | MDPVDTATYYCAYRRYNWNYENWFDPWGQGTLVTVSSGGG |
| | | GSGGGGSGGGGSDIQMTQSPSSLSASVGDRVTITCQASQDISN |
| | | YFNWYQQKPGKAPKLLIYAASSLQSGVPSRFSGSGSGTDFTLT |
| | | ISSLQPEDFATYYCQQSYITPFTFGCGTKVEIKSGGGGSEVQLV |
| | | ESGGGLVQPGGSLKLSCAASGFTFNKYAMNWVRQAPGKGLE |
| | | WVARIRSKYNNYATYYADSVKDRFTISRDDSKNTAYLQMNN |
| | | LKTEDTAVYYCVRHGNFGNSYISYWAYWGQGTLVTVSSGGG |
| | | GSGGGGSGGGGSQTVVTQEPSLTVSPGGTVTLTCGSSTGAVT |
| | | SGNYPNWVQQKPGQAPRGLIGGTKFLAPGTPARFSGSLLGGK |
| | | AALTLSGVQPEDEAEYYCVLWYSNRWVFGGGTKLTVL |
| 353 | BiTE HLE | QITLKESGPTLVKPTQTLTLTCTFSGFSFTTHKMGVDWIRQPPG |
| | | KCLEWLAGIHIYDDKRYSPSLQSRLTITKDTSKNQVVLTMTN |
| | | MDPVDTATYYCAYRRYNWNYENWFDPWGQGTLVTVSSGGG |
| | | GSGGGGSGGGGSDIQMTQSPSSLSASVGDRVTITCQASQDISN |
| | | YFNWYQQKPGKAPKLLIYAASSLQSGVPSRFSGSGSGTDFTLT |
| | | ISSLQPEDFATYYCQQSYITPFTFGCGTKVEIKSGGGGSEVQLV |
| | | ESGGGLVQPGGSLKLSCAASGFTFNKYAMNWVRQAPGKGLE |
| | | WVARIRSKYNNYATYYADSVKDRFTISRDDSKNTAYLQMNN |
| | | LKTEDTAVYYCVRHGNFGNSYISYWAYWGQGTLVTVSSGGG |
| | | GSGGGGSGGGGSQTVVTQEPSLTVSPGGTVTLTCGSSTGAVT |
| | | SGNYPNWVQQKPGQAPRGLIGGTKFLAPGTPARFSGSLLGGK |
| | | AALTLSGVQPEDEAEYYCVLWYSNRWVFGGGTKLTVLGGGG |
| | | DKTHTCPPCPAPELLGGPSVFLFPPKPKDTLMISRTPEVTCVVV |
| | | DVSHEDPEVKFNWYVDGVEVHNAKTKPCEEQYGSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHEA |
| | | LHNHYTQKSLSLSPGKGGGGSGGGGSGGGGSGGGGSGGGGS |
| | | GGGGSDKTHTCPPCPAPELLGGPSVFLFPPKPKDTLMISRTPEV |
| | | TCVVVDVSHEDPEVKFNWYVDGVEVHNAKTKPCEEQYGSTY |
| | | RCVSVLTVLHQDWLNGKEYKCKVSNKALPAPIEKTISKAKGQ |
| | | PREPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGK |

MA 09-G10 scFab × I2C × scFc

| | | |
|---|---|---|
| 354 | VH-CH1 | QITLKESGPTLVKPTQTLTLTCTFSGFSFTTHKMGVDWIRQPPG |
| | | KALEWLAGIHIYDDKRYSPSLQSRLTITKDTSKNQVVLTMTN |
| | | MDPVDTATYYCAYRRYNWNYENWFDPWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSC |
| 355 | VL-CL | DIQMTQSPSSLSASVGDRVTITCQASQDISNYFNWYQQKPGKA |
| | | PKLLIYAASSLQSGVPSRFSGSGSGTDFTLTISSLQPEDFATYYC |
| | | QQSYITPFTFGPGTKVEIKRTVAAPSVFIFPPSDEQLKSGTASVV |
| | | CLLNNFYPREAKVQWKVDNALQSGNSQESVTEQDSKDSTYS |
| | | LSSTLTLSKADYEKHKVYACEVTHQGLSSPVTKSFNRGEC |
| 356 | scFab | QITLKESGPTLVKPTQTLTLTCTFSGFSFTTHKMGVDWIRQPPG |
| | | KALEWLAGIHIYDDKRYSPSLQSRLTITKDTSKNQVVLTMTN |
| | | MDPVDTATYYCAYRRYNWNYENWFDPWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYFNWYQQKPGKAPKLLIYAASSLQSGVPSRFSGSGSGTDFTL |
| | | TISSLQPEDFATYYCQQSYITPFTFGPGTKVEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGEC |
| 357 | scFab- | QITLKESGPTLVKPTQTLTLTCTFSGFSFTTHKMGVDWIRQPPG |
| | BISPECIFIC | KALEWLAGIHIYDDKRYSPSLQSRLTITKDTSKNQVVLTMTN |
| | MOL. | MDPVDTATYYCAYRRYNWNYENWFDPWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYFNWYQQKPGKAPKLLIYAASSLQSGVPSRFSGSGSGTDFTL |
| | | TISSLQPEDFATYYCQQSYITPFTFGPGTKVEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVL |
| 358 | scFab-BiTE | QITLKESGPTLVKPTQTLTLTCTFSGFSFTTHKMGVDWIRQPPG |
| | HLE | KALEWLAGIHIYDDKRYSPSLQSRLTITKDTSKNQVVLTMTN |
| | | MDPVDTATYYCAYRRYNWNYENWFDPWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYFNWYQQKPGKAPKLLIYAASSLQSGVPSRFSGSGSGTDFTL |
| | | TISSLQPEDFATYYCQQSYITPFTFGPGTKVEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVLGGGGDKTHTCPPCPAPELLGGP |
| | | SVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKG |
| | | GGGSGGGGSGGGGSGGGGSGGGGSGGGGSDKTHTCPPCPAP |
| | | ELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

MA 09-H7 CC scFv × I2C × scFc

| | | |
|---|---|---|
| 359 | HCDR1 | THKMGVD |
| 360 | HCDR2 | LIYWNDDKRYSPSLQS |
| 361 | HCDR3 | RRYNWNYENWFDP |
| 362 | LCDR1 | QASQDISNYLN |
| 363 | LCDR2 | AASSLQS |
| 364 | LCDR3 | QQSYFPVVE |
| 365 | VH | QITLKESGPALVKPTQTLTLTCTFSGFSFTTHKMGVDWIRQPP |
| | | GKCLEWLALIYWNDDKRYSPSLQSRLTITKDTSKNQVVLTMT |
| | | NMDPVDTATYYCAYRRYNWNYENWFDPWGQGTLVTVSS |
| 366 | VL | DIQMTQSPSSLSASVGDRVTITCQASQDISNYLNWYQQKPGKA |
| | | PKLLIYAASSLQSGVPSRFSGSGSGTDFTLTISSLQPEDFATY |
| | | YCQQSYFPVVEFGCGTKVEIK |
| 367 | SCFV | QITLKESGPALVKPTQTLTLTCTFSGFSFTTHKMGVDWIRQPP |
| | | GKCLEWLALIYWNDDKRYSPSLQSRLTITKDTSKNQVVLTMT |
| | | NMDPVDTATYYCAYRRYNWNYENWFDPWGQGTLVTVSSGG |
| | | GGSGGGGSGGGGSDIQMTQSPSSLSASVGDRVTITCQASQDIS |
| | | NYLNWYQQKPGKAPKLLIYAASSLQSGVPSRFSGSGSGTDFTL |
| | | TISSLQPEDFATYYCQQSYFPVVEFGCGTKVEIK |
| 368 | BISPECIFIC | QITLKESGPALVKPTQTLTLTCTFSGFSFTTHKMGVDWIRQPP |
| | MOL. | GKCLEWLALIYWNDDKRYSPSLQSRLTITKDTSKNQVVLTMT |
| | | NMDPVDTATYYCAYRRYNWNYENWFDPWGQGTLVTVSSGG |
| | | GGSGGGGSGGGGSDIQMTQSPSSLSASVGDRVTITCQASQDIS |
| | | NYLNWYQQKPGKAPKLLIYAASSLQSGVPSRFSGSGSGTDFTL |
| | | TISSLQPEDFATYYCQQSYFPVVEFGCGTKVEIKSGGGGSEVQ |
| | | LVESGGGLVQPGGSLKLSCAASGFTFNKYAMNWVRQAPGKG |
| | | LEWVARIRSKYNNYATYYADSVKDRFTISRDDSKNTAYLQM |
| | | NNLKTEDTAVYYCVRHGNFGNSYISYWAYWGQGTLVTVSSG |
| | | GGGSGGGGSGGGGSQTVVTQEPSLTVSPGGTVTLTCGSSTGA |
| | | VTSGNYPNWVQQKPGQAPRGLIGGTKFLAPGTPARFSGSLLG |
| | | GKAALTLSGVQPEDEAEYYCVLWYSNRWVFGGGTKLTVL |
| 369 | BiTE HLE | QITLKESGPALVKPTQTLTLTCTFSGFSFTTHKMGVDWIRQPP |
| | | GKCLEWLALIYWNDDKRYSPSLQSRLTITKDTSKNQVVLTMT |
| | | NMDPVDTATYYCAYRRYNWNYENWFDPWGQGTLVTVSSGG |
| | | GGSGGGGSGGGGSDIQMTQSPSSLSASVGDRVTITCQASQDIS |
| | | NYLNWYQQKPGKAPKLLIYAASSLQSGVPSRFSGSGSGTDFTL |
| | | TISSLQPEDFATYYCQQSYFPVVEFGCGTKVEIKSGGGGSEVQ |
| | | LVESGGGLVQPGGSLKLSCAASGFTFNKYAMNWVRQAPGKG |
| | | LEWVARIRSKYNNYATYYADSVKDRFTISRDDSKNTAYLQM |
| | | NNLKTEDTAVYYCVRHGNFGNSYISYWAYWGQGTLVTVSSG |
| | | GGGSGGGGSGGGGSQTVVTQEPSLTVSPGGTVTLTCGSSTGA |
| | | VTSGNYPNWVQQKPGQAPRGLIGGTKFLAPGTPARFSGSLLG |
| | | GKAALTLSGVQPEDEAEYYCVLWYSNRWVFGGGTKLTVLGG |
| | | GGDKTHTCPPCPAPELLGGPSVFLFPPKPKDTLMISRTPEVTCV |
| | | VVDVSHEDPEVKFNWYVDGVEVHNAKTKPCEEQYGSTYRCV |
| | | SVLTVLHQDWLNGKEYKCKVSNKALPAPIEKTISKAKGQPRE |
| | | PQVYTLPPSREEMTKNQVSLTCLVKGFYPSDIAVEWESNGQPE |
| | | NNYKTTPPVLDSDGSFFLYSKLTVDKSRWQQGNVFSCSVMHE |
| | | ALHNHYTQKSLSLSPGKGGGGSGGGGSGGGGSGGGGSGGGG |
| | | SGGGGSDKTHTCPPCPAPELLGGPSVFLFPPKPKDTLMISRTPE |
| | | VTCVVVDVSHEDPEVKFNWYVDGVEVHNAKTKPCEEQYGST |
| | | YRCVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGK |

MA 09-H7 scFab × I2C × scFc

| | | |
|---|---|---|
| 370 | VH-CH1 | QITLKESGPALVKPTQTLTLTCTFSGFSFTTHKMGVDWIRQPP |
| | | GKALEWLALIYWNDDKRYSPSLQSRLTITKDTSKNQVVLTMT |
| | | NMDPVDTATYYCAYRRYNWNYENWFDPWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSC |
| 371 | VL-CL | DIQMTQSPSSLSASVGDRVTITCQASQDISNYLNWYQQKPGK |
| | | APKLLIYAASSLQSGVPSRFSGSGSGTDFTLTISSLQPEDFATYY |
| | | CQQSYFPVVEFGPGTKVEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| 372 | scFab | QITLKESGPALVKPTQTLTLTCTFSGFSFTTHKMGVDWIRQPP |
| | | GKALEWLALIYWNDDKRYSPSLQSRLTITKDTSKNQVVLTMT |
| | | NMDPVDTATYYCAYRRYNWNYENWFDPWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGSGSGTDFTL |
| | | TISSLQPEDFATYYCQQSYFPVVEFGPGTKVEIKRTVAAPSVFI |
| | | FPPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGEC |
| 373 | scFab- | QITLKESGPALVKPTQTLTLTCTFSGFSFTTHKMGVDWIRQPP |
| | BISPECIFIC | GKALEWLALIYWNDDKRYSPSLQSRLTITKDTSKNQVVLTMT |
| | MOL. | NMDPVDTATYYCAYRRYNWNYENWFDPWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGSGSGTDFTL |
| | | TISSLQPEDFATYYCQQSYFPVVEFGPGTKVEIKRTVAAPSVFI |
| | | FPPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVL |
| 374 | scFab-BiTE | QITLKESGPALVKPTQTLTLTCTFSGFSFTTHKMGVDWIRQPP |
| | HLE | GKALEWLALIYWNDDKRYSPSLQSRLTITKDTSKNQVVLTMT |
| | | NMDPVDTATYYCAYRRYNWNYENWFDPWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGSGSGTDFTL |
| | | TISSLQPEDFATYYCQQSYFPVVEFGPGTKVEIKRTVAAPSVFI |
| | | FPPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVLGGGGDKTHTCPPCPAPELLGGP |
| | | SVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKG |
| | | GGGSGGGGSGGGGSGGGGSGGGGSGGGGSDKTHTCPPCPAP |
| | | ELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

MA 09-H10 CC scFv × I2C × scFc

| | | |
|---|---|---|
| 375 | HCDR1 | THKMGVD |
| 376 | HCDR2 | LIYWNDDKRYSPSLQS |
| 377 | HCDR3 | RRYNWNYENWFDP |
| 378 | LCDR1 | QASQDISNYLN |
| 379 | LCDR2 | AASSLQS |
| 380 | LCDR3 | QQSYTPPTT |
| 381 | VH | QITLKESGPTLVKPTQTLTLTCTFSGFSFTTHKMGVDWIRQPPG |
| | | KCLEWLALIYWNDDKRYSPSLQSRLTITKDTSKNQVVLTMTN |
| | | MDPVDTATYYCAYRRYNWNYENWFDPWGQGTLVTVSS |
| 382 | VL | DIQMTQSPSSLSASVGDRVTITCQASQDISNYLNWYQQKPGK |
| | | APKLLIYAASSLQSGVPSRFSGRGSGTDFTLTISSLQPEDFATY |
| | | YCQQSYTPPTTFGCGTKVEIK |
| 383 | SCFV | QITLKESGPTLVKPTQTLTLTCTFSGFSFTTHKMGVDWIRQPPG |
| | | KCLEWLALIYWNDDKRYSPSLQSRLTITKDTSKNQVVLTMTN |
| | | MDPVDTATYYCAYRRYNWNYENWFDPWGQGTLVTVSSGGG |
| | | GSGGGGSGGGGSDIQMTQSPSSLSASVGDRVTITCQASQDISN |
| | | YLNWYQQKPGKAPKLLIYAASSLQSGVPSRFSGRGSGTDFTLT |
| | | ISSLQPEDFATYYCQQSYTPPTTFGCGTKVEIK |
| 384 | BISPECIFIC | QITLKESGPTLVKPTQTLTLTCTFSGFSFTTHKMGVDWIRQPPG |
| | MOL. | KCLEWLALIYWNDDKRYSPSLQSRLTITKDTSKNQVVLTMTN |
| | | MDPVDTATYYCAYRRYNWNYENWFDPWGQGTLVTVSSGGG |
| | | GSGGGGSGGGGSDIQMTQSPSSLSASVGDRVTITCQASQDISN |
| | | YLNWYQQKPGKAPKLLIYAASSLQSGVPSRFSGRGSGTDFTLT |
| | | ISSLQPEDFATYYCQQSYTPPTTFGCGTKVEIKSGGGGSEVQL |
| | | VESGGGLVQPGGSLKLSCAASGFTFNKYAMNWVRQAPGKGL |
| | | EWVARIRSKYNNYATYYADSVKDRFTISRDDSKNTAYLQMN |
| | | NLKTEDTAVYYCVRHGNFGNSYISYWAYWGQGTLVTVSSGG |
| | | GGSGGGGSGGGGSQTVVTQEPSLTVSPGGTVTLTCGSSTGAV |
| | | TSGNYPNWVQQKPGQAPRGLIGGTKFLAPGTPARFSGSLLGG |
| | | KAALTLSGVQPEDEAEYYCVLWYSNRWVFGGGTKLTVL |
| 385 | BiTE HLE | QITLKESGPTLVKPTQTLTLTCTFSGFSFTTHKMGVDWIRQPPG |
| | | KCLEWLALIYWNDDKRYSPSLQSRLTITKDTSKNQVVLTMTN |
| | | MDPVDTATYYCAYRRYNWNYENWFDPWGQGTLVTVSSGGG |
| | | GSGGGGSGGGGSDIQMTQSPSSLSASVGDRVTITCQASQDISN |
| | | YLNWYQQKPGKAPKLLIYAASSLQSGVPSRFSGRGSGTDFTLT |
| | | ISSLQPEDFATYYCQQSYTPPTTFGCGTKVEIKSGGGGSEVQL |
| | | VESGGGLVQPGGSLKLSCAASGFTFNKYAMNWVRQAPGKGL |
| | | EWVARIRSKYNNYATYYADSVKDRFTISRDDSKNTAYLQMN |
| | | NLKTEDTAVYYCVRHGNFGNSYISYWAYWGQGTLVTVSSGG |
| | | GGSGGGGSGGGGSQTVVTQEPSLTVSPGGTVTLTCGSSTGAV |
| | | TSGNYPNWVQQKPGQAPRGLIGGTKFLAPGTPARFSGSLLGG |
| | | KAALTLSGVQPEDEAEYYCVLWYSNRWVFGGGTKLTVLGGG |
| | | GDKTHTCPPCPAPELLGGPSVFLFPPKPKDTLMISRTPEVTCVV |
| | | VDVSHEDPEVKFNWYVDGVEVHNAKTKPCEEQYGSTYRCVS |
| | | VLTVLHQDWLNGKEYKCKVSNKALPAPIEKTISKAKGQPREP |
| | | QVYTLPPSREEMTKNQVSLTCLVKGFYPSDIAVEWESNGQPE |
| | | NNYKTTPPVLDSDGSFFLYSKLTVDKSRWQQGNVFSCSVMHE |
| | | ALHNHYTQKSLSLSPGKGGGGSGGGGSGGGGSGGGGSGGGG |
| | | SGGGGSDKTHTCPPCPAPELLGGPSVFLFPPKPKDTLMISRTPE |
| | | VTCVVVDVSHEDPEVKFNWYVDGVEVHNAKTKPCEEQYGST |
| | | YRCVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGK |

MA 09-H10 scFab × I2C × scFc

| | | |
|---|---|---|
| 386 | VH-CH1 | QITLKESGPTLVKPTQTLTLTCTFSGFSFTTHKMGVDWIRQPPG |
| | | KALEWLALIYWNDDKRYSPSLQSRLTITKDTSKNQVVLTMTN |
| | | MDPVDTATYYCAYRRYNWNYENWFDPWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSC |
| 387 | VL-CL | DIQMTQSPSSLSASVGDRVTITCQASQDISNYLNWYQQKPGK |
| | | APKLLIYAASSLQSGVPSRFSGRGSGTDFTLTISSLQPEDFATY |
| | | YCQQSYTPPTTFGPGTKVEIKRTVAAPSVFIFPPSDEQLKSGTA |
| | | SVVCLLNNFYPREAKVQWKVDNALQSGNSQESVTEQDSKDS |
| | | TYSLSSTLTLSKADYEKHKVYACEVTHQGLSSPVTKSFNRGEC |
| 388 | scFab | QITLKESGPTLVKPTQTLTLTCTFSGFSFTTHKMGVDWIRQPPG |
| | | KALEWLALIYWNDDKRYSPSLQSRLTITKDTSKNQVVLTMTN |
| | | MDPVDTATYYCAYRRYNWNYENWFDPWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYTPPTTFGPGTKVEIKRTVAAPSVFI |
| | | FPPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGEC |
| 389 | scFab- | QITLKESGPTLVKPTQTLTLTCTFSGFSFTTHKMGVDWIRQPPG |
| | BISPECIFIC | KALEWLALIYWNDDKRYSPSLQSRLTITKDTSKNQVVLTMTN |
| | MOL. | MDPVDTATYYCAYRRYNWNYENWFDPWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYTPPTTFGPGTKVEIKRTVAAPSVFI |
| | | FPPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVL |
| 390 | scFab-BiTE | QITLKESGPTLVKPTQTLTLTCTFSGFSFTTHKMGVDWIRQPPG |
| | HLE | KALEWLALIYWNDDKRYSPSLQSRLTITKDTSKNQVVLTMTN |
| | | MDPVDTATYYCAYRRYNWNYENWFDPWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCQASQDIS |
| | | NYLNWYQQKPGKAPKLLIYAASSLQSGVPSRFSGRGSGTDFT |
| | | LTISSLQPEDFATYYCQQSYTPPTTFGPGTKVEIKRTVAAPSVFI |
| | | FPPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVLGGGGDKTHTCPPCPAPELLGGP |
| | | SVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKG |
| | | GGGSGGGGSGGGGSGGGGSGGGGSGGGGSDKTHTCPPCPAP |
| | | ELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

MA 10-D3 CC scFv × I2C × scFc

| | | |
|---|---|---|
| 391 | HCDR1 | NAWMS |
| 392 | HCDR2 | RITSSRYGGTTDYAAPVKG |
| 393 | HCDR3 | PSYSGSYYNYFSVMDV |
| 394 | LCDR1 | RTSQSISSYLN |
| 395 | LCDR2 | AASSLQG |
| 396 | LCDR3 | QQTYSMPFT |
| 397 | VH | EVQLVESGGGLVQPGGSLRLSCAASGFTFSNAWMSWVRQAP |
| | | GKCLEWVGRITSSRYGGTTDYAAPVKGRFTISRDDSKNTLFLQ |
| | | MNSLKTEDTAVYYCTTPSYSGSYYNYFSVMDVWGQGTTVTV |
| | | SS |
| 398 | VL | DIQMTQSPSSLSASVGDRVTITCRTSQSISSYLNWYQQKPGRA |
| | | PKLLIFAASSLQGGVPSRFSGSGSGTDFTLTISSLQPEDFATYYC |
| | | QQTYSMPFTFGCGTKVDIK |
| 399 | SCFV | EVQLVESGGGLVQPGGSLRLSCAASGFTFSNAWMSWVRQAP |
| | | GKCLEWVGRITSSRYGGTTDYAAPVKGRFTISRDDSKNTLFLQ |
| | | MNSLKTEDTAVYYCTTPSYSGSYYNYFSVMDVWGQGTTVTV |
| | | SSGGGGGGGGSGGGGSDIQMTQSPSSLSASVGDRVTITCRTS |
| | | QSISSYLNWYQQKPGRAPKLLIFAASSLQGGVPSRFSGSGSGT |
| | | DFTLTISSLQPEDFATYYCQQTYSMPFTFGCGTKVDIK |
| 400 | BISPECIFIC | EVQLVESGGGLVQPGGSLRLSCAASGFTFSNAWMSWVRQAP |
| | MOL. | GKCLEWVGRITSSRYGGTTDYAAPVKGRFTISRDDSKNTLFLQ |
| | | MNSLKTEDTAVYYCTTPSYSGSYYNYFSVMDVWGQGTTVTV |
| | | SSGGGGGGGGSGGGGSDIQMTQSPSSLSASVGDRVTITCRTS |
| | | QSISSYLNWYQQKPGRAPKLLIFAASSLQGGVPSRFSGSGSGT |
| | | DFTLTISSLQPEDFATYYCQQTYSMPFTFGCGTKVDIKSGGGG |
| | | SEVQLVESGGGLVQPGGSLKLSCAASGFTFNKYAMNWVRQA |
| | | PGKGLEWVARIRSKYNNYATYYADSVKDRFTISRDDSKNTAY |
| | | LQMNNLKTEDTAVYYCVRHGNFGNSYISYWAYWGQGTLVT |
| | | VSSGGGGSGGGGSGGGGSQTVVTQEPSLTVSPGGTVTLTCGS |
| | | STGAVTSGNYPNWVQQKPGQAPRGLIGGTKFLAPGTPARFSG |
| | | SLLGGKAALTLSGVQPEDEAEYYCVLWYSNRWVFGGGTKLT |
| | | VL |
| 401 | BiTE HLE | EVQLVESGGGLVQPGGSLRLSCAASGFTFSNAWMSWVRQAP |
| | | GKCLEWVGRITSSRYGGTTDYAAPVKGRFTISRDDSKNTLFLQ |
| | | MNSLKTEDTAVYYCTTPSYSGSYYNYFSVMDVWGQGTTVTV |
| | | SSGGGGSGGGGSGGGGSDIQMTQSPSSLSASVGDRVTITCRTS |
| | | QSISSYLNWYQQKPGRAPKLLIFAASSLQGGVPSRFSGSGSGT |
| | | DFTLTISSLQPEDFATYYCQQTYSMPFTFGCGTKVDIKSGGGG |
| | | SEVQLVESGGGLVQPGGSLKLSCAASGFTFNKYAMNWVRQA |
| | | PGKGLEWVARIRSKYNNYATYYADSVKDRFTISRDDSKNTAY |
| | | LQMNNLKTEDTAVYYCVRHGNFGNSYISYWAYWGQGTLVT |
| | | VSSGGGGSGGGGSGGGGSQTVVTQEPSLTVSPGGTVTLTCGS |
| | | STGAVTSGNYPNWVQQKPGQAPRGLIGGTKFLAPGTPARFSG |
| | | SLLGGKAALTLSGVQPEDEAEYYCVLWYSNRWVFGGGTKLT |
| | | VLGGGGDKTHTCPPCPAPELLGGPSVFLFPPKPKDTLMISRTPE |
| | | VTCVVVDVSHEDPEVKFNWYVDGVEVHNAKTKPCEEQYGST |
| | | YRCVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGKGGGGSGGGGSGGGGSGGGGS |
| | | GGGGSGGGGSDKTHTCPPCPAPELLGGPSVFLFPPKPKDTLMI |
| | | SRTPEVTCVVVDVSHEDPEVKFNWYVDGVEVHNAKTKPCEE |
| | | QYGSTYRCVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQGN |
| | | VFSCSVMHEALHNHYTQKSLSLSPGK |

MA 10-D3 scFab × I2C × scFc

| | | |
|---|---|---|
| 402 | VH-CH1 | EVQLVESGGGLVQPGGSLRLSCAASGFTFSNAWMSWVRQAP |
| | | GKGLEWVGRITSSRYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSC |
| 403 | VL-CL | DIQMTQSPSSLSASVGDRVTITCRTSQSISSYLNWYQQKPGRA |
| | | PKLLIFAASSLQGGVPSRFSGSGSGTDFTLTISSLQPEDFATYYC |
| | | QQTYSMPFTFGPGTKVDIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| 404 | scFab | EVQLVESGGGLVQPGGSLRLSCAASGFTFSNAWMSWVRQAP |
| | | GKGLEWVGRITSSRYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGPGTKVDIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGEC |
| 405 | scFab- | EVQLVESGGGLVQPGGSLRLSCAASGFTFSNAWMSWVRQAP |
| | BISPECIFIC | GKGLEWVGRITSSRYGGTTDYAAPVKGRFTISRDDSKNTLFL |
| | MOL. | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGPGTKVDIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGECSGGGGSEVQLVESGGGLVQPGG |
| | | SLKLSCAASGFTFNKYAMNWVRQAPGKGLEWVARIRSKYNN |
| | | YATYYADSVKDRFTISRDDSKNTAYLQMNNLKTEDTAVYYC |
| | | VRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVL |
| 406 | scFab-BiTE | EVQLVESGGGLVQPGGSLRLSCAASGFTFSNAWMSWVRQAP |
| | HLE | GKGLEWVGRITSSRYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGPGTKVDIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGECSGGGGSEVQLVESGGGLVQPGG |
| | | SLKLSCAASGFTFNKYAMNWVRQAPGKGLEWVARIRSKYNN |
| | | YATYYADSVKDRFTISRDDSKNTAYLQMNNLKTEDTAVYYC |
| | | VRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVLGGGGDKTHTCPPCPA |
| | | PELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGKGGGGSGGGGSGGGGSGGGGSGGGGSGGGGSDKTHT |
| | | CPPCPAPELLGGPSVFLFPPKPKDTLMISRTPEVTCVVVDVSHE |
| | | DPEVKFNWYVDGVEVHNAKTKPCEEQYGSTYRCVSVLTVLH |
| | | QDWLNGKEYKCKVSNKALPAPIEKTISKAKGQPREPQVYTLP |
| | | PSREEMTKNQVSLTCLVKGFYPSDIAVEWESNGQPENNYKTT |
| | | PPVLDSDGSFFLYSKLTVDKSRWQQGNVFSCSVMHEALHNH |
| | | YTQKSLSLSPGK |

MA 10-D6 CC scFv × 12C × scFc

| | | |
|---|---|---|
| 407 | HCDR1 | NAWMS |
| 408 | HCDR2 | RILNNAYGGTTDYAAPVKG |
| 409 | HCDR3 | PSYSGSYYNYFSVMDV |
| 410 | LCDR1 | RTSQSISSYLN |
| 411 | LCDR2 | AASSLQG |
| 412 | LCDR3 | QQTYSMPFT |
| 413 | VH | EVQLVESGGGLVKPGGSLRLSCAASGFTFSNAWMSWVRQAP |
| | | GKCLEWVGRILNNAYGGTTDYAAPVKGRFTISRDDSKNTLYL |
| | | QMNSLKTEDTAVYYCTTPSYSGSYYNYFSVMDVWGQGTTVT |
| | | VSS |
| 414 | VL | DIQMTQSPSSLSASVGDRVTITCRTSQSISSYLNWYQQKPGRA |
| | | PKLLIFAASSLQGGVPSRFSGSGSGTDFTLTISSLQPEDFATYYC |
| | | QQTYSMPFTFGCGTKVDIK |
| 415 | SCFV | EVQLVESGGGLVKPGGSLRLSCAASGFTFSNAWMSWVRQAP |
| | | GKCLEWVGRILNNAYGGTTDYAAPVKGRFTISRDDSKNTLYL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQTYSMPFTFGCGTKVDIK |
| 416 | BISPECIFIC | EVQLVESGGGLVKPGGSLRLSCAASGFTFSNAWMSWVRQAP |
| | MOL. | GKCLEWVGRILNNAYGGTTDYAAPVKGRFTISRDDSKNTLYL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQTYSMPFTFGCGTKVDIKSGGGG |
| | | SEVQLVESGGGLVQPGGSLKLSCAASGFTFNKYAMNWVRQA |
| | | PGKGLEWVARIRSKYNNYATYYADSVKDRFTISRDDSKNTAY |
| | | LQMNNLKTEDTAVYYCVRHGNFGNSYISYWAYWGQGTLVT |
| | | VSSGGGGSGGGGSGGGGSQTVVTQEPSLTVSPGGTVTLTCGS |
| | | STGAVTSGNYPNWVQQKPGQAPRGLIGGTKFLAPGTPARFSG |
| | | SLLGGKAALTLSGVQPEDEAEYYCVLWYSNRWVFGGGTKLT |
| | | VL |
| 417 | BiTE HLE | EVQLVESGGGLVKPGGSLRLSCAASGFTFSNAWMSWVRQAP |
| | | GKCLEWVGRILNNAYGGTTDYAAPVKGRFTISRDDSKNTLYL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQTYSMPFTFGCGTKVDIKSGGGG |
| | | SEVQLVESGGGLVQPGGSLKLSCAASGFTFNKYAMNWVRQA |
| | | PGKGLEWVARIRSKYNNYATYYADSVKDRFTISRDDSKNTAY |
| | | LQMNNLKTEDTAVYYCVRHGNFGNSYISYWAYWGQGTLVT |
| | | VSSGGGGSGGGGSGGGGSQTVVTQEPSLTVSPGGTVTLTCGS |
| | | STGAVTSGNYPNWVQQKPGQAPRGLIGGTKFLAPGTPARFSG |
| | | SLLGGKAALTLSGVQPEDEAEYYCVLWYSNRWVFGGGTKLT |
| | | VLGGGGDKTHTCPPCPAPELLGGPSVFLFPPKPKDTLMISRTPE |
| | | VTCVVVDVSHEDPEVKFNWYVDGVEVHNAKTKPCEEQYGST |
| | | YRCVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGKGGGGSGGGGSGGGGSGGGGS |
| | | GGGGSGGGGSDKTHTCPPCPAPELLGGPSVFLFPPKPKDTLMI |
| | | SRTPEVTCVVVDVSHEDPEVKFNWYVDGVEVHNAKTKPCEE |
| | | QYGSTYRCVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQGN |
| | | VFSCSVMHEALHNHYTQKSLSLSPGK |

MA 10-D6 scFab × I2C × scFc

| | | |
|---|---|---|
| 418 | VH-CH1 | EVQLVESGGGLVKPGGSLRLSCAASGFTFSNAWMSWVRQAP |
| | | GKGLEWVGRILNNAYGGTTDYAAPVKGRFTISRDDSKNTLYL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSC |
| 419 | VL-CL | DIQMTQSPSSLSASVGDRVTITCRTSQSISSYLNWYQQKPGRA |
| | | PKLLIFAASSLQGGVPSRFSGSGSGTDFTLTISSLQPEDFATYYC |
| | | QQTYSMPFTFGPGTKVDIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| 420 | scFab | EVQLVESGGGLVKPGGSLRLSCAASGFTFSNAWMSWVRQAP |
| | | GKGLEWVGRILNNAYGGTTDYAAPVKGRFTISRDDSKNTLYL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGPGTKVDIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGEC |
| 421 | scFab- | EVQLVESGGGLVKPGGSLRLSCAASGFTFSNAWMSWVRQAP |
| | BISPECIFIC | GKGLEWVGRILNNAYGGTTDYAAPVKGRFTISRDDSKNTLYL |
| | MOL. | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGPGTKVDIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGECSGGGGSEVQLVESGGGLVQPGG |
| | | SLKLSCAASGFTFNKYAMNWVRQAPGKGLEWVARIRSKYNN |
| | | YATYYADSVKDRFTISRDDSKNTAYLQMNNLKTEDTAVYYC |
| | | VRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVL |
| 422 | scFab-BiTE | EVQLVESGGGLVKPGGSLRLSCAASGFTFSNAWMSWVRQAP |
| | HLE | GKGLEWVGRILNNAYGGTTDYAAPVKGRFTISRDDSKNTLYL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGPGTKVDIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGECSGGGGSEVQLVESGGGLVQPGG |
| | | SLKLSCAASGFTFNKYAMNWVRQAPGKGLEWVARIRSKYNN |
| | | YATYYADSVKDRFTISRDDSKNTAYLQMNNLKTEDTAVYYC |
| | | VRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVLGGGGDKTHTCPPCPA |
| | | PELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGKGGGGSGGGGSGGGGSGGGGSGGGGSGGGGSDKTHT |
| | | CPPCPAPELLGGPSVFLFPPKPKDTLMISRTPEVTCVVVDVSHE |
| | | DPEVKFNWYVDGVEVHNAKTKPCEEQYGSTYRCVSVLTVLH |
| | | QDWLNGKEYKCKVSNKALPAPIEKTISKAKGQPREPQVYTLP |
| | | PSREEMTKNQVSLTCLVKGFYPSDIAVEWESNGQPENNYKTT |
| | | PPVLDSDGSFFLYSKLTVDKSRWQQGNVFSCSVMHEALHNH |
| | | YTQKSLSLSPGK |

MA 10-G10 AS CC scFv × I2C × scFc

| | | |
|---|---|---|
| 423 | HCDR1 | SHAMS |
| 424 | HCDR2 | AISGSGGGTYNAASVKG |
| 425 | HCDR3 | GKGVHLGFDY |
| 426 | LCDR1 | GGNNIGSKSVH |
| 427 | LCDR2 | DDNDRPS |
| 428 | LCDR3 | QVWDYVAPRHV |
| 429 | VH | EVQLLESGGGLVQPGGSLRLSCAASGFTFSSHAMSWVRQAPG |
| | | KCLEWVSAISGSGGGTYNAASVKGRFTISRDNSKNTLYLQMN |
| | | SLRAEDTAVYYCATGKGVHLGFDYWGQGTLVTVSS |
| 430 | VL | SYVLTQPPSVSVAPGQTARITCGGNNIGSKSVHWYQQKPGQA |
| | | PVMVVYDDNDRPSGIPERFSGSNSGNTATLTISRVEAGDEADY |
| | | YCQVWDYVAPRHVFGCGTKLTVL |
| 431 | SCFV | EVQLLESGGGLVQPGGSLRLSCAASGFTFSSHAMSWVRQAPG |
| | | KCLEWVSAISGSGGGTYNAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYVAPRHVFGCGTKLTVL |
| 432 | BISPECIFIC | EVQLLESGGGLVQPGGSLRLSCAASGFTFSSHAMSWVRQAPG |
| | MOL. | KCLEWVSAISGSGGGTYNAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYVAPRHVFGCGTKLTVLSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| 433 | BiTE HLE | EVQLLESGGGLVQPGGSLRLSCAASGFTFSSHAMSWVRQAPG |
| | | KCLEWVSAISGSGGGTYNAASVKGRFTISRDNSKNTLYLQMN |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYVAPRHVFGCGTKLTVLSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

MA 10-G10 AS scFab × I2C × scFc

| | | |
|---|---|---|
| 434 | VH-CH1 | EVQLLESGGGLVQPGGSLRLSCAASGFTFSSHAMSWVRQAPG |
| | | KGLEWVSAISGSGGGTYNAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSC |
| 435 | VL-CL | SYVLTQPPSVSVAPGQTARITCGGNNIGSKSVHWYQQKPGQA |
| | | PVMVVYDDNDRPSGIPERFSGSNSGNTATLTISRVEAGDEADY |
| | | YCQVWDYVAPRHVFGGGTKLTVLGQPKAAPSVTLFPPSSEEL |
| | | QANKATLVCLISDFYPGAVTVAWKADSSPVKAGVETTTPSKQ |
| | | SNNKYAASSYLSLTPEQWKSHKSYSCQVTHEGSTVEKTVAPT |
| | | ECS |
| 436 | scFab | EVQLLESGGGLVQPGGSLRLSCAASGFTFSSHAMSWVRQAPG |
| | | KGLEWVSAISGSGGGTYNAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYVAPRHVFGGGTKLTVLGQPKAA |
| | | PSVTLFPPSSEELQANKATLVCLISDFYPGAVTVAWKADSSPV |
| | | KAGVETTTPSKQSNNKYAASSYLSLTPEQWKSHKSYSCQVTH |
| | | EGSTVEKTVAPTECS |
| 437 | scFab- | EVQLLESGGGLVQPGGSLRLSCAASGFTFSSHAMSWVRQAPG |
| | BISPECIFIC | KGLEWVSAISGSGGGTYNAASVKGRFTISRDNSKNTLYLQMN |
| | MOL. | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYVAPRHVFGGGTKLTVLGQPKAA |
| | | PSVTLFPPSSEELQANKATLVCLISDFYPGAVTVAWKADSSPV |
| | | KAGVETTTPSKQSNNKYAASSYLSLTPEQWKSHKSYSCQVTH |
| | | EGSTVEKTVAPTECSSGGGGSEVQLVESGGGLVQPGGSLKLS |
| | | CAASGFTFNKYAMNWVRQAPGKGLEWVARIRSKYNNYATY |
| | | YADSVKDRFTISRDDSKNTAYLQMNNLKTEDTAVYYCVRHG |
| | | NFGNSYISYWAYWGQGTLVTVSSGGGGSGGGGSGGGGSQTV |
| | | VTQEPSLTVSPGGTVTLTCGSSTGAVTSGNYPNWVQQKPGQA |
| | | PRGLIGGTKFLAPGTPARFSGSLLGGKAALTLSGVQPEDEAEY |
| | | YCVLWYSNRWVFGGGTKLTVL |
| 438 | scFab-BiTE | EVQLLESGGGLVQPGGSLRLSCAASGFTFSSHAMSWVRQAPG |
| | HLE | KGLEWVSAISGSGGGTYNAASVKGRFTISRDNSKNTLYLQMN |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYVAPRHVFGGGTKLTVLGQPKAA |
| | | PSVTLFPPSSEELQANKATLVCLISDFYPGAVTVAWKADSSPV |
| | | KAGVETTTPSKQSNNKYAASSYLSLTPEQWKSHKSYSCQVTH |
| | | EGSTVEKTVAPTECSSGGGGSEVQLVESGGGLVQPGGSLKLS |
| | | CAASGFTFNKYAMNWVRQAPGKGLEWVARIRSKYNNYATY |
| | | YADSVKDRFTISRDDSKNTAYLQMNNLKTEDTAVYYCVRHG |
| | | NFGNSYISYWAYWGQGTLVTVSSGGGGSGGGGSGGGGSQTV |
| | | VTQEPSLTVSPGGTVTLTCGSSTGAVTSGNYPNWVQQKPGQA |
| | | PRGLIGGTKFLAPGTPARFSGSLLGGKAALTLSGVQPEDEAEY |
| | | YCVLWYSNRWVFGGGTKLTVLGGGGDKTHTCPPCPAPELLG |
| | | GPSVFLFPPKPKDTLMISRTPEVTCVVVDVSHEDPEVKFNWYV |
| | | DGVEVHNAKTKPCEEQYGSTYRCVSVLTVLHQDWLNGKEYK |
| | | CKVSNKALPAPIEKTISKAKGQPREPQVYTLPPSREEMTKNQV |
| | | SLTCLVKGFYPSDIAVEWESNGQPENNYKTTPPVLDSDGSFFL |
| | | YSKLTVDKSRWQQGNVFSCSVMHEALHNHYTQKSLSLSPGK |
| | | GGGGSGGGGSGGGGSGGGGSGGGGSGGGGSDKTHTCPPCPA |
| | | PELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

MA 86-A4-N-F5 CC scFv × I2C × scFc

| | | |
|---|---|---|
| 439 | HCDR1 | TYGMH |
| 440 | HCDR2 | VISYDAETVKYAESVKG |
| 441 | HCDR3 | GQLLTGY |
| 442 | LCDR1 | RASQGISNYLA |
| 443 | LCDR2 | AASTLQS |
| 444 | LCDR3 | QKYNSAPFT |
| 445 | VH | QVQLVESGGGVVQPGRSLRLSCAASGFTFSTYGMHWVRQAP |
| | | GKCLEWVAVISYDAETVKYAESVKGRFTISRDNSKNTLYLQM |
| | | NSLRAEDTAVYYCARGQLLTGYWGQGTLVTVSS |
| 446 | VL | DIQMTQSPSSLSASVGDRVTITCRASQGISNYLAWYQQKPGKV |
| | | PKLLIYAASTLQSGVPSRFSGSGSGTDFTLTISSLQPEDVATYY |
| | | CQKYNSAPFTFGCGTKVEIK |
| 447 | SCFV | QVQLVESGGGVVQPGRSLRLSCAASGFTFSTYGMHWVRQAP |
| | | GKCLEWVAVISYDAETVKYAESVKGRFTISRDNSKNTLYLQM |
| | | NSLRAEDTAVYYCARGQLLTGYWGQGTLVTVSSGGGGSGGG |
| | | GSGGGGSDIQMTQSPSSLSASVGDRVTITCRASQGISNYLAWY |
| | | QQKPGKVPKLLIYAASTLQSGVPSRFSGSGSGTDFTLTISSLQP |
| | | EDVATYYCQKYNSAPFTFGCGTKVEIK |
| 448 | BISPECIFIC | QVQLVESGGGVVQPGRSLRLSCAASGFTFSTYGMHWVRQAP |
| | MOL. | GKCLEWVAVISYDAETVKYAESVKGRFTISRDNSKNTLYLQM |
| | | NSLRAEDTAVYYCARGQLLTGYWGQGTLVTVSSGGGGSGGG |
| | | GSGGGGSDIQMTQSPSSLSASVGDRVTITCRASQGISNYLAWY |
| | | QQKPGKVPKLLIYAASTLQSGVPSRFSGSGSGTDFTLTISSLQP |
| | | EDVATYYCQKYNSAPFTFGCGTKVEIKSGGGGSEVQLVESGG |
| | | GLVQPGGSLKLSCAASGFTFNKYAMNWVRQAPGKGLEWVA |
| | | RIRSKYNNYATYYADSVKDRFTISRDDSKNTAYLQMNNLKTE |
| | | DTAVYYCVRHGNFGNSYISYWAYWGQGTLVTVSSGGGGSG |
| | | GGGSGGGGSQTVVTQEPSLTVSPGGTVTLTCGSSTGAVTSGN |
| | | YPNWVQQKPGQAPRGLIGGTKFLAPGTPARFSGSLLGGKAAL |
| | | TLSGVQPEDEAEYYCVLWYSNRWVFGGGTKLTVL |
| 449 | BiTE HLE | QVQLVESGGGVVQPGRSLRLSCAASGFTFSTYGMHWVRQAP |
| | | GKCLEWVAVISYDAETVKYAESVKGRFTISRDNSKNTLYLQM |
| | | NSLRAEDTAVYYCARGQLLTGYWGQGTLVTVSSGGGGSGGG |
| | | GSGGGGSDIQMTQSPSSLSASVGDRVTITCRASQGISNYLAWY |
| | | QQKPGKVPKLLIYAASTLQSGVPSRFSGSGSGTDFTLTISSLQP |
| | | EDVATYYCQKYNSAPFTFGCGTKVEIKSGGGGSEVQLVESGG |
| | | GLVQPGGSLKLSCAASGFTFNKYAMNWVRQAPGKGLEWVA |
| | | RIRSKYNNYATYYADSVKDRFTISRDDSKNTAYLQMNNLKTE |
| | | DTAVYYCVRHGNFGNSYISYWAYWGQGTLVTVSSGGGGSG |
| | | GGGSGGGGSQTVVTQEPSLTVSPGGTVTLTCGSSTGAVTSGN |
| | | YPNWVQQKPGQAPRGLIGGTKFLAPGTPARFSGSLLGGKAAL |
| | | TLSGVQPEDEAEYYCVLWYSNRWVFGGGTKLTVLGGGGDKT |
| | | HTCPPCPAPELLGGPSVFLFPPKPKDTLMISRTPEVTCVVVDVS |
| | | HEDPEVKFNWYVDGVEVHNAKTKPCEEQYGSTYRCVSVLTV |
| | | LHQDWLNGKEYKCKVSNKALPAPIEKTISKAKGQPREPQVYT |
| | | LPPSREEMTKNQVSLTCLVKGFYPSDIAVEWESNGQPENNYK |
| | | TTPPVLDSDGSFFLYSKLTVDKSRWQQGNVFSCSVMHEALHN |
| | | HYTQKSLSLSPGKGGGGSGGGGSGGGGSGGGGSGGGGSGGG |
| | | GSDKTHTCPPCPAPELLGGPSVFLFPPKPKDTLMISRTPEVTCV |
| | | VVDVSHEDPEVKFNWYVDGVEVHNAKTKPCEEQYGSTYRCV |
| | | SVLTVLHQDWLNGKEYKCKVSNKALPAPIEKTISKAKGQPRE |
| | | PQVYTLPPSREEMTKNQVSLTCLVKGFYPSDIAVEWESNGQPE |
| | | NNYKTTPPVLDSDGSFFLYSKLTVDKSRWQQGNVFSCSVMHE |
| | | ALHNHYTQKSLSLSPGK |

MA 86-A4-N-F5 scFab × I2C × scFc

| | | |
|---|---|---|
| 450 | VH-CH1 | QVQLVESGGGVVQPGRSLRLSCAASGFTFSTYGMHWVRQAP |
| | | GKGLEWVAVISYDAETVKYAESVKGRFTISRDNSKNTLYLQM |
| | | NSLRAEDTAVYYCARGQLLTGYWGQGTLVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSC |
| 451 | VL-CL | DIQMTQSPSSLSASVGDRVTITCRASQGISNYLAWYQQKPGKV |
| | | PKLLIYAASTLQSGVPSRFSGSGSGTDFTLTISSLQPEDVATYY |
| | | CQKYNSAPFTFGPGTKVEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| 452 | scFab | QVQLVESGGGVVQPGRSLRLSCAASGFTFSTYGMHWVRQAP |
| | | GKGLEWVAVISYDAETVKYAESVKGRFTISRDNSKNTLYLQM |
| | | NSLRAEDTAVYYCARGQLLTGYWGQGTLVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCGGGGSGGGGSGGGGSGGGGSGGGGSGGGGSG |
| | | GGGSGGGGSDIQMTQSPSSLSASVGDRVTITCRASQGISNYLA |
| | | WYQQKPGKVPKLLIYAASTLQSGVPSRFSGSGSGTDFTLTISSL |
| | | QPEDVATYYCQKYNSAPFTFGPGTKVEIKRTVAAPSVFIFPPSD |
| | | EQLKSGTASVVCLLNNFYPREAKVQWKVDNALQSGNSQESV |
| | | TEQDSKDSTYSLSSTLTLSKADYEKHKVYACEVTHQGLSSPVT |
| | | KSFNRGEC |
| 453 | scFab- | QVQLVESGGGVVQPGRSLRLSCAASGFTFSTYGMHWVRQAP |
| | BISPECIFIC | GKGLEWVAVISYDAETVKYAESVKGRFTISRDNSKNTLYLQM |
| | MOL. | NSLRAEDTAVYYCARGQLLTGYWGQGTLVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCGGGGSGGGGSGGGGSGGGGSGGGGSGGGGSG |
| | | GGGSGGGGSDIQMTQSPSSLSASVGDRVTITCRASQGISNYLA |
| | | WYQQKPGKVPKLLIYAASTLQSGVPSRFSGSGSGTDFTLTISSL |
| | | QPEDVATYYCQKYNSAPFTFGPGTKVEIKRTVAAPSVFIFPPSD |
| | | EQLKSGTASVVCLLNNFYPREAKVQWKVDNALQSGNSQESV |
| | | TEQDSKDSTYSLSSTLTLSKADYEKHKVYACEVTHQGLSSPVT |
| | | KSFNRGECSGGGGSEVQLVESGGGLVQPGGSLKLSCAASGFT |
| | | FNKYAMNWVRQAPGKGLEWVARIRSKYNNYATYYADSVKD |
| | | RFTISRDDSKNTAYLQMNNLKTEDTAVYYCVRHGNFGNSYIS |
| | | YWAYWGQGTLVTVSSGGGGSGGGGSGGGGSQTVVTQEPSLT |
| | | VSPGGTVTLTCGSSTGAVTSGNYPNWVQQKPGQAPRGLIGGT |
| | | KFLAPGTPARFSGSLLGGKAALTLSGVQPEDEAEYYCVLWYS |
| | | NRWVFGGGTKLTVL |
| 454 | scFab-BiTE | QVQLVESGGGVVQPGRSLRLSCAASGFTFSTYGMHWVRQAP |
| | HLE | GKGLEWVAVISYDAETVKYAESVKGRFTISRDNSKNTLYLQM |
| | | NSLRAEDTAVYYCARGQLLTGYWGQGTLVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCGGGGSGGGGSGGGGSGGGGSGGGGSGGGGSG |
| | | GGGSGGGGSDIQMTQSPSSLSASVGDRVTITCRASQGISNYLA |
| | | WYQQKPGKVPKLLIYAASTLQSGVPSRFSGSGSGTDFTLTISSL |
| | | QPEDVATYYCQKYNSAPFTFGPGTKVEIKRTVAAPSVFIFPPSD |
| | | EQLKSGTASVVCLLNNFYPREAKVQWKVDNALQSGNSQESV |
| | | TEQDSKDSTYSLSSTLTLSKADYEKHKVYACEVTHQGLSSPVT |
| | | KSFNRGECSGGGGSEVQLVESGGGLVQPGGSLKLSCAASGFT |
| | | FNKYAMNWVRQAPGKGLEWVARIRSKYNNYATYYADSVKD |
| | | RFTISRDDSKNTAYLQMNNLKTEDTAVYYCVRHGNFGNSYIS |
| | | YWAYWGQGTLVTVSSGGGGSGGGGSGGGGSQTVVTQEPSLT |
| | | VSPGGTVTLTCGSSTGAVTSGNYPNWVQQKPGQAPRGLIGGT |
| | | KFLAPGTPARFSGSLLGGKAALTLSGVQPEDEAEYYCVLWYS |
| | | NRWVFGGGTKLTVLGGGGDKTHTCPPCPAPELLGGPSVFLFP |
| | | PKPKDTLMISRTPEVTCVVVDVSHEDPEVKFNWYVDGVEVH |
| | | NAKTKPCEEQYGSTYRCVSVLTVLHQDWLNGKEYKCKVSNK |
| | | ALPAPIEKTISKAKGQPREPQVYTLPPSREEMTKNQVSLTCLV |
| | | KGFYPSDIAVEWESNGQPENNYKTTPPVLDSDGSFFLYSKLTV |
| | | DKSRWQQGNVFSCSVMHEALHNHYTQKSLSLSPGKGGGGSG |
| | | GGGSGGGGSGGGGSGGGGSGGGGSDKTHTCPPCPAPELLGGP |
| | | SVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGK |

MA 88-B3-F9 CC scFv × I2C × scFc

| | | |
|---|---|---|
| 455 | HCDR1 | DYYMS |
| 456 | HCDR2 | YISKSSYTVTYADAVKG |
| 457 | HCDR3 | YNYGHFDY |
| 458 | LCDR1 | GGNNIGSKSVH |
| 459 | LCDR2 | DDADRPS |
| 460 | LCDR3 | QVWDASAGYGVV |
| 461 | VH | QVQLVESGGGLVKPGGSLRLSCAASGFTFSDYYMSWIRQAPG |
| | | KCLEWVSYISKSSYTVTYADAVKGRFTISRDNAKNSLYLQMN |
| | | SLRAEDTAVYYCATYNYGHFDYWGQGTLVTVSS |
| 462 | VL | SYVLTQPPSVSVAPGQTARITCGGNNIGSKSVHWYQQKPGQA |
| | | PVLVVYDDADRPSGIPERFSGSNSGNTATLTISRVEAGDEADY |
| | | YCQVWDASAGYGVVFGCGTKLTVL |
| 463 | SCFV | QVQLVESGGGLVKPGGSLRLSCAASGFTFSDYYMSWIRQAPG |
| | | KCLEWVSYISKSSYTVTYADAVKGRFTISRDNAKNSLYLQMN |
| | | SLRAEDTAVYYCATYNYGHFDYWGQGTLVTVSSGGGGSGG |
| | | GGSGGGGSSYVLTQPPSVSVAPGQTARITCGGNNIGSKSVHW |
| | | YQQKPGQAPVLVVYDDADRPSGIPERFSGSNSGNTATLTISRV |
| | | EAGDEADYYCQVWDASAGYGVVFGCGTKLTVL |
| 464 | BISPECIFIC | QVQLVESGGGLVKPGGSLRLSCAASGFTFSDYYMSWIRQAPG |
| | MOL. | KCLEWVSYISKSSYTVTYADAVKGRFTISRDNAKNSLYLQMN |
| | | SLRAEDTAVYYCATYNYGHFDYWGQGTLVTVSSGGGGSGG |
| | | GGSGGGGSSYVLTQPPSVSVAPGQTARITCGGNNIGSKSVHW |
| | | YQQKPGQAPVLVVYDDADRPSGIPERFSGSNSGNTATLTISRV |
| | | EAGDEADYYCQVWDASAGYGVVFGCGTKLTVLSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| 465 | BiTE HLE | QVQLVESGGGLVKPGGSLRLSCAASGFTFSDYYMSWIRQAPG |
| | | KCLEWVSYISKSSYTVTYADAVKGRFTISRDNAKNSLYLQMN |
| | | SLRAEDTAVYYCATYNYGHFDYWGQGTLVTVSSGGGGSGG |
| | | GGSGGGGSSYVLTQPPSVSVAPGQTARITCGGNNIGSKSVHW |
| | | YQQKPGQAPVLVVYDDADRPSGIPERFSGSNSGNTATLTISRV |
| | | EAGDEADYYCQVWDASAGYGVVFGCGTKLTVLSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

MA 88-B3-F9 scFab × I2C × scFc

| | | |
|---|---|---|
| 466 | VH-CH1 | QVQLVESGGGLVKPGGSLRLSCAASGFTFSDYYMSWIRQAPG |
| | | KGLEWVSYISKSSYTVTYADAVKGRFTISRDNAKNSLYLQMN |
| | | SLRAEDTAVYYCATYNYGHFDYWGQGTLVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSC |
| 467 | VL-CL | SYVLTQPPSVSVAPGQTARITCGGNNIGSKSVHWYQQKPGQA |
| | | PVLVVYDDADRPSGIPERFSGSNSGNTATLTISRVEAGDEADY |
| | | YCQVWDASAGYGVVFGGGTKLTVLGQPKAAPSVTLFPPSSEE |
| | | LQANKATLVCLISDFYPGAVTVAWKADSSPVKAGVETTTPSK |
| | | QSNNKYAASSYLSLTPEQWKSHKSYSCQVTHEGSTVEKTVAP |
| | | TECS |
| 468 | scFab | QVQLVESGGGLVKPGGSLRLSCAASGFTFSDYYMSWIRQAPG |
| | | KGLEWVSYISKSSYTVTYADAVKGRFTISRDNAKNSLYLQMN |
| | | SLRAEDTAVYYCATYNYGHFDYWGQGTLVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCGGGGSGGGGSGGGGSGGGGSGGGGSGGGGSG |
| | | GGGSGGGGSSYVLTQPPSVSVAPGQTARITCGGNNIGSKSVH |
| | | WYQQKPGQAPVLVVYDDADRPSGIPERFSGSNSGNTATLTISR |
| | | VEAGDEADYYCQVWDASAGYGVVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECS |
| 469 | scFab- | QVQLVESGGGLVKPGGSLRLSCAASGFTFSDYYMSWIRQAPG |
| | BISPECIFIC | KGLEWVSYISKSSYTVTYADAVKGRFTISRDNAKNSLYLQMN |
| | MOL. | SLRAEDTAVYYCATYNYGHFDYWGQGTLVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCGGGGSGGGGSGGGGSGGGGSGGGGSGGGGSG |
| | | GGGSGGGGSSYVLTQPPSVSVAPGQTARITCGGNNIGSKSVH |
| | | WYQQKPGQAPVLVVYDDADRPSGIPERFSGSNSGNTATLTISR |
| | | VEAGDEADYYCQVWDASAGYGVVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECSSGGGGSEVQLVESGGGLVQPGGSLKLSC |
| | | AASGFTFNKYAMNWVRQAPGKGLEWVARIRSKYNNYATYY |
| | | ADSVKDRFTISRDDSKNTAYLQMNNLKTEDTAVYYCVRHGN |
| | | FGNSYISYWAYWGQGTLVTVSSGGGGSGGGGSGGGGSQTVV |
| | | TQEPSLTVSPGGTVTLTCGSSTGAVTSGNYPNWVQQKPGQAP |
| | | RGLIGGTKFLAPGTPARFSGSLLGGKAALTLSGVQPEDEAEYY |
| | | CVLWYSNRWVFGGGTKLTVL |
| 470 | scFab-BiTE | QVQLVESGGGLVKPGGSLRLSCAASGFTFSDYYMSWIRQAPG |
| | HLE | KGLEWVSYISKSSYTVTYADAVKGRFTISRDNAKNSLYLQMN |
| | | SLRAEDTAVYYCATYNYGHFDYWGQGTLVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCGGGGSGGGGSGGGGSGGGGSGGGGSGGGGSG |
| | | GGGSGGGGSSYVLTQPPSVSVAPGQTARITCGGNNIGSKSVH |
| | | WYQQKPGQAPVLVVYDDADRPSGIPERFSGSNSGNTATLTISR |
| | | VEAGDEADYYCQVWDASAGYGVVFGGGTKLTVLGQPKAAP |
| | | SVTLFPPSSEELQANKATLVCLISDFYPGAVTVAWKADSSPVK |
| | | AGVETTTPSKQSNNKYAASSYLSLTPEQWKSHKSYSCQVTHE |
| | | GSTVEKTVAPTECSSGGGGSEVQLVESGGGLVQPGGSLKLSC |
| | | AASGFTFNKYAMNWVRQAPGKGLEWVARIRSKYNNYATYY |
| | | ADSVKDRFTISRDDSKNTAYLQMNNLKTEDTAVYYCVRHGN |
| | | FGNSYISYWAYWGQGTLVTVSSGGGGSGGGGSGGGGSQTVV |
| | | TQEPSLTVSPGGTVTLTCGSSTGAVTSGNYPNWVQQKPGQAP |
| | | RGLIGGTKFLAPGTPARFSGSLLGGKAALTLSGVQPEDEAEYY |
| | | CVLWYSNRWVFGGGTKLTVLGGGGDKTHTCPPCPAPELLGG |
| | | PSVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKG |
| | | GGGSGGGGSGGGGSGGGGSGGGGSGGGGSDKTHTCPPCPAP |
| | | ELLGGPSVFLFPPKPKDTLMISRTPEVTCVVVDVSHEDPEVKF |
| | | NWYVDGVEVHNAKTKPCEEQYGSTYRCVSVLTVLHQDWLNG |
| | | KEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

MA SG-F28 CC scFv × I2C × scFc

| | | |
|---|---|---|
| 471 | HCDR1 | SNPYYWS |
| 472 | HCDR2 | YISYSGITNYNPSLKS |
| 473 | HCDR3 | EKMWFGVLNYYYGMDV |
| 474 | LCDR1 | RSSQSLLHRSGYNYLD |
| 475 | LCDR2 | LGSNRAS |
| 476 | LCDR3 | MQALQTPWT |
| 477 | VH | QVQLQESGPGLVKPSQTLSLTCTVSGDSISSNPYYWSWIRQHP |
| | | GKCLEWIGYISYSGITNYNPSLKSRVTMSVDTSKNQFSLKLTS |
| | | LTAADTAVYYCAREKMWFGVLNYYYGMDVWGQGTTVTVS |
| | | S |
| 478 | VL | DIVMTQTPLSLPVTPGEPASISCRSSQSLLHRSGYNYLDWYLQ |
| | | KPGQSPQLLIYLGSNRASGVPDRFSGSGSGTDFTLKISRVEAGD |
| | | VGVYYCMQALQTPWTFGCGTKVEIK |
| 479 | SCFV | QVQLQESGPGLVKPSQTLSLTCTVSGDSISSNPYYWSWIRQHP |
| | | GKCLEWIGYISYSGITNYNPSLKSRVTMSVDTSKNQFSLKLTS |
| | | LTAADTAVYYCAREKMWFGVLNYYYGMDVWGQGTTVTVS |
| | | SGGGGSGGGGSGGGGSDIVMTQTPLSLPVTPGEPASISCRSSQS |
| | | LLHRSGYNYLDWYLQKPGQSPQLLIYLGSNRASGVPDRFSGS |
| | | GSGTDFTLKISRVEAGDVGVYYCMQALQTPWTFGCGTKVEIK |
| 480 | BISPECIFIC | QVQLQESGPGLVKPSQTLSLTCTVSGDSISSNPYYWSWIRQHP |
| | MOL. | GKCLEWIGYISYSGITNYNPSLKSRVTMSVDTSKNQFSLKLTS |
| | | LTAADTAVYYCAREKMWFGVLNYYYGMDVWGQGTTVTVS |
| | | SGGGGSGGGGSGGGGSDIVMTQTPLSLPVTPGEPASISCRSSQS |
| | | LLHRSGYNYLDWYLQKPGQSPQLLIYLGSNRASGVPDRFSGS |
| | | GSGTDFTLKISRVEAGDVGVYYCMQALQTPWTFGCGTKVEIK |
| | | SGGGGSEVQLVESGGGLVQPGGSLKLSCAASGFTFNKYAMN |
| | | WVRQAPGKGLEWVARIRSKYNNYATYYADSVKDRFTISRDD |
| | | SKNTAYLQMNNLKTEDTAVYYCVRHGNFGNSYISYWAYWG |
| | | QGTLVTVSSGGGGSGGGGSGGGGSQTVVTQEPSLTVSPGGTV |
| | | TLTCGSSTGAVTSGNYPNWVQQKPGQAPRGLIGGTKFLAPGT |
| | | PARFSGSLLGGKAALTLSGVQPEDEAEYYCVLWYSNRWVFG |
| | | GGTKLTVL |
| 481 | BiTE HLE | QVQLQESGPGLVKPSQTLSLTCTVSGDSISSNPYYWSWIRQHP |
| | | GKCLEWIGYISYSGITNYNPSLKSRVTMSVDTSKNQFSLKLTS |
| | | LTAADTAVYYCAREKMWFGVLNYYYGMDVWGQGTTVTVS |
| | | SGGGGSGGGGSGGGGSDIVMTQTPLSLPVTPGEPASISCRSSQS |
| | | LLHRSGYNYLDWYLQKPGQSPQLLIYLGSNRASGVPDRFSGS |
| | | GSGTDFTLKISRVEAGDVGVYYCMQALQTPWTFGCGTKVEIK |
| | | SGGGGSEVQLVESGGGLVQPGGSLKLSCAASGFTFNKYAMN |
| | | WVRQAPGKGLEWVARIRSKYNNYATYYADSVKDRFTISRDD |
| | | SKNTAYLQMNNLKTEDTAVYYCVRHGNFGNSYISYWAYWG |
| | | QGTLVTVSSGGGGSGGGGSGGGGSQTVVTQEPSLTVSPGGTV |
| | | TLTCGSSTGAVTSGNYPNWVQQKPGQAPRGLIGGTKFLAPGT |
| | | PARFSGSLLGGKAALTLSGVQPEDEAEYYCVLWYSNRWVFG |
| | | GGTKLTVLGGGGDKTHTCPPCPAPELLGGPSVFLFPPKPKDTL |
| | | MISRTPEVTCVVVDVSHEDPEVKFNWYVDGVEVHNAKTKPC |
| | | EEQYGSTYRCVSVLTVLHQDWLNGKEYKCKVSNKALPAPIEK |
| | | TISKAKGQPREPQVYTLPPSREEMTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGKGGGGSGGGGSGGG |
| | | GSGGGGSGGGGSGGGGSDKTHTCPPCPAPELLGGPSVFLFPPK |
| | | PKDTLMISRTPEVTCVVVDVSHEDPEVKFNWYVDGVEVHNA |
| | | KTKPCEEQYGSTYRCVSVLTVLHQDWLNGKEYKCKVSNKAL |
| | | PAPIEKTISKAKGQPREPQVYTLPPSREEMTKNQVSLTCLVKG |
| | | FYPSDIAVEWESNGQPENNYKTTPPVLDSDGSFFLYSKLTVDK |
| | | SRWQQGNVFSCSVMHEALHNHYTQKSLSLSPGK |

MA SG-F28 scFab × I2C × scFc

| | | |
|---|---|---|
| 482 | VH-CH1 | QVQLQESGPGLVKPSQTLSLTCTVSGDSISSNPYYWSWIRQHP |
| | | GKGLEWIGYISYSGITNYNPSLKSRVTMSVDTSKNQFSLKLTS |
| | | LTAADTAVYYCAREKMWFGVLNYYYGMDVWGQGTTVTVS |
| | | SASTKGPSVFPLAPSSKSTSGGTAALGCLVKDYFPEPVTVSWN |
| | | SGALTSGVHTFPAVLQSSGLYSLSSVVTVPSSSLGTQTYICNVN |
| | | HKPSNTKVDKKVEPKSC |
| 483 | VL-CL | DIVMTQTPLSLPVTPGEPASISCRSSQSLLHRSGYNYLDWYLQ |
| | | KPGQSPQLLIYLGSNRASGVPDRFSGSGSGTDFTLKISRVEAGD |
| | | VGVYYCMQALQTPWTFGQGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| 484 | scFab | QVQLQESGPGLVKPSQTLSLTCTVSGDSISSNPYYWSWIRQHP |
| | | GKGLEWIGYISYSGITNYNPSLKSRVTMSVDTSKNQFSLKLTS |
| | | LTAADTAVYYCAREKMWFGVLNYYYGMDVWGQGTTVTVS |
| | | SASTKGPSVFPLAPSSKSTSGGTAALGCLVKDYFPEPVTVSWN |
| | | SGALTSGVHTFPAVLQSSGLYSLSSVVTVPSSSLGTQTYICNVN |
| | | HKPSNTKVDKKVEPKSCGGGGSGGGGSGGGGSGGGGSGGGG |
| | | SGGGGSGGGGSGGGGSDIVMTQTPLSLPVTPGEPASISCRSSQS |
| | | LLHRSGYNYLDWYLQKPGQSPQLLIYLGSNRASGVPDRFSGS |
| | | GSGTDFTLKISRVEAGDVGVYYCMQALQTPWTFGQGTKVEIK |
| | | RTVAAPSVFIFPPSDEQLKSGTASVVCLLNNFYPREAKVQWK |
| | | VDNALQSGNSQESVTEQDSKDSTYSLSSTLTLSKADYEKHKV |
| | | YACEVTHQGLSSPVTKSFNRGEC |
| 485 | scFab- | QVQLQESGPGLVKPSQTLSLTCTVSGDSISSNPYYWSWIRQHP |
| | BISPECIFIC | GKGLEWIGYISYSGITNYNPSLKSRVTMSVDTSKNQFSLKLTS |
| | MOL. | LTAADTAVYYCAREKMWFGVLNYYYGMDVWGQGTTVTVS |
| | | SASTKGPSVFPLAPSSKSTSGGTAALGCLVKDYFPEPVTVSWN |
| | | SGALTSGVHTFPAVLQSSGLYSLSSVVTVPSSSLGTQTYICNVN |
| | | HKPSNTKVDKKVEPKSCGGGGSGGGGSGGGGSGGGGSGGGG |
| | | SGGGGSGGGGSGGGGSDIVMTQTPLSLPVTPGEPASISCRSSQS |
| | | LLHRSGYNYLDWYLQKPGQSPQLLIYLGSNRASGVPDRFSGS |
| | | GSGTDFTLKISRVEAGDVGVYYCMQALQTPWTFGQGTKVEIK |
| | | RTVAAPSVFIFPPSDEQLKSGTASVVCLLNNFYPREAKVQWK |
| | | VDNALQSGNSQESVTEQDSKDSTYSLSSTLTLSKADYEKHKV |
| | | YACEVTHQGLSSPVTKSFNRGECSGGGGSEVQLVESGGGLVQ |
| | | PGGSLKLSCAASGFTFNKYAMNWVRQAPGKGLEWVARIRSK |
| | | YNNYATYYADSVKDRFTISRDDSKNTAYLQMNNLKTEDTAV |
| | | YYCVRHGNFGNSYISYWAYWGQGTLVTVSSGGGGSGGGGSG |
| | | GGGSQTVVTQEPSLTVSPGGTVTLTCGSSTGAVTSGNYPNWV |
| | | QQKPGQAPRGLIGGTKFLAPGTPARFSGSLLGGKAALTLSGVQ |
| | | PEDEAEYYCVLWYSNRWVFGGGTKLTVL |
| 486 | scFab-BiTE | QVQLQESGPGLVKPSQTLSLTCTVSGDSISSNPYYWSWIRQHP |
| | HLE | GKGLEWIGYISYSGITNYNPSLKSRVTMSVDTSKNQFSLKLTS |
| | | LTAADTAVYYCAREKMWFGVLNYYYGMDVWGQGTTVTVS |
| | | SASTKGPSVFPLAPSSKSTSGGTAALGCLVKDYFPEPVTVSWN |
| | | SGALTSGVHTFPAVLQSSGLYSLSSVVTVPSSSLGTQTYICNVN |
| | | HKPSNTKVDKKVEPKSCGGGGSGGGGSGGGGSGGGGSGGGG |
| | | SGGGGSGGGGSGGGGSDIVMTQTPLSLPVTPGEPASISCRSSQS |
| | | LLHRSGYNYLDWYLQKPGQSPQLLIYLGSNRASGVPDRFSGS |
| | | GSGTDFTLKISRVEAGDVGVYYCMQALQTPWTFGQGTKVEIK |
| | | RTVAAPSVFIFPPSDEQLKSGTASVVCLLNNFYPREAKVQWK |
| | | VDNALQSGNSQESVTEQDSKDSTYSLSSTLTLSKADYEKHKV |
| | | YACEVTHQGLSSPVTKSFNRGECSGGGGSEVQLVESGGGLVQ |
| | | PGGSLKLSCAASGFTFNKYAMNWVRQAPGKGLEWVARIRSK |
| | | YNNYATYYADSVKDRFTISRDDSKNTAYLQMNNLKTEDTAV |
| | | YYCVRHGNFGNSYISYWAYWGQGTLVTVSSGGGGSGGGGSG |
| | | GGGSQTVVTQEPSLTVSPGGTVTLTCGSSTGAVTSGNYPNWV |
| | | QQKPGQAPRGLIGGTKFLAPGTPARFSGSLLGGKAALTLSGVQ |
| | | PEDEAEYYCVLWYSNRWVFGGGTKLTVLGGGGDKTHTCPPC |
| | | PAPELLGGPSVFLFPPKPKDTLMISRTPEVTCVVVDVSHEDPEV |
| | | KFNWYVDGVEVHNAKTKPCEEQYGSTYRCVSVLTVLHQDW |
| | | LNGKEYKCKVSNKALPAPIEKTISKAKGQPREPQVYTLPPSRE |
| | | EMTKNQVSLTCLVKGFYPSDIAVEWESNGQPENNYKTTPPVL |
| | | DSDGSFFLYSKLTVDKSRWQQGNVFSCSVMHEALHNHYTQK |
| | | SLSLSPGKGGGGSGGGGSGGGGSGGGGSGGGGSGGGGSDKT |
| | | HTCPPCPAPELLGGPSVFLFPPKPKDTLMISRTPEVTCVVVDVS |
| | | HEDPEVKFNWYVDGVEVHNAKTKPCEEQYGSTYRCVSVLTV |
| | | LHQDWLNGKEYKCKVSNKALPAPIEKTISKAKGQPREPQVYT |
| | | LPPSREEMTKNQVSLTCLVKGFYPSDIAVEWESNGQPENNYK |
| | | TTPPVLDSDGSFFLYSKLTVDKSRWQQGNVFSCSVMHEALHN |
| | | HYTQKSLSLSPGK |

MA 03-G10 AS CC scFv × I2C × scFc

| | | |
|---|---|---|
| 487 | HCDR1 | SYAMS |
| 488 | HCDR2 | AISGSGGGTYYAASVKG |
| 489 | HCDR3 | GKGVHLGFDY |
| 490 | LCDR1 | GGNNIGSKSVH |
| 491 | LCDR2 | DDNDRPS |
| 492 | LCDR3 | QVWDYSGQRQV |
| 493 | VH | EVQLLESGGGLVQPGGSLRLSCAASGFTFSSYAMSWVRQAPG |
| | | KCLEWVSAISGSGGGTYYAASVKGRFTISRDNSKNTLYLQMS |
| | | SLRAEDTAVYYCATGKGVHLGFDYWGQGTLVTVSS |
| 494 | VL | SYVLTQPPSVSVAPGQTARITCGGNNIGSKSVHWYQQKPGQA |
| | | PVMVVYDDNDRPSGIPERFSGSNSGNTATLTISRVEAGDEADY |
| | | YCQVWDYSGQRQVFGCGTKLTVL |
| 495 | SCFV | EVQLLESGGGLVQPGGSLRLSCAASGFTFSSYAMSWVRQAPG |
| | | KCLEWVSAISGSGGGTYYAASVKGRFTISRDNSKNTLYLQMS |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYSGQRQVFGCGTKLTVL |
| 496 | BISPECIFIC | EVQLLESGGGLVQPGGSLRLSCAASGFTFSSYAMSWVRQAPG |
| | MOL. | KCLEWVSAISGSGGGTYYAASVKGRFTISRDNSKNTLYLQMS |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYSGQRQVFGCGTKLTVLSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| 497 | BiTE HLE | EVQLLESGGGLVQPGGSLRLSCAASGFTFSSYAMSWVRQAPG |
| | | KCLEWVSAISGSGGGTYYAASVKGRFTISRDNSKNTLYLQMS |
| | | SLRAEDTAVYYCATGKGVHLGFDYWGQGTLVTVSSGGGGSG |
| | | GGGSGGGGSSYVLTQPPSVSVAPGQTARITCGGNNIGSKSVH |
| | | WYQQKPGQAPVMVVYDDNDRPSGIPERFSGSNSGNTATLTIS |
| | | RVEAGDEADYYCQVWDYSGQRQVFGCGTKLTVLSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

MA 03-G10 AS scFab × I2C × scFc

| | | |
|---|---|---|
| 498 | VH-CH1 | EVQLLESGGGLVQPGGSLRLSCAASGFTFSSYAMSWVRQAPG |
| | | KGLEWVSAISGSGGGTYYAASVKGRFTISRDNSKNTLYLQMS |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSC |
| 499 | VL | SYVLTQPPSVSVAPGQTARITCGGNNIGSKSVHWYQQKPGQA |
| | | PVMVVYDDNDRPSGIPERFSGSNSGNTATLTISRVEAGDEADY |
| | | YCQVWDYSGQRQVFGGGTKLTVL |
| 500 | scFab | EVQLLESGGGLVQPGGSLRLSCAASGFTFSSYAMSWVRQAPG |
| | | KGLEWVSAISGSGGGTYYAASVKGRFTISRDNSKNTLYLQMS |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYSGQRQVFGGGTKLTVL |
| 501 | scFab- | EVQLLESGGGLVQPGGSLRLSCAASGFTFSSYAMSWVRQAPG |
| | BISPECIFIC | KGLEWVSAISGSGGGTYYAASVKGRFTISRDNSKNTLYLQMS |
| | MOL. | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYSGQRQVFGGGTKLTVLSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| 502 | scFab-BiTE | MNNLKTEDTAVYYCVRHGNFGNSYISYWAYWGQGTLVTVS |
| | HLE | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | EVQLLESGGGLVQPGGSLRLSCAASGFTFSSYAMSWVRQAPG |
| | | KGLEWVSAISGSGGGTYYAASVKGRFTISRDNSKNTLYLQMS |
| | | SLRAEDTAVYYCATGKGVHLGFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCGGGGSGGGGSGGGGSGGGGSGGGGSGGGG |
| | | SGGGGSGGGGSSYVLTQPPSVSVAPGQTARITCGGNNIGSKSV |
| | | HWYQQKPGQAPVMVVYDDNDRPSGIPERFSGSNSGNTATLTI |
| | | SRVEAGDEADYYCQVWDYSGQRQVFGGGTKLTVLSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

MA 10-B5 CC scFv × I2C × scFc

| | | |
|---|---|---|
| 503 | HCDR1 | NAWMS |
| 504 | HCDR2 | RIRSRSYGGTTDYAAPVKG |
| 505 | HCDR3 | PSYSGSYYNYFSVMDV |
| 506 | LCDR1 | RTSQSISSYLN |
| 507 | LCDR2 | AASSLQG |
| 508 | LCDR3 | QQTYSMPFT |
| 509 | VH | EVQLVESGGGLVQPGGSLRLSCAASGFTFSNAWMSWVRQAP |
| | | GKCLEWVGRIRSRSYGGTTDYAAPVKGRFTISRDDSKNTLFL |
| | | QMNSLKTEDTAVYYCTTPSYSGSYYNYFSVMDVWGQGTTVT |
| | | VSS |
| 510 | VL | DIQMTQSPSSLSASVGDRVTITCRTSQSISSYLNWYQQKPGRA |
| | | PKLLIFAASSLQGGVPSRFSGSGSGTDFTLTISSLQPEDFATYYC |
| | | QQTYSMPFTFGCGTKVEIK |
| 511 | SCFV | EVQLVESGGGLVQPGGSLRLSCAASGFTFSNAWMSWVRQAP |
| | | GKCLEWVGRIRSRSYGGTTDYAAPVKGRFTISRDDSKNTLFL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQTYSMPFTFGCGTKVEIK |
| 512 | BISPECIFIC | EVQLVESGGGLVQPGGSLRLSCAASGFTFSNAWMSWVRQAP |
| | MOL. | GKCLEWVGRIRSRSYGGTTDYAAPVKGRFTISRDDSKNTLFL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQTYSMPFTFGCGTKVEIKSGGGG |
| | | SEVQLVESGGGLVQPGGSLKLSCAASGFTFNKYAMNWVRQA |
| | | PGKGLEWVARIRSKYNNYATYYADSVKDRFTISRDDSKNTAY |
| | | LQMNNLKTEDTAVYYCVRHGNFGNSYISYWAYWGQGTLVT |
| | | VSSGGGGSGGGGSGGGGSQTVVTQEPSLTVSPGGTVTLTCGS |
| | | STGAVTSGNYPNWVQQKPGQAPRGLIGGTKFLAPGTPARFSG |
| | | SLLGGKAALTLSGVQPEDEAEYYCVLWYSNRWVFGGGTKLT |
| | | VL |
| 513 | BiTE HLE | EVQLVESGGGLVQPGGSLRLSCAASGFTFSNAWMSWVRQAP |
| | | GKCLEWVGRIRSRSYGGTTDYAAPVKGRFTISRDDSKNTLFL |
| | | QMNSLKTEDTAVYYCTTPSYSGSYYNYFSVMDVWGQGTTVT |
| | | VSSGGGGSGGGGSGGGGSDIQMTQSPSSLSASVGDRVTITCRT |
| | | SQSISSYLNWYQQKPGRAPKLLIFAASSLQGGVPSRFSGSGSGT |
| | | DFTLTISSLQPEDFATYYCQQTYSMPFTFGCGTKVEIKSGGGG |
| | | SEVQLVESGGGLVQPGGSLKLSCAASGFTFNKYAMNWVRQA |
| | | PGKGLEWVARIRSKYNNYATYYADSVKDRFTISRDDSKNTAY |
| | | LQMNNLKTEDTAVYYCVRHGNFGNSYISYWAYWGQGTLVT |
| | | VSSGGGGSGGGGSGGGGSQTVVTQEPSLTVSPGGTVTLTCGS |
| | | STGAVTSGNYPNWVQQKPGQAPRGLIGGTKFLAPGTPARFSG |
| | | SLLGGKAALTLSGVQPEDEAEYYCVLWYSNRWVFGGGTKLT |
| | | VLGGGGDKTHTCPPCPAPELLGGPSVFLFPPKPKDTLMISRTPE |
| | | VTCVVVDVSHEDPEVKFNWYVDGVEVHNAKTKPCEEQYGST |
| | | YRCVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGKGGGGSGGGGSGGGGSGGGGS |
| | | GGGGSGGGGSDKTHTCPPCPAPELLGGPSVFLFPPKPKDTLMI |
| | | SRTPEVTCVVVDVSHEDPEVKFNWYVDGVEVHNAKTKPCEE |
| | | QYGSTYRCVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQGN |
| | | VFSCSVMHEALHNHYTQKSLSLSPGK |

MA 10-B5 scFab × I2C × scFc

| | | |
|---|---|---|
| 514 | VH-CH1 | EVQLVESGGGLVQPGGSLRLSCAASGFTFSNAWMSWVRQAP |
| | | GKGLEWVGRIRSRSYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSC |
| 515 | VL-CL | DIQMTQSPSSLSASVGDRVTITCRTSQSISSYLNWYQQKPGRA |
| | | PKLLIFAASSLQGGVPSRFSGSGSGTDFTLTISSLQPEDFATYYC |
| | | QQTYSMPFTFGGGTKVEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| 516 | scFab | EVQLVESGGGLVQPGGSLRLSCAASGFTFSNAWMSWVRQAP |
| | | GKGLEWVGRIRSRSYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGGGTKVEIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGEC |
| 517 | scFab- | EVQLVESGGGLVQPGGSLRLSCAASGFTFSNAWMSWVRQAP |
| | BISPECIFIC | GKGLEWVGRIRSRSYGGTTDYAAPVKGRFTISRDDSKNTLFL |
| | MOL. | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGGGTKVEIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGECSGGGGSEVQLVESGGGLVQPGG |
| | | SLKLSCAASGFTFNKYAMNWVRQAPGKGLEWVARIRSKYNN |
| | | YATYYADSVKDRFTISRDDSKNTAYLQMNNLKTEDTAVYYC |
| | | VRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVL |
| 518 | scFab-BiTE | EVQLVESGGGLVQPGGSLRLSCAASGFTFSNAWMSWVRQAP |
| | HLE | GKGLEWVGRIRSRSYGGTTDYAAPVKGRFTISRDDSKNTLFL |
| | | QMNSLKTEDTAVYYCTTPSYSGSYYNYFSVMDVWGQGTTVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKKVEPKSCGGGGSGGGGSGGGGSGGGGSG |
| | | GGGSGGGGSGGGGSGGGGSDIQMTQSPSSLSASVGDRVTITC |
| | | RTSQSISSYLNWYQQKPGRAPKLLIFAASSLQGGVPSRFSGSGS |
| | | GTDFTLTISSLQPEDFATYYCQQTYSMPFTFGGGTKVEIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGECSGGGGSEVQLVESGGGLVQPGG |
| | | SLKLSCAASGFTFNKYAMNWVRQAPGKGLEWVARIRSKYNN |
| | | YATYYADSVKDRFTISRDDSKNTAYLQMNNLKTEDTAVYYC |
| | | VRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVLGGGGDKTHTCPPCPA |
| | | PELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGKGGGGSGGGGSGGGGSGGGGSGGGGSGGGGSDKTHT |
| | | CPPCPAPELLGGPSVFLFPPKPKDTLMISRTPEVTCVVVDVSHE |
| | | DPEVKFNWYVDGVEVHNAKTKPCEEQYGSTYRCVSVLTVLH |
| | | QDWLNGKEYKCKVSNKALPAPIEKTISKAKGQPREPQVYTLP |
| | | PSREEMTKNQVSLTCLVKGFYPSDIAVEWESNGQPENNYKTT |
| | | PPVLDSDGSFFLYSKLTVDKSRWQQGNVFSCSVMHEALHNH |
| | | YTQKSLSLSPGK |

CD22 11-C3 CC scFv × I2C × scFc

| | | |
|---|---|---|
| 519 | HCDR1 | SFYWS |
| 520 | HCDR2 | RISSSGDVDYNPSLKS |
| 521 | HCDR3 | EGGFYY |
| 522 | LCDR1 | RASQSVSSSYLA |
| 523 | LCDR2 | GASSRAT |
| 524 | LCDR3 | QQYGSSPIT |
| 525 | VH | QVQLQESGPGLVKPSETLSLTCTVSGGSISSFYWSWIRQPPGK |
| | | CLEWIGRISSSGDVDYNPSLKSRVTISVDTSKNQFSLKLSSVTA |
| | | ADTAVYYCAREGGFYYWGQGTLVTVSS |
| 526 | VL | EIVLTQSPGTLSLSPGERATLSCRASQSVSSSYLAWYQQKPGQ |
| | | APRLLIYGASSRATGIPDRFSGSGSGTDFTLTISRLEPEDFAVYY |
| | | CQQYGSSPITFGCGTKLEIK |
| 527 | SCFV | QVQLQESGPGLVKPSETLSLTCTVSGGSISSFYWSWIRQPPGK |
| | | CLEWIGRISSSGDVDYNPSLKSRVTISVDTSKNQFSLKLSSVTA |
| | | ADTAVYYCAREGGFYYWGQGTLVTVSSGGGGSGGGGSGGG |
| | | GSEIVLTQSPGTLSLSPGERATLSCRASQSVSSSYLAWYQQKP |
| | | GQAPRLLIYGASSRATGIPDRFSGSGSGTDFTLTISRLEPEDFAV |
| | | YYCQQYGSSPITFGCGTKLEIK |
| 528 | BISPECIFIC | QVQLQESGPGLVKPSETLSLTCTVSGGSISSFYWSWIRQPPGK |
| | MOL. | CLEWIGRISSSGDVDYNPSLKSRVTISVDTSKNQFSLKLSSVTA |
| | | ADTAVYYCAREGGFYYWGQGTLVTVSSGGGGSGGGGSGGG |
| | | GSEIVLTQSPGTLSLSPGERATLSCRASQSVSSSYLAWYQQKP |
| | | GQAPRLLIYGASSRATGIPDRFSGSGSGTDFTLTISRLEPEDFAV |
| | | YYCQQYGSSPITFGCGTKLEIKSGGGGSEVQLVESGGGLVQPG |
| | | GSLKLSCAASGFTFNKYAMNWVRQAPGKGLEWVARIRSKYN |
| | | NYATYYADSVKDRFTISRDDSKNTAYLQMNNLKTEDTAVYY |
| | | CVRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVL |
| 529 | BiTE HLE | QVQLQESGPGLVKPSETLSLTCTVSGGSISSFYWSWIRQPPGK |
| | | CLEWIGRISSSGDVDYNPSLKSRVTISVDTSKNQFSLKLSSVTA |
| | | ADTAVYYCAREGGFYYWGQGTLVTVSSGGGGSGGGGSGGG |
| | | GSEIVLTQSPGTLSLSPGERATLSCRASQSVSSSYLAWYQQKP |
| | | GQAPRLLIYGASSRATGIPDRFSGSGSGTDFTLTISRLEPEDFAV |
| | | YYCQQYGSSPITFGCGTKLEIKSGGGGSEVQLVESGGGLVQPG |
| | | GSLKLSCAASGFTFNKYAMNWVRQAPGKGLEWVARIRSKYN |
| | | NYATYYADSVKDRFTISRDDSKNTAYLQMNNLKTEDTAVYY |
| | | CVRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVLGGGGDKTHTCPPCPA |
| | | PELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGKGGGGSGGGGSGGGGSGGGGSGGGGSGGGGSDKTHT |
| | | CPPCPAPELLGGPSVFLFPPKPKDTLMISRTPEVTCVVVDVSHE |
| | | DPEVKFNWYVDGVEVHNAKTKPCEEQYGSTYRCVSVLTVLH |
| | | QDWLNGKEYKCKVSNKALPAPIEKTISKAKGQPREPQVYTLP |
| | | PSREEMTKNQVSLTCLVKGFYPSDIAVEWESNGQPENNYKTT |
| | | PPVLDSDGSFFLYSKLTVDKSRWQQGNVFSCSVMHEALHNH |
| | | YTQKSLSLSPGK |

CD20 29-F5 CC scFv × I2C × scFc

| | | |
|---|---|---|
| 530 | HCDR1 | GYYWS |
| 531 | HCDR2 | EINHRGSTNYNPSLKS |
| 532 | HCDR3 | GYSSSWYTGYVFDY |
| 533 | LCDR1 | TGTSSDVFGYDYVS |
| 534 | LCDR2 | DVSNRPS |
| 535 | LCDR3 | SSYKSSSTVV |
| 536 | VH | QVQLQQWGAGLLKPSETLSLTCAVYGGSFSGYYWSWIRQPP |
| | | GKCLEWIGEINHRGSTNYNPSLKSRVTISGDTSKNQFSLKLSSV |
| | | NAADTAVYYCARGYSSSWYTGYVFDYWGQGTLVTVSS |
| 537 | VL | QSALTQPASVSGSPGQSITISCTGTSSDVFGYDYVSWYQQHPG |
| | | KAPKLMIYDVSNRPSGVSNRFSGSKSGNAASLTISGLQAEDEA |
| | | DYYCSSYKSSSTVVFGCGTKLTVL |
| 538 | SCFV | QVQLQQWGAGLLKPSETLSLTCAVYGGSFSGYYWSWIRQPP |
| | | GKCLEWIGEINHRGSTNYNPSLKSRVTISGDTSKNQFSLKLSSV |
| | | NAADTAVYYCARGYSSSWYTGYVFDYWGQGTLVTVSSGGG |
| | | GSGGGGSGGGGSQSALTQPASVSGSPGQSITISCTGTSSDVFGY |
| | | DYVSWYQQHPGKAPKLMIYDVSNRPSGVSNRFSGSKSGNAA |
| | | SLTISGLQAEDEADYYCSSYKSSSTVVFGCGTKLTVL |
| 539 | BISPECIFIC | QVQLQQWGAGLLKPSETLSLTCAVYGGSFSGYYWSWIRQPP |
| | MOL. | GKCLEWIGEINHRGSTNYNPSLKSRVTISGDTSKNQFSLKLSSV |
| | | NAADTAVYYCARGYSSSWYTGYVFDYWGQGTLVTVSSGGG |
| | | GSGGGGSGGGGSQSALTQPASVSGSPGQSITISCTGTSSDVFGY |
| | | DYVSWYQQHPGKAPKLMIYDVSNRPSGVSNRFSGSKSGNAA |
| | | SLTISGLQAEDEADYYCSSYKSSSTVVFGCGTKLTVLSGGGGS |
| | | EVQLVESGGGLVQPGGSLKLSCAASGFTFNKYAMNWVRQAP |
| | | GKGLEWVARIRSKYNNYATYYADSVKDRFTISRDDSKNTAYL |
| | | QMNNLKTEDTAVYYCVRHGNFGNSYISYWAYWGQGTLVTV |
| | | SSGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| 540 | BiTE HLE | QVQLQQWGAGLLKPSETLSLTCAVYGGSFSGYYWSWIRQPP |
| | | GKCLEWIGEINHRGSTNYNPSLKSRVTISGDTSKNQFSLKLSSV |
| | | NAADTAVYYCARGYSSSWYTGYVFDYWGQGTLVTVSSGGG |
| | | GSGGGGSGGGGSQSALTQPASVSGSPGQSITISCTGTSSDVFGY |
| | | DYVSWYQQHPGKAPKLMIYDVSNRPSGVSNRFSGSKSGNAA |
| | | SLTISGLQAEDEADYYCSSYKSSSTVVFGCGTKLTVLSGGGGS |
| | | EVQLVESGGGLVQPGGSLKLSCAASGFTFNKYAMNWVRQAP |
| | | GKGLEWVARIRSKYNNYATYYADSVKDRFTISRDDSKNTAYL |
| | | QMNNLKTEDTAVYYCVRHGNFGNSYISYWAYWGQGTLVTV |
| | | SSGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

CD22 28-B7 N655 CC scFv × I2C × scFc

| | | |
|---|---|---|
| 541 | HCDR1 | SYGIS |
| 542 | HCDR2 | WISAYSGNAIYAQKLQG |
| 543 | HCDR3 | DPDYYGSGSYSDY |
| 544 | LCDR1 | RASQSVSSNLA |
| 545 | LCDR2 | GASSRAT |
| 546 | LCDR3 | QQYHSWPLLT |
| 547 | VH | QVQLVQSGAEVKKPGASVKVSCKASGYTFTSYGISWVRQAP |
| | | GQCLEWMGWISAYSGNAIYAQKLQGRVTMTRDTSTSTAYME |
| | | LRSLRSDDTAVYYCARDPDYYGSGSYSDYWGQGTLVTVSS |
| 548 | VL | EIVLTQSPATLSVSPGERATLSCRASQSVSSNLAWYQQKPGQA |
| | | PRLLIYGASSRATGIPARFSGSGSGTEFTLTISSLQSEDFAVYYC |
| | | QQYHSWPLLTFGCGTKVEIK |
| 549 | SCFV | QVQLVQSGAEVKKPGASVKVSCKASGYTFTSYGISWVRQAP |
| | | GQCLEWMGWISAYSGNAIYAQKLQGRVTMTRDTSTSTAYME |
| | | LRSLRSDDTAVYYCARDPDYYGSGSYSDYWGQGTLVTVSSG |
| | | GGGSGGGGSGGGGSEIVLTQSPATLSVSPGERATLSCRASQSV |
| | | SSNLAWYQQKPGQAPRLLIYGASSRATGIPARFSGSGSGTEFT |
| | | LTISSLQSEDFAVYYCQQYHSWPLLTFGCGTKVEIK |
| 550 | BISPECIFIC | QVQLVQSGAEVKKPGASVKVSCKASGYTFTSYGISWVRQAP |
| | MOL. | GQCLEWMGWISAYSGNAIYAQKLQGRVTMTRDTSTSTAYME |
| | | LRSLRSDDTAVYYCARDPDYYGSGSYSDYWGQGTLVTVSSG |
| | | GGGSGGGGSGGGGSEIVLTQSPATLSVSPGERATLSCRASQSV |
| | | SSNLAWYQQKPGQAPRLLIYGASSRATGIPARFSGSGSGTEFT |
| | | LTISSLQSEDFAVYYCQQYHSWPLLTFGCGTKVEIKSGGGGSE |
| 551 | BiTE HLE | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | QVQLVQSGAEVKKPGASVKVSCKASGYTFTSYGISWVRQAP |
| | | GQCLEWMGWISAYSGNAIYAQKLQGRVTMTRDTSTSTAYME |
| | | LRSLRSDDTAVYYCARDPDYYGSGSYSDYWGQGTLVTVSSG |
| | | GGGSGGGGSGGGGSEIVLTQSPATLSVSPGERATLSCRASQSV |
| | | SSNLAWYQQKPGQAPRLLIYGASSRATGIPARFSGSGSGTEFT |
| | | LTISSLQSEDFAVYYCQQYHSWPLLTFGCGTKVEIKSGGGGSE |
| | | VQLVESGGGLVQPGGSLKLSCAASGFTFNKYAMNWVRQAPG |
| | | KGLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEVT |
| | | CVVVDVSHEDPEVKFNWYVDGVEVHNAKTKPCEEQYGSTYR |
| | | CVSVLTVLHQDWLNGKEYKCKVSNKALPAPIEKTISKAKGQP |
| | | REPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKGGGGSGGGGSGGGGSGGGGSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

CD20 99-E5 CC scFv × I2C × scFc

| | | |
|---|---|---|
| 552 | HCDR1 | SYWMH |
| 553 | HCDR2 | YITPSTGYTEYNQKFKG |
| 554 | HCDR3 | VHDYDRAMEY |
| 555 | LCDR1 | KASQDINKYIA |
| 556 | LCDR2 | YTSTLQP |
| 557 | LCDR3 | LQYASYPFT |
| 558 | VH | QVQLVQSGAEVKKPGASVKVSCKASGYTFTSYWMHWVRQA |
| | | PGQCLEWIGYITPSTGYTEYNQKFKGRVTMTRDKSTSTVYME |
| | | LSSLTSEDTAVYYCARVHDYDRAMEYWGQGTTVTVSS |
| 559 | VL | DIQMTQSPSSLSASVGDRVTITCKASQDINKYIAWYQQKPGKG |
| | | PKLLIYYTSTLQPGVPSRFSGSGSGTDFTFTISSLQPEDIATYYC |
| | | LQYASYPFTFGCGTRLEIK |
| 560 | SCFV | QVQLVQSGAEVKKPGASVKVSCKASGYTFTSYWMHWVRQA |
| | | PGQCLEWIGYITPSTGYTEYNQKFKGRVTMTRDKSTSTVYME |
| | | LSSLTSEDTAVYYCARVHDYDRAMEYWGQGTTVTVSSGGGG |
| | | SGGGGSGGGGSDIQMTQSPSSLSASVGDRVTITCKASQDINKY |
| | | IAWYQQKPGKGPKLLIYYTSTLQPGVPSRFSGSGSGTDFTFTIS |
| | | SLQPEDIATYYCLQYASYPFTFGCGTRLEIK |
| 561 | BISPECIFIC | QVQLVQSGAEVKKPGASVKVSCKASGYTFTSYWMHWVRQA |
| | MOL. | PGQCLEWIGYITPSTGYTEYNQKFKGRVTMTRDKSTSTVYME |
| | | LSSLTSEDTAVYYCARVHDYDRAMEYWGQGTTVTVSSGGGG |
| | | SGGGGSGGGGSDIQMTQSPSSLSASVGDRVTITCKASQDINKY |
| | | IAWYQQKPGKGPKLLIYYTSTLQPGVPSRFSGSGSGTDFTFTIS |
| | | SLQPEDIATYYCLQYASYPFTFGCGTRLEIKSGGGGSEVQLVE |
| | | SGGGLVQPGGSLKLSCAASGFTFNKYAMNWVRQAPGKGLE |
| | | WVARIRSKYNNYATYYADSVKDRFTISRDDSKNTAYLQMNN |
| | | LKTEDTAVYYCVRHGNFGNSYISYWAYWGQGTLVTVSSGGG |
| | | GSGGGGSGGGGSQTVVTQEPSLTVSPGGTVTLTCGSSTGAVT |
| | | SGNYPNWVQQKPGQAPRGLIGGTKFLAPGTPARFSGSLLGGK |
| | | AALTLSGVQPEDEAEYYCVLWYSNRWVFGGGTKLTVL |
| 562 | BiTE HLE | QVQLVQSGAEVKKPGASVKVSCKASGYTFTSYWMHWVRQA |
| | | PGQCLEWIGYITPSTGYTEYNQKFKGRVTMTRDKSTSTVYME |
| | | LSSLTSEDTAVYYCARVHDYDRAMEYWGQGTTVTVSSGGGG |
| | | SGGGGSGGGGSDIQMTQSPSSLSASVGDRVTITCKASQDINKY |
| | | IAWYQQKPGKGPKLLIYYTSTLQPGVPSRFSGSGSGTDFTFTIS |
| | | SLQPEDIATYYCLQYASYPFTFGCGTRLEIKSGGGGSEVQLVE |
| | | SGGGLVQPGGSLKLSCAASGFTFNKYAMNWVRQAPGKGLE |
| | | WVARIRSKYNNYATYYADSVKDRFTISRDDSKNTAYLQMNN |
| | | LKTEDTAVYYCVRHGNFGNSYISYWAYWGQGTLVTVSSGGG |
| | | GSGGGGSGGGGSQTVVTQEPSLTVSPGGTVTLTCGSSTGAVT |
| | | SGNYPNWVQQKPGQAPRGLIGGTKFLAPGTPARFSGSLLGGK |
| | | AALTLSGVQPEDEAEYYCVLWYSNRWVFGGGTKLTVLGGGG |
| | | DKTHTCPPCPAPELLGGPSVFLFPPKPKDTLMISRTPEVTCVVV |
| | | DVSHEDPEVKFNWYVDGVEVHNAKTKPCEEQYGSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHEA |
| | | LHNHYTQKSLSLSPGKGGGGSGGGGSGGGGSGGGGSGGGGS |
| | | GGGGSDKTHTCPPCPAPELLGGPSVFLFPPKPKDTLMISRTPEV |
| | | TCVVVDVSHEDPEVKFNWYVDGVEVHNAKTKPCEEQYGSTY |
| | | RCVSVLTVLHQDWLNGKEYKCKVSNKALPAPIEKTISKAKGQ |
| | | PREPQVYTLPPSREEMTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGK |

CD20 99-E5 scFab × I2C × scFc

| | | |
|---|---|---|
| 563 | VH-CH1 | QVQLVQSGAEVKKPGASVKVSCKASGYTFTSYWMHWVRQA |
| | | PGQGLEWIGYITPSTGYTEYNQKFKGRVTMTRDKSTSTVYME |
| | | LSSLTSEDTAVYYCARVHDYDRAMEYWGQGTTVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSC |
| 564 | VL-CL | DIQMTQSPSSLSASVGDRVTITCKASQDINKYIAWYQQKPGKG |
| | | PKLLIYYTSTLQPGVPSRFSGSGSGTDFTFTISSLQPEDIATYYC |
| | | LQYASYPFTFGQGTRLEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| 565 | scFab | QVQLVQSGAEVKKPGASVKVSCKASGYTFTSYWMHWVRQA |
| | | PGQGLEWIGYITPSTGYTEYNQKFKGRVTMTRDKSTSTVYME |
| | | LSSLTSEDTAVYYCARVHDYDRAMEYWGQGTTVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCKASQDIN |
| | | KYIAWYQQKPGKGPKLLIYYTSTLQPGVPSRFSGSGSGTDFTF |
| | | TISSLQPEDIATYYCLQYASYPFTFGQGTRLEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGEC |
| 566 | scFab- | QVQLVQSGAEVKKPGASVKVSCKASGYTFTSYWMHWVRQA |
| | BISPECIFIC | PGQGLEWIGYITPSTGYTEYNQKFKGRVTMTRDKSTSTVYME |
| | MOL. | LSSLTSEDTAVYYCARVHDYDRAMEYWGQGTTVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCKASQDIN |
| | | KYIAWYQQKPGKGPKLLIYYTSTLQPGVPSRFSGSGSGTDFTF |
| | | TISSLQPEDIATYYCLQYASYPFTFGQGTRLEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVL |
| 567 | scFab-BiTE | QVQLVQSGAEVKKPGASVKVSCKASGYTFTSYWMHWVRQA |
| | HLE | PGQGLEWIGYITPSTGYTEYNQKFKGRVTMTRDKSTSTVYME |
| | | LSSLTSEDTAVYYCARVHDYDRAMEYWGQGTTVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCKASQDIN |
| | | KYIAWYQQKPGKGPKLLIYYTSTLQPGVPSRFSGSGSGTDFTF |
| | | TISSLQPEDIATYYCLQYASYPFTFGQGTRLEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSEVQLVESGGGLVQPGGSLKLSCA |
| | | ASGFTFNKYAMNWVRQAPGKGLEWVARIRSKYNNYATYYA |
| | | DSVKDRFTISRDDSKNTAYLQMNNLKTEDTAVYYCVRHGNF |
| | | GNSYISYWAYWGQGTLVTVSSGGGGSGGGGSGGGGSQTVVT |
| | | QEPSLTVSPGGTVTLTCGSSTGAVTSGNYPNWVQQKPGQAPR |
| | | GLIGGTKFLAPGTPARFSGSLLGGKAALTLSGVQPEDEAEYYC |
| | | VLWYSNRWVFGGGTKLTVLGGGGDKTHTCPPCPAPELLGGP |
| | | SVFLFPPKPKDTLMISRTPEVTCVVVDVSHEDPEVKFNWYVD |
| | | GVEVHNAKTKPCEEQYGSTYRCVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKG |
| | | GGGSGGGGSGGGGSGGGGSGGGGSGGGGSDKTHTCPPCPAP |
| | | ELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |

CD20 29-F5 scFab × I2C × scFc

| | | |
|---|---|---|
| 568 | VH-CH1 | QVQLQQWGAGLLKPSETLSLTCAVYGGSFSGYYWSWIRQPP |
| | | GKGLEWIGEINHRGSTNYNPSLKSRVTISGDTSKNQFSLKLSSV |
| | | NAADTAVYYCARGYSSSWYTGYVFDYWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSC |
| 569 | VL-CL | QSALTQPASVSGSPGQSITISCTGTSSDVFGYDYVSWYQQHPG |
| | | KAPKLMIYDVSNRPSGVSNRFSGSKSGNAASLTISGLQAEDEA |
| | | DYYCSSYKSSSTVVFGGGTKLTVLGQPKAAPSVTLFPPSSEEL |
| | | QANKATLVCLISDFYPGAVTVAWKADSSPVKAGVETTTPSKQ |
| | | SNNKYAASSYLSLTPEQWKSHRSYSCQVTHEGSTVEKTVAPT |
| | | ECS |
| 570 | scFab | QVQLQQWGAGLLKPSETLSLTCAVYGGSFSGYYWSWIRQPP |
| | | GKGLEWIGEINHRGSTNYNPSLKSRVTISGDTSKNQFSLKLSSV |
| | | NAADTAVYYCARGYSSSWYTGYVFDYWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSQSALTQPASVSGSPGQSITISCTGTSSDVFG |
| | | YDYVSWYQQHPGKAPKLMIYDVSNRPSGVSNRFSGSKSGNA |
| | | ASLTISGLQAEDEADYYCSSYKSSSTVVFGGGTKLTVLGQPKA |
| | | APSVTLFPPSSEELQANKATLVCLISDFYPGAVTVAWKADSSP |
| | | VKAGVETTTPSKQSNNKYAASSYLSLTPEQWKSHRSYSCQVT |
| | | HEGSTVEKTVAPTECS |
| 571 | scFab- | QVQLQQWGAGLLKPSETLSLTCAVYGGSFSGYYWSWIRQPP |
| | BISPECIFIC | GKGLEWIGEINHRGSTNYNPSLKSRVTISGDTSKNQFSLKLSSV |
| | MOL. | NAADTAVYYCARGYSSSWYTGYVFDYWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSQSALTQPASVSGSPGQSITISCTGTSSDVFG |
| | | YDYVSWYQQHPGKAPKLMIYDVSNRPSGVSNRFSGSKSGNA |
| | | ASLTISGLQAEDEADYYCSSYKSSSTVVFGGGTKLTVLGQPKA |
| | | APSVTLFPPSSEELQANKATLVCLISDFYPGAVTVAWKADSSP |
| | | VKAGVETTTPSKQSNNKYAASSYLSLTPEQWKSHRSYSCQVT |
| | | HEGSTVEKTVAPTECSSGGGGSEVQLVESGGGLVQPGGSLKL |
| | | SCAASGFTFNKYAMNWVRQAPGKGLEWVARIRSKYNNYAT |
| | | YYADSVKDRFTISRDDSKNTAYLQMNNLKTEDTAVYYCVRH |
| | | GNFGNSYISYWAYWGQGTLVTVSSGGGGSGGGGSGGGGSQT |
| | | VVTQEPSLTVSPGGTVTLTCGSSTGAVTSGNYPNWVQQKPGQ |
| | | APRGLIGGTKFLAPGTPARFSGSLLGGKAALTLSGVQPEDEAE |
| | | YYCVLWYSNRWVFGGGTKLTVL |
| 572 | scFab-BiTE | QVQLQQWGAGLLKPSETLSLTCAVYGGSFSGYYWSWIRQPP |
| | HLE | GKGLEWIGEINHRGSTNYNPSLKSRVTISGDTSKNQFSLKLSSV |
| | | NAADTAVYYCARGYSSSWYTGYVFDYWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSQSALTQPASVSGSPGQSITISCTGTSSDVFG |
| | | YDYVSWYQQHPGKAPKLMIYDVSNRPSGVSNRFSGSKSGNA |
| | | ASLTISGLQAEDEADYYCSSYKSSSTVVFGGGTKLTVLGQPKA |
| | | APSVTLFPPSSEELQANKATLVCLISDFYPGAVTVAWKADSSP |
| | | VKAGVETTTPSKQSNNKYAASSYLSLTPEQWKSHRSYSCQVT |
| | | HEGSTVEKTVAPTECSSGGGGSEVQLVESGGGLVQPGGSLKL |
| | | SCAASGFTFNKYAMNWVRQAPGKGLEWVARIRSKYNNYAT |
| | | YYADSVKDRFTISRDDSKNTAYLQMNNLKTEDTAVYYCVRH |
| | | GNFGNSYISYWAYWGQGTLVTVSSGGGGSGGGGSGGGGSQT |
| | | VVTQEPSLTVSPGGTVTLTCGSSTGAVTSGNYPNWVQQKPGQ |
| | | APRGLIGGTKFLAPGTPARFSGSLLGGKAALTLSGVQPEDEAE |
| | | YYCVLWYSNRWVFGGGTKLTVLGGGGDKTHTCPPCPAPELL |
| | | GGPSVFLFPPKPKDTLMISRTPEVTCVVVDVSHEDPEVKFNWY |
| | | VDGVEVHNAKTKPCEEQYGSTYRCVSVLTVLHQDWLNGKEY |
| | | KCKVSNKALPAPIEKTISKAKGQPREPQVYTLPPSREEMTKNQ |
| | | VSLTCLVKGFYPSDIAVEWESNGQPENNYKTTPPVLDSDGSFF |
| | | LYSKLTVDKSRWQQGNVFSCSVMHEALHNHYTQKSLSLSPG |
| | | KGGGGSGGGGSGGGGSGGGGSGGGGSGGGGSDKTHTCPPCP |
| | | APELLGGPSVFLFPPKPKDTLMISRTPEVTCVVVDVSHEDPEV |
| | | KFNWYVDGVEVHNAKTKPCEEQYGSTYRCVSVLTVLHQDW |
| | | LNGKEYKCKVSNKALPAPIEKTISKAKGQPREPQVYTLPPSRE |
| | | EMTKNQVSLTCLVKGFYPSDIAVEWESNGQPENNYKTTPPVL |
| | | DSDGSFFLYSKLTVDKSRWQQGNVFSCSVMHEALHNHYTQK |
| | | SLSLSPGK |

CD22 11-C3 scFab × I2C × scFc

| | | |
|---|---|---|
| 573 | VH-CH1 | QVQLQESGPGLVKPSETLSLTCTVSGGSISSFYWSWIRQPPGK |
| | | GLEWIGRISSSGDVDYNPSLKSRVTISVDTSKNQFSLKLSSVTA |
| | | ADTAVYYCAREGGFYYWGQGTLVTVSSASTKGPSVFPLAPSS |
| | | KSTSGGTAALGCLVKDYFPEPVTVSWNSGALTSGVHTFPAVL |
| | | QSSGLYSLSSVVTVPSSSLGTQTYICNVNHKPSNTKVDKKVEP |
| | | KSC |
| 574 | VL-CL | EIVLTQSPGTLSLSPGERATLSCRASQSVSSSYLAWYQQKPGQ |
| | | APRLLIYGASSRATGIPDRFSGSGSGTDFTLTISRLEPEDFAVYY |
| | | CQQYGSSPITFGQGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| 575 | scFab | QVQLQESGPGLVKPSETLSLTCTVSGGSISSFYWSWIRQPPGK |
| | | GLEWIGRISSSGDVDYNPSLKSRVTISVDTSKNQFSLKLSSVTA |
| | | ADTAVYYCAREGGFYYWGQGTLVTVSSASTKGPSVFPLAPSS |
| | | KSTSGGTAALGCLVKDYFPEPVTVSWNSGALTSGVHTFPAVL |
| | | QSSGLYSLSSVVTVPSSSLGTQTYICNVNHKPSNTKVDKKVEP |
| | | KSCGGGGSGGGGSGGGGSGGGGSGGGGSGGGGSGGGGSGG |
| | | GGSEIVLTQSPGTLSLSPGERATLSCRASQSVSSSYLAWYQQK |
| | | PGQAPRLLIYGASSRATGIPDRFSGSGSGTDFTLTISRLEPEDFA |
| | | VYYCQQYGSSPITFGQGTKLEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 576 | scFab- | QVQLQESGPGLVKPSETLSLTCTVSGGSISSFYWSWIRQPPGK |
| | BISPECIFIC | GLEWIGRISSSGDVDYNPSLKSRVTISVDTSKNQFSLKLSSVTA |
| | MOL. | ADTAVYYCAREGGFYYWGQGTLVTVSSASTKGPSVFPLAPSS |
| | | KSTSGGTAALGCLVKDYFPEPVTVSWNSGALTSGVHTFPAVL |
| | | QSSGLYSLSSVVTVPSSSLGTQTYICNVNHKPSNTKVDKKVEP |
| | | KSCGGGGSGGGGSGGGGSGGGGSGGGGSGGGGSGGGGSGG |
| | | GGSEIVLTQSPGTLSLSPGERATLSCRASQSVSSSYLAWYQQK |
| | | PGQAPRLLIYGASSRATGIPDRFSGSGSGTDFTLTISRLEPEDFA |
| | | VYYCQQYGSSPITFGQGTKLEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNRG |
| | | ECSGGGGSEVQLVESGGGLVQPGGSLKLSCAASGFTFNKYAM |
| | | NWVRQAPGKGLEWVARIRSKYNNYATYYADSVKDRFTISRD |
| | | DSKNTAYLQMNNLKTEDTAVYYCVRHGNFGNSYISYWAYW |
| | | GQGTLVTVSSGGGGSGGGGSGGGGSQTVVTQEPSLTVSPGGT |
| | | VTLTCGSSTGAVTSGNYPNWVQQKPGQAPRGLIGGTKFLAPG |
| | | TPARFSGSLLGGKAALTLSGVQPEDEAEYYCVLWYSNRWVF |
| | | GGGTKLTVL |
| 577 | scFab-BiTE | QVQLQESGPGLVKPSETLSLTCTVSGGSISSFYWSWIRQPPGK |
| | HLE | GLEWIGRISSSGDVDYNPSLKSRVTISVDTSKNQFSLKLSSVTA |
| | | ADTAVYYCAREGGFYYWGQGTLVTVSSASTKGPSVFPLAPSS |
| | | KSTSGGTAALGCLVKDYFPEPVTVSWNSGALTSGVHTFPAVL |
| | | QSSGLYSLSSVVTVPSSSLGTQTYICNVNHKPSNTKVDKKVEP |
| | | KSCGGGGSGGGGSGGGGSGGGGSGGGGSGGGGSGGGGSGG |
| | | GGSEIVLTQSPGTLSLSPGERATLSCRASQSVSSSYLAWYQQK |
| | | PGQAPRLLIYGASSRATGIPDRFSGSGSGTDFTLTISRLEPEDFA |
| | | VYYCQQYGSSPITFGQGTKLEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNRG |
| | | ECSGGGGSEVQLVESGGGLVQPGGSLKLSCAASGFTFNKYAM |
| | | NWVRQAPGKGLEWVARIRSKYNNYATYYADSVKDRFTISRD |
| | | DSKNTAYLQMNNLKTEDTAVYYCVRHGNFGNSYISYWAYW |
| | | GQGTLVTVSSGGGGSGGGGSGGGGSQTVVTQEPSLTVSPGGT |
| | | VTLTCGSSTGAVTSGNYPNWVQQKPGQAPRGLIGGTKFLAPG |
| | | TPARFSGSLLGGKAALTLSGVQPEDEAEYYCVLWYSNRWVF |
| | | GGGTKLTVLGGGGDKTHTCPPCPAPELLGGPSVFLFPPKPKDT |
| | | LMISRTPEVTCVVVDVSHEDPEVKFNWYVDGVEVHNAKTKP |
| | | CEEQYGSTYRCVSVLTVLHQDWLNGKEYKCKVSNKALPAPIE |
| | | KTISKAKGQPREPQVYTLPPSREEMTKNQVSLTCLVKGFYPSD |
| | | IAVEWESNGQPENNYKTTPPVLDSDGSFFLYSKLTVDKSRWQ |
| | | QGNVFSCSVMHEALHNHYTQKSLSLSPGKGGGGSGGGGSGG |
| | | GGSGGGGSGGGGSGGGGSDKTHTCPPCPAPELLGGPSVFLFPP |
| | | KPKDTLMISRTPEVTCVVVDVSHEDPEVKFNWYVDGVEVHN |
| | | AKTKPCEEQYGSTYRCVSVLTVLHQDWLNGKEYKCKVSNKA |
| | | LPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 578 | CD20 99-E5 | QVQLVQSGAEVKKPGASVKVSCKASGYTFTSYWMHWVRQA |
| | CC scFv × | PGQCLEWIGYITPSTGYTEYNQKFKGRVTMTRDKSTSTVYME |
| | CD22 28-B7 | LSSLTSEDTAVYYCARVHDYDRAMEYWGQGTTVTVSSGGGG |
| | N655 CC | SGGGGSGGGGSDIQMTQSPSSLSASVGDRVTITCKASQDINKY |
| | scFv | IAWYQQKPGKGPKLLIYYTSTLQPGVPSRFSGSGSGTDFTFTIS |
| | | SLQPEDIATYYCLQYASYPFTFGCGTRLEIKSGGGGSQVQLVQ |
| | | SGAEVKKPGASVKVSCKASGYTFTSYGISWVRQAPGQCLEW |
| | | MGWISAYSGNAIYAQKLQGRVTMTRDTSTSTAYMELRSLRSD |
| | | DTAVYYCARDPDYYGSGSYSDYWGQGTLVTVSSGGGGSGG |
| | | GGSGGGGSEIVLTQSPATLSVSPGERATLSCRASQSVSSNLAW |
| | | YQQKPGQAPRLLIYGASSRATGIPARFSGSGSGTEFTLTISSLQS |
| | | EDFAVYYCQQYHSWPLLTFGCGTKVEIK |
| 579 | CD20 99-E5 | QVQLVQSGAEVKKPGASVKVSCKASGYTFTSYWMHWVRQA |
| | CC scFv × | PGQCLEWIGYITPSTGYTEYNQKFKGRVTMTRDKSTSTVYME |
| | CD22 28-B7 | LSSLTSEDTAVYYCARVHDYDRAMEYWGQGTTVTVSSGGGG |
| | N655 CC | SGGGGSGGGGSDIQMTQSPSSLSASVGDRVTITCKASQDINKY |
| | scFv × I2C | IAWYQQKPGKGPKLLIYYTSTLQPGVPSRFSGSGSGTDFTFTIS |
| | | SLQPEDIATYYCLQYASYPFTFGCGTRLEIKSGGGGSQVQLVQ |
| | | SGAEVKKPGASVKVSCKASGYTFTSYGISWVRQAPGQCLEW |
| | | MGWISAYSGNAIYAQKLQGRVTMTRDTSTSTAYMELRSLRSD |
| | | DTAVYYCARDPDYYGSGSYSDYWGQGTLVTVSSGGGGSGG |
| | | GGSGGGGSEIVLTQSPATLSVSPGERATLSCRASQSVSSNLAW |
| | | YQQKPGQAPRLLIYGASSRATGIPARFSGSGSGTEFTLTISSLQS |
| | | EDFAVYYCQQYHSWPLLTFGCGTKVEIKSGGGGSEVQLVESG |
| | | GGLVQPGGSLKLSCAASGFTFNKYAMNWVRQAPGKGLEWV |
| | | ARIRSKYNNYATYYADSVKDRFTISRDDSKNTAYLQMNNLKT |
| | | EDTAVYYCVRHGNFGNSYISYWAYWGQGTLVTVSSGGGGSG |
| | | GGGSGGGGSQTVVTQEPSLTVSPGGTVTLTCGSSTGAVTSGN |
| | | YPNWVQQKPGQAPRGLIGGTKFLAPGTPARFSGSLLGGKAAL |
| | | TLSGVQPEDEAEYYCVLWYSNRWVFGGGTKLTVL |
| 580 | CD20 99-E5 | QVQLVQSGAEVKKPGASVKVSCKASGYTFTSYWMHWVRQA |
| | CC scFv × | PGQCLEWIGYITPSTGYTEYNQKFKGRVTMTRDKSTSTVYME |
| | CD22 28-B7 | LSSLTSEDTAVYYCARVHDYDRAMEYWGQGTTVTVSSGGGG |
| | N655 CC | SGGGGSGGGGSDIQMTQSPSSLSASVGDRVTITCKASQDINKY |
| | scFv × I2C × | IAWYQQKPGKGPKLLIYYTSTLQPGVPSRFSGSGSGTDFTFTIS |
| | scFc | SLQPEDIATYYCLQYASYPFTFGCGTRLEIKSGGGGSQVQLVQ |
| | | SGAEVKKPGASVKVSCKASGYTFTSYGISWVRQAPGQCLEW |
| | | MGWISAYSGNAIYAQKLQGRVTMTRDTSTSTAYMELRSLRSD |
| | | DTAVYYCARDPDYYGSGSYSDYWGQGTLVTVSSGGGGSGG |
| | | GGSGGGGSEIVLTQSPATLSVSPGERATLSCRASQSVSSNLAW |
| | | YQQKPGQAPRLLIYGASSRATGIPARFSGSGSGTEFTLTISSLQS |
| | | EDFAVYYCQQYHSWPLLTFGCGTKVEIKSGGGGSEVQLVESG |
| | | GGLVQPGGSLKLSCAASGFTFNKYAMNWVRQAPGKGLEWV |
| | | ARIRSKYNNYATYYADSVKDRFTISRDDSKNTAYLQMNNLKT |
| | | EDTAVYYCVRHGNFGNSYISYWAYWGQGTLVTVSSGGGGSG |
| | | GGGSGGGGSQTVVTQEPSLTVSPGGTVTLTCGSSTGAVTSGN |
| | | YPNWVQQKPGQAPRGLIGGTKFLAPGTPARFSGSLLGGKAAL |
| | | TLSGVQPEDEAEYYCVLWYSNRWVFGGGTKLTVLGGGGDKT |
| | | HTCPPCPAPELLGGPSVFLFPPKPKDTLMISRTPEVTCVVVDVS |
| | | HEDPEVKFNWYVDGVEVHNAKTKPCEEQYGSTYRCVSVLTV |
| | | LHQDWLNGKEYKCKVSNKALPAPIEKTISKAKGQPREPQVYT |
| | | LPPSREEMTKNQVSLTCLVKGFYPSDIAVEWESNGQPENNYK |
| | | TTPPVLDSDGSFFLYSKLTVDKSRWQQGNVFSCSVMHEALHN |
| | | HYTQKSLSLSPGKGGGGSGGGGSGGGGSGGGGSGGGGSGGG |
| | | GSDKTHTCPPCPAPELLGGPSVFLFPPKPKDTLMISRTPEVTCV |
| | | VVDVSHEDPEVKFNWYVDGVEVHNAKTKPCEEQYGSTYRCV |
| | | SVLTVLHQDWLNGKEYKCKVSNKALPAPIEKTISKAKGQPRE |
| | | PQVYTLPPSREEMTKNQVSLTCLVKGFYPSDIAVEWESNGQPE |
| | | NNYKTTPPVLDSDGSFFLYSKLTVDKSRWQQGNVFSCSVMHE |
| | | ALHNHYTQKSLSLSPGK |
| 581 | CD22 11-C3 | QVQLQESGPGLVKPSETLSLTCTVSGGSISSFYWSWIRQPPGK |
| | CC scFv × | CLEWIGRISSSGDVDYNPSLKSRVTISVDTSKNQFSLKLSSVTA |
| | CD20 29-F5 | ADTAVYYCAREGGFYYWGQGTLVTVSSGGGGSGGGGSGGG |
| | CC scFv | GSEIVLTQSPGTLSLSPGERATLSCRASQSVSSSYLAWYQQKP |
| | | GQAPRLLIYGASSRATGIPDRFSGSGSGTDFTLTISRLEPEDFAV |
| | | YYCQQYGSSPITFGCGTKLEIKSGGGGSQVQLQQWGAGLLKP |
| | | SETLSLTCAVYGGSFSGYYWSWIRQPPGKCLEWIGEINHRGST |
| | | NYNPSLKSRVTISGDTSKNQFSLKLSSVNAADTAVYYCARGY |
| | | SSSWYTGYVFDYWGQGTLVTVSSGGGGSGGGGSGGGGSQSA |
| | | LTQPASVSGSPGQSITISCTGTSSDVFGYDYVSWYQQHPGKAP |
| | | KLMIYDVSNRPSGVSNRFSGSKSGNAASLTISGLQAEDEADYY |
| | | CSSYKSSSTVVFGCGTKLTVL |
| 582 | CD22 11-C3 | QVQLQESGPGLVKPSETLSLTCTVSGGSISSFYWSWIRQPPGK |
| | CC scFv × | CLEWIGRISSSGDVDYNPSLKSRVTISVDTSKNQFSLKLSSVTA |
| | CD20 29-F5 | ADTAVYYCAREGGFYYWGQGTLVTVSSGGGGSGGGGSGGG |
| | CC scFv × | GSEIVLTQSPGTLSLSPGERATLSCRASQSVSSSYLAWYQQKP |
| | 12C | GQAPRLLIYGASSRATGIPDRFSGSGSGTDFTLTISRLEPEDFAV |
| | | YYCQQYGSSPITFGCGTKLEIKSGGGGSQVQLQQWGAGLLKP |
| | | SETLSLTCAVYGGSFSGYYWSWIRQPPGKCLEWIGEINHRGST |
| | | NYNPSLKSRVTISGDTSKNQFSLKLSSVNAADTAVYYCARGY |
| | | SSSWYTGYVFDYWGQGTLVTVSSGGGGSGGGGSGGGGSQSA |
| | | LTQPASVSGSPGQSITISCTGTSSDVFGYDYVSWYQQHPGKAP |
| | | KLMIYDVSNRPSGVSNRFSGSKSGNAASLTISGLQAEDEADYY |
| | | CSSYKSSSTVVFGCGTKLTVLSGGGGSEVQLVESGGGLVQPG |
| | | GSLKLSCAASGFTFNKYAMNWVRQAPGKGLEWVARIRSKYN |
| | | NYATYYADSVKDRFTISRDDSKNTAYLQMNNLKTEDTAVYY |
| | | CVRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVL |
| 583 | CD22 11-C3 | QVQLQESGPGLVKPSETLSLTCTVSGGSISSFYWSWIRQPPGK |
| | CC scFv × | CLEWIGRISSSGDVDYNPSLKSRVTISVDTSKNQFSLKLSSVTA |
| | CD20 29-F5 | ADTAVYYCAREGGFYYWGQGTLVTVSSGGGGSGGGGSGGG |
| | CC scFv × | GSEIVLTQSPGTLSLSPGERATLSCRASQSVSSSYLAWYQQKP |
| | I2C × scFc | GQAPRLLIYGASSRATGIPDRFSGSGSGTDFTLTISRLEPEDFAV |
| | | YYCQQYGSSPITFGCGTKLEIKSGGGGSQVQLQQWGAGLLKP |
| | | SETLSLTCAVYGGSFSGYYWSWIRQPPGKCLEWIGEINHRGST |
| | | NYNPSLKSRVTISGDTSKNQFSLKLSSVNAADTAVYYCARGY |
| | | SSSWYTGYVFDYWGQGTLVTVSSGGGGSGGGGSGGGGSQSA |
| | | LTQPASVSGSPGQSITISCTGTSSDVFGYDYVSWYQQHPGKAP |
| | | KLMIYDVSNRPSGVSNRFSGSKSGNAASLTISGLQAEDEADYY |
| | | CSSYKSSSTVVFGCGTKLTVLSGGGGSEVQLVESGGGLVQPG |
| | | GSLKLSCAASGFTFNKYAMNWVRQAPGKGLEWVARIRSKYN |
| | | NYATYYADSVKDRFTISRDDSKNTAYLQMNNLKTEDTAVYY |
| | | CVRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVLGGGGDKTHTCPPCPA |
| | | PELLGGPSVFLFPPKPKDTLMISRTPEVTCVVVDVSHEDPEVKF |
| | | NWYVDGVEVHNAKTKPCEEQYGSTYRCVSVLTVLHQDWLN |
| | | GKEYKCKVSNKALPAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGKGGGGSGGGGSGGGGSGGGGSGGGGSGGGGSDKTHT |
| | | CPPCPAPELLGGPSVFLFPPKPKDTLMISRTPEVTCVVVDVSHE |
| | | DPEVKFNWYVDGVEVHNAKTKPCEEQYGSTYRCVSVLTVLH |
| | | QDWLNGKEYKCKVSNKALPAPIEKTISKAKGQPREPQVYTLP |
| | | PSREEMTKNQVSLTCLVKGFYPSDIAVEWESNGQPENNYKTT |
| | | PPVLDSDGSFFLYSKLTVDKSRWQQGNVFSCSVMHEALHNH |
| | | YTQKSLSLSPGK |
| 584 | CD20 99-E5 | QVQLVQSGAEVKKPGASVKVSCKASGYTFTSYWMHWVRQA |
| | scFab × | PGQGLEWIGYITPSTGYTEYNQKFKGRVTMTRDKSTSTVYME |
| | CD22 28-B7 | LSSLTSEDTAVYYCARVHDYDRAMEYWGQGTTVTVSSASTK |
| | N655 CC | GPSVFPLAPSSKSTSGGTAALGCLVKDYFPEPVTVSWNSGALT |
| | scFv | SGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCKASQDIN |
| | | KYIAWYQQKPGKGPKLLIYYTSTLQPGVPSRFSGSGSGTDFTF |
| | | TISSLQPEDIATYYCLQYASYPFTFGQGTRLEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSQVQLVQSGAEVKKPGASVKVSCK |
| | | ASGYTFTSYGISWVRQAPGQCLEWMGWISAYSGNAIYAQKL |
| | | QGRVTMTRDTSTSTAYMELRSLRSDDTAVYYCARDPDYYGS |
| | | GSYSDYWGQGTLVTVSSGGGGSGGGGSGGGGSEIVLTQSPAT |
| | | LSVSPGERATLSCRASQSVSSNLAWYQQKPGQAPRLLIYGASS |
| | | RATGIPARFSGSGSGTEFTLTISSLQSEDFAVYYCQQYHSWPLL |
| | | TFGCGTKVEIK |
| 585 | CD20 99-E5 | QVQLVQSGAEVKKPGASVKVSCKASGYTFTSYWMHWVRQA |
| | scFab × | PGQGLEWIGYITPSTGYTEYNQKFKGRVTMTRDKSTSTVYME |
| | CD22 28-B7 | LSSLTSEDTAVYYCARVHDYDRAMEYWGQGTTVTVSSASTK |
| | N655 CC | GPSVFPLAPSSKSTSGGTAALGCLVKDYFPEPVTVSWNSGALT |
| | scFv × I2C | SGVHTFPAVLQSSGLYSLSSVVTVPSSSLGTQTYICNVNHKPS |
| | | NTKVDKKVEPKSCGGGGSGGGGSGGGGSGGGGSGGGGSGG |
| | | GGSGGGGSGGGGSDIQMTQSPSSLSASVGDRVTITCKASQDIN |
| | | KYIAWYQQKPGKGPKLLIYYTSTLQPGVPSRFSGSGSGTDFTF |
| | | TISSLQPEDIATYYCLQYASYPFTFGQGTRLEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSQVQLVQSGAEVKKPGASVKVSCK |
| | | ASGYTFTSYGISWVRQAPGQCLEWMGWISAYSGNAIYAQKL |
| | | QGRVTMTRDTSTSTAYMELRSLRSDDTAVYYCARDPDYYGS |
| | | GSYSDYWGQGTLVTVSSGGGGSGGGGSGGGGSEIVLTQSPAT |
| | | LSVSPGERATLSCRASQSVSSNLAWYQQKPGQAPRLLIYGASS |
| | | RATGIPARFSGSGSGTEFTLTISSLQSEDFAVYYCQQYHSWPLL |
| | | TFGCGTKVEIKSGGGGSEVQLVESGGGLVQPGGSLKLSCAAS |
| | | GFTFNKYAMNWVRQAPGKGLEWVARIRSKYNNYATYYADS |
| | | VKDRFTISRDDSKNTAYLQMNNLKTEDTAVYYCVRHGNFGN |
| | | SYISYWAYWGQGTLVTVSSGGGGSGGGGSGGGGSQTVVTQE |
| | | PSLTVSPGGTVTLTCGSSTGAVTSGNYPNWVQQKPGQAPRGL |
| | | IGGTKFLAPGTPARFSGSLLGGKAALTLSGVQPEDEAEYYCVL |
| | | WYSNRWVFGGGTKLTVL |
| 586 | CD20 99-E5 | QVQLVQSGAEVKKPGASVKVSCKASGYTFTSYWMHWVRQA |
| | scFab × | PGQGLEWIGYITPSTGYTEYNQKFKGRVTMTRDKSTSTVYME |
| | CD22 28-B7 | LSSLTSEDTAVYYCARVHDYDRAMEYWGQGTTVTVSSASTK |
| | N655 CC | GPSVFPLAPSSKSTSGGTAALGCLVKDYFPEPVTVSWNSGALT |
| | scFv × I2C × | SGVHTFPAVLQSSGLYSLSSVVTVPSSSLGTQTYICNVNHKPS |
| | scFc | NTKVDKKVEPKSCGGGGSGGGGSGGGGSGGGGSGGGGSGG |
| | | GGSGGGGSGGGGSDIQMTQSPSSLSASVGDRVTITCKASQDIN |
| | | KYIAWYQQKPGKGPKLLIYYTSTLQPGVPSRFSGSGSGTDFTF |
| | | TISSLQPEDIATYYCLQYASYPFTFGQGTRLEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQGL |
| | | SSPVTKSFNRGECSGGGGSQVQLVQSGAEVKKPGASVKVSCK |
| | | ASGYTFTSYGISWVRQAPGQCLEWMGWISAYSGNAIYAQKL |
| | | QGRVTMTRDTSTSTAYMELRSLRSDDTAVYYCARDPDYYGS |
| | | GSYSDYWGQGTLVTVSSGGGGSGGGGSGGGGSEIVLTQSPAT |
| | | LSVSPGERATLSCRASQSVSSNLAWYQQKPGQAPRLLIYGASS |
| | | RATGIPARFSGSGSGTEFTLTISSLQSEDFAVYYCQQYHSWPLL |
| | | TFGCGTKVEIKSGGGGSEVQLVESGGGLVQPGGSLKLSCAAS |
| | | GFTFNKYAMNWVRQAPGKGLEWVARIRSKYNNYATYYADS |
| | | VKDRFTISRDDSKNTAYLQMNNLKTEDTAVYYCVRHGNFGN |
| | | SYISYWAYWGQGTLVTVSSGGGGSGGGGSGGGGSQTVVTQE |
| | | PSLTVSPGGTVTLTCGSSTGAVTSGNYPNWVQQKPGQAPRGL |
| | | IGGTKFLAPGTPARFSGSLLGGKAALTLSGVQPEDEAEYYCVL |
| | | WYSNRWVFGGGTKLTVLGGGGDKTHTCPPCPAPELLGGPSV |
| | | FLFPPKPKDTLMISRTPEVTCVVVDVSHEDPEVKFNWYVDGV |
| | | EVHNAKTKPCEEQYGSTYRCVSVLTVLHQDWLNGKEYKCKV |
| | | SNKALPAPIEKTISKAKGQPREPQVYTLPPSREEMTKNQVSLT |
| | | CLVKGFYPSDIAVEWESNGQPENNYKTTPPVLDSDGSFFLYSK |
| | | LTVDKSRWQQGNVFSCSVMHEALHNHYTQKSLSLSPGKGGG |
| | | GSGGGGSGGGGSGGGGSGGGGSGGGGSDKTHTCPPCPAPELL |
| | | GGPSVFLFPPKPKDTLMISRTPEVTCVVVDVSHEDPEVKFNWY |
| | | VDGVEVHNAKTKPCEEQYGSTYRCVSVLTVLHQDWLNGKEY |
| | | KCKVSNKALPAPIEKTISKAKGQPREPQVYTLPPSREEMTKNQ |
| | | VSLTCLVKGFYPSDIAVEWESNGQPENNYKTTPPVLDSDGSFF |
| | | LYSKLTVDKSRWQQGNVFSCSVMHEALHNHYTQKSLSLSPG |
| | | K |
| 587 | CD22 11-C3 | QVQLQESGPGLVKPSETLSLTCTVSGGSISSFYWSWIRQPPGK |
| | scFab × | GLEWIGRISSSGDVDYNPSLKSRVTISVDTSKNQFSLKLSSVTA |
| | CD20 29-F5 | ADTAVYYCAREGGFYYWGQGTLVTVSSASTKGPSVFPLAPSS |
| | scFab | KSTSGGTAALGCLVKDYFPEPVTVSWNSGALTSGVHTFPAVL |
| | | QSSGLYSLSSVVTVPSSSLGTQTYICNVNHKPSNTKVDKKVEP |
| | | KSCGGGGSGGGGSGGGGSGGGGSGGGGSGGGGSGGGGSGG |
| | | GGSEIVLTQSPGTLSLSPGERATLSCRASQSVSSSYLAWYQQK |
| | | PGQAPRLLIYGASSRATGIPDRFSGSGSGTDFTLTISRLEPEDFA |
| | | VYYCQQYGSSPITFGQGTKLEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNRG |
| | | ECSGGGGSQVQLQQWGAGLLKPSETLSLTCAVYGGSFSGYY |
| | | WSWIRQPPGKGLEWIGEINHRGSTNYNPSLKSRVTISGDTSKN |
| | | QFSLKLSSVNAADTAVYYCARGYSSSWYTGYVFDYWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTY |
| | | ICNVNHKPSNTKVDKKVEPKSCGGGGSGGGGSGGGGSGGGG |
| | | SGGGGSGGGGSGGGGSGGGGSQSALTQPASVSGSPGQSITISC |
| | | TGTSSDVFGYDYVSWYQQHPGKAPKLMIYDVSNRPSGVSNRF |
| | | SGSKSGNAASLTISGLQAEDEADYYCSSYKSSSTVVFGGGTKL |
| | | TVLGQPKAAPSVTLFPPSSEELQANKATLVCLISDFYPGAVTV |
| | | AWKADSSPVKAGVETTTPSKQSNNKYAASSYLSLTPEQWKSH |
| | | RSYSCQVTHEGSTVEKTVAPTECS |
| 588 | CD22 11-C3 | QVQLQESGPGLVKPSETLSLTCTVSGGSISSFYWSWIRQPPGK |
| | scFab × | GLEWIGRISSSGDVDYNPSLKSRVTISVDTSKNQFSLKLSSVTA |
| | CD20 29-F5 | ADTAVYYCAREGGFYYWGQGTLVTVSSASTKGPSVFPLAPSS |
| | scFab × I2C | KSTSGGTAALGCLVKDYFPEPVTVSWNSGALTSGVHTFPAVL |
| | | QSSGLYSLSSVVTVPSSSLGTQTYICNVNHKPSNTKVDKKVEP |
| | | KSCGGGGSGGGGSGGGGSGGGGSGGGGSGGGGSGGGGSGG |
| | | GGSEIVLTQSPGTLSLSPGERATLSCRASQSVSSSYLAWYQQK |
| | | PGQAPRLLIYGASSRATGIPDRFSGSGSGTDFTLTISRLEPEDFA |
| | | VYYCQQYGSSPITFGQGTKLEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNRG |
| | | ECSGGGGSQVQLQQWGAGLLKPSETLSLTCAVYGGSFSGYY |
| | | WSWIRQPPGKGLEWIGEINHRGSTNYNPSLKSRVTISGDTSKN |
| | | QFSLKLSSVNAADTAVYYCARGYSSSWYTGYVFDYWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTY |
| | | ICNVNHKPSNTKVDKKVEPKSCGGGGSGGGGSGGGGSGGGG |
| | | SGGGGSGGGGSGGGGSGGGGSQSALTQPASVSGSPGQSITISC |
| | | TGTSSDVFGYDYVSWYQQHPGKAPKLMIYDVSNRPSGVSNRF |
| | | SGSKSGNAASLTISGLQAEDEADYYCSSYKSSSTVVFGGGTKL |
| | | TVLGQPKAAPSVTLFPPSSEELQANKATLVCLISDFYPGAVTV |
| | | AWKADSSPVKAGVETTTPSKQSNNKYAASSYLSLTPEQWKSH |
| | | RSYSCQVTHEGSTVEKTVAPTECSSGGGGSEVQLVESGGGLV |
| | | QPGGSLKLSCAASGFTFNKYAMNWVRQAPGKGLEWVARIRS |
| | | KYNNYATYYADSVKDRFTISRDDSKNTAYLQMNNLKTEDTA |
| | | VYYCVRHGNFGNSYISYWAYWGQGTLVTVSSGGGGSGGGGS |
| | | GGGGSQTVVTQEPSLTVSPGGTVTLTCGSSTGAVTSGNYPNW |
| | | VQQKPGQAPRGLIGGTKFLAPGTPARFSGSLLGGKAALTLSGV |
| | | QPEDEAEYYCVLWYSNRWVFGGGTKLTVL |
| 589 | CD22 11-C3 | QVQLQESGPGLVKPSETLSLTCTVSGGSISSFYWSWIRQPPGK |
| | scFab × | GLEWIGRISSSGDVDYNPSLKSRVTISVDTSKNQFSLKLSSVTA |
| | CD20 29-F5 | ADTAVYYCAREGGFYYWGQGTLVTVSSASTKGPSVFPLAPSS |
| | scFab × I2C | KSTSGGTAALGCLVKDYFPEPVTVSWNSGALTSGVHTFPAVL |
| | × scFc | QSSGLYSLSSVVTVPSSSLGTQTYICNVNHKPSNTKVDKKVEP |
| | | KSCGGGGSGGGGSGGGGSGGGGSGGGGSGGGGSGGGGSGG |
| | | GGSEIVLTQSPGTLSLSPGERATLSCRASQSVSSSYLAWYQQK |
| | | PGQAPRLLIYGASSRATGIPDRFSGSGSGTDFTLTISRLEPEDFA |
| | | VYYCQQYGSSPITFGQGTKLEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNRG |
| | | ECSGGGGSQVQLQQWGAGLLKPSETLSLTCAVYGGSFSGYY |
| | | WSWIRQPPGKGLEWIGEINHRGSTNYNPSLKSRVTISGDTSKN |
| | | QFSLKLSSVNAADTAVYYCARGYSSSWYTGYVFDYWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTY |
| | | ICNVNHKPSNTKVDKKVEPKSCGGGGSGGGGSGGGGSGGGG |
| | | SGGGGSGGGGSGGGGSGGGGSQSALTQPASVSGSPGQSITISC |
| | | TGTSSDVFGYDYVSWYQQHPGKAPKLMIYDVSNRPSGVSNRF |
| | | SGSKSGNAASLTISGLQAEDEADYYCSSYKSSSTVVFGGGTKL |
| | | TVLGQPKAAPSVTLFPPSSEELQANKATLVCLISDFYPGAVTV |
| | | AWKADSSPVKAGVETTTPSKQSNNKYAASSYLSLTPEQWKSH |
| | | RSYSCQVTHEGSTVEKTVAPTECSSGGGGSEVQLVESGGGLV |
| | | QPGGSLKLSCAASGFTFNKYAMNWVRQAPGKGLEWVARIRS |
| | | KYNNYATYYADSVKDRFTISRDDSKNTAYLQMNNLKTEDTA |
| | | VYYCVRHGNFGNSYISYWAYWGQGTLVTVSSGGGGSGGGGS |
| | | GGGGSQTVVTQEPSLTVSPGGTVTLTCGSSTGAVTSGNYPNW |
| | | VQQKPGQAPRGLIGGTKFLAPGTPARFSGSLLGGKAALTLSGV |
| | | QPEDEAEYYCVLWYSNRWVFGGGTKLTVLGGGGDKTHTCPP |
| | | CPAPELLGGPSVFLFPPKPKDTLMISRTPEVTCVVVDVSHEDPE |
| | | VKFNWYVDGVEVHNAKTKPCEEQYGSTYRCVSVLTVLHQD |
| | | WLNGKEYKCKVSNKALPAPIEKTISKAKGQPREPQVYTLPPSR |
| | | EEMTKNQVSLTCLVKGFYPSDIAVEWESNGQPENNYKTTPPV |
| | | LDSDGSFFLYSKLTVDKSRWQQGNVFSCSVMHEALHNHYTQ |
| | | KSLSLSPGKGGGGSGGGGSGGGGSGGGGSGGGGSGGGGSDK |
| | | THTCPPCPAPELLGGPSVFLFPPKPKDTLMISRTPEVTCVVVDV |
| | | SHEDPEVKFNWYVDGVEVHNAKTKPCEEQYGSTYRCVSVLT |
| | | VLHQDWLNGKEYKCKVSNKALPAPIEKTISKAKGQPREPQVY |
| | | TLPPSREEMTKNQVSLTCLVKGFYPSDIAVEWESNGQPENNY |
| | | KTTPPVLDSDGSFFLYSKLTVDKSRWQQGNVFSCSVMHEALH |
| | | NHYTQKSLSLSPGK |

DNA encoding CCR8 TCE 1.1 HLE of SEQ ID NO: 227
(SEQ ID NO: 590)
GAGGTGCAGCTGGTGGAATCTGGCGGAGGACTTGTGAAGCCTGGCGGCTCTCTGAGACTGTCT
TGTGCCGCTTCTGGCTTCACCTTCAGCAACGCCAGAATGGGCTGGGTCCGACAGGCTCCTGGA
AAGTGTCTGGAATGGGTCGGACGGATCAAGTCCAAGACCGAAGGCGGCACCAGAGACTACGCC
GCTCCTGTGAAGGGCAGATTCACCATCTCTCGGGACGACTCCAAGAACACCCTGTACCTGCAG
ATGAACTCCCTGAAAACCGAGGACACCGCCGTGTACTACTGCACCTCTTACTCTGGCGTGTGG
GGCCAGGGCACAATGGTCACAGTTTCTAGCGGCGGAGGTGGACAAGGTGGTGGCGGACAAGGC
GGCGGTGGTCAAGAGATTGTGATGACCCAGTCTCCTGACAGCCTGGCCGTGTCTCTGGGAGAG
AGAGCCACCATCAACTGCAAGTCCAGCCAGTCCGTGCTGTACTCCTCCAACAACAAGAACTAC
CTGGCCTGGTATCACCAGAAGCCAGGCCAGTCTCCAAAGCTGCTGATCTCCTGGGCCTCCACC
AGAGAAAGCGGCGTGCCCGATAGATTCTCCGGCTCTGGCTCTGGCACCGACTTCACCCTGACC
ATCAATTCCCTGCAGGCCGAGGATGTGGCTGTGTACTATTGCCAGCAGTACTACAGCATCCCC
ATCACCTTCGGCTGCGGCACCAAGGTGGAAATCAAGTCCGGAGGTGGAGGGCAGGAAGTGCAG
CTGGTTGAATCTGGCGGCGGATTGGTTCAGCCTGGCGGATCTCTGAAGCTGTCTTGTGCCGCC
TCTGGCTTCACCTTCAACAAATACGCCATCAACTGGGTCCGACAGGCCCCTGGCAAAGGACTG
GAATGGGTCGCCCGGATCAGATCCAAGTACAACAACTACGCTACCTACTACGCCGACGCCGTG
AAGGACCGGTTCACCATCTCCAGAGATGACTCCAAGAACACCGTGTACCTGCAGATGAACAAC
CTCAAGACCGAGGACACCGCCGTGTACTACTGTGCCAGAGCCGGCAACTTCGGCTCCTCCTAC
ATCAGCTACTGGGCCTATTGGGGCCAGGGCACACTGGTCACAGTTAGTTCAGGTGGCGGTGGA
CAGGGCGGCGGAGGTCAGGGTGGCGGAGGCCAGCAAACAGTGGTCACCCAAGAGCCTAGCCTG
ACCGTTTCTCCTGGCGGCACCGTGACCATCACCTGTGGATCTTCTACCGGCGCTGTGACCTCC
GGCAACTACCCTAATTGGGTGCAGAAGAAGCCCGGCCAGGCTCCTAGAGGACTGATCGGAGGC
ACCAAGTTTCTGGCTCCCGGCACTCCTGCCAGATTCTCCGGTTCTCTGTCTGGCGGAAAGGCC
GCTCTGACATTGTCTGGCGTGCAGCCTGAGGATGAGGCTGAGTACTATTGCGTGCTGTGGTAC
TCCAACAGATGGGTGTTCGGCTCCGGCACCAAGCTGACAGTTCTCGGCGGAGGTGGATGCCCT
CCTTGTCCTGCTCCTGAATTGCTCGGCGGACCCTCCGTGTTCCTGTTTCCTCCAAAGCCTAAG
GACACCCTGATGATCTCTCGTACGCCTGAAGTGACCTGCGTGGTGGTGGATGTGTCCCACGAG
GAACCCGAAGTGAAGTTCAATTGGTACGTGGACGGCGTGGAAGTGCACAACGCCAAGACAAAG
CCCTGCGAGGAACAGTACGGCTCCACCTACAGATGCGTGTCCGTGCTGACAGTGCTGCACCAG
GATTGGCTGAACGGCAAAGAGTACAAGTGCAAGGTGTCCAACAAGGCCCTGCCTGCTCCTATC
GAAAAGACCATCTCCAAGGCCAAGGGCCAGCCTAGAGAGCCCCAGGTTTACACCCTGCCTCCA
AGCAGAGAAGAGATGACCAAGAACCAGGTGTCCCTGACCTGCCTGGTCAAGGGCTTCTACCCT
TCCGATATCGCCGTGGAATGGGAGAGCAATGGACAGCCCGAGAACAACTACAAGACCACACCT
CCTGTGCTGGACTCCGACGGCTCATTCTTCCTGTACTCCAAGCTGACCGTGGACAAGTCCAGA
TGGCAGCAGGGCAACGTGTTCTCCTGCTCCGTGATGCACGAGGCCCTGCACAATCACTACACC
CAGAAGTCCCTGTCTCTGTCCCCTGGAGGCGGAGGACAAGGCGGAGGTGGTCAAGGTGGTGGT
GGCCAAGGCGGAGGCGGACAAGGCGGCGGAGGACAAGGTGGCGGTGGACAGTGTCCTCCATGT
CCAGCACCTGAGCTTCTCGGAGGCCCTTCTGTGTTTCTGTTCCCACCTAAGCCAAAGGATACA
CTCATGATCAGCCGCACACCTGAAGTCACATGTGTCGTCGTGGATGTCTCTCATGAAGAACCA
GAAGTCAAGTTTAATTGGTATGTCGATGGCGTCGAGGTCCACAATGCTAAGACCAAGCCTTGT
GAAGAACAATATGGCAGCACCTATCGCTGTGTGTCTGTCCTGACCGTCCTGCATCAAGACTGG
CTCAATGGGAAAGAATACAAATGCAAAGTCTCTAACAAAGCTCTGCCCGCACCAATCGAGAAA
ACCATCAGCAAGGCTAAAGGACAGCCTCGCGAGCCTCAAGTGTATACCCTGCCACCTTCTCGC
GAGGAAATGACAAAAAATCAAGTCTCCCTCACCTGTCTCGTGAAGGGATTCTATCCCAGCGAC
ATTGCCGTCGAGTGGGAGTCTAATGGCCAGCCTGAAAACAATTATAAGACAACCCCACCTGTC
CTGGACAGCGACGGCTCATTTTTTCTCTACTCTAAACTCACCGTGGATAAGAGCCGGTGGCAA
CAGGGAAATGTGTTCAGCTGTAGCGTGATGCATGAAGCTCTCCACAACCATTATACACAGAAG
AGTCTGAGCCTGTCTCCTGGCAAATGA
DNA encoding CCR8 TCE 1.2 HLE of SEQ ID NO: 238
(SEQ ID NO: 591)
GAGGTGCAGCTGGTGGAATCTGGCGGAGGACTTGTGAAGCCTGGCGGCTCTCTGAGACTGTCT
TGTGCCGCTTCTGGCTTCACCTTCAGCAACGCCAGAATGGGCTGGGTCCGACAGGCTCCTGGA
AAGTGTCTGGAATGGGTCGGACGGATCAAGTCCAAGACCGAAGGCGGCACCAGAGACTACGCC
GCTCCTGTGAAGGGCAGATTCACCATCTCTCGGGACGACTCCAAGAACACCCTGTACCTGCAG
ATGAACTCCCTGAAAACCGAGGACACCGCCGTGTACTACTGCACCTCTTACTCTGGCGTGTGG
GGCCAGGGCACAATGGTCACAGTTTCTAGCGGCGGAGGTGGACAAGGTGGTGGCGGACAAGGC
GGCGGTGGTCAAGAGATTGTGATGACCCAGTCTCCTGACAGCCTGGCCGTGTCTCTGGGAGAG
AGAGCCACCATCAACTGCAAGTCCAGCCAGTCCGTGCTGTACTCCTCCAACAACAAGAACTAC
CTGGCCTGGTATCACCAGAAGCCAGGCCAGTCTCCAAAGCTGCTGATCTCCTGGGCCTCCACC
AGAGAAAGCGGCGTGCCCGATAGATTCTCCGGCTCTGGCTCTGGCACCGACTTCACCCTGACC
ATCAATTCCCTGCAGGCCGAGGATGTGGCTGTGTACTATTGCCAGCAGTACTACAGCATCCCC
ATCACCTTCGGCTGCGGCACCAAGGTGGAAATCAAGTCCGGAGGTGGAGGGCAGGAAGTGCAG
CTGGTTGAATCTGGCGGCGGATTGGTTCAGCCTGGCGGATCTCTGAAGCTGTCTTGTGCCGCC
TCTGGCTTCACCTTCAACAAATACGCCATCAACTGGGTCCGACAGGCCCCTGGCAAAGGACTG
GAATGGGTCGCCCGGATCAGATCCAAGTACAACAACTACGCTACCTACTACGCCGACGCCGTG
AAGGACCGGTTCACCATCTCCAGAGATGACTCCAAGAACACCGTGTACCTGCAGATGAACAAC
CTCAAGACCGAGGACACCGCCGTGTACTACTGTGCCAGAGCCGGCAACTTCGGCTCCTCCTAC
ATCAGCTACTGGGCCTATTGGGGCCAGGGCACACTGGTCACAGTTAGTTCAGGTGGCGGTGGA
CAGGGCGGCGGAGGTCAGGGTGGCGGAGGCCAGCAAACAGTGGTCACCCAAGAGCCTAGCCTG
ACCGTTTCTCCTGGCGGCACCGTGACCATCACCTGTGGATCTTCTACCGGCGCTGTGACCTCC
GGCAACTACCCTAATTGGGTGCAGAAGAAGCCCGGCCAGGCTCCTAGAGGACTGATCGGAGGC
ACCAAGTTTCTGGCTCCCGGCACTCCTGCCAGATTCTCCGGTTCTCTGTCTGGCGGAAAGGCC
GCTCTGACATTGTCTGGCGTGCAGCCTGAGGATGAGGCTGAGTACTATTGCGTGCTGTGGTAC
TCCAACAGATGGGTGTTCGGCTCCGGCACCAAGCTGACAGTTCTCGGCGGAGGTGGATGCCCT
CCTTGTCCTGCTCCTGAATTGCTCGGCGGACCCTCCGTGTTCCTGTTTCCTCCAAAGCCTAAG
GACACCCTGTACATCACCCGCGAGCCTGAAGTGACCTGCGTGGTGGTGGATGTGTCCCACGAG
GAACCCGAAGTGAAGTTCAATTGGTACGTGGACGGCGTGGAAGTGCACAACGCCAAGACAAAG
CCCTGCGAGGAACAGTACGGCTCCACCTACAGATGCGTGTCCGTGCTGACAGTGCTGCACCAG
GATTGGCTGAACGGCAAAGAGTACAAGTGCAAGGTGTCCAACAAGGCCCTGCCTGCTCCTATC
GAAAAGACCATCTCCAAGGCCAAGGGCCAGCCTAGAGAGCCCCAGGTTTACACCCTGCCTCCA
AGCAGAGAAGAGATGACCAAGAACCAGGTGTCCCTGACCTGCCTGGTCAAGGGCTTCTACCCT
TCCGATATCGCCGTGGAATGGGAGAGCAATGGACAGCCCGAGAACAACTACAAGACCACACCT
CCTGTGCTGGACTCCGACGGCTCATTCTTCCTGTACTCCAAGCTGACCGTGGACAAGTCCAGA
TGGCAGCAGGGCAACGTGTTCTCCTGCTCCGTGATGCACGAGGCCCTGCACAATCACTACACC
CAGAAGTCCCTGTCTCTGTCCCCTGGAGGCGGAGGACAAGGCGGAGGTGGTCAAGGTGGTGGT
GGCCAAGGCGGAGGCGGACAAGGCGGCGGAGGACAAGGTGGCGGTGGACAGTGTCCTCCATGT
CCAGCACCTGAGCTTCTCGGAGGCCCTTCTGTGTTTCTGTTCCCACCTAAGCCAAAGGATACA
CTCTACATCACCCGCGAGCCTGAAGTCACATGTGTCGTCGTGGATGTCTCTCATGAAGAACCA
GAAGTCAAGTTTAATTGGTATGTCGATGGCGTCGAGGTCCACAATGCTAAGACCAAGCCTTGT
GAAGAACAATATGGCAGCACCTATCGCTGTGTGTCTGTCCTGACCGTCCTGCATCAAGACTGG
CTCAATGGGAAAGAATACAAATGCAAAGTCTCTAACAAAGCTCTGCCCGCACCAATCGAGAAA
ACCATCAGCAAGGCTAAAGGACAGCCTCGCGAGCCTCAAGTGTATACCCTGCCACCTTCTCGC
GAGGAAATGACAAAAAATCAAGTCTCCCTCACCTGTCTCGTGAAGGGATTCTATCCCAGCGAC
ATTGCCGTCGAGTGGGAGTCTAATGGCCAGCCTGAAAACAATTATAAGACAACCCCACCTGTC
CTGGACAGCGACGGCTCATTTTTTCTCTACTCTAAACTCACCGTGGATAAGAGCCGGTGGCAA
CAGGGAAATGTGTTCAGCTGTAGCGTGATGCATGAAGCTCTCCACAACCATTATACACAGAAG
AGTCTGAGCCTGTCTCCTGGCAAATGA
DNA encoding CCR8 TCE 1.3 HLE of SEQ ID NO: 249
(SEQ ID NO: 592)
GAGGTGCAGCTGGTGGAATCTGGCGGAGGACTTGTGAAGCCTGGCGGCTCTCTGAGACTGTCT
TGTGCCGCTTCTGGCTTCACCTTCAGCAACGCCAGAATGGGCTGGGTCCGACAGGCTCCTGGA
AAAGGACTGGAATGGGTCGGACGGATCAAGTCCAAGACCGAAGGCGGCACCAGAGACTACGCC
GCTCCTGTGAAGGGCAGATTCACCATCTCTCGGGACGACTCCAAGAACACCCTGTACCTGCAG
ATGAACTCCCTGAAAACCGAGGACACCGCCGTGTACTACTGCACCTCTTACTCTGGCGTGTGG
GGCCAGGGCACAATGGTCACAGTTTCTTCCGCCTCCACCAAGGGACCCAGCGTTTTCCCTCTG
GCTCCATCCTCCAAGTCTACCTCTGGCGGAACAGCTGCTCTGGGCTGCCTGGTCAAGGACTAC
TTTCCTGAGCCTGTGACCGTGTCCTGGAACTCTGGCGCTCTGACATCTGGCGTGCACACCTTT
CCAGCTGTGCTGCAGTCCTCCGGCCTGTACTCTCTGTCCTCTGTCGTGACCGTGCCTTCCAGC
TCTCTGGGAACCCAGACCTACATCTGCAATGTGAACCACAAGCCTTCCAACACCAAGGTGGAC
AAGAAGGTGGAACCCAAGTCTTGTGGCGGAGGCGGACAAGGTGGTGGTGGTCAAGGTGGCGGA
GGACAAGGCGGTGGCGGCCAAGGCGGAGGTGGACAAGGCGGCGGAGGCCAAGGTGGCGGCGGT
CAAGGCGGCGGTGGTCAAGAAATTGTGATGACCCAGTCTCCTGACAGCCTGGCCGTGTCTCTG
GGAGAGAGAGCCACCATCAACTGCAAGTCCAGCCAGTCCGTGCTGTACTCCTCCAACAACAAG
AACTACCTGGCCTGGTATCACCAGAAGCCAGGCCAGTCTCCAAAGCTGCTGATCTCCTGGGCC
TCTACCAGAGAATCCGGCGTGCCCGACAGATTTTCCGGCTCTGGCTCTGGCACCGACTTCACC
CTGACCATCAATTCCCTGCAGGCCGAGGATGTGGCTGTGTACTATTGCCAGCAGTACTACAGC
ATCCCCATCACCTTCGGAGGTGGCACCAAGGTCGAGATCAAGAGAACCGTGGCCGCTCCTTCC
GTGTTCATCTTCCCACCTTCCGACGAGCAGCTGAAGTCCGGCACAGCTTCTGTCGTGTGCCTG
CTGAACAACTTCTACCCTCGGGAAGCCAAGGTGCAGTGGAAAGTGGATAACGCCCTGCAGTCC
GGCAACTCCCAAGAGTCTGTGACCGAGCAGGACTCCAAGGACAGCACCTACAGCCTGTCCTCC
ACACTGACCCTGTCCAAGGCCGACTACGAGAAGCACAAGGTGTACGCCTGCGAAGTGACCCAT
CAGGGCCTGTCTAGCCCTGTGACCAAGTCTTTCAACCGGGGCGAGTGTTCCGGAGGTGGAGGG
CAGGAAGTGCAGCTGGTTGAATCTGGCGGCGGATTGGTTCAGCCTGGCGGATCTCTGAAGCTG
TCTTGTGCCGCCTCTGGCTTCACCTTCAACAAATACGCCATCAACTGGGTCCGACAGGCCCCT
GGCAAAGGACTGGAATGGGTCGCCCGGATCAGATCCAAGTACAACAACTACGCTACCTACTAC
GCCGACGCCGTGAAGGACCGGTTCACCATCTCCAGAGATGACTCCAAGAACACCGTGTACCTG
CAGATGAACAACCTCAAGACCGAGGACACCGCCGTGTACTACTGTGCCAGAGCCGGCAACTTC
GGCTCCTCCTACATCAGCTACTGGGCCTATTGGGGCCAGGGCACACTGGTCACAGTTAGTTCA
GGTGGCGGTGGACAGGGCGGCGGAGGTCAGGGTGGCGGAGGCCAGCAAACAGTGGTCACCCAA
GAGCCTAGCCTGACCGTTTCTCCTGGCGGCACCGTGACCATCACCTGTGGATCTTCTACCGGC
GCTGTGACCTCCGGCAACTACCCTAATTGGGTGCAGAAGAAGCCCGGCCAGGCTCCTAGAGGA
CTGATCGGAGGCACCAAGTTTCTGGCTCCCGGCACTCCTGCCAGATTCTCCGGTTCTCTGTCT
GGCGGAAAGGCCGCTCTGACATTGTCTGGCGTGCAGCCTGAGGATGAGGCTGAGTACTATTGC
GTGCTGTGGTACTCCAACAGATGGGTGTTCGGCTCCGGCACCAAGCTGACAGTTCTCGGCGGA
GGTGGATGCCCTCCTTGTCCTGCTCCTGAATTGCTCGGCGGACCCTCCGTGTTCCTGTTTCCT
CCAAAGCCTAAGGACACCCTGATGATCTCTCGTACGCCTGAAGTGACCTGCGTGGTGGTGGAT
GTGTCCCACGAGGAACCCGAAGTGAAGTTCAATTGGTACGTGGACGGCGTGGAAGTGCACAAC
GCCAAGACAAAGCCCTGCGAGGAACAGTACGGCTCCACCTACAGATGCGTGTCCGTGCTGACA
GTGCTGCACCAGGATTGGCTGAACGGCAAAGAGTACAAGTGCAAGGTGTCCAACAAGGCCCTG
CCTGCTCCTATCGAAAAGACCATCTCCAAGGCCAAGGGCCAGCCTAGAGAGCCCCAGGTTTAC
ACCCTGCCTCCAAGCAGAGAAGAGATGACCAAGAACCAGGTGTCCCTGACCTGCCTGGTCAAG
GGCTTCTACCCTTCCGATATCGCCGTGGAATGGGAGAGCAATGGACAGCCCGAGAACAACTAC
AAGACCACACCTCCTGTGCTGGACTCCGACGGCTCATTCTTCCTGTACTCCAAGCTGACCGTG
GACAAGTCCAGATGGCAGCAGGGCAACGTGTTCTCCTGCTCCGTGATGCACGAGGCCCTGCAC
AATCACTACACCCAGAAGTCCCTGTCTCTGTCCCCTGGAGGCGGAGGACAAGGCGGAGGTGGT
CAAGGTGGTGGTGGCCAAGGCGGAGGCGGACAAGGCGGCGGAGGACAAGGTGGCGGTGGACAG
TGTCCTCCATGTCCAGCACCTGAGCTTCTCGGAGGCCCTTCTGTGTTTCTGTTCCCACCTAAG
CCAAAGGATACACTCATGATCAGCCGCACACCTGAAGTCACATGTGTCGTCGTGGATGTCTCT
CATGAAGAACCAGAAGTCAAGTTTAATTGGTATGTCGATGGCGTCGAGGTCCACAATGCTAAG
ACCAAGCCTTGTGAAGAACAATATGGCAGCACCTATCGCTGTGTGTCTGTCCTGACCGTCCTG
CATCAAGACTGGCTCAATGGGAAAGAATACAAATGCAAAGTCTCTAACAAAGCTCTGCCCGCA
CCAATCGAGAAAACCATCAGCAAGGCTAAAGGACAGCCTCGCGAGCCTCAAGTGTATACCCTG
CCACCTTCTCGCGAGGAAATGACAAAAAATCAAGTCTCCCTCACCTGTCTCGTGAAGGGATTC
TATCCCAGCGACATTGCCGTCGAGTGGGAGTCTAATGGCCAGCCTGAAAACAATTATAAGACA
ACCCCACCTGTCCTGGACAGCGACGGCTCATTTTTTCTCTACTCTAAACTCACCGTGGATAAG
AGCCGGTGGCAACAGGGAAATGTGTTCAGCTGTAGCGTGATGCATGAAGCTCTCCACAACCAT
TATACACAGAAGAGTCTGAGCCTGTCTCCTGGCAAATGA
DNA encoding the CCR8 TCE 1.4 HLE of SEQ ID NO: 260
(SEQ ID NO: 593)
GAGGTGCAGCTGGTGGAATCTGGCGGAGGACTTGTGAAGCCTGGCGGCTCTCTGAGACTGTCT
TGTGCCGCTTCTGGCTTCACCTTCAGCAACGCCAGAATGGGCTGGGTCCGACAGGCTCCTGGA
AAAGGACTGGAATGGGTCGGACGGATCAAGTCCAAGACCGAAGGCGGCACCAGAGACTACGCC
GCTCCTGTGAAGGGCAGATTCACCATCTCTCGGGACGACTCCAAGAACACCCTGTACCTGCAG
ATGAACTCCCTGAAAACCGAGGACACCGCCGTGTACTACTGCACCTCTTACTCTGGCGTGTGG
GGCCAGGGCACAATGGTCACAGTTTCTTCCGCCTCCACCAAGGGACCCAGCGTTTTCCCTCTG
GCTCCATCCTCCAAGTCTACCTCTGGCGGAACAGCTGCTCTGGGCTGCCTGGTCAAGGACTAC
TTTCCTGAGCCTGTGACCGTGTCCTGGAACTCTGGCGCTCTGACATCTGGCGTGCACACCTTT
CCAGCTGTGCTGCAGTCCTCCGGCCTGTACTCTCTGTCCTCTGTCGTGACCGTGCCTTCCAGC
TCTCTGGGAACCCAGACCTACATCTGCAATGTGAACCACAAGCCTTCCAACACCAAGGTGGAC
AAGAAGGTGGAACCCAAGTCTTGTGGCGGAGGCGGACAAGGTGGTGGTGGTCAAGGTGGCGGA
GGACAAGGCGGTGGCGGCCAAGGCGGAGGTGGACAAGGCGGCGGAGGCCAAGGTGGCGGCGGT
CAAGGCGGCGGTGGTCAAGAAATTGTGATGACCCAGTCTCCTGACAGCCTGGCCGTGTCTCTG
GGAGAGAGAGCCACCATCAACTGCAAGTCCAGCCAGTCCGTGCTGTACTCCTCCAACAACAAG
AACTACCTGGCCTGGTATCACCAGAAGCCAGGCCAGTCTCCAAAGCTGCTGATCTCCTGGGCC
TCTACCAGAGAATCCGGCGTGCCCGACAGATTTTCCGGCTCTGGCTCTGGCACCGACTTCACC
CTGACCATCAATTCCCTGCAGGCCGAGGATGTGGCTGTGTACTATTGCCAGCAGTACTACAGC
ATCCCCATCACCTTCGGAGGTGGCACCAAGGTCGAGATCAAGAGAACCGTGGCCGCTCCTTCC
GTGTTCATCTTCCCACCTTCCGACGAGCAGCTGAAGTCCGGCACAGCTTCTGTCGTGTGCCTG
CTGAACAACTTCTACCCTCGGGAAGCCAAGGTGCAGTGGAAAGTGGATAACGCCCTGCAGTCC
GGCAACTCCCAAGAGTCTGTGACCGAGCAGGACTCCAAGGACAGCACCTACAGCCTGTCCTCC
ACACTGACCCTGTCCAAGGCCGACTACGAGAAGCACAAGGTGTACGCCTGCGAAGTGACCCAT
CAGGGCCTGTCTAGCCCTGTGACCAAGTCTTTCAACCGGGGCGAGTGTTCCGGAGGTGGAGGG
CAGGAAGTGCAGCTGGTTGAATCTGGCGGCGGATTGGTTCAGCCTGGCGGATCTCTGAAGCTG
TCTTGTGCCGCCTCTGGCTTCACCTTCAACAAATACGCCATCAACTGGGTCCGACAGGCCCCT
GGCAAAGGACTGGAATGGGTCGCCCGGATCAGATCCAAGTACAACAACTACGCTACCTACTAC
GCCGACGCCGTGAAGGACCGGTTCACCATCTCCAGAGATGACTCCAAGAACACCGTGTACCTG
CAGATGAACAACCTCAAGACCGAGGACACCGCCGTGTACTACTGTGCCAGAGCCGGCAACTTC
GGCTCCTCCTACATCAGCTACTGGGCCTATTGGGGCCAGGGCACACTGGTCACAGTTAGTTCA
GGTGGCGGTGGACAGGGCGGCGGAGGTCAGGGTGGCGGAGGCCAGCAAACAGTGGTCACCCAA
GAGCCTAGCCTGACCGTTTCTCCTGGCGGCACCGTGACCATCACCTGTGGATCTTCTACCGGC
GCTGTGACCTCCGGCAACTACCCTAATTGGGTGCAGAAGAAGCCCGGCCAGGCTCCTAGAGGA
CTGATCGGAGGCACCAAGTTTCTGGCTCCCGGCACTCCTGCCAGATTCTCCGGTTCTCTGTCT
GGCGGAAAGGCCGCTCTGACATTGTCTGGCGTGCAGCCTGAGGATGAGGCTGAGTACTATTGC
GTGCTGTGGTACTCCAACAGATGGGTGTTCGGCTCCGGCACCAAGCTGACAGTTCTCGGCGGA
GGTGGATGCCCTCCTTGTCCTGCTCCTGAATTGCTCGGCGGACCCTCCGTGTTCCTGTTTCCT
CCAAAGCCTAAGGACACCCTGTACATCACCCGCGAGCCTGAAGTGACCTGCGTGGTGGTGGAT
GTGTCCCACGAGGAACCCGAAGTGAAGTTCAATTGGTACGTGGACGGCGTGGAAGTGCACAAC
GCCAAGACAAAGCCCTGCGAGGAACAGTACGGCTCCACCTACAGATGCGTGTCCGTGCTGACA
GTGCTGCACCAGGATTGGCTGAACGGCAAAGAGTACAAGTGCAAGGTGTCCAACAAGGCCCTG
CCTGCTCCTATCGAAAAGACCATCTCCAAGGCCAAGGGCCAGCCTAGAGAGCCCCAGGTTTAC
ACCCTGCCTCCAAGCAGAGAAGAGATGACCAAGAACCAGGTGTCCCTGACCTGCCTGGTCAAG
GGCTTCTACCCTTCCGATATCGCCGTGGAATGGGAGAGCAATGGACAGCCCGAGAACAACTAC
AAGACCACACCTCCTGTGCTGGACTCCGACGGCTCATTCTTCCTGTACTCCAAGCTGACCGTG
GACAAGTCCAGATGGCAGCAGGGCAACGTGTTCTCCTGCTCCGTGATGCACGAGGCCCTGCAC
AATCACTACACCCAGAAGTCCCTGTCTCTGTCCCCTGGAGGCGGAGGACAAGGCGGAGGTGGT
CAAGGTGGTGGTGGCCAAGGCGGAGGCGGACAAGGCGGCGGAGGACAAGGTGGCGGTGGACAG
TGTCCTCCATGTCCAGCACCTGAGCTTCTCGGAGGCCCTTCTGTGTTTCTGTTCCCACCTAAG
CCAAAGGATACACTCTACATCACCCGCGAGCCTGAAGTCACATGTGTCGTCGTGGATGTCTCT
CATGAAGAACCAGAAGTCAAGTTTAATTGGTATGTCGATGGCGTCGAGGTCCACAATGCTAAG
ACCAAGCCTTGTGAAGAACAATATGGCAGCACCTATCGCTGTGTGTCTGTCCTGACCGTCCTG
CATCAAGACTGGCTCAATGGGAAAGAATACAAATGCAAAGTCTCTAACAAAGCTCTGCCCGCA
CCAATCGAGAAAACCATCAGCAAGGCTAAAGGACAGCCTCGCGAGCCTCAAGTGTATACCCTG
CCACCTTCTCGCGAGGAAATGACAAAAAATCAAGTCTCCCTCACCTGTCTCGTGAAGGGATTC
TATCCCAGCGACATTGCCGTCGAGTGGGAGTCTAATGGCCAGCCTGAAAACAATTATAAGACA
ACCCCACCTGTCCTGGACAGCGACGGCTCATTTTTTCTCTACTCTAAACTCACCGTGGATAAG
AGCCGGTGGCAACAGGGAAATGTGTTCAGCTGTAGCGTGATGCATGAAGCTCTCCACAACCAT
TATACACAGAAGAGTCTGAGCCTGTCTCCTGGCAAATGA
Leader polynucleotide sequence
(SEQ ID NO: 594)
ATGGACATGAGAGTGCCTGCACAGCTGCTGGGCCTGCTGCTGCTGTGGCTGAGAGGCGCCAGA
TG

TABLE 26
Anti-CCR8 molecule sequences.

| SEQ | | |
|---|---|---|
| ID NO: | Designation | Sequence |
| 595 | MPK20298-A4_SCFV huCCR8 HV hv_cdr1 | NNGMH |
| 596 | MPK20298-A4_SCFV huCCR8 HV hv_cdr2 | VISNDGSNKYYADSVKG |
| 597 | MPK20298-A4_SCFV huCCR8 HV hv_cdr3 | VYYGSGIYYKNRNYYGMDV |
| 598 | MPK20298-A4_SCFV huCCR8 LV lv_cdr1 | GGNNIGSQNVH |
| 599 | MPK20298-A4_SCFV huCCR8 LV lv_cdr2 | RDSNRPS |
| 600 | MPK20298-A4_SCFV huCCR8 LV lv_cdr3 | QVWDSSTVV |
| 601 | MPK20299-D2_SCFV huCCR8 HV hv_cdr1 | NYGMH |
| 602 | MPK20299-D2_SCFV huCCR8 HV hv_cdr2 | VISYDGSNKYYADSVKG |
| 603 | MPK20299-D2_SCFV huCCR8 HV hv_cdr3 | VYYGSGIYYKKRYYYGMDV |
| 604 | MPK20299-D2_SCFV huCCR8 LV lv_cdr1 | GGHNIGSKGVH |
| 605 | MPK20299-D2_SCFV huCCR8 LV lv_cdr2 | RNSNRPS |
| 606 | MPK20299-D2_SCFV huCCR8 LV lv_cdr3 | QVWDSSTVV |
| 607 | MPK20299-F11_SCFV huCCR8 HV hv_cdr1 | NYGMH |
| 608 | MPK20299-F11_SCFV huCCR8 HV hv_cdr2 | VISYDGSNKYYADSVKG |
| 609 | MPK20299-F11_SCFV huCCR8 HV hv_cdr3 | VYYGSGSYYKKRYYYGMDV |
| 610 | MPK20299-F11_SCFV huCCR8 LV lv_cdr1 | GGNNIGSQNVH |
| 611 | MPK20299-F11_SCFV huCCR8 LV lv_cdr2 | RDSNRPS |
| 612 | MPK20299-F11_SCFV huCCR8 LV lv_cdr3 | QVWDSSTVV |
| 613 | MPK20298-H6_SCFV huCCR8 HV hv_cdr1 | SSGMH |
| 614 | MPK20298-H6_SCFV huCCR8 HV hv_cdr2 | VISYDGTNKYYADSVKG |
| 615 | MPK20298-H6_SCFV huCCR8 HV hv_cdr3 | VYYGSGIYYKNRYYYGMDV |
| 616 | MPK20298-H6_SCFV huCCR8 LV lv_cdr1 | GGHNIGSKGVH |
| 617 | MPK20298-H6_SCFV huCCR8 LV lv_cdr2 | RNSNRPS |
| 618 | MPK20298-H6_SCFV huCCR8 LV lv_cdr3 | QVWDSSTVV |
| 619 | MPK20297-A4_SCFV huCCR8 HV hv_cdr1 | NYGMH |
| 620 | MPK20297-A4_SCFV huCCR8 HV hv_cdr2 | VISNDGSNKYYADSVKG |
| 621 | MPK20297-A4_SCFV huCCR8 HV hv_cdr3 | VYYGSGIYYKKRYYYGMDV |
| 622 | MPK20297-A4_SCFV huCCR8 LV lv_cdr1 | GGHNIGSQNVH |
| 623 | MPK20297-A4_SCFV huCCR8 LV lv_cdr2 | RDSNRPS |
| 624 | MPK20297-A4_SCFV huCCR8 LV lv_cdr3 | QVWDSSTVV |
| 625 | MPK20299-H8_SCFV huCCR8 HV hv_cdr1 | NYGMH |
| 626 | MPK20299-H8_SCFV huCCR8 HV hv_cdr2 | VISYDGSNKYYADSVKG |
| 627 | MPK20299-H8_SCFV huCCR8 HV hv_cdr3 | VYYGSGIYYKKRYYYGMDV |
| 628 | MPK20299-H8_SCFV huCCR8 LV lv_cdr1 | GGNNIGSKNVH |
| 629 | MPK20299-H8_SCFV huCCR8 LV lv_cdr2 | RNSNRPS |
| 630 | MPK20299-H8_SCFV huCCR8 LV lv_cdr3 | QVWDSSTVV |
| 631 | MPK20300-C11_SCFV huCCR8 HV | SYGMH |
| | hv_cdr1 | |
| 632 | MPK20300-C11_SCFV huCCR8 HV | VISYDGSNKYYADSVKG |
| | hv_cdr2 | |
| 633 | MPK20300-C11_SCFV huCCR8 HV | VYYGSGSYYKNRYYYGMDV |
| | hv_cdr3 | |
| 634 | MPK20300-C11_SCFV huCCR8 LV lv_cdr1 | GGNNIGSKNVH |
| 635 | MPK20300-C11_SCFV huCCR8 LV lv_cdr2 | RDINRPS |
| 636 | MPK20300-C11_SCFV huCCR8 LV lv_cdr3 | QVWDSSVV |
| 637 | MPK20298-B1_SCFV huCCR8 HV hv_cdr1 | NYGMH |
| 638 | MPK20298-B1_SCFV huCCR8 HV hv_cdr2 | VISYDGSNKYYADSVKG |
| 639 | MPK20298-B1_SCFV huCCR8 HV hv_cdr3 | VYYGSGIYYKKRYYYGMDV |
| 640 | MPK20298-B1_SCFV huCCR8 LV lv_cdr1 | EGNNIGSKNVH |
| 641 | MPK20298-B1_SCFV huCCR8 LV lv_cdr2 | RNSNRPS |
| 642 | MPK20298-B1_SCFV huCCR8 LV lv_cdr3 | QAWDSSTVV |
| 643 | MPK20297-E5_SCFV huCCR8 HV hv_cdr1 | NNGMH |
| 644 | MPK20297-E5_SCFV huCCR8 HV hv_cdr2 | VISYDGSNKYYTDSVKG |
| 645 | MPK20297-E5_SCFV huCCR8 HV hv_cdr3 | VYYGSGIYYKKRYYYGMDV |
| 646 | MPK20297-E5_SCFV huCCR8 LV lv_cdr1 | GGNNIGSKNVH |
| 647 | MPK20297-E5_SCFV huCCR8 LV lv_cdr2 | RDSNRPS |
| 648 | MPK20297-E5_SCFV huCCR8 LV lv_cdr3 | QVWDSSSDHVV |
| 649 | MPK20299-A3_SCFV huCCR8 HV hv_cdr1 | NYGMH |
| 650 | MPK20299-A3_SCFV huCCR8 HV hv_cdr2 | VISYDGSNKYYADSVKG |
| 651 | MPK20299-A3_SCFV huCCR8 HV hv_cdr3 | VYYGSGIYYKKRYYYGMDV |
| 652 | MPK20299-A3_SCFV huCCR8 LV lv_cdr1 | GGNNIGSKNVH |
| 653 | MPK20299-A3_SCFV huCCR8 LV lv_cdr2 | RNSNRPS |
| 654 | MPK20299-A3_SCFV huCCR8 LV lv_cdr3 | QAWDSSNVV |
| 655 | MPK20297-B4_SCFV huCCR8 HV hv_cdr1 | RNGMH |
| 656 | MPK20297-B4_SCFV huCCR8 HV hv_cdr2 | VISNDGSNKYYADSVKG |
| 657 | MPK20297-B4_SCFV huCCR8 HV hv_cdr3 | VYYGSGIYYKNNYYYGMDV |
| 658 | MPK20297-B4_SCFV huCCR8 LV lv_cdr1 | GGNNIGSQNVH |
| 659 | MPK20297-B4_SCFV huCCR8 LV lv_cdr2 | RDSNRPS |
| 660 | MPK20297-B4_SCFV huCCR8 LV lv_cdr3 | QVWDSSTVV |
| 661 | MPK20298-F6_SCFV huCCR8 HV hv_cdr1 | RNGMH |
| 662 | MPK20298-F6_SCFV huCCR8 HV hv_cdr2 | VISNDGSNKYYADSVKG |
| 663 | MPK20298-F6_SCFV huCCR8 HV hv_cdr3 | VYYGSGIYYKNRYYYGMDV |
| 664 | MPK20298-F6_SCFV huCCR8 LV lv_cdr1 | GGNNIGSKNVH |
| 665 | MPK20298-F6_SCFV huCCR8 LV lv_cdr2 | RDSNRPS |
| 666 | MPK20298-F6_SCFV huCCR8 LV lv_cdr3 | QVWDSSTVV |
| 667 | MPK20299-H3_SCFV huCCR8 HV hv_cdr1 | NYGMH |
| 668 | MPK20299-H3_SCFV huCCR8 HV hv_cdr2 | VISYDGSNKYYADSVKG |
| 669 | MPK20299-H3_SCFV huCCR8 HV hv_cdr3 | VYYGSGIYYKKRYYYGMDV |
| 670 | MPK20299-H3_SCFV huCCR8 LV lv_cdr1 | GGNNIGSKNVH |
| 671 | MPK20299-H3_SCFV huCCR8 LV lv_cdr2 | RNSNRPS |
| 672 | MPK20299-H3_SCFV huCCR8 LV lv_cdr3 | QIWDSSTVV |
| 673 | MPK20298-B9_SCFV huCCR8 HV hv_cdr1 | RNGMH |
| 674 | MPK20298-B9_SCFV huCCR8 HV hv_cdr2 | VISNDGSNKYYADSVKG |
| 675 | MPK20298-B9_SCFV huCCR8 HV hv_cdr3 | VYYGSGIYYKKNYYYGMDV |
| 676 | MPK20298-B9_SCFV huCCR8 LV lv_cdr1 | GGNNIGSKNVH |
| 677 | MPK20298-B9_SCFV huCCR8 LV lv_cdr2 | RDSNRPS |
| 678 | MPK20298-B9_SCFV huCCR8 LV lv_cdr3 | QVWDSSTVV |
| 679 | MPK20299-E2_SCFV huCCR8 HV hv_cdr1 | NNGMH |
| 680 | MPK20299-E2_SCFV huCCR8 HV hv_cdr2 | VISYDGSNKYYTDSVKG |
| 681 | MPK20299-E2_SCFV huCCR8 HV hv_cdr3 | VYYGSGIYYKKRYYYGMDV |
| 682 | MPK20299-E2_SCFV huCCR8 LV lv_cdr1 | EGNNIGSQNVH |
| 683 | MPK20299-E2_SCFV huCCR8 LV lv_cdr2 | RDSNRPS |
| 684 | MPK20299-E2_SCFV huCCR8 LV lv_cdr3 | QVWDGSAVV |
| 685 | MPK20299-D6_SCFV huCCR8 HV hv_cdr1 | SYGMH |
| 686 | MPK20299-D6_SCFV huCCR8 HV hv_cdr2 | VISYDGSNKYYADSVKG |
| 687 | MPK20299-D6_SCFV huCCR8 HV hv_cdr3 | VYYGSGIYYKKRYYYGMDV |
| 688 | MPK20299-D6_SCFV huCCR8 LV lv_cdr1 | EGNNIGSQNVH |
| 689 | MPK20299-D6_SCFV huCCR8 LV lv_cdr2 | RDSNRPS |
| 690 | MPK20299-D6_SCFV huCCR8 LV lv_cdr3 | QVWDGSAVV |
| 691 | MPK20299-A4_SCFV huCCR8 HV hv_cdr1 | NYGFH |
| 692 | MPK20299-A4_SCFV huCCR8 HV hv_cdr2 | VISYDGSNRYYADSVKG |
| 693 | MPK20299-A4_SCFV huCCR8 HV hv_cdr3 | VYYGSGTYYKNRYYYGMDV |
| 694 | MPK20299-A4_SCFV huCCR8 LV lv_cdr1 | GGHNIGSKGVH |
| 695 | MPK20299-A4_SCFV huCCR8 LV lv_cdr2 | RNSNRPS |
| 696 | MPK20299-A4_SCFV huCCR8 LV lv_cdr3 | QAWDSGTVV |
| 697 | MPK20300-G5_SCFV huCCR8 HV hv_cdr1 | NYGFH |
| 698 | MPK20300-G5_SCFV huCCR8 HV hv_cdr2 | VISYDGSNRYYADSVKG |
| 699 | MPK20300-G5_SCFV huCCR8 HV hv_cdr3 | VYYGSGTYYKNRYYYGMDV |
| 700 | MPK20300-G5_SCFV huCCR8 LV lv_cdrl | GANNIGSKNVH |
| 701 | MPK20300-G5_SCFV huCCR8 LV lv_cdr2 | RDFNRPS |
| 702 | MPK20300-G5_SCFV huCCR8 LV lv_cdr3 | QVWDSSTGNVV |
| 703 | MPK20299-C3_SCFV huCCR8 HV hv_cdr1 | NYGFH |
| 704 | MPK20299-C3_SCFV huCCR8 HV hv_cdr2 | VISYDGSNKYYADSVKG |
| 705 | MPK20299-C3_SCFV huCCR8 HV hv_cdr3 | VYYGSGSYYKNRYYYGMDV |
| 706 | MPK20299-C3_SCFV huCCR8 LV lv_cdr1 | GGNNIGSKNVH |
| 707 | MPK20299-C3_SCFV huCCR8 LV lv_cdr2 | RDSNRPS |
| 708 | MPK20299-C3_SCFV huCCR8 LV lv_cdr3 | QVWDSSTVV |
| 709 | MPK20299-B7_SCFV huCCR8 HV hv_cdr1 | NYGMH |
| 710 | MPK20299-B7_SCFV huCCR8 HV hv_cdr2 | VISYDGSNKYYADSVKG |
| 711 | MPK20299-B7_SCFV huCCR8 HV hv_cdr3 | VYYGSGIYYKKRYYYGMDV |
| 712 | MPK20299-B7_SCFV huCCR8 LV lv_cdr1 | GGNNIGSKNVH |
| 713 | MPK20299-B7_SCFV huCCR8 LV lv_cdr2 | RDSNRPS |
| 714 | MPK20299-B7_SCFV huCCR8 LV lv_cdr3 | QVWDSSSAHVI |
| 715 | MPK20299-A5_SCFV huCCR8 HV hv_cdr1 | GYGMH |
| 716 | MPK20299-A5_SCFV huCCR8 HV hv_cdr2 | VISYDGSNKYYADSVKG |
| 717 | MPK20299-A5_SCFV huCCR8 HV hv_cdr3 | VYYGSGIYYKNRYYYGMDV |
| 718 | MPK20299-A5_SCFV huCCR8 LV lv_cdr1 | GGNNLGSKNVH |
| 719 | MPK20299-A5_SCFV huCCR8 LV lv_cdr2 | RNSNRPS |
| 720 | MPK20299-A5_SCFV huCCR8 LV lv_cdr3 | QVWDSSTVV |
| 721 | MPK20299-D1_SCFV huCCR8 HV hv_cdr1 | NNGMH |
| 722 | MPK20299-D1_SCFV huCCR8 HV hv_cdr2 | VISYDGSNKYYADSVKG |
| 723 | MPK20299-D1_SCFV huCCR8 HV hv_cdr3 | VYYGSGIYYKNRYYYGMDV |
| 724 | MPK20299-D1_SCFV huCCR8 LV lv_cdr1 | GGNRIGSKNVH |
| 725 | MPK20299-D1_SCFV huCCR8 LV lv_cdr2 | RDSNRPS |
| 726 | MPK20299-D1_SCFV huCCR8 LV lv_cdr3 | QVWDSSTVV |
| 727 | MPK20299-C5_SCFV huCCR8 HV hv_cdr1 | NYGFH |
| 728 | MPK20299-C5_SCFV huCCR8 HV hv_cdr2 | VISYDGSNRYYADSVKG |
| 729 | MPK20299-C5_SCFV huCCR8 HV hv_cdr3 | VYYGSGTYYKNRYYYGMDV |
| 730 | MPK20299-C5_SCFV huCCR8 LV lv_cdr1 | GGHNIGSKGVH |
| 731 | MPK20299-C5_SCFV huCCR8 LV lv_cdr2 | RNSNRPS |
| 732 | MPK20299-C5_SCFV huCCR8 LV lv_cdr3 | QVWDSSTVV |
| 733 | MPK20299-B5_SCFV huCCR8 HV hv_cdr1 | NYGMH |
| 734 | MPK20299-B5_SCFV huCCR8 HV hv_cdr2 | VISYDGSNKYYADSVKG |
| 735 | MPK20299-B5_SCFV huCCR8 HV hv_cdr3 | VYYGSGIYYKNRYYYGMDV |
| 736 | MPK20299-B5_SCFV huCCR8 LV lv_cdr1 | GGHNIGSKGVH |
| 737 | MPK20299-B5_SCFV huCCR8 LV lv_cdr2 | RNSNRPS |
| 738 | MPK20299-B5_SCFV huCCR8 LV lv_cdr3 | QVWDSSTVV |
| 739 | MPK20299-G9_SCFV huCCR8 HV hv_cdr1 | NNGMH |
| 740 | MPK20299-G9_SCFV huCCR8 HV hv_cdr2 | VISNDGSNKYYADSVRG |
| 741 | MPK20299-G9_SCFV huCCR8 HV hv_cdr3 | VYYGSGIYYKNRYYYGMDV |
| 742 | MPK20299-G9_SCFV huCCR8 LV lv_cdr1 | GGNNIGSKNVH |
| 743 | MPK20299-G9_SCFV huCCR8 LV lv_cdr2 | RNSNRPS |
| 744 | MPK20299-G9_SCFV huCCR8 LV lv_cdr3 | QVWDSSTVV |
| 745 | MPK20299-G5_SCFV huCCR8 HV hv_cdr1 | NNGMH |
| 746 | MPK20299-G5_SCFV huCCR8 HV hv_cdr2 | VISNDGSNKYYADSVRG |
| 747 | MPK20299-G5_SCFV huCCR8 HV hv_cdr3 | VYYGSGIYYKNRYYYGMDV |
| 748 | MPK20299-G5_SCFV huCCR8 LV lv_cdr1 | EGNNIGSKNVH |
| 749 | MPK20299-G5_SCFV huCCR8 LV lv_cdr2 | RDSNRPS |
| 750 | MPK20299-G5_SCFV huCCR8 LV lv_cdr3 | QVWDSSAVV |
| 751 | MPK20298-C10_SCFV huCCR8 HV | SSGMH |
| | hv_cdr1 | |
| 752 | MPK20298-C10_SCFV huCCR8 HV | VISNDGSNKYYADSVKG |
| | hv_cdr2 | |
| 753 | MPK20298-C10_SCFV huCCR8 HV | VYYGSGIYYKNNYYYGMDV |
| | hv_cdr3 | |
| 754 | MPK20298-C10_SCFV huCCR8 LV lv_cdr1 | GGNNIGSKNVH |
| 755 | MPK20298-C10_SCFV huCCR8 LV lv_cdr2 | RNSNRPS |
| 756 | MPK20298-C10_SCFV huCCR8 LV lv_cdr3 | QAWDSSTVV |
| 757 | MPK20298-B5_SCFV huCCR8 HV hv_cdr1 | NYGMH |
| 758 | MPK20298-B5_SCFV huCCR8 HV hv_cdr2 | VISYDGSNKYYADSVKG |
| 759 | MPK20298-B5_SCFV huCCR8 HV hv_cdr3 | VYYGSGIYYKKRYYYGMDV |
| 760 | MPK20298-B5_SCFV huCCR8 LV lv_cdr1 | GGNNIGSQNVH |
| 761 | MPK20298-B5_SCFV huCCR8 LV lv_cdr2 | RDSNRPS |
| 762 | MPK20298-B5_SCFV huCCR8 LV lv_cdr3 | QVWDSSAVV |
| 763 | MPK20299-F2_SCFV huCCR8 HV hv_cdr1 | SSGMH |
| 764 | MPK20299-F2_SCFV huCCR8 HV hv_cdr2 | VISNDGSNKYYADSVKG |
| 765 | MPK20299-F2_SCFV huCCR8 HV hv_cdr3 | VYYGSGIYYKNRYYYGMDV |
| 766 | MPK20299-F2_SCFV huCCR8 LV lv_cdr1 | GGNNIGSKNVH |
| 767 | MPK20299-F2_SCFV huCCR8 LV lv_cdr2 | RDSNRPS |
| 768 | MPK20299-F2_SCFV huCCR8 LV lv_cdr3 | QAWDSGTVV |
| 769 | MPK20298-D4_SCFV huCCR8 HV hv_cdr1 | NYGMH |
| 770 | MPK20298-D4_SCFV huCCR8 HV hv_cdr2 | VISYDGSNKYYADSVKG |
| 771 | MPK20298-D4_SCFV huCCR8 HV hv_cdr3 | VYYGSGIYYKKRYYYGMDV |
| 772 | MPK20298-D4_SCFV huCCR8 LV lv_cdr1 | GGNNIGGKNVH |
| 773 | MPK20298-D4_SCFV huCCR8 LV lv_cdr2 | RDSNRPS |
| 774 | MPK20298-D4_SCFV huCCR8 LV lv_cdr3 | QVWDSSTVV |
| 775 | MPK20297-F5_SCFV huCCR8 HV hv_cdr1 | RNGMH |
| 776 | MPK20297-F5_SCFV huCCR8 HV hv_cdr2 | VISNDGSNKYYADSVKG |
| 777 | MPK20297-F5_SCFV huCCR8 HV hv_cdr3 | VYYGSGIYYKNNYYYGMDV |
| 778 | MPK20297-F5_SCFV huCCR8 LV lv_cdr1 | GGNNIGSKNVH |
| 779 | MPK20297-F5_SCFV huCCR8 LV lv_cdr2 | RNSNRPS |
| 780 | MPK20297-F5_SCFV huCCR8 LV lv_cdr3 | QVWDSSTVV |
| 781 | MPK20299-D9_SCFV huCCR8 HV hv_cdr1 | RNGMH |
| 782 | MPK20299-D9_SCFV huCCR8 HV hv_cdr2 | VISNDGSNKYYADSVKG |
| 783 | MPK20299-D9_SCFV huCCR8 HV hv_cdr3 | VYYGSGIYYKNNYYYGMDV |
| 784 | MPK20299-D9_SCFV huCCR8 LV lv_cdr1 | GGNNIESKNVH |
| 785 | MPK20299-D9_SCFV huCCR8 LV lv_cdr2 | RDSNRPS |
| 786 | MPK20299-D9_SCFV huCCR8 LV lv_cdr3 | QVWDSSTVV |
| 787 | huCCR8_32360_huIgG1z | NARMG |
| | mAb(LC: K38R) HC huCCR8 HV hv_cdr1 | |
| 788 | huCCR8_32360_huIgG1z | RIKSKTEGGTRDYAAPVKG |
| | mAb(LC: K38R) HC huCCR8 HV hv_cdr2 | |
| 789 | huCCR8_32360_huIgG1z | YSGV |
| | mAb(LC: K38R) HC huCCR8 HV hv_cdr3 | |
| 790 | huCCR8_32360_huIgG1z | KSSQSVLYSSNNRNYLA |
| | mAb(LC: K38R) LC huCCR8 LV lv_cdr1 | |
| 791 | huCCR8_32360_huIgG1z | WASTRES |
| | mAb(LC: K38R) LC huCCR8 LV lv_cdr2 | |
| 792 | huCCR8_32360_huIgG1z | QQYYSIPIT |
| | mAb(LC: K38R) LC huCCR8 LV lv_cdr3 | |
| 793 | anti- | NYGFH |
| | huCCR8_44379(VH: D72S, VL: N67A_S68A_ | |
| | M99G_W109F_S111A)_huIgG1z (mAb) HC | |
| | huCCR8 HV hv_cdr1 | |
| 794 | anti- | VISYDGSNRYYASSVKG |
| | huCCR8_44379(VH: D72S, VL: N67A_S68A_ | |
| | M99G_W109F_S111A)_huIgG1z (mAb)_HC | |
| | huCCR8 HV hv_cdr2 | |
| 795 | anti- | VYYGSGTYYKNRYYYGMDV |
| | huCCR8_44379(VH: D72S,VL: N67A_S68A_ | |
| | M99G_W109F_S111A)_huIgG1z (mAb)_HC | |
| | huCCR8 HV hv_cdr3 | |
| 796 | anti- | GGHNIGSKGVH |
| | huCCR8_44379(VH: D72S, VL: N67A_S68A_ | |
| | M99G_W109F_S111A) huIgG1z (mAb)_LC | |
| | huCCR8 LV lv_cdr1 | |
| 797 | anti- | RAANRPS |
| | huCCR8_44379(VH: D72S, VL: N67A_S68A_ | |
| | M99G_W109F_S111A)_huIgG1z (mAb)_LC | |
| | huCCR8 LV lv_cdr2 | |
| 798 | anti- | QAFDAGTVV |
| | huCCR8 44379(VH: D72S, VL: N67A_S68A_ | |
| | M99G_W109F_S111A)_huIgG1z (mAb)_LC | |
| | huCCR8 LV lv_cdr3 | |
| 799 | anti- | NYGFH |
| | huCCR8_44379(VH: D61A_D72A, VL: N67Q_ | |
| | M99E_W109F_S111A)_huIgG1z | |
| | (mAb) HC huCCR8 HV hv_cdr1 | |
| 800 | anti- | VISYAGSNRYYAASVKG |
| | huCCR8_44379(VH: D61A_D72A, VL: N67Q_ | |
| | M99E_W109F_S111A)_huIgG1z | |
| | (mAb) HC huCCR8 HV hv_cdr2 | |
| 801 | anti- | VYYGSGTYYKNRYYYGMDV |
| | huCCR8_44379(VH: D61A_D72A, VL: N67Q_ | |
| | M99E_W109F_S111A)_huIgG1z | |
| | (mAb) HC huCCR8 HV hv_cdr3 | |
| 802 | anti- | GGHNIGSKGVH |
| | huCCR8_44379(VH: D61A_D72A, VL: N67Q_ | |
| | M99E_W109F_S111A) huIgG1z | |
| | (mAb) LC huCCR8 LV lv_cdr1 | |
| 803 | anti- | RQSNRPS |
| | huCCR8_44379(VH: D61A_D72A, VL: N67Q_ | |
| | M99E_W109F_S111A)_huIgG1z | |
| | (mAb) LC huCCR8 LV lv_cdr2 | |
| 804 | anti- | QAFDAGTVV |
| | huCCR8_44379(VH: D61A_D72A, VL: N67Q_ | |
| | M99E_W109F_S111A)_huIgG1z | |
| | (mAb) LC huCCR8 LV lv_cdr3 | |
| 805 | anti- | NYGFH |
| | huCCR8_44379(VH: D61S, VL: N67Q_M99G | |
| | _W109F_S111A)_huIgG1z (mAb)_HC | |
| | huCCR8 HV hv_cdr1 | |
| 806 | anti- | VISYSGSNRYYADSVKG |
| | huCCR8_44379(VH: D61S, VL: N67Q_M99G | |
| | _W109F_S111A) huIgG1z (mAb)_HC | |
| | huCCR8 HV hv_cdr2 | |
| 807 | anti- | VYYGSGTYYKNRYYYGMDV |
| | huCCR8_44379(VH: D61S,VL: N67Q_M99G | |
| | _W109F_S111A)_huIgG1z (mAb)_HC | |
| | huCCR8 HV hv_cdr3 | |
| 808 | anti- | GGHNIGSKGVH |
| | huCCR8_44379(VH: D61S,VL: N67Q_M99G | |
| | _W109F_S111A)_huIgG1z (mAb)_LC | |
| | huCCR8 LV lv_cdr1 | |
| 809 | anti- | RQSNRPS |
| | huCCR8_44379(VH: D61S, VL: N67Q_M99G | |
| | _W109F_S111A)_huIgG1z (mAb)_LC | |
| | huCCR8 LV lv_cdr2 | |
| 810 | anti- | QAFDAGTVV |
| | huCCR8_44379(VH: D61S,VL: N67Q_M99G | |
| | _W109F_S111A)_huIgG1z (mAb)_LC | |
| | huCCR8 LV lv_cdr3 | |
| 811 | Hu anti-huCCR8 LIBC315615-1 HuIgG1z | GGHNIGSKGVH |
| | mAb_LC huCCR8 LV lv_cdr1 | |
| 812 | Hu anti-huCCR8 LIBC315615-1 HuIgG1z | RNSNRPS |
| | mAb_LC huCCR8 LV lv_cdr2 | |
| 813 | Hu anti-huCCR8 LIBC315615-1 HuIgG1z | QVWDISTVV |
| | mAb_LC huCCR8 LV lv_cdr3 | |
| 814 | Hu anti-huCCR8 LIBC315615-1 HuIgG1z | NCGMH |
| | mAb_HC huCCR8 HV hv_cdr1 | |
| 815 | Hu anti-huCCR8 LIBC315615-1 HuIgG1z | VISYDGGNKYHADSVKG |
| | mAb_HC huCCR8 HV hv_cdr2 | |
| 816 | Hu anti-huCCR8 LIBC315615-1 HuIgG1z | VYYGSGIYYKNRYYYGMDV |
| | mAb_HC huCCR8 HV hv_cdr3 | |
| 817 | Hu anti-huCCR8 LIBC317152-1 HuIgG1z | GGHNIGSKGVH |
| | mAb_LC huCCR8 LV lv_cdr1 | |
| 818 | Hu anti-huCCR8 LIBC317152-1 HuIgG1z | RNSNRPS |
| | mAb_LC huCCR8 LV lv_cdr2 | |
| 819 | Hu anti-huCCR8 LIBC317152-1 HuIgG1z | QVWDSSTVV |
| | mAb_LC huCCR8 LV lv_cdr3 | |
| 820 | Hu anti-huCCR8 LIBC317152-1 HuIgG1z | NCGMH |
| | mAb_HC huCCR8 HV hv_cdrl | |
| 821 | Hu anti-huCCR8 LIBC317152-1 HuIgG1z | VISYDGGNKYYADSVKG |
| | mAb_HC huCCR8 HV hv_cdr2 | |
| 822 | Hu anti-huCCR8 LIBC317152-1 HuIgG1z | VYYGSGIYYKNRYYYGMDV |
| | mAb_HC huCCR8 HV hv_cdr3 | |
| 823 | Hu anti-huCCR8 LIBC317471-1 HuIgG1z | GGNNIGSKNVH |
| | mAb_LC huCCR8 LV lv_cdrl | |
| 824 | Hu anti-huCCR8 LIBC317471-1 HuIgG1z | RDSNRPS |
| | mAb_LC huCCR8 LV lv_cdr2 | |
| 825 | Hu anti-huCCR8 LIBC317471-1 HuIgG1z | QVWDSNTVV |
| | mAb_LC huCCR8 LV lv_cdr3 | |
| 826 | Hu anti-huCCR8 LIBC317471-1 HuIgG1z | NNGMH |
| | mAb_HC huCCR8 HV hv_cdr1 | |
| 827 | Hu anti-huCCR8 LIBC317471-1 HuIgG1z | VISNDGSNKYYADSVRG |
| | mAb_HC huCCR8 HV hv_cdr2 | |
| 828 | Hu anti-huCCR8 LIBC317471-1 HuIgG1z | VYYGSGIYYKNNYYYGMDV |
| | mAb_HC huCCR8 HV hv_cdr3 | |
| 829 | Hu anti-huCCR8 LIBC317977-1 HuIgG1z | GGNNIGSKNVH |
| | mAb_LC huCCR8 LV lv_cdr1 | |
| 830 | Hu anti-huCCR8 LIBC317977-1 HuIgG1z | RNSNRPS |
| | mAb_LC huCCR8 LV lv_cdr2 | |
| 831 | Hu anti-huCCR8 LIBC317977-1 HuIgG1z | QVWDSSTVV |
| | mAb_LC huCCR8 LV lv_cdr3 | |
| 832 | Hu anti-huCCR8 LIBC317977-1 HuIgG1z | TYGMH |
| | mAb_HC huCCR8 HV hv_cdr1 | |
| 833 | Hu anti-huCCR8 LIBC317977-1 HuIgG1z | VISYDGSNKYYADSVKG |
| | mAb_HC huCCR8 HV hv_cdr2 | |
| 834 | Hu anti-huCCR8 LIBC317977-1 HuIgG1z | VYYGSGSYYKKNYYYGMDV |
| | mAb_HC huCCR8 HV hv_cdr3 | |
| 835 | Hu anti-huCCR8 LIBC318774-1 HuIgG1z | GGNNIGGKNVH |
| | mAb_LC huCCR8 LV lv_cdr1 | |
| 836 | Hu anti-huCCR8 LIBC318774-1 HuIgG1z | RDSNRPS |
| | mAb_LC huCCR8 LV lv_cdr2 | |
| 837 | Hu anti-huCCR8 LIBC318774-1 HuIgG1z | QVWDSSTVV |
| | mAb_LC huCCR8 LV lv_cdr3 | |
| 838 | Hu anti-huCCR8 LIBC318774-1 HuIgG1z | SYGFH |
| | mAb_HC huCCR8 HV hv_cdr1 | |
| 839 | Hu anti-huCCR8 LIBC318774-1 HuIgG1z | VISYDGSNKYYADSVKG |
| | mAb_HC huCCR8 HV hv_cdr2 | |
| 840 | Hu anti-huCCR8 LIBC318774-1 HuIgG1z | VYYGSGTYYKNRYYYGMDV |
| | mAb_HC huCCR8 HV hv_cdr3 | |
| 841 | Hu anti-huCCR8 LIBC319840-1 HuIgG1z | GGNNIGSKNVH |
| | mAb_LC huCCR8 LV lv_cdr1 | |
| 842 | Hu anti-huCCR8 LIBC319840-1 HuIgG1z | RDSNRPS |
| | mAb_LC huCCR8 LV lv_cdr2 | |
| 843 | Hu anti-huCCR8 LIBC319840-1 HuIgG1z | QVWDSSTVV |
| | mAb_LC huCCR8 LV lv_cdr3 | |
| 844 | Hu anti-huCCR8 LIBC319840-1 HuIgG1z | NNGMH |
| | mAb_HC huCCR8 HV hv_cdr1 | |
| 845 | Hu anti-huCCR8 LIBC319840-1 HuIgG1z | VISNDGSNKYYPDSVKG |
| | mAb_HC huCCR8 HV hv_cdr2 | |
| 846 | Hu anti-huCCR8 LIBC319840-1 HuIgG1z | VYYGSGNYYKNNYYYGMDV |
| | mAb_HC huCCR8 HV hv_cdr3 | |
| 847 | Hu anti-huCCR8 LIBC320212-1 HuIgG1z | EGNNIGSQNVH |
| | mAb_LC huCCR8 LV lv_cdr1 | |
| 848 | Hu anti-huCCR8 LIBC320212-1 HuIgG1z | RDSNRPS |
| | mAb_LC huCCR8 LV lv_cdr2 | |
| 849 | Hu anti-huCCR8 LIBC320212-1 HuIgG1z | QVWDGSAVV |
| | mAb_LC huCCR8 LV lv_cdr3 | |
| 850 | Hu anti-huCCR8 LIBC320212-1 HuIgG1z | SSGMH |
| | mAb_HC huCCR8 HV hv_cdr1 | |
| 851 | Hu anti-huCCR8 LIBC320212-1 HuIgG1z | VISHDGSNKYYADSVKG |
| | mAb_HC huCCR8 HV hv_cdr2 | |
| 852 | Hu anti-huCCR8 LIBC320212-1 HuIgG1z | VYYGSGIYYKNRYYYGMDV |
| | mAb_HC huCCR8 HV hv_cdr3 | |
| 853 | Hu anti-huCCR8 LIBC320384-1 HuIgG1z | GGHNIGSKGVH |
| | mAb_LC huCCR8 LV lv_cdr1 | |
| 854 | Hu anti-huCCR8 LIBC320384-1 HuIgG1z | RNSNRPS |
| | mAb_LC huCCR8 LV lv_cdr2 | |
| 855 | Hu anti-huCCR8 LIBC320384-1 HuIgG1z | QVWDSSTVV |
| | mAb_LC huCCR8 LV lv_cdr3 | |
| 856 | Hu anti-huCCR8 LIBC320384-1 HuIgG1z | DCGMH |
| | mAb_HC huCCR8 HV hv_cdr1 | |
| 857 | Hu anti-huCCR8 LIBC320384-1 HuIgG1z | VISYDGGNKYYADSVKG |
| | mAb_HC huCCR8 HV hv_cdr2 | |
| 858 | Hu anti-huCCR8 LIBC320384-1 HuIgG1z | VYYGSGIYYKNRYYYGMDV |
| | mAb_HC huCCR8 HV hv_cdr3 | |
| 859 | Hu anti-huCCR8 LIBC320689-1 HuIgG1z | GGNNIGSKNVH |
| | mAb_LC huCCR8 LV lv_cdr1 | |
| 860 | Hu anti-huCCR8 LIBC320689-1 HuIgG1z | RSSNRPS |
| | mAb_LC huCCR8 LV lv_cdr2 | |
| 861 | Hu anti-huCCR8 LIBC320689-1 HuIgG1z | QIWDSSTVV |
| | mAb_LC huCCR8 LV lv_cdr3 | |
| 862 | Hu anti-huCCR8 LIBC320689-1 HuIgG1z | SYGMH |
| | mAb_HC huCCR8 HV hv_cdr1 | |
| 863 | Hu anti-huCCR8 LIBC320689-1 HuIgG1z | VISFDGNNKYYADSVKG |
| | mAb_HC huCCR8 HV hv_cdr2 | |
| 864 | Hu anti-huCCR8 LIBC320689-1 HuIgG1z | VYYGSGSYYKNRYYYGMDV |
| | mAb_HC huCCR8 HV hv_cdr3 | |
| 865 | Hu anti-huCCR8 LIBC321408-1 HuIgG1z | GGNNIGSKNVH |
| | mAb_LC huCCR8 LV lv_cdr1 | |
| 866 | Hu anti-huCCR8 LIBC321408-1 HuIgG1z | RDSNRPS |
| | mAb_LC huCCR8 LV lv_cdr2 | |
| 867 | Hu anti-huCCR8 LIBC321408-1 HuIgG1z | QVWDSSTVV |
| | mAb_LC huCCR8 LV lv_cdr3 | |
| 868 | Hu anti-huCCR8 LIBC321408-1 HuIgG1z | SNGMH |
| | mAb_HC huCCR8 HV hv_cdr1 | |
| 869 | Hu anti-huCCR8 LIBC321408-1 HuIgG1z | VISNDGSNKYYGDSVKG |
| | mAb_HC huCCR8 HV hv_cdr2 | |
| 870 | Hu anti-huCCR8 LIBC321408-1 HuIgG1z | VYYGSGIYYRNNYYYGMDV |
| | mAb_HC huCCR8 HV hv_cdr3 | |
| 871 | Hu anti-huCCR8 LIBC321824-1 HuIgG1z | GGNNIGSKNVH |
| | mAb_LC huCCR8 LV lv_cdr1 | |
| 872 | Hu anti-huCCR8 LIBC321824-1 HuIgG1z | RNTNRPS |
| | mAb_LC huCCR8 LV lv_cdr2 | |
| 873 | Hu anti-huCCR8 LIBC321824-1 HuIgG1z | QVWDSSTVV |
| | mAb_LC huCCR8 LV lv_cdr3 | |
| 874 | Hu anti-huCCR8 LIBC321824-1 HuIgG1z | GYGMH |
| | mAb_HC huCCR8 HV hv_cdr1 | |
| 875 | Hu anti-huCCR8 LIBC321824-1 HuIgG1z | VISYDGSNKYYADSVKG |
| | mAb_HC huCCR8 HV hv_cdr2 | |
| 876 | Hu anti-huCCR8 LIBC321824-1 HuIgG1z | VYYGSGIYYKNRYYYGMDV |
| | mAb_HC huCCR8 HV hv_cdr3 | |
| 877 | Hu anti-huCCR8 LIBC321845-1 HuIgG1z | GGNNIGSKNVH |
| | mAb_LC huCCR8 LV lv_cdr1 | |
| 878 | Hu anti-huCCR8 LIBC321845-1 HuIgG1z | RNTNRPS |
| | mAb_LC huCCR8 LV lv_cdr2 | |
| 879 | Hu anti-huCCR8 LIBC321845-1 HuIgG1z | QVWDSSTVV |
| | mAb_LC huCCR8 LV lv_cdr3 | |
| 880 | Hu anti-huCCR8 LIBC321845-1 HuIgG1z | GYGMH |
| | mAb_HC huCCR8 HV hv_cdr1 | |
| 881 | Hu anti-huCCR8 LIBC321845-1 HuIgG1z | VISYDGSNKYYADSVKG |
| | mAb_HC huCCR8 HV hv_cdr2 | |
| 882 | Hu anti-huCCR8 LIBC321845-1 HuIgG1z | VYYGSGIYYKNRYYYGMDV |
| | mAb_HC huCCR8 HV hv_cdr3 | |
| 883 | Hu anti-huCCR8 LIBC322176-1 HuIgG1z | GGNNIGDKNVH |
| | mAb_LC huCCR8 LV lv_cdr1 | |
| 884 | Hu anti-huCCR8 LIBC322176-1 HuIgG1z | RNNVRPS |
| | mAb_LC huCCR8 LV lv_cdr2 | |
| 885 | Hu anti-huCCR8 LIBC322176-1 HuIgG1z | QVWDSSTVV |
| | mAb_LC huCCR8 LV lv_cdr3 | |
| 886 | Hu anti-huCCR8 LIBC322176-1 HuIgG1z | NFGMH |
| | mAb_HC huCCR8 HV hv_cdr1 | |
| 887 | Hu anti-huCCR8 LIBC322176-1 HuIgG1z | VISYDGGNKYYADSVKG |
| | mAb_HC huCCR8 HV hv_cdr2 | |
| 888 | Hu anti-huCCR8 LIBC322176-1 HuIgG1z | VYYGSGSYYKKRYYYGMDV |
| | mAb_HC huCCR8 HV hv_cdr3 | |
| 889 | Hu anti-huCCR8 LIBC323412-1 HuIgG1z | GGNNIGSKNVH |
| | mAb_LC huCCR8 LV lv_cdr1 | |
| 890 | Hu anti-huCCR8 LIBC323412-1 HuIgG1z | RDSNRPS |
| | mAb_LC huCCR8 LV lv_cdr2 | |
| 891 | Hu anti-huCCR8 LIBC323412-1 HuIgG1z | QVWDSSTVV |
| | mAb_LC huCCR8 LV lv_cdr3 | |
| 892 | Hu anti-huCCR8 LIBC323412-1 HuIgG1z | SCGMH |
| | mAb_HC huCCR8 HV hv_cdr1 | |
| 893 | Hu anti-huCCR8 LIBC323412-1 HuIgG1z | VISYDGTNKYYADSVKG |
| | mAb_HC huCCR8 HV hv_cdr2 | |
| 894 | Hu anti-huCCR8 LIBC323412-1 HuIgG1z | VYYGSGIYYKKNYYYGMDV |
| | mAb_HC huCCR8 HV hv_cdr3 | |
| 895 | huCCR8_32360_huIgG1z mAb_HC huCCR8 | NARMG |
| | HV hv_cdr1 | |
| 896 | huCCR8_32360_huIgG1z mAb_HC huCCR8 | RIKSKTEGGTRDYAAPVKG |
| | HV hv_cdr2 | |
| 897 | huCCR8_32360_huIgG1z mAb_HC huCCR8 | YSGV |
| | HV hv_cdr3 | |
| 898 | huCCR8_32360_huIgG1z mAb_LC huCCR8 | KSSQSVLYSSNNKNYLA |
| | LV lv_cdr1 | |
| 899 | huCCR8 32360_huIgG1z mAb_LC huCCR8 | WASTRES |
| | LV lv_cdr2 | |
| 900 | huCCR8 32360_huIgG1z mAb_LC huCCR8 | QQYYSIPIT |
| | LV lv_cdr3 | |
| 901 | MPK20298-A4_SCFV HV huCCR8 | QVQLVESGGGVVQPGRSLRLSCVVS |
| | | GFNFSNNGMHWVRQAPGKGLEWVA |
| | | VISNDGSNKYYADSVKGRFTISRDNS |
| | | KNTLYLQMNSLRTEDTAVYYCAKV |
| | | YYGSGIYYKNRNYYGMDVWGQGTT |
| | | VTVSS |
| 902 | MPK20298-A4_SCFV LV huCCR8 | SYELTQPPSVSVALGQTARITCGGNN |
| | | IGSQNVHWYQQKPGQAPVLVIYRDS |
| | | NRPSGIPDRFSGSKSGNTATLTISRAQ |
| | | AGDEADYYCQVWDSSTVVFGGGTK |
| | | LTVL |
| 903 | MPK20299-D2_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 904 | MPK20299-D2_SCFV LV huCCR8 | SYELTQPPSVSVALGQTARITCGGHN |
| | | IGSKGVHWYQQKPGQAPVLVIYRNS |
| | | NRPSGIPERFSGSNSGNTATLTITRAQ |
| | | AGDEADYYCQVWDSSTVVFGGGTK |
| | | LTVL |
| 905 | MPK20299-F11_SCFV HV huCCR8 | QVQLVESGGGVVQPGRSLRLSCAPS |
| | | GFNFSNYGMHWVRQAPGKGLEWVA |
| | | VISYDGSNKYYADSVKGRFTISRDNS |
| | | KNTLFLQMNSLRAEDTAVYFCARVY |
| | | YGSGSYYKKRYYYGMDVWGQGTT |
| | | VTVSS |
| 906 | MPK20299-F11_SCFV LV huCCR8 | SYELTQPPSVSVALGQTARITCGGNN |
| | | IGSQNVHWYQQKPGQAPVLVIYRDS |
| | | NRPSGIPERFSGSKSGNTATLTISRAQ |
| | | AGDEADYYCQVWDSSTVVFGGGTQ |
| | | LTVL |
| 907 | MPK20298-H6_SCFV HV huCCR8 | QVQLVESGGGVVQPGRSLRLSCAAS |
| | | GFTFSSSGMHWVRQAPGKGLEWVA |
| | | VISYDGTNKYYADSVKGRFTISRDNS |
| | | KNTLYLQMNSLRAEDTAVYYCAKV |
| | | YYGSGIYYKNRYYYGMDVWGQGTT |
| | | VTVSS |
| 908 | MPK20298-H6_SCFV LV huCCR8 | SYELTQPPSVSVALGQTARITCGGHN |
| | | IGSKGVHWYQQKPGQAPVLVIYRNS |
| | | NRPSGIPERFSGSNSGNTATLTISRAQ |
| | | AGDEADYYCQVWDSSTVVFGGGTQ |
| | | LTVL |
| 909 | MPK20297-A4_SCFV HV huCCR8 | QVQLVESGGGVVQPGRSLRLSCAVS |
| | | GFNFSNYGMHWVRQVPGRGLDWVA |
| | | VISNDGSNKYYADSVKGRFTISRDNS |
| | | KNTLYLQMDSLRTEDTAVYYCAKV |
| | | YYGSGIYYKKRYYYGMDVWGQGTT |
| | | VTVSS |
| 910 | MPK20297-A4_SCFV LV huCCR8 | SYELTQPPSVSVALGQTARITCGGHN |
| | | IGSQNVHWYQQKPGQAPVLVIYRDS |
| | | NRPSGIPERFSGSKSGNTATLTISRAQ |
| | | AGDEADYYCQVWDSSTVVFGGGTQ |
| | | LTVL |
| 911 | MPK20299-H8_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 912 | MPK20299-H8_SCFV LV huCCR8 | SYELTQPPSVSVAPGQTARITCGGNNI |
| | | GSKNVHWYQQKAGQAPVQVIYRNS |
| | | NRPSGIPARFSGSNSGNTATLTISRAQ |
| | | AGDEADYYCQVWDSSTVVFGGGTK |
| | | LTVL |
| 913 | MPK20300-C11_SCFV HV huCCR8 | QVQLVESGGGVVQPGRSLRLSCAAS |
| | | GFTFSSYGMHWVRQAPGKGLEWVA |
| | | VISYDGSNKYYADSVKGRFTISRDNS |
| | | KNTLYLQMNSLRGEDTAVYYCARV |
| | | YYGSGSYYKNRYYYGMDVWGQGT |
| | | TVTVSS |
| 914 | MPK20300-C11_SCFV LV huCCR8 | SYELTQPPSVSVAPGQTARIPCGGNNI |
| | | GSKNVHWYQQKPGQAPVLVIYRDIN |
| | | RPSGIPERFSGSNSGNTATLTISRAQA |
| | | GDEADYYCQVWDSSVVFGGGTKLT |
| | | VL |
| 915 | MPK20298-B1_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 916 | MPK20298-B1_SCFV LV huCCR8 | SYELTQPPSVSVALGQTARLTCEGNN |
| | | IGSKNVHWYQQKPGQAPVLVIYRNS |
| | | NRPSGIPERFSGSNSGNTATLTISRVQ |
| | | AGDEADYYCQAWDSSTVVFGGGTQ |
| | | LTVL |
| 917 | MPK20297-E5_SCFV HV huCCR8 | QVQLVESGGGLVKPGGSLRLSCAVS |
| | | GFNFSNNGMHWVRQAPGKGLEWVA |
| | | VISYDGSNKYYTDSVKGRFTISRDNS |
| | | KNTLYLQMNSLRTEDTAVYYCAKV |
| | | YYGSGIYYKKRYYYGMDVWGQGTT |
| | | VTVSS |
| 918 | MPK20297-E5_SCFV LV huCCR8 | SYELTQPLSVSEALGQTARITCGGNNI |
| | | GSKNVHWYQQKPGQAPVLVIYRDSN |
| | | RPSGIPERFSGSNSGNAATLTISRVEA |
| | | GDEADYYCQVWDSSSDHVVFGGGT |
| | | QLTVL |
| 919 | MPK20299-A3_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 920 | MPK20299-A3_SCFV LV huCCR8 | SYELTQPPSVSVAPGQTARITCGGNNI |
| | | GSKNVHWYQQKPGQAPVLVIYRNSN |
| | | RPSGIPERFSGSNSGNTATLTISGTQA |
| | | MDEADYYCQAWDSSNVVFGGGTQL |
| | | TVL |
| 921 | MPK20297-B4_SCFV HV huCCR8 | QVQLVESGGGVVQPGRSLRLSCVVS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKNNYYYGMDVWGQGTT |
| | | VTVSS |
| 922 | MPK20297-B4_SCFV LV huCCR8 | SYELTQPLSVSVALGQTARITCGGNN |
| | | IGSQNVHWYQQKPGQAPVLVIYRDS |
| | | NRPSGIPDRFSGSKSGNTATLTISRAQ |
| | | AGDEADYYCQVWDSSTVVFGGGTQ |
| | | LTVL |
| 923 | MPK20298-F6_SCFV HV huCCR8 | QVQLVESGGGVVQPGRSLRLSCVVS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKNRYYYGMDVWGQGTT |
| | | VTVSS |
| 924 | MPK20298-F6_SCFV LV huCCR8 | SYELTQPPSVSVAPGQTARITCGGNNI |
| | | GSKNVHWYQQKPGQAPVLVIYRDSN |
| | | RPSGIPERFSGSKSGTTATLTISRAQA |
| | | GDEAEYYCQVWDSSTVVFGGGTELT |
| | | VL |
| 925 | MPK20299-H3_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 926 | MPK20299-H3_SCFV LV huCCR8 | SYELTQPLSVSVALGQTARITCGGNN |
| | | IGSKNVHWYQQKPGQAPVLAIYRNS |
| | | NRPSGIPERFTGSNSGNTATLTISRAQ |
| | | AGDESDYYCQIWDSSTVVFGGGTKL |
| | | TVL |
| 927 | MPK20298-B9_SCFV HV huCCR8 | QVQLVESGGGVVQPGRSLRLSCAAS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKKNYYYGMDVWGQGTT |
| | | VTVSS |
| 928 | MPK20298-B9_SCFV LV huCCR8 | SYELTQPPSVSVALGQTARISCGGNNI |
| | | GSKNVHWYQQKPGQAPVLVIYRDSN |
| | | RPSGIPERFSGSKSGTTATLTISRAQA |
| | | GDEAEYYCQVWDSSTVVFGGGTQLT |
| | | VL |
| 929 | MPK20299-E2_SCFV HV huCCR8 | QVQLVESGGGVVQPGRSLRLSCAVS |
| | | GFNFSNNGMHWVRQAPGKGLEWVA |
| | | VISYDGSNKYYTDSVKGRFTISRDNS |
| | | KNTLYLQMNSLRTEDTAVYYCAKV |
| | | YYGSGIYYKKRYYYGMDVWGQGTT |
| | | VTVSS |
| 930 | MPK20299-E2_SCFV LV huCCR8 | SYELTQPPSVSVALGQTARITCEGNNI |
| | | GSQNVHWYQQKPGQAPVLVMYRDS |
| | | NRPSGIPERFSGSKSGNTATLAISRAQ |
| | | AGDESDYYCQVWDGSAVVFGGGTK |
| | | LTVL |
| 931 | MPK20299-D6_SCFV HV huCCR8 | QVQLVESGGGVVQPGRSLRLSCAAS |
| | | GFTFSSYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 932 | MPK20299-D6_SCFV LV huCCR8 | SYELTQPLSVSVALGQTARITCEGNNI |
| | | GSQNVHWYQQKPGQAPVLVMYRDS |
| | | NRPSGIPERFSGSKSGNTATLAISRAQ |
| | | AGDESDYYCQVWDGSAVVFGGGTQ |
| | | LTVL |
| 933 | MPK20299-A4_SCFV HV huCCR8 | QVQLVESGGGVVQPGRSLRLSCAAS |
| | | GFTFSNYGFHWVRQTPGKGLEWVA |
| | | VISYDGSNRYYADSVKGRFTISRDNS |
| | | KNTLYLQMNSLRGEDTALYYCARV |
| | | YYGSGTYYKNRYYYGMDVWGQGT |
| | | TVTVSS |
| 934 | MPK20299-A4_SCFV LV huCCR8 | SYELTQPPSVSVALGQTARITCGGHN |
| | | IGSKGVHWYQQKPGQAPVLVIYRNS |
| | | NRPSGIPERFSGSNSGNTATLTISGTQ |
| | | AMDEADYYCQAWDSGTVVFGGGTQ |
| | | LTVL |
| 935 | MPK20300-G5_SCFV HV huCCR8 | QVQLVESGGGVVQPGRSLRLSCAAS |
| | | GFTFSNYGFHWVRQTPGKGLEWVA |
| | | VISYDGSNRYYADSVKGRFTISRDNS |
| | | KNTLYLQMNSLRGEDTALYYCARV |
| | | YYGSGTYYKNRYYYGMDVWGQGT |
| | | TVTVSS |
| 936 | MPK20300-G5_SCFV LV huCCR8 | SYELTQPPSVSVALGQTARITCGANN |
| | | IGSKNVHWYQQKPGQPPVLVIYRDF |
| | | NRPSGIPERFSASNSGNTATLTISRGQ |
| | | AGDEADYYCQVWDSSTGNVVFGGG |
| | | TKLTVL |
| 937 | MPK20299-C3_SCFV HV huCCR8 | QVQLVESGGGVVQPGRSLRLSCAAS |
| | | GFIFSNYGFHWVRQTPGKGLEWVAV |
| | | ISYDGSNKYYADSVKGRFTISRDNSK |
| | | NTLYLQMNSLRGEDTAVYYCARVY |
| | | YGSGSYYKNRYYYGMDVWGQGTT |
| | | VTVSS |
| 938 | MPK20299-C3_SCFV LV huCCR8 | SYELTQPPSVSVAPGQTARITCGGNNI |
| | | GSKNVHWYQQKPGQAPVLVIYRDSN |
| | | RPSGIPERFSGSKSGTTATLTISRAQA |
| | | GDEADYYCQVWDSSTVVFGGGTELT |
| | | VL |
| 939 | MPK20299-B7_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 940 | MPK20299-B7_SCFV LV huCCR8 | SYELTQSSSVSVAPGQTARITCGGNNI |
| | | GSKNVHWYQQKPGQAPVLVIYRDSN |
| | | RPSGIPERFSGSKSGTTATLTISRVEA |
| | | GDEADYYCQVWDSSSAHVIFGGGTK |
| | | LTVL |
| 941 | MPK20299-A5_SCFV HV huCCR8 | QVQLVESGGGVVQPGRSLRLSCGAS |
| | | GFTFSGYGMHWVRQAPGKGLEWVA |
| | | VISYDGSNKYYADSVKGRFTISRDNS |
| | | KNTLYLQMNSLRGEDTAVYYCARV |
| | | YYGSGIYYKNRYYYGMDVWGQGTT |
| | | VTVSS |
| 942 | MPK20299-A5_SCFV LV huCCR8 | SYELTQPPSGSVALGQTARITCGGNN |
| | | LGSKNVHWYQQKPGQAPVLVIYRNS |
| | | NRPSGIPERFSGSNSGNTATLTISRAQ |
| | | AGDEADYYCQVWDSSTVVFGGGTK |
| | | LTVL |
| 943 | MPK20299-D1_SCFV HV huCCR8 | QVQLVESGGGLVKPGGSLRLSCAAS |
| | | GFTFSNNGMHWVRQAPGKGLEWVA |
| | | VISYDGSNKYYADSVKGRFTISRDNS |
| | | KNTLYLQMNSLRAEDTAVYYCAKV |
| | | YYGSGIYYKNRYYYGMDVWGQGTT |
| | | VTVSS |
| 944 | MPK20299-D1_SCFV LV huCCR8 | SYELTQPPSVSVALGQTARITCGGNRI |
| | | GSKNVHWYQQKPGQAPVLVIYRDSN |
| | | RPSGIPERFSGSKSGTTATLTISRAQA |
| | | GDEAEYYCQVWDSSTVVFGGGTKLT |
| | | VL |
| 945 | MPK20299-C5_SCFV HV huCCR8 | QVQLVESGGGVVQPGRSLRLSCAAS |
| | | GFTFSNYGFHWVRQTPGKGLEWVA |
| | | VISYDGSNRYYADSVKGRFTISRDNS |
| | | KNTLYLQMNSLRGEDTALYYCARV |
| | | YYGSGTYYKNRYYYGMDVWGQGT |
| | | TVTVSS |
| 946 | MPK20299-C5_SCFV LV huCCR8 | SYELTQLPSVSVALGQTARITCGGHN |
| | | IGSKGVHWYQQKPGQAPVLVIYRNS |
| | | NRPSGIPERFSGSNSGNTATLTISRAQ |
| | | AGDEADYYCQVWDSSTVVFGGGTE |
| | | LTVL |
| 947 | MPK20299-B5_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKNRYYYGMDVWGQGTTV |
| | | TVSS |
| 948 | MPK20299-B5_SCFV LV huCCR8 | SYELTQPPSVSVALGQTARITCGGHN |
| | | IGSKGVHWYQQKPGQAPVLVIYRNS |
| | | NRPSGIPERFSGSNSGNTATLTISRAQ |
| | | AGDEADYYCQVWDSSTVVFGGGTQ |
| | | LTVL |
| 949 | MPK20299-G9_SCFV HV huCCR8 | QVQLVESGGDLVQPGRSLRLSCAAS |
| | | GFTFSNNGMHWVRQAPGKGLEWVA |
| | | VISNDGSNKYYADSVRGRFTISRDNS |
| | | KNTLYLQMNSLRAEDTAVYYCAKV |
| | | YYGSGIYYKNRYYYGMDVWGQGTT |
| | | VTVSS |
| 950 | MPK20299-G9_SCFV LV huCCR8 | SYELTQPLSVSVALGQTARITCGGNN |
| | | IGSKNVHWYQQKPGQAPVLVIYRNS |
| | | NRPSGIPERFSGSNSGNTATLTLSRVQ |
| | | AGDEADYYCQVWDSSTVVFGGGTK |
| | | LTVL |
| 951 | MPK20299-G5_SCFV HV huCCR8 | QVQLVESGGGVVQPGRSLRLSCAVS |
| | | GFNFSNNGMHWVRQAPGKGLEWVA |
| | | VISNDGSNKYYADSVRGRFTISRDNS |
| | | KNTLYLQMDSLRTEDTAVYYCAKV |
| | | YYGSGIYYKNRYYYGMDVWGQGTT |
| | | VTVSS |
| 952 | MPK20299-G5_SCFV LV huCCR8 | SYELTQPPSVSVALGQTARLTCEGNN |
| | | IGSKNVHWYQQKPGQAPVLVIYRDS |
| | | NRPSGIPERFSGSKSGNTATLAISRAQ |
| | | AGDESDYYCQVWDSSAVVFGGGTK |
| | | LTVL |
| 953 | MPK20298-C10_SCFV HV huCCR8 | QVQLVESGGGVVQPGRSLRLSCAAS |
| | | GFTFSSSGMHWVRQAPGKGLEWVA |
| | | VISNDGSNKYYADSVKGRFTISRDNS |
| | | KNTLYLQMNSLRAEDTAVYYCAKV |
| | | YYGSGIYYKNNYYYGMDVWGQGTT |
| | | VTVSS |
| 954 | MPK20298-C10_SCFV LV huCCR8 | SYELTQPPSVSVALGQTARITCGGNN |
| | | IGSKNVHWYQQKPGQAPVLAIYRNS |
| | | NRPSGIPERFTGSNSGNTATLTISGTQ |
| | | AMDEADYYCQAWDSSTVVFGGGTK |
| | | LTVL |
| 955 | MPK20298-B5_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 956 | MPK20298-B5_SCFV LV huCCR8 | SYELTQPPSVSVALGQTARITCGGNN |
| | | IGSQNVHWYQQKPGQAPVLVIYRDS |
| | | NRPSGIPERFSGSKSGNTATLAISRAQ |
| | | AGDESDYYCQVWDSSAVVFGGGTQ |
| | | LTVL |
| 957 | MPK20299-F2_SCFV HV huCCR8 | QVQLVESGGGVVQPGRSLRLSCAAS |
| | | GFTLSSSGMHWVRQAPGKGLEWVA |
| | | VISNDGSNKYYADSVKGRFTISRDDS |
| | | KNTLYLQMDSLRTEDTAVYYCAKV |
| | | YYGSGIYYKNRYYYGMDVWGQGTT |
| | | VTVSS |
| 958 | MPK20299-F2_SCFV LV huCCR8 | SYELTQPPSVSVALGQTARISCGGNNI |
| | | GSKNVHWYQQKPGQAPVLVMYRDS |
| | | NRPSGIPERFSGSNSGNTATLTISGTQ |
| | | AMDEADYYCQAWDSGTVVFGGGTK |
| | | LTVL |
| 959 | MPK20298-D4_SCFV HV huCCR8 | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSS |
| 960 | MPK20298-D4_SCFV LV huCCR8 | SYELTQPPSVSVALGQTARITCGGNN |
| | | IGGKNVHWYQQKPGQAPVLVIYRDS |
| | | NRPSGIPERFSGSKSGNTATLTISRAQ |
| | | AGDESDYYCQVWDSSTVVFGGGTQ |
| | | LTVL |
| 961 | MPK20297-F5_SCFV HV huCCR8 | QVQLVESGGGVVQPGRSLRLSCVVS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKNNYYYGMDVWGQGTT |
| | | VTVSS |
| 962 | MPK20297-F5_SCFV LV huCCR8 | SYELTQPLSVSVALGQTARITCGGNN |
| | | IGSKNVHWYQQKPGQAPVLVIYRNS |
| | | NRPSGIPERFSGSNSGNTATLTISRAQ |
| | | AGDEADYYCQVWDSSTVVFGGGTK |
| | | LTVL |
| 963 | MPK20299-D9_SCFV HV huCCR8 | QVQLVESGGGLVKPGGSLRLSCAAS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKNNYYYGMDVWGQGTT |
| | | VTVSS |
| 964 | MPK20299-D9_SCFV LV huCCR8 | SYELTQPPSVSVALGQTARISCGGNNI |
| | | ESKNVHWYQQKPGQAPVLVIYRDSN |
| | | RPSGIPERFSGSKSGTTATLTISRAQA |
| | | GDEAEYYCQVWDSSTVVFGGGTQLT |
| | | VI |
| 965 | huCCR8_32360_huIgG1z | EVQLVESGGGLVKPGGSLRLSCAAS |
| | mAb(LC: K38R)_HC HV huCCR8 | GFTFSNARMGWVRQAPGKGLEWVG |
| | | RIKSKTEGGTRDYAAPVKGRFTISRD |
| | | DSKNTLYLQMNSLKTEDTAVYYCTS |
| | | YSGVWGQGTMVTVSS |
| 966 | huCCR8_32360_huIgG1z | DIVMTQSPDSLAVSLGERATINCKSS |
| | mAb(LC: K38R)_LC LV huCCR8 | QSVLYSSNNRNYLAWYHQKPGQSPK |
| | | LLISWASTRESGVPDRFSGSGSGTDFT |
| | | LTINSLQAEDVAVYYCQQYYSIPITFG |
| | | GGTKVEIKR |
| 967 | anti- | QVQLVESGGGVVQPGRSLRLSCAAS |
| | huCCR8_44379(VH: D72S, VL: N67A_S68A_ | GFTFSNYGFHWVRQTPGKGLEWVA |
| | M99G_W109F_S111A)_huIgG1z (mAb)_HC | VISYDGSNRYYASSVKGRFTISRDNS |
| | HV huCCR8 | KNTLYLQMNSLRGEDTALYYCARV |
| | | YYGSGTYYKNRYYYGMDVWGQGT |
| | | TVTVSS |
| 968 | anti- | SYELTQPPSVSVALGQTARITCGGHN |
| | huCCR8_44379(VH: D72S, VL: N67A_S68A_ | IGSKGVHWYQQKPGQAPVLVIYRAA |
| | M99G_W109F_S111A)_huIgG1z (mAb)_LC | NRPSGIPERFSGSNSGNTATLTISGTQ |
| | LV huCCR8 | AGDEADYYCQAFDAGTVVFGGGTQ |
| | | LTVLG |
| 969 | anti- | QVQLVESGGGVVQPGRSLRLSCAAS |
| | huCCR8_44379(VH: D61A_D72A, VL: N67Q_ | GFTFSNYGFHWVRQTPGKGLEWVA |
| | M99E_W109F_S111A)_huIgG1z | VISYAGSNRYYAASVKGRFTISRDNS |
| | (mAb) HC HV huCCR8 | KNTLYLQMNSLRGEDTALYYCARV |
| | | YYGSGTYYKNRYYYGMDVWGQGT |
| | | TVTVSS |
| 970 | anti- | SYELTQPPSVSVALGQTARITCGGHN |
| | huCCR8_44379(VH: D61A_D72A, VL: N67Q_ | IGSKGVHWYQQKPGQAPVLVIYRQS |
| | M99E_W109F_S111A)_huIgG1z | NRPSGIPERFSGSNSGNTATLTISGTQ |
| | (mAb) LC LV huCCR8 | AEDEADYYCQAFDAGTVVFGGGTQ |
| | | LTVLG |
| 971 | anti- | QVQLVESGGGVVQPGRSLRLSCAAS |
| | huCCR8_44379(VH: D61S, VL: N67Q_M99G | GFTFSNYGFHWVRQTPGKGLEWVA |
| | _W109F_S111A)_huIgG1z (mAb)_HC HV | VISYSGSNRYYADSVKGRFTISRDNS |
| | huCCR8 | KNTLYLQMNSLRGEDTALYYCARV |
| | | YYGSGTYYKNRYYYGMDVWGQGT |
| | | TVTVSS |
| 972 | anti- | SYELTQPPSVSVALGQTARITCGGHN |
| | huCCR8_44379(VH: D61S,VL: N67Q_M99G | IGSKGVHWYQQKPGQAPVLVIYRQS |
| | _W109F_S111A)_huIgG1z (mAb)_LC LV | NRPSGIPERFSGSNSGNTATLTISGTQ |
| | huCCR8 | AGDEADYYCQAFDAGTVVFGGGTQ |
| | | LTVLG |
| 973 | Hu anti-huCCR8 LIBC315615-1 HuIgG1z | SYELTQPLSVSVALGQTARITCGGHN |
| | mAb_LC LV huCCR8 | IGSKGVHWYQQKPGQAPVLVIYRNS |
| | | NRPSGIPERFSGSNSGNTATLTISRAQ |
| | | AGDEADYYCQVWDISTVVFGGGTEL |
| | | TVLG |
| 974 | Hu anti-huCCR8 LIBC315615-1 HuIgG1z | QVQLVESGGGVAQPGRSLRLSCAAS |
| | mAb_HC HV huCCR8 | GFNFSNCGMHWVRQAPGKGLEWVA |
| | | VISYDGGNKYHADSVKGRFTISRDDS |
| | | KNTLYLQMDSLRTEDTAVYYCAKV |
| | | YYGSGIYYKNRYYYGMDVWGQGTT |
| | | VTVSS |
| 975 | Hu anti-huCCR8 LIBC317152-1 HuIgG1z | SYELTQPLSVSVALGQTARITCGGHN |
| | mAb_LC LV huCCR8 | IGSKGVHWYQQKPGQAPVLVIYRNS |
| | | NRPSGIPERFSGSNSGKTATLTISRAQ |
| | | AGDEADYYCQVWDSSTVVFGGGTE |
| | | LTVLG |
| 976 | Hu anti-huCCR8 LIBC317152-1 HuIgG1z | QVQLVESGGGVAQPGRSLRLSCAAS |
| | mAb_HC HV huCCR8 | GFNFSNCGMHWVRQAPGKGLEWVA |
| | | VISYDGGNKYYADSVKGRFTISRDDS |
| | | KNTLYLQMDSLRTEDTAVYYCAKV |
| | | YYGSGIYYKNRYYYGMDVWGQGTT |
| | | VTVSS |
| 977 | Hu anti-huCCR8 LIBC317471-1 HuIgG1z | SYELTQPLSVSVALGQTARITCGGNN |
| | mAb_LC LV huCCR8 | IGSKNVHWYQKRPGQAPVLVIYRDS |
| | | NRPSGIPERFSGSKSGNTATLTISRAQ |
| | | AGDEADYYCQVWDSNTVVFGGGTN |
| | | LTVLG |
| 978 | Hu anti-huCCR8 LIBC317471-1 HuIgG1z | QVQLVESGGGVVQPGRSLRLSCVVS |
| | mAb_HC HV huCCR8 | GFNFSNNGMHWVRQAPGKGLEWVA |
| | | VISNDGSNKYYADSVRGRFTISRDNS |
| | | KNTLYLQMNSLRAEDTAVYSCAKV |
| | | YYGSGIYYKNNYYYGMDVWGQGTT |
| | | VTVSS |
| 979 | Hu anti-huCCR8 LIBC317977-1 HuIgG1z | SYELTQPLSVSVALGQTARITCGGNN |
| | mAb_LC LV huCCR8 | IGSKNVHWYQQKAGQAPVQVIYRNS |
| | | NRPSGIPERFSGSNSGNTATLTISRAQ |
| | | AGDEADYYCQVWDSSTVVFGGGTK |
| | | LTVLG |
| 980 | Hu anti-huCCR8 LIBC317977-1 HuIgG1z | QVQLVESGGGVVQPGRSLRLSCAAS |
| | mAb_HC HV huCCR8 | GFNFNTYGMHWVRQAPGKGLEWVA |
| | | VISYDGSNKYYADSVKGRFTISRDNS |
| | | KSTLYLQMNSLRAEDTAVYYCARVY |
| | | YGSGSYYKKNYYYGMDVWGQGTT |
| | | VTVSS |
| 981 | Hu anti-huCCR8 LIBC318774-1 HuIgG1z | SYELTQPLSVSVALGQTARITCGGNN |
| | mAb_LC LV huCCR8 | IGGKNVHWYQQKPGQAPVLVIYRDS |
| | | NRPSGIPERFSGSKSGNTATLTISRAQ |
| | | AGDESDYYCQVWDSSTVVFGGGTTL |
| | | TVLG |
| 982 | Hu anti-huCCR8 LIBC318774-1 HuIgG1z | QVQVVESGGGVVQPGRSLRLSCAAS |
| | mAb_HC HV huCCR8 | GFTLSSYGFHWVRQTPGKGLEWVAV |
| | | ISYDGSNKYYADSVKGRFTISRDNSK |
| | | NTLYLQMNSLRGEDTAVYYCARVY |
| | | YGSGTYYKNRYYYGMDVWGQGTT |
| | | VTVSS |
| 983 | Hu anti-huCCR8 LIBC319840-1 HuIgG1z | SYELTQPLSVSEALGQTARITCGGNNI |
| | mAb_LC LV huCCR8 | GSKNVHWYQQKPGQAPVLVIYRDSN |
| | | RPSGIPERFSGSKSGNTATLTISRAQA |
| | | GDEADYYCQVWDSSTVVFGGGTKV |
| | | TVLG |
| 984 | Hu anti-huCCR8 LIBC319840-1 HuIgG1z | QVQLVESGGGVVQPGRSLRLSCVVS |
| | mAb_HC HV huCCR8 | GFNFINNGMHWVRQAPGKGLDWVA |
| | | VISNDGSNKYYPDSVKGRFTISRDNS |
| | | KNTLYLQMNSLRAEDSAVYYCAKV |
| | | YYGSGNYYKNNYYYGMDVWGQGT |
| | | TVTVSS |
| 985 | Hu anti-huCCR8 LIBC320212-1 HuIgG1z | SYELTQPLSVSVALGQTARITCEGNNI |
| | mAb_LC LV huCCR8 | GSQNVHWYQQKPGQAPVLVMYRDS |
| | | NRPSGIPERFSGSKSGNTATLAISRAQ |
| | | AGDESDYYCQVWDGSAVVFGGGTT |
| | | LTVLG |
| 986 | Hu anti-huCCR8 LIBC320212-1 HuIgG1z | QMQVVESGGGVVQPGRSLRLSCAAS |
| | mAb_HC HV huCCR8 | GFTFSSSGMHWVRQAPGKGLEWVA |
| | | VISHDGSNKYYADSVKGRFTISRDNS |
| | | KNTLYLQMNSLGGEDTAVYYCAKV |
| | | YYGSGIYYKNRYYYGMDVWGQGTT |
| | | VIVSS |
| 987 | Hu anti-huCCR8 LIBC320384-1 HuIgG1z | SYELTQPLSVSVALGQTARITCGGHN |
| | mAb_LC LV huCCR8 | IGSKGVHWYQQKPGQAPVLVIYRNS |
| | | NRPSGIPERFSGSNSGNTATLTISRAQ |
| | | AGDEADYYCQVWDSSTVVFGGGTE |
| | | LTVLG |
| 988 | Hu anti-huCCR8 LIBC320384-1 HuIgG1z | QVQLVESGGGVAQPGRSLRLSCAAS |
| | mAb_HC HV huCCR8 | GFNFSDCGMHWVRQAPGKGLEWVA |
| | | VISYDGGNKYYADSVKGRFTISRDDS |
| | | KNTLYLQTDSLRTEDTAVYYCAKVY |
| | | YGSGIYYKNRYYYGMDVWGQGTTV |
| | | TVSS |
| 989 | Hu anti-huCCR8 LIBC320689-1 HuIgG1z | SYELTQPLSVSVALGQTGRITCGGNN |
| | mAb_LC LV huCCR8 | IGSKNVHWYQQKPGQAPVLVIYRSS |
| | | NRPSGIPERFSGSNSGNTATLTISRAQ |
| | | AGDESDYYCQIWDSSTVVFGGGTKL |
| | | TVLG |
| 990 | Hu anti-huCCR8 LIBC320689-1 HuIgG1z | QVQVVESGGGVVQPGRSLRLSCAAS |
| | mAb_HC HV huCCR8 | GFTFSSYGMHWVRQAPGKGLEWVA |
| | | VISFDGNNKYYADSVKGRFTISRDNS |
| | | KNTLYLQMNSLRGEDTAVYYCARV |
| | | YYGSGSYYKNRYYYGMDVWGQGT |
| | | TVTVST |
| 991 | Hu anti-huCCR8 LIBC321408-1 HuIgG1z | SYELTQPLSVSVALGQTARITCGGNN |
| | mAb_LC LV huCCR8 | IGSKNVHWYQQRPGQAPVLVIYRDS |
| | | NRPSGIPERLSGSKAGNTATLTISRAH |
| | | AGDEADYYCQVWDSSTVVFGGGTE |
| | | LTVQG |
| 992 | Hu anti-huCCR8 LIBC321408-1 HuIgG1z | QVQLVESGGGVVQPGRSLRLSCAVS |
| | mAb_HC HV huCCR8 | GFTFSSNGMHWVRQAPGKGLEWVA |
| | | VISNDGSNKYYGDSVKGRFTISRDNS |
| | | KNTLYLQMNSLRAEDTAVYYCAKV |
| | | YYGSGIYYRNNYYYGMDVWGQGTT |
| | | VTVSS |
| 993 | Hu anti-huCCR8 LIBC321824-1 HuIgG1z | SYELTQPLSVSVALGQTARITCGGNN |
| | mAb_LC LV huCCR8 | IGSKNVHWYQQKPGQAPILVIYRNTN |
| | | RPSGIPERFSGSNSGNTATLTISRAQV |
| | | GDESDYFCQVWDSSTVVFGGGTKLT |
| | | VLG |
| 994 | Hu anti-huCCR8 LIBC321824-1 HuIgG1z | QVQVVESGGGVVQPGRSLRLSCGAS |
| | mAb_HC HV huCCR8 | GFTFSGYGMHWVRQAPGKGLEWVA |
| | | VISYDGSNKYYADSVKGRFPISRDNS |
| | | KNTLYLQMNSLRGEDTAVYYCARV |
| | | YYGSGIYYKNRYYYGMDVWGQGTT |
| | | VAVSS |
| 995 | Hu anti-huCCR8 LIBC321845-1 HuIgG1z | SYELTQPLSVSVALGQTARITCGGNN |
| | mAb_LC LV huCCR8 | IGSKNVHWYQQKPGQAPILVIYRNTN |
| | | RPSGIPERFSGSNSGNTATLTISRAQV |
| | | GDESDYFCQVWDSSTVVFGGGTKLT |
| | | VLG |
| 996 | Hu anti-huCCR8 LIBC321845-1 HuIgG1z | QVQVVESGGGVVQPGRSLRLSCGAS |
| | mAb_HC HV huCCR8 | GFTFSGYGMHWVRQAPGKGLEWVA |
| | | VISYDGSNKYYADSVKGRFTISRDNS |
| | | KNTLYLQMNSLRGEDTAVYYCARV |
| | | YYGSGIYYKNRYYYGMDVWGQGTT |
| | | VAVSS |
| 997 | Hu anti-huCCR8 LIBC322176-1 HuIgG1z | SYDLTQPLSVSVALGQTARITCGGNN |
| | mAb_LC LV huCCR8 | IGDKNVHWYQQKPGQAPVLVIYRNN |
| | | VRPSGIPERFSGSNSGNTATLTISRAQ |
| | | AGDEADYYCQVWDSSTVVFGGGTK |
| | | LTVLG |
| 998 | Hu anti-huCCR8 LIBC322176-1 HuIgG1z | QVQLVESGGGVVQPGRSLRLSCAAS |
| | mAb_HC HV huCCR8 | GLNFSNFGMHWVRQAPGKGLDWVA |
| | | VISYDGGNKYYADSVKGRFTVSRDN |
| | | SKNTLFLQMNSLRAEDTALYYCAKV |
| | | YYGSGSYYKKRYYYGMDVWGQGT |
| | | TVTVSS |
| 999 | Hu anti-huCCR8 LIBC323412-1 HuIgG1z | SYELTQPLSVSVALGQTARITCGGNN |
| | mAb_LC LV huCCR8 | IGSKNVHWYQQKPGQAPVLVIYRDS |
| | | NRPSGIPERFSGSKSGNTATLTISRAQ |
| | | AGDEADYYCQVWDSSTVVFGGGAK |
| | | LTVLG |
| 1000 | Hu anti-huCCR8 LIBC323412-1 HuIgG1z | QVQLVESGGGVVQPGRSLRLSCAAS |
| | mAb_HC HV huCCR8 | GFNFSSCGMHWVRQAPGKGLEWVA |
| | | VISYDGTNKYYADSVKGRFTISRDNS |
| | | KNTLYLQMNSLRAEDTAVYYCAKV |
| | | YYGSGIYYKKNYYYGMDVWGQGTT |
| | | VTVSS |
| 1001 | huCCR8_32360_huIgG1z mAb_HC HV | EVQLVESGGGLVKPGGSLRLSCAAS |
| | huCCR8 | GFTFSNARMGWVRQAPGKGLEWVG |
| | | RIKSKTEGGTRDYAAPVKGRFTISRD |
| | | DSKNTLYLQMNSLKTEDTAVYYCTS |
| | | YSGVWGQGTMVTVSS |
| 1002 | huCCR8_32360_huIgG1z mAb_LC LV | DIVMTQSPDSLAVSLGERATINCKSS |
| | huCCR8 | QSVLYSSNNKNYLAWYHQKPGQSPK |
| | | LLISWASTRESGVPDRFSGSGSGTDFT |
| | | LTINSLQAEDVAVYYCQQYYSIPITFG |
| | | GGTKVEIKR |
| 1003 | huCCR8_32360_huIgG1z | ASTKGPSVFPLAPSSKSTSGGTAALG |
| | mAb(LC: K38R) HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1004 | huCCR8_32360_huIgG1z | TVAAPSVFIFPPSDEQLKSGTASVVCL |
| | mAb(LC: K38R)_LC Constant | LNNFYPREAKVQWKVDNALQSGNS |
| | | QESVTEQDSKDSTYSLSSTLTLSKAD |
| | | YEKHKVYACEVTHQGLSSPVTKSFN |
| | | RGEC |
| 1005 | anti- | ASTKGPSVFPLAPSSKSTSGGTAALG |
| | huCCR8_44379(VH: D72S, VL: N67A_S68A_ | CLVKDYFPEPVTVSWNSGALTSGVH |
| | M99G_W109F_S111A)_huIgG1z (mAb)_HC | TFPAVLQSSGLYSLSSVVTVPSSSLGT |
| | Constant | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1006 | anti- | QPKAAPSVTLFPPSSEELQANKATLV |
| | huCCR8_44379(VH: D72S, VL: N67A_S68A_ | CLISDFYPGAVTVAWKADSSPVKAG |
| | M99G_W109F_S111A)_huIgG1z (mAb)_LC | VETTTPSKQSNNKYAASSYLSLTPEQ |
| | Constant | WKSHRSYSCQVTHEGSTVEKTVAPT |
| | | ECS |
| 1007 | anti- | ASTKGPSVFPLAPSSKSTSGGTAALG |
| | huCCR8_44379(VH: D61A_D72A, VL: N67Q_ | CLVKDYFPEPVTVSWNSGALTSGVH |
| | M99E_W109F_S111A)_huIgG1z | TFPAVLQSSGLYSLSSVVTVPSSSLGT |
| | (mAb)_HC Constant | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1008 | anti- | QPKAAPSVTLFPPSSEELQANKATLV |
| | huCCR8_44379(VH: D61A_D72A, VL: N67Q_ | CLISDFYPGAVTVAWKADSSPVKAG |
| | M99E_W109F_S111A)_huIgG1z | VETTTPSKQSNNKYAASSYLSLTPEQ |
| | (mAb)_LC Constant | WKSHRSYSCQVTHEGSTVEKTVAPT |
| | | ECS |
| 1009 | anti- | ASTKGPSVFPLAPSSKSTSGGTAALG |
| | huCCR8_44379(VH: D61S, VL: N67Q_M99G | CLVKDYFPEPVTVSWNSGALTSGVH |
| | _W109F_S111A)_huIgG1z (mAb)_HC | TFPAVLQSSGLYSLSSVVTVPSSSLGT |
| | Constant | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1010 | anti- | QPKAAPSVTLFPPSSEELQANKATLV |
| | huCCR8_44379(VH: D61S, VL: N67Q_M99G | CLISDFYPGAVTVAWKADSSPVKAG |
| | _W109F_S111A)_huIgG1z (mAb)_LC | VETTTPSKQSNNKYAASSYLSLTPEQ |
| | Constant | WKSHRSYSCQVTHEGSTVEKTVAPT |
| | | ECS |
| 1011 | Hu anti-huCCR8 LIBC315615-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1012 | Hu anti-huCCR8 LIBC315615-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1013 | Hu anti-huCCR8 LIBC317152-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1014 | Hu anti-huCCR8 LIBC317152-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1015 | Hu anti-huCCR8 LIBC317471-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1016 | Hu anti-huCCR8 LIBC317471-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1017 | Hu anti-huCCR8 LIBC317977-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1018 | Hu anti-huCCR8 LIBC317977-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1019 | Hu anti-huCCR8 LIBC318774-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1020 | Hu anti-huCCR8 LIBC318774-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1021 | Hu anti-huCCR8 LIBC319840-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1022 | Hu anti-huCCR8 LIBC319840-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1023 | Hu anti-huCCR8 LIBC320212-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1024 | Hu anti-huCCR8 LIBC320212-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1025 | Hu anti-huCCR8 LIBC320384-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1026 | Hu anti-huCCR8 LIBC320384-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1027 | Hu anti-huCCR8 LIBC320689-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1028 | Hu anti-huCCR8 LIBC320689-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1029 | Hu anti-huCCR8 LIBC321408-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1030 | Hu anti-huCCR8 LIBC321408-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1031 | Hu anti-huCCR8 LIBC321824-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1032 | Hu anti-huCCR8 LIBC321824-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1033 | Hu anti-huCCR8 LIBC321845-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1034 | Hu anti-huCCR8 LIBC321845-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1035 | Hu anti-huCCR8 LIBC322176-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1036 | Hu anti-huCCR8 LIBC322176-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1037 | Hu anti-huCCR8 LIBC323412-1 HuIgG1z | QPKAAPSVTLFPPSSEELQANKATLV |
| | mAb_LC Constant | CLISDFYPGAVTVAWKADSSPVKAG |
| | | VETTTPSKQSNNKYAASSYLSLTPEQ |
| | | WKSHRSYSCQVTHEGSTVEKTVAPT |
| | | ECS |
| 1038 | Hu anti-huCCR8 LIBC323412-1 HuIgG1z | ASTKGPSVFPLAPSSKSTSGGTAALG |
| | mAb_HC Constant | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1039 | huCCR8_32360_huIgG1z mAb_HC Constant | ASTKGPSVFPLAPSSKSTSGGTAALG |
| | | CLVKDYFPEPVTVSWNSGALTSGVH |
| | | TFPAVLQSSGLYSLSSVVTVPSSSLGT |
| | | QTYICNVNHKPSNTKVDKKVEPKSC |
| | | DKTHTCPPCPAPELLGGPSVFLFPPKP |
| | | KDTLMISRTPEVTCVVVDVSHEDPEV |
| | | KFNWYVDGVEVHNAKTKPREEQYN |
| | | STYRVVSVLTVLHQDWLNGKEYKC |
| | | KVSNKALPAPIEKTISKAKGQPREPQ |
| | | VYTLPPSREEMTKNQVSLTCLVKGF |
| | | YPSDIAVEWESNGQPENNYKTTPPVL |
| | | DSDGSFFLYSKLTVDKSRWQQGNVF |
| | | SCSVMHEALHNHYTQKSLSLSPGK |
| 1040 | huCCR8_32360_huIgG1z mAb_LC Constant | TVAAPSVFIFPPSDEQLKSGTASVVCL |
| | | LNNFYPREAKVQWKVDNALQSGNS |
| | | QESVTEQDSKDSTYSLSSTLTLSKAD |
| | | YEKHKVYACEVTHQGLSSPVTKSFN |
| | | RGEC |
| 1041 | MPK20298-A4_SCFV | QVQLVESGGGVVQPGRSLRLSCVVS |
| | | GFNFSNNGMHWVRQAPGKGLEWVA |
| | | VISNDGSNKYYADSVKGRFTISRDNS |
| | | KNTLYLQMNSLRTEDTAVYYCAKV |
| | | YYGSGIYYKNRNYYGMDVWGQGTT |
| | | VTVSSGGGGSGGGGSGGGGSSYELT |
| | | QPPSVSVALGQTARITCGGNNIGSQN |
| | | VHWYQQKPGQAPVLVIYRDSNRPSG |
| | | IPDRFSGSKSGNTATLTISRAQAGDEA |
| | | DYYCQVWDSSTVVFGGGTKLTVL |
| 1042 | MPK20299-D2_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | PPSVSVALGQTARITCGGHNIGSKGV |
| | | HWYQQKPGQAPVLVIYRNSNRPSGIP |
| | | ERFSGSNSGNTATLTITRAQAGDEAD |
| | | YYCQVWDSSTVVFGGGTKLTVL |
| 1043 | MPK20299-F11_SCFV | QVQLVESGGGVVQPGRSLRLSCAPS |
| | | GFNFSNYGMHWVRQAPGKGLEWVA |
| | | VISYDGSNKYYADSVKGRFTISRDNS |
| | | KNTLFLQMNSLRAEDTAVYFCARVY |
| | | YGSGSYYKKRYYYGMDVWGQGTT |
| | | VTVSSGGGGSGGGGSGGGGSSYELT |
| | | QPPSVSVALGQTARITCGGNNIGSQN |
| | | VHWYQQKPGQAPVLVIYRDSNRPSG |
| | | IPERFSGSKSGNTATLTISRAQAGDEA |
| | | DYYCQVWDSSTVVFGGGTQLTVL |
| 1044 | MPK20298-H6_SCFV | QVQLVESGGGVVQPGRSLRLSCAAS |
| | | GFTFSSSGMHWVRQAPGKGLEWVA |
| | | VISYDGTNKYYADSVKGRFTISRDNS |
| | | KNTLYLQMNSLRAEDTAVYYCAKV |
| | | YYGSGIYYKNRYYYGMDVWGQGTT |
| | | VTVSSGGGGSGGGGSGGGGSSYELT |
| | | QPPSVSVALGQTARITCGGHNIGSKG |
| | | VHWYQQKPGQAPVLVIYRNSNRPSG |
| | | IPERFSGSNSGNTATLTISRAQAGDEA |
| | | DYYCQVWDSSTVVFGGGTQLTVL |
| 1045 | MPK20297-A4_SCFV | QVQLVESGGGVVQPGRSLRLSCAVS |
| | | GFNFSNYGMHWVRQVPGRGLDWVA |
| | | VISNDGSNKYYADSVKGRFTISRDNS |
| | | KNTLYLQMDSLRTEDTAVYYCAKV |
| | | YYGSGIYYKKRYYYGMDVWGQGTT |
| | | VTVSSGGGGSGGGGSGGGGSSYELT |
| | | QPPSVSVALGQTARITCGGHNIGSQN |
| | | VHWYQQKPGQAPVLVIYRDSNRPSG |
| | | IPERFSGSKSGNTATLTISRAQAGDEA |
| | | DYYCQVWDSSTVVFGGGTQLTVL |
| 1046 | MPK20299-H8_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | PPSVSVAPGQTARITCGGNNIGSKNV |
| | | HWYQQKAGQAPVQVIYRNSNRPSGI |
| | | PARFSGSNSGNTATLTISRAQAGDEA |
| | | DYYCQVWDSSTVVFGGGTKLTVL |
| 1047 | MPK20300-C11_SCFV | QVQLVESGGGVVQPGRSLRLSCAAS |
| | | GFTFSSYGMHWVRQAPGKGLEWVA |
| | | VISYDGSNKYYADSVKGRFTISRDNS |
| | | KNTLYLQMNSLRGEDTAVYYCARV |
| | | YYGSGSYYKNRYYYGMDVWGQGT |
| | | TVTVSSGGGGSGGGGSGGGGSSYEL |
| | | TQPPSVSVAPGQTARIPCGGNNIGSK |
| | | NVHWYQQKPGQAPVLVIYRDINRPS |
| | | GIPERFSGSNSGNTATLTISRAQAGDE |
| | | ADYYCQVWDSSVVFGGGTKLTVL |
| 1048 | MPK20298-B1_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | PPSVSVALGQTARLTCEGNNIGSKNV |
| | | HWYQQKPGQAPVLVIYRNSNRPSGIP |
| | | ERFSGSNSGNTATLTISRVQAGDEAD |
| | | YYCQAWDSSTVVFGGGTQLTVL |
| 1049 | MPK20297-E5_SCFV | QVQLVESGGGLVKPGGSLRLSCAVS |
| | | GFNFSNNGMHWVRQAPGKGLEWVA |
| | | VISYDGSNKYYTDSVKGRFTISRDNS |
| | | KNTLYLQMNSLRTEDTAVYYCAKV |
| | | YYGSGIYYKKRYYYGMDVWGQGTT |
| | | VTVSSGGGGSGGGGSGGGGSSYELT |
| | | QPLSVSEALGQTARITCGGNNIGSKN |
| | | VHWYQQKPGQAPVLVIYRDSNRPSG |
| | | IPERFSGSNSGNAATLTISRVEAGDEA |
| | | DYYCQVWDSSSDHVVFGGGTQLTV |
| | | L |
| 1050 | MPK20299-A3_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | PPSVSVAPGQTARITCGGNNIGSKNV |
| | | HWYQQKPGQAPVLVIYRNSNRPSGIP |
| | | ERFSGSNSGNTATLTISGTQAMDEAD |
| | | YYCQAWDSSNVVFGGGTQLTVL |
| 1051 | MPK20297-B4_SCFV | QVQLVESGGGVVQPGRSLRLSCVVS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKNNYYYGMDVWGQGTT |
| | | VTVSSGGGGSGGGGSGGGGSSYELT |
| | | QPLSVSVALGQTARITCGGNNIGSQN |
| | | VHWYQQKPGQAPVLVIYRDSNRPSG |
| | | IPDRFSGSKSGNTATLTISRAQAGDEA |
| | | DYYCQVWDSSTVVFGGGTQLTVL |
| 1052 | MPK20298-F6_SCFV | QVQLVESGGGVVQPGRSLRLSCVVS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKNRYYYGMDVWGQGTT |
| | | VTVSSGGGGSGGGGSGGGGSSYELT |
| | | QPPSVSVAPGQTARITCGGNNIGSKN |
| | | VHWYQQKPGQAPVLVIYRDSNRPSG |
| | | IPERFSGSKSGTTATLTISRAQAGDEA |
| | | EYYCQVWDSSTVVFGGGTELTVL |
| 1053 | MPK20299-H3_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | PLSVSVALGQTARITCGGNNIGSKNV |
| | | HWYQQKPGQAPVLAIYRNSNRPSGIP |
| | | ERFTGSNSGNTATLTISRAQAGDESD |
| | | YYCQIWDSSTVVFGGGTKLTVL |
| 1054 | MPK20298-B9_SCFV | QVQLVESGGGVVQPGRSLRLSCAAS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKKNYYYGMDVWGQGTT |
| | | VTVSSGGGGSGGGGSGGGGSSYELT |
| | | QPPSVSVALGQTARISCGGNNIGSKN |
| | | VHWYQQKPGQAPVLVIYRDSNRPSG |
| | | IPERFSGSKSGTTATLTISRAQAGDEA |
| | | EYYCQVWDSSTVVFGGGTQLTVL |
| 1055 | MPK20299-E2_SCFV | QVQLVESGGGVVQPGRSLRLSCAVS |
| | | GFNFSNNGMHWVRQAPGKGLEWVA |
| | | VISYDGSNKYYTDSVKGRFTISRDNS |
| | | KNTLYLQMNSLRTEDTAVYYCAKV |
| | | YYGSGIYYKKRYYYGMDVWGQGTT |
| | | VTVSSGGGGSGGGGSGGGGSSYELT |
| | | QPPSVSVALGQTARITCEGNNIGSQN |
| | | VHWYQQKPGQAPVLVMYRDSNRPS |
| | | GIPERFSGSKSGNTATLAISRAQAGDE |
| | | SDYYCQVWDGSAVVFGGGTKLTVL |
| 1056 | MPK20299-D6_SCFV | QVQLVESGGGVVQPGRSLRLSCAAS |
| | | GFTFSSYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | PLSVSVALGQTARITCEGNNIGSQNV |
| | | HWYQQKPGQAPVLVMYRDSNRPSGI |
| | | PERFSGSKSGNTATLAISRAQAGDES |
| | | DYYCQVWDGSAVVFGGGTQLTVL |
| 1057 | MPK20299-A4_SCFV | QVQLVESGGGVVQPGRSLRLSCAAS |
| | | GFTFSNYGFHWVRQTPGKGLEWVA |
| | | VISYDGSNRYYADSVKGRFTISRDNS |
| | | KNTLYLQMNSLRGEDTALYYCARV |
| | | YYGSGTYYKNRYYYGMDVWGQGT |
| | | TVTVSSGGGGSGGGGSGGGGSSYEL |
| | | TQPPSVSVALGQTARITCGGHNIGSK |
| | | GVHWYQQKPGQAPVLVIYRNSNRPS |
| | | GIPERFSGSNSGNTATLTISGTQAMDE |
| | | ADYYCQAWDSGTVVFGGGTQLTVL |
| 1058 | MPK20300-G5_SCFV | QVQLVESGGGVVQPGRSLRLSCAAS |
| | | GFTFSNYGFHWVRQTPGKGLEWVA |
| | | VISYDGSNRYYADSVKGRFTISRDNS |
| | | KNTLYLQMNSLRGEDTALYYCARV |
| | | YYGSGTYYKNRYYYGMDVWGQGT |
| | | TVTVSSGGGGSGGGGSGGGGSSYEL |
| | | TQPPSVSVALGQTARITCGANNIGSK |
| | | NVHWYQQKPGQPPVLVIYRDFNRPS |
| | | GIPERFSASNSGNTATLTISRGQAGDE |
| | | ADYYCQVWDSSTGNVVFGGGTKLT |
| | | VL |
| 1059 | MPK20299-C3_SCFV | QVQLVESGGGVVQPGRSLRLSCAAS |
| | | GFIFSNYGFHWVRQTPGKGLEWVAV |
| | | ISYDGSNKYYADSVKGRFTISRDNSK |
| | | NTLYLQMNSLRGEDTAVYYCARVY |
| | | YGSGSYYKNRYYYGMDVWGQGTT |
| | | VTVSSGGGGSGGGGSGGGGSSYELT |
| | | QPPSVSVAPGQTARITCGGNNIGSKN |
| | | VHWYQQKPGQAPVLVIYRDSNRPSG |
| | | IPERFSGSKSGTTATLTISRAQAGDEA |
| | | DYYCQVWDSSTVVFGGGTELTVL |
| 1060 | MPK20299-B7_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | SSSVSVAPGQTARITCGGNNIGSKNV |
| | | HWYQQKPGQAPVLVIYRDSNRPSGIP |
| | | ERFSGSKSGTTATLTISRVEAGDEAD |
| | | YYCQVWDSSSAHVIFGGGTKLTVL |
| 1061 | MPK20299-A5_SCFV | QVQLVESGGGVVQPGRSLRLSCGAS |
| | | GFTFSGYGMHWVRQAPGKGLEWVA |
| | | VISYDGSNKYYADSVKGRFTISRDNS |
| | | KNTLYLQMNSLRGEDTAVYYCARV |
| | | YYGSGIYYKNRYYYGMDVWGQGTT |
| | | VTVSSGGGGSGGGGSGGGGSSYELT |
| | | QPPSGSVALGQTARITCGGNNLGSKN |
| | | VHWYQQKPGQAPVLVIYRNSNRPSG |
| | | IPERFSGSNSGNTATLTISRAQAGDEA |
| | | DYYCQVWDSSTVVFGGGTKLTVL |
| 1062 | MPK20299-D1_SCFV | QVQLVESGGGLVKPGGSLRLSCAAS |
| | | GFTFSNNGMHWVRQAPGKGLEWVA |
| | | VISYDGSNKYYADSVKGRFTISRDNS |
| | | KNTLYLQMNSLRAEDTAVYYCAKV |
| | | YYGSGIYYKNRYYYGMDVWGQGTT |
| | | VTVSSGGGGSGGGGSGGGGSSYELT |
| | | QPPSVSVALGQTARITCGGNRIGSKN |
| | | VHWYQQKPGQAPVLVIYRDSNRPSG |
| | | IPERFSGSKSGTTATLTISRAQAGDEA |
| | | EYYCQVWDSSTVVFGGGTKLTVL |
| 1063 | MPK20299-C5_SCFV | QVQLVESGGGVVQPGRSLRLSCAAS |
| | | GFTFSNYGFHWVRQTPGKGLEWVA |
| | | VISYDGSNRYYADSVKGRFTISRDNS |
| | | KNTLYLQMNSLRGEDTALYYCARV |
| | | YYGSGTYYKNRYYYGMDVWGQGT |
| | | TVTVSSGGGGSGGGGSGGGGSSYEL |
| | | TQLPSVSVALGQTARITCGGHNIGSK |
| | | GVHWYQQKPGQAPVLVIYRNSNRPS |
| | | GIPERFSGSNSGNTATLTISRAQAGDE |
| | | ADYYCQVWDSSTVVFGGGTELTVL |
| 1064 | MPK20299-B5_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKNRYYYGMDVWGQGTTV |
| | | TVSSGGGGSGGGGSGGGGSSYELTQ |
| | | PPSVSVALGQTARITCGGHNIGSKGV |
| | | HWYQQKPGQAPVLVIYRNSNRPSGIP |
| | | ERFSGSNSGNTATLTISRAQAGDEAD |
| | | YYCQVWDSSTVVFGGGTQLTVL |
| 1065 | MPK20299-G9_SCFV | QVQLVESGGDLVQPGRSLRLSCAAS |
| | | GFTFSNNGMHWVRQAPGKGLEWVA |
| | | VISNDGSNKYYADSVRGRFTISRDNS |
| | | KNTLYLQMNSLRAEDTAVYYCAKV |
| | | YYGSGIYYKNRYYYGMDVWGQGTT |
| | | VTVSSGGGGSGGGGSGGGGSSYELT |
| | | QPLSVSVALGQTARITCGGNNIGSKN |
| | | VHWYQQKPGQAPVLVIYRNSNRPSG |
| | | IPERFSGSNSGNTATLTLSRVQAGDE |
| | | ADYYCQVWDSSTVVFGGGTKLTVL |
| 1066 | MPK20299-G5_SCFV | QVQLVESGGGVVQPGRSLRLSCAVS |
| | | GFNFSNNGMHWVRQAPGKGLEWVA |
| | | VISNDGSNKYYADSVRGRFTISRDNS |
| | | KNTLYLQMDSLRTEDTAVYYCAKV |
| | | YYGSGIYYKNRYYYGMDVWGQGTT |
| | | VTVSSGGGGSGGGGSGGGGSSYELT |
| | | QPPSVSVALGQTARLTCEGNNIGSKN |
| | | VHWYQQKPGQAPVLVIYRDSNRPSG |
| | | IPERFSGSKSGNTATLAISRAQAGDES |
| | | DYYCQVWDSSAVVFGGGTKLTVL |
| 1067 | MPK20298-C10_SCFV | QVQLVESGGGVVQPGRSLRLSCAAS |
| | | GFTFSSSGMHWVRQAPGKGLEWVA |
| | | VISNDGSNKYYADSVKGRFTISRDNS |
| | | KNTLYLQMNSLRAEDTAVYYCAKV |
| | | YYGSGIYYKNNYYYGMDVWGQGTT |
| | | VTVSSGGGGGGGGSGGGGSSYELT |
| | | QPPSVSVALGQTARITCGGNNIGSKN |
| | | VHWYQQKPGQAPVLAIYRNSNRPSG |
| | | IPERFTGSNSGNTATLTISGTQAMDE |
| | | ADYYCQAWDSSTVVFGGGTKLTVL |
| 1068 | MPK20298-B5_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | PPSVSVALGQTARITCGGNNIGSQNV |
| | | HWYQQKPGQAPVLVIYRDSNRPSGIP |
| | | ERFSGSKSGNTATLAISRAQAGDESD |
| | | YYCQVWDSSAVVFGGGTQLTVL |
| 1069 | MPK20299-F2_SCFV | QVQLVESGGGVVQPGRSLRLSCAAS |
| | | GFTLSSSGMHWVRQAPGKGLEWVA |
| | | VISNDGSNKYYADSVKGRFTISRDDS |
| | | KNTLYLQMDSLRTEDTAVYYCAKV |
| | | YYGSGIYYKNRYYYGMDVWGQGTT |
| | | VTVSSGGGGSGGGGSGGGGSSYELT |
| | | QPPSVSVALGQTARISCGGNNIGSKN |
| | | VHWYQQKPGQAPVLVMYRDSNRPS |
| | | GIPERFSGSNSGNTATLTISGTQAMDE |
| | | ADYYCQAWDSGTVVFGGGTKLTVL |
| 1070 | MPK20298-D4_SCFV | QVQLVESGGGVVQPGRSLRLSCAAS |
| | | GFNFSNYGMHWVRQAPGKGLEWVA |
| | | VISYDGSNKYYADSVKGRFTISRDNS |
| | | KNTLYLQMNSLRAEDTAVYFCARVY |
| | | YGSGIYYKKRYYYGMDVWGQGTTV |
| | | TVSSGGGGSGGGGSGGGGSSYELTQ |
| | | PPSVSVALGQTARITCGGNNIGGKNV |
| | | HWYQQKPGQAPVLVIYRDSNRPSGIP |
| | | ERFSGSKSGNTATLTISRAQAGDESD |
| | | YYCQVWDSSTVVFGGGTQLTVL |
| 1071 | MPK20297-F5_SCFV | QVQLVESGGGVVQPGRSLRLSCVVS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKNNYYYGMDVWGQGTT |
| | | VTVSSGGGGSGGGGSGGGGSSYELT |
| | | QPLSVSVALGQTARITCGGNNIGSKN |
| | | VHWYQQKPGQAPVLVIYRNSNRPSG |
| | | IPERFSGSNSGNTATLTISRAQAGDEA |
| | | DYYCQVWDSSTVVFGGGTKLTVL |
| 1072 | MPK20299-D9_SCFV | QVQLVESGGGLVKPGGSLRLSCAAS |
| | | GFNFSRNGMHWVRQVPGRGLDWVA |
| | | VISNDGSNKYYADSVKGRFTISRDNS |
| | | KNTLYLQMNSLRAEDTAVYYCAKV |
| | | YYGSGIYYKNNYYYGMDVWGQGTT |
| | | VTVSSGGGGSGGGGSGGGGSSYELT |
| | | QPPSVSVALGQTARISCGGNNIESKN |
| | | VHWYQQKPGQAPVLVIYRDSNRPSG |
| | | IPERFSGSKSGTTATLTISRAQAGDEA |
| | | EYYCQVWDSSTVVFGGGTQLTVL |
| 1073 | huCCR8_32360_huIgG1z | EVQLVESGGGLVKPGGSLRLSCAAS |
| | mAb(LC: K38R)_HC | GFTFSNARMGWVRQAPGKGLEWVG |
| | | RIKSKTEGGTRDYAAPVKGRFTISRD |
| | | DSKNTLYLQMNSLKTEDTAVYYCTS |
| | | YSGVWGQGTMVTVSSASTKGPSVFP |
| | | LAPSSKSTSGGTAALGCLVKDYFPEP |
| | | VTVSWNSGALTSGVHTFPAVLQSSG |
| | | LYSLSSVVTVPSSSLGTQTYICNVNH |
| | | KPSNTKVDKKVEPKSCDKTHTCPPCP |
| | | APELLGGPSVFLFPPKPKDTLMISRTP |
| | | EVTCVVVDVSHEDPEVKFNWYVDG |
| | | VEVHNAKTKPREEQYNSTYRVVSVL |
| | | TVLHQDWLNGKEYKCKVSNKALPA |
| | | PIEKTISKAKGQPREPQVYTLPPSREE |
| | | MTKNQVSLTCLVKGFYPSDIAVEWE |
| | | SNGQPENNYKTTPPVLDSDGSFFLYS |
| | | KLTVDKSRWQQGNVFSCSVMHEAL |
| | | HNHYTQKSLSLSPGK |
| 1074 | huCCR8_32360_huIgG1z | DIVMTQSPDSLAVSLGERATINCKSS |
| | mAb(LC: K38R)_LC | QSVLYSSNNRNYLAWYHQKPGQSPK |
| | | LLISWASTRESGVPDRFSGSGSGTDFT |
| | | LTINSLQAEDVAVYYCQQYYSIPITFG |
| | | GGTKVEIKRTVAAPSVFIFPPSDEQLK |
| | | SGTASVVCLLNNFYPREAKVQWKVD |
| | | NALQSGNSQESVTEQDSKDSTYSLSS |
| | | TLTLSKADYEKHKVYACEVTHQGLS |
| | | SPVTKSFNRGEC |
| 1075 | anti- | QVQLVESGGGVVQPGRSLRLSCAAS |
| | huCCR8_44379(VH: D72S, VL: N67A_S68A_ | GFTFSNYGFHWVRQTPGKGLEWVA |
| | M99G_W109F_S111A)_huIgG1z (mAb)_HC | VISYDGSNRYYASSVKGRFTISRDNS |
| | | KNTLYLQMNSLRGEDTALYYCARV |
| | | YYGSGTYYKNRYYYGMDVWGQGT |
| | | TVTVSSASTKGPSVFPLAPSSKSTSGG |
| | | TAALGCLVKDYFPEPVTVSWNSGAL |
| | | TSGVHTFPAVLQSSGLYSLSSVVTVP |
| | | SSSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1076 | anti- | SYELTQPPSVSVALGQTARITCGGHN |
| | huCCR8_44379(VH: D72S, VL: N67A_S68A_ | IGSKGVHWYQQKPGQAPVLVIYRAA |
| | M99G_W109F_S111A)_huIgG1z (mAb)_LC | NRPSGIPERFSGSNSGNTATLTISGTQ |
| | | AGDEADYYCQAFDAGTVVFGGGTQ |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1077 | anti- | QVQLVESGGGVVQPGRSLRLSCAAS |
| | huCCR8_44379(VH: D61A_D72A, VL: N67Q_ | GFTFSNYGFHWVRQTPGKGLEWVA |
| | M99E_W109F_S111A)_huIgG1z | VISYAGSNRYYAASVKGRFTISRDNS |
| | (mAb) HC | KNTLYLQMNSLRGEDTALYYCARV |
| | | YYGSGTYYKNRYYYGMDVWGQGT |
| | | TVTVSSASTKGPSVFPLAPSSKSTSGG |
| | | TAALGCLVKDYFPEPVTVSWNSGAL |
| | | TSGVHTFPAVLQSSGLYSLSSVVTVP |
| | | SSSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1078 | anti- | SYELTQPPSVSVALGQTARITCGGHN |
| | huCCR8_44379(VH: D61A_D72A, VL: N67Q_ | IGSKGVHWYQQKPGQAPVLVIYRQS |
| | M99E_W109F_S111A)_huIgG1z | NRPSGIPERFSGSNSGNTATLTISGTQ |
| | (mAb)_LC | AEDEADYYCQAFDAGTVVFGGGTQ |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1079 | anti- | QVQLVESGGGVVQPGRSLRLSCAAS |
| | huCCR8_44379(VH: D61S, VL: N67Q_M99G | GFTFSNYGFHWVRQTPGKGLEWVA |
| | _W109F_S111A)_huIgG1z (mAb)_HC | VISYSGSNRYYADSVKGRFTISRDNS |
| | | KNTLYLQMNSLRGEDTALYYCARV |
| | | YYGSGTYYKNRYYYGMDVWGQGT |
| | | TVTVSSASTKGPSVFPLAPSSKSTSGG |
| | | TAALGCLVKDYFPEPVTVSWNSGAL |
| | | TSGVHTFPAVLQSSGLYSLSSVVTVP |
| | | SSSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1080 | anti- | SYELTQPPSVSVALGQTARITCGGHN |
| | huCCR8_44379(VH: D61S, VL: N67Q_M99G | IGSKGVHWYQQKPGQAPVLVIYRQS |
| | _W109F_S111A)_huIgG1z (mAb)_LC | NRPSGIPERFSGSNSGNTATLTISGTQ |
| | | AGDEADYYCQAFDAGTVVFGGGTQ |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1081 | Hu anti-huCCR8 LIBC315615-1 HuIgG1z | SYELTQPLSVSVALGQTARITCGGHN |
| | mAb_LC | IGSKGVHWYQQKPGQAPVLVIYRNS |
| | | NRPSGIPERFSGSNSGNTATLTISRAQ |
| | | AGDEADYYCQVWDISTVVFGGGTEL |
| | | TVLGQPKAAPSVTLFPPSSEELQANK |
| | | ATLVCLISDFYPGAVTVAWKADSSP |
| | | VKAGVETTTPSKQSNNKYAASSYLS |
| | | LTPEQWKSHRSYSCQVTHEGSTVEK |
| | | TVAPTECS |
| 1082 | Hu anti-huCCR8 LIBC315615-1 HuIgG1z | QVQLVESGGGVAQPGRSLRLSCAAS |
| | mAb_HC | GFNFSNCGMHWVRQAPGKGLEWVA |
| | | VISYDGGNKYHADSVKGRFTISRDDS |
| | | KNTLYLQMDSLRTEDTAVYYCAKV |
| | | YYGSGIYYKNRYYYGMDVWGQGTT |
| | | VTVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1083 | Hu anti-huCCR8 LIBC317152-1 HuIgG1z | SYELTQPLSVSVALGQTARITCGGHN |
| | mAb_LC | IGSKGVHWYQQKPGQAPVLVIYRNS |
| | | NRPSGIPERFSGSNSGKTATLTISRAQ |
| | | AGDEADYYCQVWDSSTVVFGGGTE |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1084 | Hu anti-huCCR8 LIBC317152-1 HuIgG1z | QVQLVESGGGVAQPGRSLRLSCAAS |
| | mAb_HC | GFNFSNCGMHWVRQAPGKGLEWVA |
| | | VISYDGGNKYYADSVKGRFTISRDDS |
| | | KNTLYLQMDSLRTEDTAVYYCAKV |
| | | YYGSGIYYKNRYYYGMDVWGQGTT |
| | | VTVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1085 | Hu anti-huCCR8 LIBC317471-1 HuIgG1z | SYELTQPLSVSVALGQTARITCGGNN |
| | mAb_LC | IGSKNVHWYQKRPGQAPVLVIYRDS |
| | | NRPSGIPERFSGSKSGNTATLTISRAQ |
| | | AGDEADYYCQVWDSNTVVFGGGTN |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1086 | Hu anti-huCCR8 LIBC317471-1 HuIgG1z | QVQLVESGGGVVQPGRSLRLSCVVS |
| | mAb_HC | GFNFSNNGMHWVRQAPGKGLEWVA |
| | | VISNDGSNKYYADSVRGRFTISRDNS |
| | | KNTLYLQMNSLRAEDTAVYSCAKV |
| | | YYGSGIYYKNNYYYGMDVWGQGTT |
| | | VTVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1087 | Hu anti-huCCR8 LIBC317977-1 HuIgG1z | SYELTQPLSVSVALGQTARITCGGNN |
| | mAb_LC | IGSKNVHWYQQKAGQAPVQVIYRNS |
| | | NRPSGIPERFSGSNSGNTATLTISRAQ |
| | | AGDEADYYCQVWDSSTVVFGGGTK |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1088 | Hu anti-huCCR8 LIBC317977-1 HuIgG1z | QVQLVESGGGVVQPGRSLRLSCAAS |
| | mAb_HC | GFNFNTYGMHWVRQAPGKGLEWVA |
| | | VISYDGSNKYYADSVKGRFTISRDNS |
| | | KSTLYLQMNSLRAEDTAVYYCARVY |
| | | YGSGSYYKKNYYYGMDVWGQGTT |
| | | VTVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1089 | Hu anti-huCCR8 LIBC318774-1 HuIgG1z | SYELTQPLSVSVALGQTARITCGGNN |
| | mAb_LC | IGGKNVHWYQQKPGQAPVLVIYRDS |
| | | NRPSGIPERFSGSKSGNTATLTISRAQ |
| | | AGDESDYYCQVWDSSTVVFGGGTTL |
| | | TVLGQPKAAPSVTLFPPSSEELQANK |
| | | ATLVCLISDFYPGAVTVAWKADSSP |
| | | VKAGVETTTPSKQSNNKYAASSYLS |
| | | LTPEQWKSHRSYSCQVTHEGSTVEK |
| | | TVAPTECS |
| 1090 | Hu anti-huCCR8 LIBC318774-1 HuIgG1z | QVQVVESGGGVVQPGRSLRLSCAAS |
| | mAb_HC | GFTLSSYGFHWVRQTPGKGLEWVAV |
| | | ISYDGSNKYYADSVKGRFTISRDNSK |
| | | NTLYLQMNSLRGEDTAVYYCARVY |
| | | YGSGTYYKNRYYYGMDVWGQGTT |
| | | VTVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1091 | Hu anti-huCCR8 LIBC319840-1 HuIgG1z | SYELTQPLSVSEALGQTARITCGGNNI |
| | mAb_LC | GSKNVHWYQQKPGQAPVLVIYRDSN |
| | | RPSGIPERFSGSKSGNTATLTISRAQA |
| | | GDEADYYCQVWDSSTVVFGGGTKV |
| | | TVLGQPKAAPSVTLFPPSSEELQANK |
| | | ATLVCLISDFYPGAVTVAWKADSSP |
| | | VKAGVETTTPSKQSNNKYAASSYLS |
| | | LTPEQWKSHRSYSCQVTHEGSTVEK |
| | | TVAPTECS |
| 1092 | Hu anti-huCCR8 LIBC319840-1 HuIgG1z | QVQLVESGGGVVQPGRSLRLSCVVS |
| | mAb_HC | GFNFINNGMHWVRQAPGKGLDWVA |
| | | VISNDGSNKYYPDSVKGRFTISRDNS |
| | | KNTLYLQMNSLRAEDSAVYYCAKV |
| | | YYGSGNYYKNNYYYGMDVWGQGT |
| | | TVTVSSASTKGPSVFPLAPSSKSTSGG |
| | | TAALGCLVKDYFPEPVTVSWNSGAL |
| | | TSGVHTFPAVLQSSGLYSLSSVVTVP |
| | | SSSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1093 | Hu anti-huCCR8 LIBC320212-1 HuIgG1z | SYELTQPLSVSVALGQTARITCEGNNI |
| | mAb_LC | GSQNVHWYQQKPGQAPVLVMYRDS |
| | | NRPSGIPERFSGSKSGNTATLAISRAQ |
| | | AGDESDYYCQVWDGSAVVFGGGTT |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1094 | Hu anti-huCCR8 LIBC320212-1 HuIgG1z | QMQVVESGGGVVQPGRSLRLSCAAS |
| | mAb_HC | GFTFSSSGMHWVRQAPGKGLEWVA |
| | | VISHDGSNKYYADSVKGRFTISRDNS |
| | | KNTLYLQMNSLGGEDTAVYYCAKV |
| | | YYGSGIYYKNRYYYGMDVWGQGTT |
| | | VIVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1095 | Hu anti-huCCR8 LIBC320384-1 HuIgG1z | SYELTQPLSVSVALGQTARITCGGHN |
| | mAb_LC | IGSKGVHWYQQKPGQAPVLVIYRNS |
| | | NRPSGIPERFSGSNSGNTATLTISRAQ |
| | | AGDEADYYCQVWDSSTVVFGGGTE |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1096 | Hu anti-huCCR8 LIBC320384-1 HuIgG1z | QVQLVESGGGVAQPGRSLRLSCAAS |
| | mAb_HC | GFNFSDCGMHWVRQAPGKGLEWVA |
| | | VISYDGGNKYYADSVKGRFTISRDDS |
| | | KNTLYLQTDSLRTEDTAVYYCAKVY |
| | | YGSGIYYKNRYYYGMDVWGQGTTV |
| | | TVSSASTKGPSVFPLAPSSKSTSGGTA |
| | | ALGCLVKDYFPEPVTVSWNSGALTS |
| | | GVHTFPAVLQSSGLYSLSSVVTVPSS |
| | | SLGTQTYICNVNHKPSNTKVDKKVE |
| | | PKSCDKTHTCPPCPAPELLGGPSVFLF |
| | | PPKPKDTLMISRTPEVTCVVVDVSHE |
| | | DPEVKFNWYVDGVEVHNAKTKPRE |
| | | EQYNSTYRVVSVLTVLHQDWLNGK |
| | | EYKCKVSNKALPAPIEKTISKAKGQP |
| | | REPQVYTLPPSREEMTKNQVSLTCLV |
| | | KGFYPSDIAVEWESNGQPENNYKTTP |
| | | PVLDSDGSFFLYSKLTVDKSRWQQG |
| | | NVFSCSVMHEALHNHYTQKSLSLSP |
| | | GK |
| 1097 | Hu anti-huCCR8 LIBC320689-1 HuIgG1z | SYELTQPLSVSVALGQTGRITCGGNN |
| | mAb_LC | IGSKNVHWYQQKPGQAPVLVIYRSS |
| | | NRPSGIPERFSGSNSGNTATLTISRAQ |
| | | AGDESDYYCQIWDSSTVVFGGGTKL |
| | | TVLGQPKAAPSVTLFPPSSEELQANK |
| | | ATLVCLISDFYPGAVTVAWKADSSP |
| | | VKAGVETTTPSKQSNNKYAASSYLS |
| | | LTPEQWKSHRSYSCQVTHEGSTVEK |
| | | TVAPTECS |
| 1098 | Hu anti-huCCR8 LIBC320689-1 HuIgG1z | QVQVVESGGGVVQPGRSLRLSCAAS |
| | mAb_HC | GFTFSSYGMHWVRQAPGKGLEWVA |
| | | VISFDGNNKYYADSVKGRFTISRDNS |
| | | KNTLYLQMNSLRGEDTAVYYCARV |
| | | YYGSGSYYKNRYYYGMDVWGQGT |
| | | TVTVSTASTKGPSVFPLAPSSKSTSGG |
| | | TAALGCLVKDYFPEPVTVSWNSGAL |
| | | TSGVHTFPAVLQSSGLYSLSSVVTVP |
| | | SSSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1099 | Hu anti-huCCR8 LIBC321408-1 HuIgG1z | SYELTQPLSVSVALGQTARITCGGNN |
| | mAb_LC | IGSKNVHWYQQRPGQAPVLVIYRDS |
| | | NRPSGIPERLSGSKAGNTATLTISRAH |
| | | AGDEADYYCQVWDSSTVVFGGGTE |
| | | LTVQGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1100 | Hu anti-huCCR8 LIBC321408-1 HuIgG1z | QVQLVESGGGVVQPGRSLRLSCAVS |
| | mAb_HC | GFTFSSNGMHWVRQAPGKGLEWVA |
| | | VISNDGSNKYYGDSVKGRFTISRDNS |
| | | KNTLYLQMNSLRAEDTAVYYCAKV |
| | | YYGSGIYYRNNYYYGMDVWGQGTT |
| | | VTVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1101 | Hu anti-huCCR8 LIBC321824-1 HuIgG1z | SYELTQPLSVSVALGQTARITCGGNN |
| | mAb_LC | IGSKNVHWYQQKPGQAPILVIYRNTN |
| | | RPSGIPERFSGSNSGNTATLTISRAQV |
| | | GDESDYFCQVWDSSTVVFGGGTKLT |
| | | VLGQPKAAPSVTLFPPSSEELQANKA |
| | | TLVCLISDFYPGAVTVAWKADSSPV |
| | | KAGVETTTPSKQSNNKYAASSYLSLT |
| | | PEQWKSHRSYSCQVTHEGSTVEKTV |
| | | APTECS |
| 1102 | Hu anti-huCCR8 LIBC321824-1 HuIgG1z | QVQVVESGGGVVQPGRSLRLSCGAS |
| | mAb_HC | GFTFSGYGMHWVRQAPGKGLEWVA |
| | | VISYDGSNKYYADSVKGRFPISRDNS |
| | | KNTLYLQMNSLRGEDTAVYYCARV |
| | | YYGSGIYYKNRYYYGMDVWGQGTT |
| | | VAVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1103 | Hu anti-huCCR8 LIBC321845-1 HuIgG1z | SYELTQPLSVSVALGQTARITCGGNN |
| | mAb_LC | IGSKNVHWYQQKPGQAPILVIYRNTN |
| | | RPSGIPERFSGSNSGNTATLTISRAQV |
| | | GDESDYFCQVWDSSTVVFGGGTKLT |
| | | VLGQPKAAPSVTLFPPSSEELQANKA |
| | | TLVCLISDFYPGAVTVAWKADSSPV |
| | | KAGVETTTPSKQSNNKYAASSYLSLT |
| | | PEQWKSHRSYSCQVTHEGSTVEKTV |
| | | APTECS |
| 1104 | Hu anti-huCCR8 LIBC321845-1 HuIgG1z | QVQVVESGGGVVQPGRSLRLSCGAS |
| | mAb_HC | GFTFSGYGMHWVRQAPGKGLEWVA |
| | | VISYDGSNKYYADSVKGRFTISRDNS |
| | | KNTLYLQMNSLRGEDTAVYYCARV |
| | | YYGSGIYYKNRYYYGMDVWGQGTT |
| | | VAVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1105 | Hu anti-huCCR8 LIBC322176-1 HuIgG1z | SYDLTQPLSVSVALGQTARITCGGNN |
| | mAb_LC | IGDKNVHWYQQKPGQAPVLVIYRNN |
| | | VRPSGIPERFSGSNSGNTATLTISRAQ |
| | | AGDEADYYCQVWDSSTVVFGGGTK |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1106 | Hu anti-huCCR8 LIBC322176-1 HuIgG1z | QVQLVESGGGVVQPGRSLRLSCAAS |
| | mAb_HC | GLNFSNFGMHWVRQAPGKGLDWVA |
| | | VISYDGGNKYYADSVKGRFTVSRDN |
| | | SKNTLFLQMNSLRAEDTALYYCAKV |
| | | YYGSGSYYKKRYYYGMDVWGQGT |
| | | TVTVSSASTKGPSVFPLAPSSKSTSGG |
| | | TAALGCLVKDYFPEPVTVSWNSGAL |
| | | TSGVHTFPAVLQSSGLYSLSSVVTVP |
| | | SSSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1107 | Hu anti-huCCR8 LIBC323412-1 HuIgG1z | SYELTQPLSVSVALGQTARITCGGNN |
| | mAb_LC | IGSKNVHWYQQKPGQAPVLVIYRDS |
| | | NRPSGIPERFSGSKSGNTATLTISRAQ |
| | | AGDEADYYCQVWDSSTVVFGGGAK |
| | | LTVLGQPKAAPSVTLFPPSSEELQAN |
| | | KATLVCLISDFYPGAVTVAWKADSS |
| | | PVKAGVETTTPSKQSNNKYAASSYLS |
| | | LTPEQWKSHRSYSCQVTHEGSTVEK |
| | | TVAPTECS |
| 1108 | Hu anti-huCCR8 LIBC323412-1 HuIgG1z | QVQLVESGGGVVQPGRSLRLSCAAS |
| | mAb_HC | GFNFSSCGMHWVRQAPGKGLEWVA |
| | | VISYDGTNKYYADSVKGRFTISRDNS |
| | | KNTLYLQMNSLRAEDTAVYYCAKV |
| | | YYGSGIYYKKNYYYGMDVWGQGTT |
| | | VTVSSASTKGPSVFPLAPSSKSTSGGT |
| | | AALGCLVKDYFPEPVTVSWNSGALT |
| | | SGVHTFPAVLQSSGLYSLSSVVTVPS |
| | | SSLGTQTYICNVNHKPSNTKVDKKV |
| | | EPKSCDKTHTCPPCPAPELLGGPSVFL |
| | | FPPKPKDTLMISRTPEVTCVVVDVSH |
| | | EDPEVKFNWYVDGVEVHNAKTKPR |
| | | EEQYNSTYRVVSVLTVLHQDWLNG |
| | | KEYKCKVSNKALPAPIEKTISKAKGQ |
| | | PREPQVYTLPPSREEMTKNQVSLTCL |
| | | VKGFYPSDIAVEWESNGQPENNYKT |
| | | TPPVLDSDGSFFLYSKLTVDKSRWQQ |
| | | GNVFSCSVMHEALHNHYTQKSLSLS |
| | | PGK |
| 1109 | huCCR8_32360_huIgG1z mAb_HC | EVQLVESGGGLVKPGGSLRLSCAAS |
| | | GFTFSNARMGWVRQAPGKGLEWVG |
| | | RIKSKTEGGTRDYAAPVKGRFTISRD |
| | | DSKNTLYLQMNSLKTEDTAVYYCTS |
| | | YSGVWGQGTMVTVSSASTKGPSVFP |
| | | LAPSSKSTSGGTAALGCLVKDYFPEP |
| | | VTVSWNSGALTSGVHTFPAVLQSSG |
| | | LYSLSSVVTVPSSSLGTQTYICNVNH |
| | | KPSNTKVDKKVEPKSCDKTHTCPPCP |
| | | APELLGGPSVFLFPPKPKDTLMISRTP |
| | | EVTCVVVDVSHEDPEVKFNWYVDG |
| | | VEVHNAKTKPREEQYNSTYRVVSVL |
| | | TVLHQDWLNGKEYKCKVSNKALPA |
| | | PIEKTISKAKGQPREPQVYTLPPSREE |
| | | MTKNQVSLTCLVKGFYPSDIAVEWE |
| | | SNGQPENNYKTTPPVLDSDGSFFLYS |
| | | KLTVDKSRWQQGNVFSCSVMHEAL |
| | | HNHYTQKSLSLSPGK |
| 1110 | huCCR8_32360_huIgG1z mAb_LC | DIVMTQSPDSLAVSLGERATINCKSS |
| | | QSVLYSSNNKNYLAWYHQKPGQSPK |
| | | LLISWASTRESGVPDRFSGSGSGTDFT |
| | | LTINSLQAEDVAVYYCQQYYSIPITFG |
| | | GGTKVEIKRTVAAPSVFIFPPSDEQLK |
| | | SGTASVVCLLNNFYPREAKVQWKVD |
| | | NALQSGNSQESVTEQDSKDSTYSLSS |
| | | TLTLSKADYEKHKVYACEVTHQGLS |
| | | SPVTKSFNRGEC |
| 1111 | MPK20298-A4_SCFV | CAGGTGCAGCTGGTGGAGTCTGGG |
| | | GGAGGCGTGGTCCAGCCTGGGAGG |
| | | TCCCTGAGACTCTCCTGTGTAGTCT |
| | | CTGGATTCAACTTCAGTAACAATGG |
| | | CATGCACTGGGTCCGCCAGGCTCCA |
| | | GGCAAGGGACTGGAGTGGGTGGCA |
| | | GTTATTTCAAATGATGGAAGTAATA |
| | | AATACTATGCAGACTCCGTGAAGG |
| | | GCCGATTCACCATCTCCAGAGACA |
| | | ATTCCAAGAACACGCTGTATCTACA |
| | | AATGAACAGCCTGAGAACTGAGGA |
| | | CACGGCTGTGTATTACTGTGCGAAA |
| | | GTTTACTATGGTTCGGGTATTTATT |
| | | ATAAAAACAGGAACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGAAACAACATTGGAAG |
| | | TCAAAATGTGCACTGGTACCAGCA |
| | | GAAGCCAGGCCAGGCCCCTGTGCT |
| | | GGTCATCTATAGGGATAGCAACCG |
| | | GCCCTCTGGGATCCCTGACCGATTC |
| | | TCTGGCTCCAAGTCGGGGAACACG |
| | | GCCACCCTGACCATCAGCAGAGCC |
| | | CAAGCCGGGGATGAGGCTGACTAT |
| | | TACTGTCAGGTGTGGGACAGCAGC |
| | | ACTGTGGTTTTCGGCGGAGGGACC |
| | | AAGCTGACCGTCCTA |
| 1112 | MPK20299-D2_SCFV | CAGGTGCAGCTGGTGGAGTCTGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGACACAACATTGGAAG |
| | | TAAAGGTGTGCACTGGTACCAGCA |
| | | GAAGCCAGGCCAGGCCCCTGTGCT |
| | | GGTCATCTATAGGAATAGCAACCG |
| | | GCCCTCTGGGATCCCTGAGCGATTC |
| | | TCTGGCTCCAACTCGGGGAACACG |
| | | GCCACCCTGACCATCACCAGAGCC |
| | | CAAGCCGGGGATGAGGCTGACTAT |
| | | TACTGTCAGGTGTGGGACAGCAGC |
| | | ACTGTGGTTTTCGGCGGAGGGACC |
| | | AAGCTGACCGTCCTA |
| 1113 | MPK20299-F11_SCFV | CAGGTGCAGCTGGTGGAGTCCGGG |
| | | GGAGGCGTGGTCCAGCCTGGGAGG |
| | | TCCCTGAGACTCTCCTGTGCACCCT |
| | | CTGGATTCAACTTCAGTAACTATGG |
| | | CATGCACTGGGTCCGCCAGGCTCCA |
| | | GGCAAGGGGCTGGAGTGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAAAACACGCTGTTTCTGCA |
| | | AATGAACAGCCTGAGAGCTGAGGA |
| | | CACGGCTGTGTATTTCTGTGCGAGA |
| | | GTTTACTATGGTTCGGGGAGTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGAAACAACATTGGAAG |
| | | TCAAAATGTGCACTGGTACCAGCA |
| | | GAAGCCAGGCCAGGCCCCTGTGCT |
| | | GGTCATCTATAGGGATAGCAACCG |
| | | GCCCTCTGGGATCCCTGAGCGATTC |
| | | TCTGGCTCCAAGTCGGGGAACACG |
| | | GCCACCCTGACCATCAGCAGAGCC |
| | | CAAGCCGGGGATGAGGCTGACTAT |
| | | TACTGTCAGGTGTGGGACAGCAGC |
| | | ACTGTGGTTTTCGGCGGAGGCACCC |
| | | AGCTGACCGTCCTA |
| 1114 | MPK20298-H6_SCFV | CAGGTGCAGCTGGTGGAGTCCGGG |
| | | GGAGGCGTGGTCCAGCCTGGGAGG |
| | | TCCCTGAGACTCTCCTGTGCAGCGT |
| | | CTGGATTCACCTTCAGTAGCTCTGG |
| | | CATGCACTGGGTCCGCCAGGCTCCA |
| | | GGCAAGGGGCTGGAGTGGGTGGCA |
| | | GTTATATCATATGATGGAACTAATA |
| | | AATACTATGCGGACTCCGTGAAGG |
| | | GCCGATTCACCATCTCCAGAGACA |
| | | ATTCCAAGAACACGCTGTATCTGCA |
| | | AATGAACAGCCTGAGAGCTGAGGA |
| | | CACGGCTGTGTATTACTGTGCGAAA |
| | | GTTTACTATGGTTCGGGTATTTATT |
| | | ATAAAAACAGGTACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGACACAACATTGGAAG |
| | | TAAAGGTGTGCACTGGTACCAGCA |
| | | GAAGCCAGGCCAGGCCCCTGTGCT |
| | | GGTCATCTATAGAAATAGCAACCG |
| | | GCCCTCTGGGATCCCTGAGCGATTC |
| | | TCTGGCTCCAACTCGGGGAACACG |
| | | GCCACCCTGACCATCAGCAGAGCC |
| | | CAAGCCGGGGATGAGGCTGACTAT |
| | | TACTGTCAGGTGTGGGACAGCAGC |
| | | ACTGTGGTTTTCGGCGGAGGCACCC |
| | | AGCTGACCGTCCTA |
| 1115 | MPK20297-A4_SCFV | CAGGTGCAGCTGGTGGAGTCTGGG |
| | | GGAGGCGTGGTCCAGCCTGGGAGG |
| | | TCCCTGAGACTCTCCTGTGCAGTCT |
| | | CTGGATTCAACTTCAGTAACTATGG |
| | | CATGCACTGGGTCCGCCAGGTTCCA |
| | | GGCAGGGGGCTAGATTGGGTGGCA |
| | | GTTATATCAAATGATGGAAGTAAT |
| | | AAATACTATGCAGACTCCGTGAAG |
| | | GGCCGATTCACCATTTCCAGAGACA |
| | | ATTCCAAGAACACACTGTATCTGCA |
| | | AATGGACAGCCTGAGAACTGAGGA |
| | | CACGGCTGTGTATTACTGTGCGAAA |
| | | GTTTACTATGGTTCGGGTATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGACACAACATTGGAAG |
| | | TCAAAATGTGCACTGGTACCAGCA |
| | | GAAGCCAGGCCAGGCCCCTGTGCT |
| | | GGTCATCTATAGGGATAGCAACCG |
| | | GCCCTCTGGGATCCCTGAGCGATTC |
| | | TCTGGCTCCAAGTCGGGGAACACG |
| | | GCCACCCTGACCATCAGCAGAGCC |
| | | CAAGCCGGGGATGAGGCTGACTAT |
| | | TACTGTCAGGTGTGGGACAGCAGC |
| | | ACTGTGGTTTTCGGCGGAGGCACCC |
| | | AGCTGACCGTCCTA |
| 1116 | MPK20299-H8_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGGA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCGGTGTCAGTGGC |
| | | CCCAGGACAGACGGCCAGGATTAC |
| | | CTGTGGGGGAAACAACATTGGAAG |
| | | TAAAAATGTGCACTGGTACCAGCA |
| | | GAAGGCAGGCCAGGCCCCTGTGCA |
| | | GGTCATCTATAGAAATAGCAACCG |
| | | GCCCTCTGGGATCCCTGCGCGATTC |
| | | TCTGGCTCCAACTCGGGGAACACG |
| | | GCCACCCTGACCATCAGCAGAGCC |
| | | CAGGCCGGGGATGAGGCTGACTAT |
| | | TACTGTCAGGTGTGGGACAGCAGC |
| | | ACTGTGGTTTTCGGCGGTGGGACCA |
| | | AGCTGACCGTCCTA |
| 1117 | MPK20300-C11_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCACCTTCAGTAGCTATGG |
| | | CATGCACTGGGTCCGCCAGGCTCCA |
| | | GGCAAGGGGCTGGAGTGGGTGGCA |
| | | GTTATATCATATGATGGAAGTAATA |
| | | AATACTATGCAGACTCCGTGAAGG |
| | | GCCGATTCACCATCTCCAGAGACA |
| | | ATTCCAAGAACACGCTGTATCTGCA |
| | | AATGAACAGCCTGAGAGGTGAGGA |
| | | CACGGCGGTGTATTACTGTGCGAG |
| | | AGTTTACTATGGTTCGGGGAGTTAT |
| | | TATAAAAACCGCTACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCGGTGTCAGTGGC |
| | | CCCAGGACAGACGGCCAGGATTCC |
| | | CTGTGGGGGAAACAACATTGGAAG |
| | | TAAAAATGTGCACTGGTACCAGCA |
| | | GAAGCCAGGCCAGGCCCCTGTACT |
| | | GGTCATCTATAGGGATATCAACCG |
| | | GCCCTCTGGGATCCCTGAGCGATTC |
| | | TCTGGCTCCAACTCGGGGAACACG |
| | | GCCACCCTGACCATCAGCAGAGCC |
| | | CAAGCCGGGGATGAGGCTGACTAT |
| | | TACTGTCAGGTGTGGGACAGCAGC |
| | | GTGGTATTCGGCGGAGGGACCAAG |
| | | CTGACCGTCCTC |
| 1118 | MPK20298-B1_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGCTTAC |
| | | CTGTGAGGGAAACAACATTGGAAG |
| | | TAAAAATGTGCACTGGTACCAGCA |
| | | GAAGCCAGGCCAGGCCCCTGTGCT |
| | | GGTCATCTATAGGAATAGCAACCG |
| | | GCCCTCTGGGATCCCTGAGCGATTC |
| | | TCTGGCTCCAACTCGGGGAACACG |
| | | GCCACCCTGACTATTAGCAGAGTCC |
| | | AAGCCGGGGATGAGGCTGACTATT |
| | | ACTGTCAGGCGTGGGACAGCAGCA |
| | | CTGTGGTATTCGGCGGAGGCACCC |
| | | AGCTGACCGTCCTA |
| 1119 | MPK20297-E5_SCFV | CAGGTGCAGCTGGTGGAGTCCGGG |
| | | GGAGGCCTGGTCAAGCCTGGGGGG |
| | | TCCCTGAGACTCTCCTGTGCAGTCT |
| | | CTGGATTCAACTTCAGTAACAATGG |
| | | CATGCACTGGGTCCGCCAGGCTCCA |
| | | GGCAAGGGGCTGGAGTGGGTGGCA |
| | | GTCATATCGTATGATGGAAGTAATA |
| | | AATACTATACAGACTCCGTGAAGG |
| | | GCCGATTCACCATCTCCAGAGACA |
| | | ATTCCAAGAACACGCTGTATCTGCA |
| | | AATGAACAGCCTGAGAACTGAGGA |
| | | CACGGCTGTGTATTACTGTGCGAAA |
| | | GTTTACTATGGTTCGGGTATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACTCTCAGTGTCAGAGGC |
| | | CCTGGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTACT |
| | | GGTCATCTATAGGGATAGCAACCG |
| | | GCCCTCAGGGATCCCTGAGCGATTC |
| | | TCTGGCTCCAACTCTGGGAACGCGG |
| | | CCACCCTGACCATCAGTAGGGTCG |
| | | AAGCCGGGGATGAGGCCGACTATT |
| | | ACTGTCAGGTGTGGGATAGTAGCA |
| | | GTGATCATGTGGTATTCGGCGGAG |
| | | GCACCCAGCTGACCGTCCTA |
| 1120 | MPK20299-A3_SCFV | CAGGTGCAGCTGGTGGAGTCTGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCGGTGTCAGTGGC |
| | | CCCAGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTACT |
| | | GGTCATCTATAGAAATAGCAACCG |
| | | GCCCTCTGGGATCCCTGAGCGATTC |
| | | TCTGGCTCCAACTCTGGGAACACAG |
| | | CCACTCTGACCATCAGCGGGACCC |
| | | AGGCTATGGATGAGGCTGACTATT |
| | | ACTGTCAGGCGTGGGACAGCAGCA |
| | | ATGTGGTATTCGGCGGAGGCACCC |
| | | AGCTGACCGTCCTA |
| 1121 | MPK20297-B4_SCFV | CAGGTGCAGCTGGTGGAGTCCGGG |
| | | GGAGGCGTGGTCCAGCCTGGGAGG |
| | | TCCCTGAGACTCTCCTGTGTAGTCT |
| | | CTGGATTCAACTTCAGTAGGAATGG |
| | | CATGCACTGGGTCCGCCAGGTTCCA |
| | | GGCAGGGGGCTAGATTGGGTGGCA |
| | | GTTATATCAAATGATGGAAGTAAT |
| | | AAATACTATGCAGACTCCGTGAAG |
| | | GGCCGATTCACCATCTCCAGAGAC |
| | | AATTCCAAGAACACGCTGTATCTGC |
| | | AAATGAACAGCCTGAGAGCTGAGG |
| | | ACACGGCTGTGTATTACTGTGCGAA |
| | | AGTTTACTATGGTTCGGGGATTTAT |
| | | TATAAAAATAACTACTATTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACTCTCAGTGTCAGTGGC |
| | | CCTGGGACAGACGGCCAGGATTAC |
| | | CTGTGGGGGAAACAACATTGGAAG |
| | | TCAAAATGTGCACTGGTACCAGCA |
| | | GAAGCCAGGCCAGGCCCCTGTGCT |
| | | GGTCATCTATAGGGATAGCAACCG |
| | | GCCCTCTGGGATCCCTGACCGATTC |
| | | TCTGGCTCCAAGTCGGGGAACACG |
| | | GCCACCCTGACCATCAGCAGAGCC |
| | | CAAGCCGGGGATGAGGCTGACTAT |
| | | TACTGTCAGGTGTGGGACAGCAGC |
| | | ACTGTGGTTTTCGGCGGAGGCACCC |
| | | AGCTGACCGTCCTA |
| 1122 | MPK20298-F6_SCFV | CAGGTGCAGCTGGTGGAGTCTGGG |
| | | GGAGGCGTGGTCCAGCCTGGGAGG |
| | | TCCCTGAGACTCTCCTGTGTAGTCT |
| | | CTGGATTCAACTTCAGTAGGAATGG |
| | | CATGCACTGGGTCCGCCAGGTTCCA |
| | | GGCAGGGGGCTAGATTGGGTGGCA |
| | | GTTATATCAAATGATGGAAGTAAT |
| | | AAATACTATGCAGACTCCGTGAAG |
| | | GGCCGATTCACCATCTCCAGAGAC |
| | | AATTCCAAGAACACGCTGTATCTGC |
| | | AAATGAACAGCCTGAGAGCTGAGG |
| | | ACACGGCTGTGTATTACTGTGCGAA |
| | | AGTTTACTATGGTTCGGGGATTTAT |
| | | TATAAAAACCGCTATTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCGGTGTCAGTGGC |
| | | CCCAGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTGCT |
| | | GGTCATCTATAGGGATAGCAACCG |
| | | GCCCTCTGGGATCCCTGAGCGATTC |
| | | TCTGGCTCCAAGTCGGGGACCACG |
| | | GCCACCCTGACCATCAGCAGAGCC |
| | | CAAGCCGGGGATGAGGCTGAGTAT |
| | | TACTGTCAGGTGTGGGACAGCAGC |
| | | ACTGTGGTTTTCGGCGGAGGGACC |
| | | GAGCTGACCGTCCTA |
| 1123 | MPK20299-H3_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CACAGCCACTCTCAGTGTCAGTGGC |
| | | CCTGGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTGCT |
| | | GGCCATCTATAGGAATAGCAACCG |
| | | GCCCTCTGGGATCCCTGAGCGATTC |
| | | ACTGGCTCCAACTCGGGGAACACG |
| | | GCCACCCTGACCATCAGCAGAGCC |
| | | CAAGCCGGGGATGAGTCTGACTAT |
| | | TACTGTCAAATATGGGACAGCAGC |
| | | ACTGTGGTATTCGGCGGAGGCACC |
| | | AAGCTGACCGTCCTA |
| 1124 | MPK20298-B9_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCAACTTCAGTAGGAATGG |
| | | CATGCACTGGGTCCGCCAGGTTCCA |
| | | GGCAGGGGGCTAGATTGGGTGGCA |
| | | GTTATATCAAATGATGGAAGTAAT |
| | | AAATACTATGCGGACTCCGTGAAG |
| | | GGCCGATTCACCATCTCCAGAGAC |
| | | AATTCCAAGAACACGCTGTATCTGC |
| | | AAATGAACAGCCTGAGAGCTGAGG |
| | | ACACGGCTGTGTATTACTGTGCGAA |
| | | AGTTTACTATGGTTCGGGTATTTAT |
| | | TATAAAAAGAACTACTACTACGGT |
| | | ATGGACGTCTGGGGCCAAGGGACC |
| | | ACGGTCACCGTCTCCTCAGGTGGTG |
| | | GTGGTTCTGGCGGCGGCGGCTCCG |
| | | GTGGTGGTGGTTCTTCATATGAGCT |
| | | GACTCAGCCACCCTCGGTGTCAGTG |
| | | GCCCTGGGACAGACGGCCAGGATT |
| | | TCCTGTGGGGGAAACAACATTGGA |
| | | AGTAAAAATGTGCACTGGTACCAG |
| | | CAGAAGCCAGGCCAGGCCCCTGTG |
| | | CTGGTCATCTATAGGGATAGCAACC |
| | | GGCCCTCTGGGATCCCTGAGCGATT |
| | | CTCTGGCTCCAAGTCGGGGACCAC |
| | | GGCCACCCTGACCATCAGCAGAGC |
| | | CCAAGCCGGGGATGAGGCTGAGTA |
| | | TTACTGTCAGGTGTGGGACAGCAG |
| | | CACTGTGGTTTTCGGCGGAGGCACC |
| | | CAGCTGACCGTCCTA |
| 1125 | MPK20299-E2_SCFV | CAGGTGCAGCTGGTGGAGTCCGGG |
| | | GGAGGCGTGGTCCAGCCTGGGAGG |
| | | TCCCTGAGACTCTCCTGTGCAGTCT |
| | | CTGGATTCAACTTCAGTAACAATGG |
| | | CATGCACTGGGTCCGCCAGGCTCCA |
| | | GGCAAGGGGCTGGAGTGGGTGGCA |
| | | GTCATATCGTATGATGGAAGTAATA |
| | | AATACTATACAGACTCCGTGAAGG |
| | | GCCGATTCACCATCTCCAGAGACA |
| | | ATTCCAAGAACACGCTGTATCTGCA |
| | | AATGAACAGCCTGAGAACTGAGGA |
| | | CACGGCTGTGTATTACTGTGCGAAA |
| | | GTTTACTATGGTTCGGGTATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGAGGGAAACAACATTGGAAG |
| | | TCAAAATGTGCACTGGTACCAGCA |
| | | GAAGCCAGGCCAGGCCCCTGTGCT |
| | | GGTCATGTATAGGGATAGCAACCG |
| | | GCCCTCTGGGATCCCTGAACGATTC |
| | | TCTGGCTCCAAGTCGGGGAACACG |
| | | GCCACCCTGGCCATCAGCAGAGCC |
| | | CAAGCCGGGGATGAGTCTGACTAT |
| | | TACTGTCAGGTGTGGGACGGCAGT |
| | | GCCGTGGTATTCGGCGGAGGGACC |
| | | AAGCTGACCGTCCTA |
| 1126 | MPK20299-D6_SCFV | CAGGTGCAGCTGGTGGAGTCTGGG |
| | | GGAGGCGTGGTCCAGCCTGGGAGG |
| | | TCCCTGAGACTCTCCTGTGCAGCGT |
| | | CTGGATTCACCTTCAGTAGCTATGG |
| | | CATGCACTGGGTCCGCCAGGCTCCA |
| | | GGCAAGGGGCTGGAGTGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACTCTCAGTGTCAGTGGC |
| | | CCTGGGACAGACGGCCAGGATTAC |
| | | CTGTGAGGGAAACAACATTGGAAG |
| | | TCAAAATGTGCACTGGTACCAGCA |
| | | GAAGCCAGGCCAGGCCCCTGTGCT |
| | | GGTCATGTATAGGGATAGCAACCG |
| | | GCCCTCTGGGATCCCTGAACGATTC |
| | | TCTGGCTCCAAGTCGGGGAACACG |
| | | GCCACCCTGGCCATCAGCAGAGCC |
| | | CAAGCCGGGGATGAGTCTGACTAT |
| | | TACTGTCAGGTGTGGGACGGCAGT |
| | | GCCGTGGTATTCGGCGGAGGCACC |
| | | CAGCTGACCGTCCTA |
| 1127 | MPK20299-A4_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCACCTTCAGTAACTATGG |
| | | CTTTCACTGGGTCCGCCAGACTCCA |
| | | GGCAAGGGGCTGGAGTGGGTGGCA |
| | | GTTATATCATATGATGGAAGTAATA |
| | | GATACTATGCAGACTCCGTGAAGG |
| | | GCCGATTCACCATCTCCAGAGACA |
| | | ATTCCAAGAACACGCTGTATCTCCA |
| | | AATGAACAGCCTGAGAGGTGAGGA |
| | | CACGGCGCTATATTACTGTGCGAGA |
| | | GTTTACTATGGTTCGGGGACTTATT |
| | | ATAAAAACCGCTACTACTACGGTAT |
| | | GGACGTCTGGGGCCAAGGGACCAC |
| | | GGTCACCGTCTCCTCAGGTGGTGGT |
| | | GGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGACACAACATTGGAAG |
| | | TAAAGGTGTGCACTGGTACCAGCA |
| | | GAAGCCAGGCCAGGCCCCTGTACT |
| | | GGTCATCTATAGAAATAGCAACCG |
| | | GCCCTCTGGGATCCCTGAGCGATTC |
| | | TCTGGCTCCAACTCTGGGAACACAG |
| | | CCACTCTGACCATCAGCGGGACCC |
| | | AGGCTATGGATGAGGCTGACTATT |
| | | ACTGTCAGGCGTGGGACAGCGGCA |
| | | CTGTGGTATTCGGCGGAGGCACCC |
| | | AGCTGACCGTCCTA |
| 1128 | MPK20300-G5_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCACCTTCAGTAACTATGG |
| | | CTTTCACTGGGTCCGCCAGACTCCA |
| | | GGCAAGGGGCTGGAGTGGGTGGCA |
| | | GTTATATCATATGATGGAAGTAATA |
| | | GATACTATGCAGACTCCGTGAAGG |
| | | GCCGATTCACCATCTCCAGAGACA |
| | | ATTCCAAGAACACGCTGTATCTCCA |
| | | AATGAACAGCCTGAGAGGTGAGGA |
| | | CACGGCGCTATATTACTGTGCGAGA |
| | | GTTTACTATGGTTCGGGGACTTATT |
| | | ATAAAAACCGCTACTACTACGGTAT |
| | | GGACGTCTGGGGCCAAGGGACCAC |
| | | GGTCACCGTCTCCTCAGGTGGTGGT |
| | | GGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGCAAACAACATTGGAAG |
| | | TAAAAATGTTCACTGGTACCAGCA |
| | | GAAGCCAGGCCAGCCCCCTGTGCT |
| | | GGTCATCTATAGAGATTTCAACCGG |
| | | CCCTCTGGGATCCCTGAGCGATTCT |
| | | CTGCCTCCAACTCGGGGAACACGG |
| | | CCACCCTGACCATCAGCAGAGGCC |
| | | AAGCCGGGGATGAGGCTGACTATT |
| | | ACTGTCAGGTGTGGGACAGCAGCA |
| | | CTGGGAATGTGGTATTCGGCGGAG |
| | | GGACCAAGCTGACCGTCCTA |
| 1129 | MPK20299-C3_SCFV | CAGGTGCAGCTGGTGGAGTCTGGG |
| | | GGAGGCGTGGTCCAGCCTGGGAGG |
| | | TCCCTGAGACTCTCCTGTGCAGCCT |
| | | CTGGATTCATCTTCAGTAACTATGG |
| | | CTTTCACTGGGTCCGCCAGACTCCA |
| | | GGCAAGGGGCTGGAGTGGGTGGCA |
| | | GTTATATCATATGATGGAAGTAATA |
| | | AATACTATGCAGACTCCGTGAAGG |
| | | GCCGATTCACCATCTCCAGAGACA |
| | | ATTCCAAGAACACGCTGTATCTGCA |
| | | AATGAACAGCCTGAGAGGTGAGGA |
| | | CACGGCGGTGTATTACTGTGCGAG |
| | | AGTTTACTATGGTTCGGGGAGTTAT |
| | | TATAAAAACCGCTACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCGGTGTCAGTGGC |
| | | CCCAGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTGCT |
| | | GGTCATCTATAGGGATAGCAACCG |
| | | GCCCTCTGGGATCCCTGAGCGATTC |
| | | TCTGGCTCCAAGTCGGGGACCACG |
| | | GCCACCCTGACCATCAGCAGAGCC |
| | | CAAGCCGGGGATGAGGCTGACTAT |
| | | TACTGTCAGGTGTGGGACAGCAGC |
| | | ACTGTGGTTTTCGGCGGAGGGACC |
| | | GAGCTGACCGTCCTA |
| 1130 | MPK20299-B7_SCFV | CAGGTGCAGCTGGTGGAGTCTGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGTCATCCTCGGTGTCAGTGGC |
| | | CCCAGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTGTT |
| | | GGTCATCTATAGGGATAGCAACCG |
| | | GCCCTCTGGGATCCCTGAGCGATTC |
| | | TCTGGCTCCAAGTCGGGGACCACG |
| | | GCCACCCTGACCATCAGCAGGGTC |
| | | GAAGCCGGGGATGAGGCCGACTAT |
| | | TACTGTCAGGTGTGGGATAGTAGTA |
| | | GTGCTCATGTGATATTCGGCGGAGG |
| | | GACCAAGCTGACCGTCCTA |
| 1131 | MPK20299-A5_SCFV | CAGGTGCAGCTGGTGGAGTCCGGG |
| | | GGAGGCGTGGTCCAGCCTGGGAGG |
| | | TCCCTGAGACTCTCCTGTGGAGCCT |
| | | CTGGATTCACCTTCAGTGGCTATGG |
| | | CATGCACTGGGTCCGCCAGGCTCCA |
| | | GGCAAGGGGCTGGAGTGGGTGGCA |
| | | GTTATATCATATGATGGAAGTAATA |
| | | AATACTATGCAGACTCCGTGAAGG |
| | | GCCGATTCACCATCTCAAGAGACA |
| | | ATTCCAAGAACACGCTGTATCTGCA |
| | | AATGAACAGCCTGAGAGGTGAGGA |
| | | CACGGCGGTGTATTACTGTGCGAG |
| | | AGTTTATTATGGTTCGGGGATTTAT |
| | | TATAAAAACCGCTACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGGGTCAGTGGC |
| | | CCTGGGACAGACGGCCAGGATCAC |
| | | CTGTGGGGGAAACAACCTTGGAAG |
| | | TAAAAATGTGCACTGGTACCAACA |
| | | GAAGCCAGGCCAGGCCCCTGTGCT |
| | | GGTCATCTATAGAAATAGCAACCG |
| | | GCCCTCTGGGATCCCTGAGCGATTC |
| | | TCTGGCTCCAACTCGGGGAACACG |
| | | GCCACCCTGACCATCAGCAGAGCC |
| | | CAGGCCGGGGATGAGGCTGACTAT |
| | | TACTGTCAGGTGTGGGACAGCAGC |
| | | ACTGTGGTATTCGGCGGTGGGACC |
| | | AAGCTGACCGTCCTA |
| 1132 | MPK20299-D1_SCFV | CAGGTGCAGCTGGTGGAGTCTGGG |
| | | GGAGGCCTGGTCAAGCCTGGGGGG |
| | | TCCCTGAGACTCTCCTGTGCAGCCT |
| | | CTGGATTCACCTTCAGTAACAATGG |
| | | CATGCACTGGGTCCGCCAGGCTCCA |
| | | GGCAAGGGGCTGGAGTGGGTGGCA |
| | | GTCATATCGTATGATGGAAGTAATA |
| | | AATACTATGCGGACTCCGTGAAGG |
| | | GCCGATTCACCATCTCCAGAGACA |
| | | ATTCCAAGAACACGCTGTATCTGCA |
| | | AATGAACAGCCTGAGAGCTGAGGA |
| | | CACGGCTGTGTATTACTGTGCGAAA |
| | | GTTTATTATGGTTCGGGGATTTATT |
| | | ATAAAAACAGGTATTACTACGGGA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGAAACAGAATTGGAAG |
| | | TAAAAATGTGCACTGGTACCAGCA |
| | | GAAGCCAGGCCAGGCCCCTGTGTT |
| | | GGTCATCTATAGGGATAGCAACCG |
| | | GCCCTCTGGGATCCCTGAGCGATTC |
| | | TCTGGCTCCAAGTCGGGGACCACG |
| | | GCCACCCTGACCATCAGCAGAGCC |
| | | CAAGCCGGGGATGAGGCTGAGTAT |
| | | TACTGTCAGGTGTGGGACAGCAGC |
| | | ACTGTGGTTTTCGGCGGAGGGACC |
| | | AAGCTGACCGTCCTA |
| 1133 | MPK20299-C5_SCFV | CAGGTGCAGCTGGTGGAGTCTGGG |
| | | GGAGGCGTGGTCCAGCCTGGGAGG |
| | | TCCCTGAGACTCTCCTGTGCAGCCT |
| | | CTGGATTCACCTTCAGTAACTATGG |
| | | CTTTCACTGGGTCCGCCAGACTCCA |
| | | GGCAAGGGGCTGGAGTGGGTGGCA |
| | | GTTATATCATATGATGGAAGTAATA |
| | | GATACTATGCAGACTCCGTGAAGG |
| | | GCCGATTCACCATCTCCAGAGACA |
| | | ATTCCAAGAACACGCTGTATCTCCA |
| | | AATGAACAGCCTGAGAGGTGAGGA |
| | | CACGGCGCTATATTACTGTGCGAGA |
| | | GTTTACTATGGTTCGGGGACTTATT |
| | | ATAAAAACCGCTACTACTACGGTAT |
| | | GGACGTCTGGGGCCAAGGGACCAC |
| | | GGTCACCGTCTCCTCAGGTGGTGGT |
| | | GGTTCTGGCGGCGGCGGCTCCGGT |
| | | GGTGGTGGTTCTTCATATGAGCTGA |
| | | CACAGCTACCTTCAGTGTCAGTGGC |
| | | CCTGGGACAGACGGCCAGGATTAC |
| | | CTGTGGGGGACACAACATTGGAAG |
| | | TAAAGGTGTGCACTGGTACCAGCA |
| | | GAAGCCAGGCCAGGCCCCTGTGCT |
| | | GGTCATCTATAGAAATAGCAACCG |
| | | GCCCTCTGGGATCCCTGAGCGATTC |
| | | TCTGGCTCCAACTCGGGGAACACG |
| | | GCCACCCTGACCATCAGCAGAGCC |
| | | CAAGCCGGGGATGAGGCTGACTAT |
| | | TACTGTCAGGTGTGGGACAGCAGC |
| | | ACTGTGGTTTTCGGCGGAGGGACC |
| | | GAGCTGACCGTCCTA |
| 1134 | MPK20299-B5_SCFV | CAGGTGCAGCTGGTGGAGTCTGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAACCGCTATTACTACGGTAT |
| | | GGACGTCTGGGGCCAAGGGACCAC |
| | | GGTCACCGTCTCCTCAGGTGGTGGT |
| | | GGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGACACAACATTGGAAG |
| | | TAAAGGTGTGCACTGGTACCAGCA |
| | | GAAGCCAGGCCAGGCCCCTGTGCT |
| | | GGTCATCTATAGAAATAGCAACCG |
| | | GCCCTCTGGGATCCCTGAGCGATTC |
| | | TCTGGCTCCAACTCGGGGAACACG |
| | | GCCACCCTGACCATCAGCAGAGCC |
| | | CAAGCCGGGGATGAGGCTGACTAT |
| | | TACTGTCAGGTGTGGGACAGTAGT |
| | | ACTGTGGTTTTCGGCGGAGGCACCC |
| | | AGCTGACCGTCCTA |
| 1135 | MPK20299-G9_SCFV | CAGGTGCAGCTGGTGGAGTCTGGG |
| | | GGAGACTTGGTACAGCCTGGGAGG |
| | | TCCCTGAGACTCTCCTGTGCAGCGT |
| | | CTGGATTCACCTTCAGTAACAATGG |
| | | CATGCACTGGGTCCGCCAGGCTCCA |
| | | GGCAAGGGACTGGAGTGGGTGGCA |
| | | GTTATTTCAAATGATGGCAGTAATA |
| | | AATATTATGCAGATTCCGTGAGGG |
| | | GCCGATTCACCATCTCCAGAGACA |
| | | ATTCCAAGAACACGCTGTATCTGCA |
| | | AATGAACAGCCTGAGAGCTGAGGA |
| | | CACGGCTGTGTATTATTGTGCGAAA |
| | | GTTTACTATGGTTCGGGTATTTATT |
| | | ATAAAAACAGGTACTACTACGGGA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACTCTCAGTGTCAGTGGC |
| | | CCTGGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTGCT |
| | | GGTCATCTATAGGAATAGCAACCG |
| | | GCCCTCTGGGATCCCTGAGCGATTC |
| | | TCTGGCTCCAACTCGGGGAACACG |
| | | GCCACCCTGACTCTTAGCAGAGTCC |
| | | AAGCCGGGGATGAGGCTGACTATT |
| | | ACTGTCAGGTGTGGGACAGCAGCA |
| | | CTGTGGTTTTCGGCGGAGGGACCA |
| | | AGCTGACCGTCCTA |
| 1136 | MPK20299-G5_SCFV | CAGGTGCAGCTGGTGGAGTCCGGG |
| | | GGAGGCGTGGTCCAGCCTGGGAGG |
| | | TCCCTGAGACTCTCCTGTGCAGTCT |
| | | CTGGATTCAACTTCAGTAACAATGG |
| | | CATGCACTGGGTCCGCCAGGCTCCA |
| | | GGCAAGGGACTGGAGTGGGTGGCA |
| | | GTTATTTCAAATGATGGCAGTAATA |
| | | AATATTATGCAGATTCCGTGAGGG |
| | | GCCGATTCACCATCTCCAGAGACA |
| | | ATTCCAAGAACACACTGTATCTGCA |
| | | AATGGACAGCCTGAGAACTGAGGA |
| | | CACGGCTGTGTATTACTGTGCGAAA |
| | | GTTTACTATGGTTCGGGTATTTATT |
| | | ATAAAAACAGGTACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGCTTAC |
| | | CTGTGAGGGAAACAACATTGGAAG |
| | | TAAAAATGTGCACTGGTACCAGCA |
| | | GAAGCCAGGCCAGGCCCCTGTGTT |
| | | GGTCATCTATAGGGATAGCAACCG |
| | | GCCCTCTGGGATCCCTGAGCGCTTC |
| | | TCTGGCTCCAAGTCGGGGAACACG |
| | | GCCACCCTGGCCATCAGCAGAGCC |
| | | CAAGCCGGGGATGAGTCTGACTAT |
| | | TACTGTCAGGTGTGGGACAGCAGT |
| | | GCCGTGGTATTCGGCGGAGGCACC |
| | | AAGCTGACCGTCCTA |
| 1137 | MPK20298-C10_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCACCTTCAGTAGCTCTGG |
| | | CATGCACTGGGTCCGCCAGGCTCCA |
| | | GGCAAGGGGCTGGAGTGGGTGGCA |
| | | GTTATATCAAATGATGGAAGTAAT |
| | | AAATACTATGCAGACTCCGTGAAG |
| | | GGCCGATTCACCATCTCCAGAGAC |
| | | AATTCCAAGAACACGCTGTATCTGC |
| | | AAATGAACAGCCTGAGAGCTGAGG |
| | | ACACGGCTGTGTATTACTGTGCGAA |
| | | AGTTTACTATGGTTCGGGGATTTAT |
| | | TATAAAAATAACTACTATTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTGCT |
| | | GGCCATCTATAGGAATAGCAACCG |
| | | GCCCTCTGGGATCCCTGAGCGATTC |
| | | ACTGGCTCCAACTCGGGGAACACG |
| | | GCCACCCTGACCATCAGCGGGACC |
| | | CAGGCTATGGATGAGGCTGACTATT |
| | | ACTGTCAGGCGTGGGACAGCAGCA |
| | | CTGTGGTATTCGGCGGAGGGACCA |
| | | AGCTGACCGTCCTA |
| 1138 | MPK20298-B5_SCFV | CAGGTGCAGCTGGTGGAGTCTGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTAC |
| | | CTGTGGGGGAAACAACATTGGAAG |
| | | TCAAAATGTGCACTGGTACCAGCA |
| | | GAAGCCAGGCCAGGCCCCTGTGCT |
| | | GGTCATCTATAGGGATAGCAACCG |
| | | GCCCTCTGGGATCCCTGAGCGCTTC |
| | | TCTGGCTCCAAGTCGGGGAACACG |
| | | GCCACCCTGGCCATCAGCAGAGCC |
| | | CAAGCCGGGGATGAGTCTGACTAT |
| | | TACTGTCAGGTGTGGGACAGCAGT |
| | | GCCGTGGTATTCGGCGGAGGCACC |
| | | CAGCTGACCGTCCTA |
| 1139 | MPK20299-F2_SCFV | CAGGTGCAGCTGGTGGAGTCCGGG |
| | | GGAGGCGTGGTCCAGCCTGGGAGG |
| | | TCCCTGAGACTCTCCTGTGCAGCCT |
| | | CTGGATTCACCCTCAGTAGCTCTGG |
| | | CATGCACTGGGTCCGCCAGGCTCCA |
| | | GGCAAGGGGCTGGAGTGGGTGGCA |
| | | GTTATATCAAATGATGGAAGTAAT |
| | | AAATACTATGCGGACTCCGTGAAG |
| | | GGCCGGTTCACCATCTCCAGAGAC |
| | | GATTCCAAGAACACACTGTATCTGC |
| | | AAATGGACAGCCTGAGAACTGAGG |
| | | ACACGGCTGTGTATTACTGTGCGAA |
| | | AGTTTACTATGGTTCGGGTATTTAT |
| | | TATAAAAACAGGTACTACTACGGG |
| | | ATGGACGTCTGGGGCCAAGGGACC |
| | | ACGGTCACCGTCTCCTCAGGTGGTG |
| | | GTGGTTCTGGCGGCGGCGGCTCCG |
| | | GTGGTGGTGGTTCTTCATATGAGCT |
| | | GACTCAGCCACCCTCAGTGTCAGTG |
| | | GCCCTGGGACAGACGGCCAGGATT |
| | | TCCTGTGGGGGAAACAACATTGGA |
| | | AGTAAAAATGTGCACTGGTACCAG |
| | | CAGAAGCCAGGCCAGGCCCCTGTG |
| | | CTGGTCATGTATAGGGATAGCAAC |
| | | CGGCCCTCAGGGATCCCTGAGCGA |
| | | TTCTCTGGCTCCAACTCTGGGAACA |
| | | CAGCCACTCTGACCATCAGCGGGA |
| | | CCCAGGCTATGGATGAGGCTGACT |
| | | ATTACTGTCAGGCGTGGGACAGCG |
| | | GCACTGTGGTATTCGGCGGAGGGA |
| | | CCAAGCTGACCGTCCTA |
| 1140 | MPK20298-D4_SCFV | CAGGTGCAGCTGGTGGAGTCTGGG |
| | | GGAGGCGTGGTCCAGCCTGGGAGG |
| | | TCCCTGAGACTCTCCTGTGCAGCCT |
| | | CTGGATTCAACTTCAGTAACTATGG |
| | | CATGCACTGGGTCCGCCAGGCTCCA |
| | | GGCAAGGGGCTGGAATGGGTGGCA |
| | | GTTATATCATATGATGGAAGTAATA |
| | | AATATTATGCAGACTCCGTGAAGG |
| | | GCCGATTCACCATCTCCAGAGACA |
| | | ATTCCAAGAACACGCTGTATCTACA |
| | | AATGAACAGCCTGAGAGCTGAGGA |
| | | CACGGCTGTGTATTTCTGTGCGAGA |
| | | GTTTACTATGGTTCGGGGATTTATT |
| | | ATAAAAAGAGATACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGAATTAC |
| | | CTGTGGGGGAAACAACATTGGAGG |
| | | TAAAAATGTGCACTGGTACCAGCA |
| | | GAAGCCAGGCCAGGCCCCTGTGCT |
| | | GGTCATCTATAGGGATAGCAACCG |
| | | GCCCTCTGGGATCCCTGAGCGATTC |
| | | TCTGGCTCCAAGTCGGGGAACACG |
| | | GCCACCCTGACCATCAGCAGAGCC |
| | | CAAGCCGGGGATGAGTCTGACTAT |
| | | TACTGTCAGGTGTGGGACAGCAGC |
| | | ACTGTGGTATTCGGCGGAGGCACC |
| | | CAGCTGACCGTCCTA |
| 1141 | MPK20297-F5_SCFV | CAGGTGCAGCTGGTGGAGTCTGGG |
| | | GGAGGCGTGGTCCAGCCTGGGAGG |
| | | TCCCTGAGACTCTCCTGTGTAGTCT |
| | | CTGGATTCAACTTCAGTAGGAATGG |
| | | CATGCACTGGGTCCGCCAGGTTCCA |
| | | GGCAGGGGGCTAGATTGGGTGGCA |
| | | GTTATATCAAATGATGGAAGTAAT |
| | | AAATACTATGCAGACTCCGTGAAG |
| | | GGCCGATTCACCATCTCCAGAGAC |
| | | AATTCCAAGAACACGCTGTATCTGC |
| | | AAATGAACAGCCTGAGAGCTGAGG |
| | | ACACGGCTGTGTATTACTGTGCGAA |
| | | AGTTTACTATGGTTCGGGGATTTAT |
| | | TATAAAAATAACTACTATTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACTCTCAGTGTCAGTGGC |
| | | CCTGGGACAGACGGCCAGGATTAC |
| | | CTGTGGGGGAAACAACATTGGAAG |
| | | TAAAAATGTGCACTGGTACCAGCA |
| | | GAAGCCAGGCCAGGCCCCTGTGCT |
| | | GGTCATCTATAGAAATAGCAACCG |
| | | GCCCTCTGGGATCCCTGAGCGATTC |
| | | TCTGGCTCCAACTCGGGGAACACG |
| | | GCCACCCTGACCATCAGCAGAGCC |
| | | CAGGCCGGGGATGAGGCTGACTAT |
| | | TACTGTCAGGTGTGGGACAGCAGC |
| | | ACTGTGGTTTTCGGCGGTGGGACCA |
| | | AGCTGACCGTCCTA |
| 1142 | MPK20299-D9_SCFV | CAGGTGCAGCTGGTGGAGTCCGGG |
| | | GGAGGCTTGGTCAAGCCTGGAGGG |
| | | TCCCTGAGACTCTCCTGTGCAGCCT |
| | | CTGGATTCAACTTCAGTAGGAATGG |
| | | CATGCACTGGGTCCGCCAGGTTCCA |
| | | GGCAGGGGGCTAGATTGGGTGGCA |
| | | GTTATATCAAATGATGGAAGTAAT |
| | | AAATACTATGCAGACTCCGTGAAG |
| | | GGCCGATTCACCATCTCCAGAGAC |
| | | AATTCCAAGAACACGCTGTATCTGC |
| | | AAATGAACAGCCTGAGAGCTGAGG |
| | | ACACGGCTGTGTATTACTGTGCGAA |
| | | AGTTTACTATGGTTCGGGGATTTAT |
| | | TATAAAAATAACTACTACTACGGTA |
| | | TGGACGTCTGGGGCCAAGGGACCA |
| | | CGGTCACCGTCTCCTCAGGTGGTGG |
| | | TGGTTCTGGCGGCGGCGGCTCCGGT |
| | | GGTGGTGGTTCTTCATATGAGCTGA |
| | | CTCAGCCACCCTCAGTGTCAGTGGC |
| | | CCTGGGACAGACGGCCAGGATTTC |
| | | CTGTGGGGGAAACAACATTGAAAG |
| | | TAAAAATGTGCACTGGTACCAGCA |
| | | GAAGCCAGGCCAGGCCCCTGTGTT |
| | | GGTCATCTATAGGGATAGCAACCG |
| | | GCCCTCTGGGATCCCTGAGCGATTC |
| | | TCTGGCTCCAAGTCGGGGACCACG |
| | | GCCACCCTGACCATCAGCAGAGCC |
| | | CAAGCCGGGGATGAGGCTGAGTAT |
| | | TACTGTCAGGTGTGGGACAGCAGC |
| | | ACTGTGGTTTTCGGCGGAGGCACCC |
| | | AGCTGACCGTCCTA |
| 1143 | huCCR8_32360_huIgG1z | ATGGACATGAGGGTGCCCGCTCAG |
| | mAb(LC: K38R)_HC | CTCCTGGGGCTCCTGCTGCTGTGGC |
| | | TGAGAGGTGCGCGCTGTGAGGTGC |
| | | AGCTGGTGGAGTCTGGGGGAGGCT |
| | | TGGTAAAGCCTGGGGGGTCCCTGA |
| | | GACTCTCCTGTGCAGCCTCTGGATT |
| | | TACTTTCAGTAACGCCCGGATGGGC |
| | | TGGGTCCGCCAGGCTCCAGGGAAG |
| | | GGGCTGGAGTGGGTTGGCCGTATT |
| | | AAAAGCAAAACTGAAGGTGGGACA |
| | | AGAGACTACGCTGCACCCGTGAAA |
| | | GGCAGATTCACCATCTCAAGAGAT |
| | | GATTCAAAAAACACGCTGTATCTGC |
| | | AAATGAACAGCCTGAAAACCGAGG |
| | | ACACAGCCGTGTATTATTGTACCTC |
| | | GTATAGTGGGGTCTGGGGCCAAGG |
| | | GACAATGGTCACCGTGTCTTCAGCC |
| | | TCCACCAAGGGCCCATCGGTCTTCC |
| | | CCCTGGCACCCTCCTCCAAGAGCAC |
| | | CTCTGGGGGCACAGCGGCCCTGGG |
| | | CTGCCTGGTCAAGGACTACTTCCCC |
| | | GAACCGGTGACGGTGTCGTGGAAC |
| | | TCAGGCGCCCTGACCAGCGGCGTG |
| | | CACACCTTCCCGGCTGTCCTACAGT |
| | | CCTCAGGACTCTACTCCCTCAGCAG |
| | | CGTGGTGACCGTGCCCTCCAGCAGC |
| | | TTGGGCACCCAGACCTACATCTGCA |
| | | ACGTGAATCACAAGCCCAGCAACA |
| | | CCAAGGTGGACAAGAAAGTTGAGC |
| | | CCAAATCTTGTGACAAAACTCACAC |
| | | ATGCCCACCGTGCCCAGCACCTGA |
| | | ACTCCTGGGGGGACCGTCAGTCTTC |
| | | CTCTTCCCCCCAAAACCCAAGGACA |
| | | CCCTCATGATCTCCCGGACCCCTGA |
| | | GGTCACATGCGTGGTGGTGGACGT |
| | | GAGCCACGAAGACCCTGAGGTCAA |
| | | GTTCAACTGGTACGTGGACGGCGT |
| | | GGAGGTGCATAATGCCAAGACAAA |
| | | GCCGCGGGAGGAGCAGTACAACAG |
| | | CACGTACCGTGTGGTCAGCGTCCTC |
| | | ACCGTCCTGCACCAGGACTGGCTG |
| | | AATGGCAAGGAGTACAAGTGCAAG |
| | | GTGTCCAACAAAGCCCTCCCAGCCC |
| | | CCATCGAGAAAACCATCTCCAAAG |
| | | CCAAAGGGCAGCCCCGAGAACCAC |
| | | AGGTGTACACCCTGCCCCCATCCCG |
| | | GGAGGAGATGACCAAGAACCAGGT |
| | | CAGCCTGACCTGCCTGGTCAAAGG |
| | | CTTCTATCCCAGCGACATCGCCGTG |
| | | GAGTGGGAGAGCAATGGGCAGCCG |
| | | GAGAACAACTACAAGACCACGCCT |
| | | CCCGTGCTGGACTCCGACGGCTCCT |
| | | TCTTCCTCTATAGCAAGCTCACCGT |
| | | GGACAAGAGCAGGTGGCAGCAGGG |
| | | GAACGTCTTCTCATGCTCCGTGATG |
| | | CATGAGGCTCTGCACAACCACTAC |
| | | ACGCAGAAGAGCCTCTCCCTGTCTC |
| | | CGGGCAAATAG |
| 1144 | huCCR8_32360_huIgG1z | ATGGACATGAGGGTGCCCGCTCAG |
| | mAb(LC: K38R)_LC | CTCCTGGGGCTCCTGCTGCTGTGGC |
| | | TGAGAGGTGCGCGCTGTGACATCG |
| | | TGATGACCCAGTCTCCAGACTCCCT |
| | | GGCTGTGTCTCTGGGCGAGAGGGC |
| | | CACCATCAACTGCAAGTCCAGCCA |
| | | GAGTGTTTTATACAGTTCCAACAAT |
| | | AGAAACTACTTAGCTTGGTACCATC |
| | | AGAAACCAGGACAGTCTCCTAAGC |
| | | TGCTCATTTCCTGGGCATCTACCCG |
| | | GGAATCCGGGGTCCCTGACCGATTC |
| | | AGTGGCAGCGGGTCTGGGACAGAT |
| | | TTCACTCTCACCATCAACAGCCTGC |
| | | AGGCTGAAGATGTGGCAGTTTATTA |
| | | CTGTCAACAATATTATAGTATTCCG |
| | | ATCACTTTCGGCGGAGGGACCAAG |
| | | GTGGAGATCAAACGAACGGTGGCT |
| | | GCACCATCTGTCTTCATCTTCCCGC |
| | | CATCTGATGAGCAGTTGAAATCTGG |
| | | AACTGCCTCTGTTGTGTGCCTGCTG |
| | | AATAACTTCTATCCCAGAGAGGCC |
| | | AAAGTACAGTGGAAGGTGGATAAC |
| | | GCCCTCCAATCGGGTAACTCCCAGG |
| | | AGAGTGTCACAGAGCAGGACAGCA |
| | | AGGACAGCACCTACAGCCTCAGCA |
| | | GCACCCTGACGCTGAGCAAAGCAG |
| | | ACTACGAGAAACACAAAGTCTACG |
| | | CCTGCGAAGTCACCCATCAGGGCCT |
| | | GAGCTCGCCCGTCACAAAGAGCTT |
| | | CAACAGGGGAGAGTGTTAG |
| 1145 | anti- | ATGGACATGAGGGTGCCCGCTCAG |
| | huCCR8_44379(VH: D72S,VL: N67A_S68A_ | CTCCTGGGGCTCCTGCTGCTGTGGC |
| | M99G_W109F_S111A)_huIgG1z (mAb)_HC | TGAGAGGTGCGCGCTGTCAGGTGC |
| | | AGCTGGTGGAGTCCGGGGGAGGCG |
| | | TGGTCCAGCCTGGGAGGTCCCTGA |
| | | GACTCTCCTGTGCAGCCTCTGGATT |
| | | CACCTTCAGTAACTATGGCTTTCAC |
| | | TGGGTCCGCCAGACTCCAGGCAAG |
| | | GGGCTGGAGTGGGTGGCAGTTATC |
| | | TCATATGATGGAAGTAATAGATACT |
| | | ATGCAAGCTCCGTGAAGGGCCGAT |
| | | TCACCATCTCCAGAGACAATTCCAA |
| | | GAACACGCTGTATCTCCAAATGAA |
| | | CAGCCTGAGAGGTGAGGACACGGC |
| | | GCTATATTACTGTGCGAGAGTTTAC |
| | | TATGGTTCGGGGACTTATTATAAAA |
| | | ACCGCTACTACTACGGTATGGACGT |
| | | CTGGGGCCAAGGGACCACGGTCAC |
| | | CGTGTCCTCAGCCTCCACCAAGGGC |
| | | CCATCGGTCTTCCCCCTGGCACCCT |
| | | CCTCCAAGAGCACCTCTGGGGGCA |
| | | CAGCGGCCCTGGGCTGCCTGGTCA |
| | | AGGACTACTTCCCCGAACCGGTGA |
| | | CGGTGTCGTGGAACTCAGGCGCCCT |
| | | GACCAGCGGCGTGCACACCTTCCC |
| | | GGCTGTCCTACAGTCCTCAGGACTC |
| | | TACTCCCTCAGCAGCGTGGTGACCG |
| | | TGCCCTCCAGCAGCTTGGGCACCCA |
| | | GACCTACATCTGCAACGTGAATCAC |
| | | AAGCCCAGCAACACCAAGGTGGAC |
| | | AAGAAAGTTGAGCCCAAATCTTGT |
| | | GACAAAACTCACACATGCCCACCG |
| | | TGCCCAGCACCTGAACTCCTGGGG |
| | | GGACCGTCAGTCTTCCTCTTCCCCC |
| | | CAAAACCCAAGGACACCCTCATGA |
| | | TCTCCCGGACCCCTGAGGTCACATG |
| | | CGTGGTGGTGGACGTGAGCCACGA |
| | | AGACCCTGAGGTCAAGTTCAACTG |
| | | GTACGTGGACGGCGTGGAGGTGCA |
| | | TAATGCCAAGACAAAGCCGCGGGA |
| | | GGAGCAGTACAACAGCACGTACCG |
| | | TGTGGTCAGCGTCCTCACCGTCCTG |
| | | CACCAGGACTGGCTGAATGGCAAG |
| | | GAGTACAAGTGCAAGGTGTCCAAC |
| | | AAAGCCCTCCCAGCCCCCATCGAG |
| | | AAAACCATCTCCAAAGCCAAAGGG |
| | | CAGCCCCGAGAACCACAGGTGTAC |
| | | ACCCTGCCCCCATCCCGGGAGGAG |
| | | ATGACCAAGAACCAGGTCAGCCTG |
| | | ACCTGCCTGGTCAAAGGCTTCTATC |
| | | CCAGCGACATCGCCGTGGAGTGGG |
| | | AGAGCAATGGGCAGCCGGAGAACA |
| | | ACTACAAGACCACGCCTCCCGTGCT |
| | | GGACTCCGACGGCTCCTTCTTCCTC |
| | | TATAGCAAGCTCACCGTGGACAAG |
| | | AGCAGGTGGCAGCAGGGGAACGTC |
| | | TTCTCATGCTCCGTGATGCATGAGG |
| | | CTCTGCACAACCACTACACGCAGA |
| | | AGAGCCTCTCCCTGTCTCCGGGCAA |
| | | ATAG |
| 1146 | anti- | ATGGCCTGGGCTCTGCTGCTCCTCA |
| | huCCR8_44379(VH: D72S, VL: N67A_S68A_ | CCCTCCTCACTCAGGGCACAGGGTC |
| | M99G_W109F_S111A)_huIgG1z (mAb)_LC | CTGGGCCTCATATGAGCTGACTCAG |
| | | CCACCCTCAGTGTCAGTGGCCCTGG |
| | | GACAGACGGCCAGGATTACCTGTG |
| | | GGGGACACAACATTGGAAGTAAAG |
| | | GTGTGCACTGGTACCAGCAGAAGC |
| | | CAGGCCAGGCCCCTGTACTGGTCAT |
| | | CTATAGAGCCGCCAACCGGCCCTCT |
| | | GGGATCCCTGAGCGATTCTCTGGCT |
| | | CCAACTCTGGGAACACAGCCACTCT |
| | | GACCATCAGCGGGACCCAGGCTGG |
| | | CGATGAGGCTGACTATTACTGTCAG |
| | | GCGTTCGACGCCGGCACTGTGGTAT |
| | | TCGGCGGAGGCACCCAGCTGACCG |
| | | TCCTAGGTCAGCCCAAGGCTGCACC |
| | | CTCGGTCACTCTGTTCCCGCCCTCC |
| | | TCTGAGGAGCTTCAAGCCAACAAG |
| | | GCCACACTGGTGTGTCTCATCAGTG |
| | | ACTTCTACCCGGGAGCCGTGACAGT |
| | | GGCCTGGAAGGCAGATAGCAGCCC |
| | | CGTCAAGGCGGGAGTGGAAACCAC |
| | | CACACCCTCCAAACAAAGCAACAA |
| | | CAAGTACGCGGCCAGCAGCTATCT |
| | | GAGCCTGACGCCTGAGCAGTGGAA |
| | | GTCCCACAGAAGCTACAGCTGCCA |
| | | GGTCACGCATGAAGGGAGCACCGT |
| | | GGAGAAGACAGTGGCCCCTACAGA |
| | | ATGTTCATAG |
| 1147 | anti- | ATGGACATGAGGGTGCCCGCTCAG |
| | huCCR8_44379(VH: D61A_D72A, VL: N67Q_ | CTCCTGGGGCTCCTGCTGCTGTGGC |
| | M99E_W109F_S111A)_huIgG1z | TGAGAGGTGCGCGCTGTCAGGTGC |
| | (mAb)_HC | AGCTGGTGGAGTCCGGGGGAGGCG |
| | | TGGTCCAGCCTGGGAGGTCCCTGA |
| | | GACTCTCCTGTGCAGCCTCTGGATT |
| | | CACCTTCAGTAACTATGGCTTTCAC |
| | | TGGGTCCGCCAGACTCCAGGCAAG |
| | | GGGCTGGAGTGGGTGGCAGTTATC |
| | | TCATATGCCGGAAGTAATAGATACT |
| | | ATGCAGCCTCCGTGAAGGGCCGAT |
| | | TCACCATCTCCAGAGACAATTCCAA |
| | | GAACACGCTGTATCTCCAAATGAA |
| | | CAGCCTGAGAGGTGAGGACACGGC |
| | | GCTATATTACTGTGCGAGAGTTTAC |
| | | TATGGTTCGGGGACTTATTATAAAA |
| | | ACCGCTACTACTACGGTATGGACGT |
| | | CTGGGGCCAAGGGACCACGGTCAC |
| | | CGTGTCCTCAGCCTCCACCAAGGGC |
| | | CCATCGGTCTTCCCCCTGGCACCCT |
| | | CCTCCAAGAGCACCTCTGGGGGCA |
| | | CAGCGGCCCTGGGCTGCCTGGTCA |
| | | AGGACTACTTCCCCGAACCGGTGA |
| | | CGGTGTCGTGGAACTCAGGCGCCCT |
| | | GACCAGCGGCGTGCACACCTTCCC |
| | | GGCTGTCCTACAGTCCTCAGGACTC |
| | | TACTCCCTCAGCAGCGTGGTGACCG |
| | | TGCCCTCCAGCAGCTTGGGCACCCA |
| | | GACCTACATCTGCAACGTGAATCAC |
| | | AAGCCCAGCAACACCAAGGTGGAC |
| | | AAGAAAGTTGAGCCCAAATCTTGT |
| | | GACAAAACTCACACATGCCCACCG |
| | | TGCCCAGCACCTGAACTCCTGGGG |
| | | GGACCGTCAGTCTTCCTCTTCCCCC |
| | | CAAAACCCAAGGACACCCTCATGA |
| | | TCTCCCGGACCCCTGAGGTCACATG |
| | | CGTGGTGGTGGACGTGAGCCACGA |
| | | AGACCCTGAGGTCAAGTTCAACTG |
| | | GTACGTGGACGGCGTGGAGGTGCA |
| | | TAATGCCAAGACAAAGCCGCGGGA |
| | | GGAGCAGTACAACAGCACGTACCG |
| | | TGTGGTCAGCGTCCTCACCGTCCTG |
| | | CACCAGGACTGGCTGAATGGCAAG |
| | | GAGTACAAGTGCAAGGTGTCCAAC |
| | | AAAGCCCTCCCAGCCCCCATCGAG |
| | | AAAACCATCTCCAAAGCCAAAGGG |
| | | CAGCCCCGAGAACCACAGGTGTAC |
| | | ACCCTGCCCCCATCCCGGGAGGAG |
| | | ATGACCAAGAACCAGGTCAGCCTG |
| | | ACCTGCCTGGTCAAAGGCTTCTATC |
| | | CCAGCGACATCGCCGTGGAGTGGG |
| | | AGAGCAATGGGCAGCCGGAGAACA |
| | | ACTACAAGACCACGCCTCCCGTGCT |
| | | GGACTCCGACGGCTCCTTCTTCCTC |
| | | TATAGCAAGCTCACCGTGGACAAG |
| | | AGCAGGTGGCAGCAGGGGAACGTC |
| | | TTCTCATGCTCCGTGATGCATGAGG |
| | | CTCTGCACAACCACTACACGCAGA |
| | | AGAGCCTCTCCCTGTCTCCGGGCAA |
| | | ATAG |
| 1148 | anti- | ATGGCCTGGGCTCTGCTGCTCCTCA |
| | huCCR8_44379(VH: D61A_D72A, VL: N67Q_ | CCCTCCTCACTCAGGGCACAGGGTC |
| | M99E_W109F_S111A)_huIgG1z | CTGGGCCTCATATGAGCTGACTCAG |
| | (mAb)_LC | CCACCCTCAGTGTCAGTGGCCCTGG |
| | | GACAGACGGCCAGGATTACCTGTG |
| | | GGGGACACAACATTGGAAGTAAAG |
| | | GTGTGCACTGGTACCAGCAGAAGC |
| | | CAGGCCAGGCCCCTGTACTGGTCAT |
| | | CTATAGACAGAGCAACCGGCCCTC |
| | | TGGGATCCCTGAGCGATTCTCTGGC |
| | | TCCAACTCTGGGAACACAGCCACTC |
| | | TGACCATCAGCGGGACCCAGGCTG |
| | | AAGATGAGGCTGACTATTACTGTCA |
| | | GGCGTTCGACGCCGGCACTGTGGT |
| | | ATTCGGCGGAGGCACCCAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1149 | anti- | ATGGACATGAGGGTGCCCGCTCAG |
| | huCCR8_44379(VH: D61S, VL: N67Q_M99G | CTCCTGGGGCTCCTGCTGCTGTGGC |
| | _W109F_S111A) huIgG1z (mAb)_HC | TGAGAGGTGCGCGCTGTCAGGTGC |
| | | AGCTGGTGGAGTCCGGGGGAGGCG |
| | | TGGTCCAGCCTGGGAGGTCCCTGA |
| | | GACTCTCCTGTGCAGCCTCTGGATT |
| | | CACCTTCAGTAACTATGGCTTTCAC |
| | | TGGGTCCGCCAGACTCCAGGCAAG |
| | | GGGCTGGAGTGGGTGGCAGTTATC |
| | | TCATATAGCGGAAGTAATAGATAC |
| | | TATGCAGACTCCGTGAAGGGCCGA |
| | | TTCACCATCTCCAGAGACAATTCCA |
| | | AGAACACGCTGTATCTCCAAATGA |
| | | ACAGCCTGAGAGGTGAGGACACGG |
| | | CGCTATATTACTGTGCGAGAGTTTA |
| | | CTATGGTTCGGGGACTTATTATAAA |
| | | AACCGCTACTACTACGGTATGGAC |
| | | GTCTGGGGCCAAGGGACCACGGTC |
| | | ACCGTGTCCTCAGCCTCCACCAAGG |
| | | GCCCATCGGTCTTCCCCCTGGCACC |
| | | CTCCTCCAAGAGCACCTCTGGGGGC |
| | | ACAGCGGCCCTGGGCTGCCTGGTC |
| | | AAGGACTACTTCCCCGAACCGGTG |
| | | ACGGTGTCGTGGAACTCAGGCGCC |
| | | CTGACCAGCGGCGTGCACACCTTCC |
| | | CGGCTGTCCTACAGTCCTCAGGACT |
| | | CTACTCCCTCAGCAGCGTGGTGACC |
| | | GTGCCCTCCAGCAGCTTGGGCACCC |
| | | AGACCTACATCTGCAACGTGAATC |
| | | ACAAGCCCAGCAACACCAAGGTGG |
| | | ACAAGAAAGTTGAGCCCAAATCTT |
| | | GTGACAAAACTCACACATGCCCAC |
| | | CGTGCCCAGCACCTGAACTCCTGGG |
| | | GGGACCGTCAGTCTTCCTCTTCCCC |
| | | CCAAAACCCAAGGACACCCTCATG |
| | | ATCTCCCGGACCCCTGAGGTCACAT |
| | | GCGTGGTGGTGGACGTGAGCCACG |
| | | AAGACCCTGAGGTCAAGTTCAACT |
| | | GGTACGTGGACGGCGTGGAGGTGC |
| | | ATAATGCCAAGACAAAGCCGCGGG |
| | | AGGAGCAGTACAACAGCACGTACC |
| | | GTGTGGTCAGCGTCCTCACCGTCCT |
| | | GCACCAGGACTGGCTGAATGGCAA |
| | | GGAGTACAAGTGCAAGGTGTCCAA |
| | | CAAAGCCCTCCCAGCCCCCATCGA |
| | | GAAAACCATCTCCAAAGCCAAAGG |
| | | GCAGCCCCGAGAACCACAGGTGTA |
| | | CACCCTGCCCCCATCCCGGGAGGA |
| | | GATGACCAAGAACCAGGTCAGCCT |
| | | GACCTGCCTGGTCAAAGGCTTCTAT |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTATAGCAAGCTCACCGTGGACA |
| | | AGAGCAGGTGGCAGCAGGGGAACG |
| | | TCTTCTCATGCTCCGTGATGCATGA |
| | | GGCTCTGCACAACCACTACACGCA |
| | | GAAGAGCCTCTCCCTGTCTCCGGGC |
| | | AAATAG |
| 1150 | anti- | ATGGCCTGGGCTCTGCTGCTCCTCA |
| | huCCR8_44379(VH: D61S, VL: N67Q_M99G | CCCTCCTCACTCAGGGCACAGGGTC |
| | _109F_S111A)_huIgG1z (mAb)_LC | CTGGGCCTCATATGAGCTGACTCAG |
| | | CCACCCTCAGTGTCAGTGGCCCTGG |
| | | GACAGACGGCCAGGATTACCTGTG |
| | | GGGGACACAACATTGGAAGTAAAG |
| | | GTGTGCACTGGTACCAGCAGAAGC |
| | | CAGGCCAGGCCCCTGTACTGGTCAT |
| | | CTATAGACAGAGCAACCGGCCCTC |
| | | TGGGATCCCTGAGCGATTCTCTGGC |
| | | TCCAACTCTGGGAACACAGCCACTC |
| | | TGACCATCAGCGGGACCCAGGCTG |
| | | GCGATGAGGCTGACTATTACTGTCA |
| | | GGCGTTCGACGCCGGCACTGTGGT |
| | | ATTCGGCGGAGGCACCCAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1151 | Hu anti-huCCR8 LIBC315615-1 HuIgG1z | ATGGCCTGGGCTCTGCTGCTCCTCA |
| | mAb_LC | CCCTCCTCACTCAGGGCACAGGGTC |
| | | CTGGGCCTCCTATGAACTGACTCAG |
| | | CCACTCTCAGTGTCAGTGGCCCTGG |
| | | GACAGACGGCCAGGATTACCTGTG |
| | | GGGGACACAACATTGGAAGTAAAG |
| | | GTGTGCACTGGTACCAGCAGAAGC |
| | | CAGGCCAGGCCCCTGTGCTGGTCAT |
| | | CTATAGAAATAGCAACCGGCCCTCT |
| | | GGGATCCCTGAGCGATTCTCTGGCT |
| | | CCAACTCGGGGAACACGGCCACCC |
| | | TGACCATCAGCAGAGCCCAAGCCG |
| | | GGGATGAGGCTGACTATTACTGTCA |
| | | GGTGTGGGACATCAGCACTGTGGTT |
| | | TTCGGCGGAGGGACCGAGCTGACC |
| | | GTCCTAGGTCAGCCCAAGGCTGCA |
| | | CCCTCGGTCACTCTGTTCCCGCCCT |
| | | CCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1152 | Hu anti-huCCR8 LIBC315615-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGCTGGTGGAGTCTGGGGGAGGCG |
| | | TGGCCCAGCCTGGGAGGTCCCTGA |
| | | GACTCTCCTGTGCAGCCTCTGGATT |
| | | CAACTTCAGTAACTGTGGCATGCAC |
| | | TGGGTCCGCCAGGCTCCAGGCAAG |
| | | GGGCTGGAGTGGGTGGCAGTTATA |
| | | TCATATGATGGAGGTAATAAATATC |
| | | ATGCGGACTCCGTGAAGGGCCGGT |
| | | TCACCATCTCCAGAGACGATTCCAA |
| | | GAACACACTGTATCTGCAAATGGA |
| | | CAGCCTGAGAACTGAGGACACGGC |
| | | TGTGTATTACTGTGCGAAAGTTTAC |
| | | TATGGTTCGGGTATTTATTATAAAA |
| | | ACAGGTACTACTACGGGATGGACG |
| | | TCtGGGGCCAAGGGACCACGGTCAC |
| | | CGTCTCCTCAGCCTCCACCAAGGGC |
| | | CCATCGGTCTTCCCCCTGGCACCCT |
| | | CCTCCAAGAGCACCTCTGGGGGCA |
| | | CAGCGGCCCTGGGCTGCCTGGTCA |
| | | AGGACTACTTCCCCGAACCGGTGA |
| | | CGGTGTCGTGGAACTCAGGCGCCCT |
| | | GACCAGCGGCGTGCACACCTTCCC |
| | | GGCTGTCCTACAGTCCTCAGGACTC |
| | | TACTCCCTCAGCAGCGTGGTGACCG |
| | | TGCCCTCCAGCAGCTTGGGCACCCA |
| | | GACCTACATCTGCAACGTGAATCAC |
| | | AAGCCCAGCAACACCAAGGTGGAC |
| | | AAGAAAGTTGAGCCCAAATCTTGT |
| | | GACAAAACTCACACATGCCCACCG |
| | | TGCCCAGCACCTGAACTCCTGGGG |
| | | GGACCGTCAGTCTTCCTCTTCCCCC |
| | | CAAAACCCAAGGACACCCTCATGA |
| | | TCTCCCGGACCCCTGAGGTCACATG |
| | | CGTGGTGGTGGACGTGAGCCACGA |
| | | AGACCCTGAGGTCAAGTTCAACTG |
| | | GTACGTGGACGGCGTGGAGGTGCA |
| | | TAATGCCAAGACAAAGCCGCGGGA |
| | | GGAGCAGTACAACAGCACGTACCG |
| | | TGTGGTCAGCGTCCTCACCGTCCTG |
| | | CACCAGGACTGGCTGAATGGCAAG |
| | | GAGTACAAGTGCAAGGTGTCCAAC |
| | | AAAGCCCTCCCAGCCCCCATCGAG |
| | | AAAACCATCTCCAAAGCCAAAGGG |
| | | CAGCCCCGAGAACCACAGGTGTAC |
| | | ACCCTGCCCCCATCCCGGGAGGAG |
| | | ATGACCAAGAACCAGGTCAGCCTG |
| | | ACCTGCCTGGTCAAAGGCTTCTATC |
| | | CCAGCGACATCGCCGTGGAGTGGG |
| | | AGAGCAATGGGCAGCCGGAGAACA |
| | | ACTACAAGACCACGCCTCCCGTGCT |
| | | GGACTCCGACGGCTCCTTCTTCCTC |
| | | TATAGCAAGCTCACCGTGGACAAG |
| | | AGCAGGTGGCAGCAGGGGAACGTC |
| | | TTCTCATGCTCCGTGATGCATGAGG |
| | | CTCTGCACAACCACTACACGCAGA |
| | | AGAGCCTCTCCCTGTCTCCGGGCAA |
| | | ATAG |
| 1153 | Hu anti-huCCR8 LIBC317152-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGCCAGGATTACCTGTG |
| | | GGGGACACAACATTGGAAGTAAAG |
| | | GTGTGCACTGGTACCAGCAGAAGC |
| | | CAGGCCAGGCCCCTGTGCTGGTCAT |
| | | CTATAGAAATAGCAACCGGCCCTCT |
| | | GGGATCCCTGAGCGATTCTCTGGCT |
| | | CCAACTCGGGGAAAACGGCCACCC |
| | | TGACCATCAGCAGAGCCCAAGCCG |
| | | GGGATGAGGCTGACTATTACTGTCA |
| | | GGTGTGGGACAGCAGCACTGTGGT |
| | | TTTCGGCGGAGGGACCGAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1154 | Hu anti-huCCR8 LIBC317152-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGCTGGTGGAGTCTGGGGGAGGCG |
| | | TGGCCCAGCCTGGGAGGTCCCTGA |
| | | GACTCTCCTGTGCAGCCTCTGGATT |
| | | CAACTTCAGTAACTGTGGCATGCAC |
| | | TGGGTCCGCCAGGCTCCAGGCAAG |
| | | GGGCTGGAGTGGGTGGCAGTTATA |
| | | TCATATGATGGAGGTAATAAATATT |
| | | ATGCGGACTCCGTGAAGGGCCGGT |
| | | TCACCATCTCCAGAGACGATTCCAA |
| | | GAACACACTGTATCTGCAAATGGA |
| | | CAGCCTGAGAACTGAGGACACGGC |
| | | TGTGTATTACTGTGCGAAAGTTTAC |
| | | TATGGTTCGGGTATTTATTATAAAA |
| | | ACAGGTATTACTACGGGATGGACG |
| | | TCTGGGGCCAAGGGACCACGGTCA |
| | | CCGTCTCCTCAGCCTCCACCAAGGG |
| | | CCCATCGGTCTTCCCCCTGGCACCC |
| | | TCCTCCAAGAGCACCTCTGGGGGC |
| | | ACAGCGGCCCTGGGCTGCCTGGTC |
| | | AAGGACTACTTCCCCGAACCGGTG |
| | | ACGGTGTCGTGGAACTCAGGCGCC |
| | | CTGACCAGCGGCGTGCACACCTTCC |
| | | CGGCTGTCCTACAGTCCTCAGGACT |
| | | CTACTCCCTCAGCAGCGTGGTGACC |
| | | GTGCCCTCCAGCAGCTTGGGCACCC |
| | | AGACCTACATCTGCAACGTGAATC |
| | | ACAAGCCCAGCAACACCAAGGTGG |
| | | ACAAGAAAGTTGAGCCCAAATCTT |
| | | GTGACAAAACTCACACATGCCCAC |
| | | CGTGCCCAGCACCTGAACTCCTGGG |
| | | GGGACCGTCAGTCTTCCTCTTCCCC |
| | | CCAAAACCCAAGGACACCCTCATG |
| | | ATCTCCCGGACCCCTGAGGTCACAT |
| | | GCGTGGTGGTGGACGTGAGCCACG |
| | | AAGACCCTGAGGTCAAGTTCAACT |
| | | GGTACGTGGACGGCGTGGAGGTGC |
| | | ATAATGCCAAGACAAAGCCGCGGG |
| | | AGGAGCAGTACAACAGCACGTACC |
| | | GTGTGGTCAGCGTCCTCACCGTCCT |
| | | GCACCAGGACTGGCTGAATGGCAA |
| | | GGAGTACAAGTGCAAGGTGTCCAA |
| | | CAAAGCCCTCCCAGCCCCCATCGA |
| | | GAAAACCATCTCCAAAGCCAAAGG |
| | | GCAGCCCCGAGAACCACAGGTGTA |
| | | CACCCTGCCCCCATCCCGGGAGGA |
| | | GATGACCAAGAACCAGGTCAGCCT |
| | | GACCTGCCTGGTCAAAGGCTTCTAT |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTATAGCAAGCTCACCGTGGACA |
| | | AGAGCAGGTGGCAGCAGGGGAACG |
| | | TCTTCTCATGCTCCGTGATGCATGA |
| | | GGCTCTGCACAACCACTACACGCA |
| | | GAAGAGCCTCTCCCTGTCTCCGGGC |
| | | AAATAG |
| 1155 | Hu anti-huCCR8 LIBC317471-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGCCAGGATTACCTGTG |
| | | GGGGAAACAACATTGGAAGTAAAA |
| | | ATGTGCACTGGTACCAGAAGAGGC |
| | | CAGGCCAGGCCCCTGTGCTGGTCAT |
| | | CTATAGGGATAGCAACCGGCCCTCT |
| | | GGGATCCCTGAGCGATTCTCTGGCT |
| | | CCAAGTCGGGGAACACGGCCACCC |
| | | TGACCATCAGCAGAGCCCAAGCCG |
| | | GGGATGAGGCTGACTATTACTGTCA |
| | | GGTGTGGGACAGCAACACTGTGGT |
| | | TTTCGGCGGAGGGACCAACCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1156 | Hu anti-huCCR8 LIBC317471-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGCTGGTGGAGTCTGGGGGAGGCG |
| | | TGGTCCAGCCTGGGAGGTCCCTGA |
| | | GACTCTCCTGTGTAGTCTCTGGATT |
| | | CAACTTCAGTAACAATGGCATGCA |
| | | CTGGGTCCGCCAGGCTCCAGGCAA |
| | | GGGACTGGAGTGGGTGGCAGTTAT |
| | | TTCAAATGATGGCAGTAATAAATAT |
| | | TATGCAGATTCCGTGAGGGGCCGA |
| | | TTCACCATCTCCAGAGACAATTCCA |
| | | AGAACACGCTGTATCTGCAAATGA |
| | | ACAGCCTGAGAGCTGAGGACACGG |
| | | CTGTGTATTCCTGTGCGAAAGTTTA |
| | | CTATGGTTCGGGAATTTATTACAAA |
| | | AATAACTACTACTACGGTATGGAC |
| | | GTCTGGGGCCAAGGGACCACGGTC |
| | | ACCGTCTCCTCAGCCTCCACCAAGG |
| | | GCCCATCGGTCTTCCCCCTGGCACC |
| | | CTCCTCCAAGAGCACCTCTGGGGGC |
| | | ACAGCGGCCCTGGGCTGCCTGGTC |
| | | AAGGACTACTTCCCCGAACCGGTG |
| | | ACGGTGTCGTGGAACTCAGGCGCC |
| | | CTGACCAGCGGCGTGCACACCTTCC |
| | | CGGCTGTCCTACAGTCCTCAGGACT |
| | | CTACTCCCTCAGCAGCGTGGTGACC |
| | | GTGCCCTCCAGCAGCTTGGGCACCC |
| | | AGACCTACATCTGCAACGTGAATC |
| | | ACAAGCCCAGCAACACCAAGGTGG |
| | | ACAAGAAAGTTGAGCCCAAATCTT |
| | | GTGACAAAACTCACACATGCCCAC |
| | | CGTGCCCAGCACCTGAACTCCTGGG |
| | | GGGACCGTCAGTCTTCCTCTTCCCC |
| | | CCAAAACCCAAGGACACCCTCATG |
| | | ATCTCCCGGACCCCTGAGGTCACAT |
| | | GCGTGGTGGTGGACGTGAGCCACG |
| | | AAGACCCTGAGGTCAAGTTCAACT |
| | | GGTACGTGGACGGCGTGGAGGTGC |
| | | ATAATGCCAAGACAAAGCCGCGGG |
| | | AGGAGCAGTACAACAGCACGTACC |
| | | GTGTGGTCAGCGTCCTCACCGTCCT |
| | | GCACCAGGACTGGCTGAATGGCAA |
| | | GGAGTACAAGTGCAAGGTGTCCAA |
| | | CAAAGCCCTCCCAGCCCCCATCGA |
| | | GAAAACCATCTCCAAAGCCAAAGG |
| | | GCAGCCCCGAGAACCACAGGTGTA |
| | | CACCCTGCCCCCATCCCGGGAGGA |
| | | GATGACCAAGAACCAGGTCAGCCT |
| | | GACCTGCCTGGTCAAAGGCTTCTAT |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTATAGCAAGCTCACCGTGGACA |
| | | AGAGCAGGTGGCAGCAGGGGAACG |
| | | TCTTCTCATGCTCCGTGATGCATGA |
| | | GGCTCTGCACAACCACTACACGCA |
| | | GAAGAGCCTCTCCCTGTCTCCGGGC |
| | | AAATAG |
| 1157 | Hu anti-huCCR8 LIBC317977-1 HuIgG1z | ATGGCCTGGGCTCTGCTGCTCCTCA |
| | mAb_LC | CCCTCCTCACTCAGGGCACAGGGTC |
| | | CTGGGCCTcctATGAGCTGACTCAGC |
| | | CACTCTCAGTGTCAGTGGCCCTGGG |
| | | ACAGACGGCCAGGATTACCTGTGG |
| | | GGGAAACAACATTGGAAGTAAAAA |
| | | TGTGCACTGGTACCAGCAGAAGGC |
| | | AGGCCAGGCCCCTGTGCAGGTCAT |
| | | CTATAGAAATAGCAACCGGCCCTCT |
| | | GGGATCCCTGAGCGATTCTCTGGCT |
| | | CCAACTCGGGGAACACGGCCACCC |
| | | TGACCATCAGCAGAGCCCAGGCCG |
| | | GGGATGAGGCTGACTATTACTGTCA |
| | | GGTGTGGGACAGCAGCACTGTGGT |
| | | TTTCGGCGGTGGGACCAAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1158 | Hu anti-huCCR8 LIBC317977-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGCTGGTGGAGTCTGGGGGAGGCG |
| | | TGGTCCAGCCTGGGAGGTCCCTGA |
| | | GACTCTCCTGTGCAGCCTCTGGATT |
| | | CAACTTCAATACCTATGGCATGCAC |
| | | TGGGTCCGCCAGGCTCCAGGCAAG |
| | | GGGCTGGAGTGGGTGGCAGTTATA |
| | | TCATATGATGGAAGTAATAAATATT |
| | | ATGCAGACTCCGTGAAGGGCCGAT |
| | | TCACCATCTCCAGAGACAATTCCAA |
| | | GAGCACGCTGTATCTGCAAATGAA |
| | | CAGCCTGAGAGCTGAGGACACGGC |
| | | TGTGTATTACTGTGCGAGAGTTTAC |
| | | TATGGTTCGGGGAGTTATTATAAAA |
| | | AGAATTACTACTACGGTATGGACGT |
| | | CTGGGGCCAAGGGACCACGGTCAC |
| | | CGTCTCCTCAGCCTCCACCAAGGGC |
| | | CCATCGGTCTTCCCCCTGGCACCCT |
| | | CCTCCAAGAGCACCTCTGGGGGCA |
| | | CAGCGGCCCTGGGCTGCCTGGTCA |
| | | AGGACTACTTCCCCGAACCGGTGA |
| | | CGGTGTCGTGGAACTCAGGCGCCCT |
| | | GACCAGCGGCGTGCACACCTTCCC |
| | | GGCTGTCCTACAGTCCTCAGGACTC |
| | | TACTCCCTCAGCAGCGTGGTGACCG |
| | | TGCCCTCCAGCAGCTTGGGCACCCA |
| | | GACCTACATCTGCAACGTGAATCAC |
| | | AAGCCCAGCAACACCAAGGTGGAC |
| | | AAGAAAGTTGAGCCCAAATCTTGT |
| | | GACAAAACTCACACATGCCCACCG |
| | | TGCCCAGCACCTGAACTCCTGGGG |
| | | GGACCGTCAGTCTTCCTCTTCCCCC |
| | | CAAAACCCAAGGACACCCTCATGA |
| | | TCTCCCGGACCCCTGAGGTCACATG |
| | | CGTGGTGGTGGACGTGAGCCACGA |
| | | AGACCCTGAGGTCAAGTTCAACTG |
| | | GTACGTGGACGGCGTGGAGGTGCA |
| | | TAATGCCAAGACAAAGCCGCGGGA |
| | | GGAGCAGTACAACAGCACGTACCG |
| | | TGTGGTCAGCGTCCTCACCGTCCTG |
| | | CACCAGGACTGGCTGAATGGCAAG |
| | | GAGTACAAGTGCAAGGTGTCCAAC |
| | | AAAGCCCTCCCAGCCCCCATCGAG |
| | | AAAACCATCTCCAAAGCCAAAGGG |
| | | CAGCCCCGAGAACCACAGGTGTAC |
| | | ACCCTGCCCCCATCCCGGGAGGAG |
| | | ATGACCAAGAACCAGGTCAGCCTG |
| | | ACCTGCCTGGTCAAAGGCTTCTATC |
| | | CCAGCGACATCGCCGTGGAGTGGG |
| | | AGAGCAATGGGCAGCCGGAGAACA |
| | | ACTACAAGACCACGCCTCCCGTGCT |
| | | GGACTCCGACGGCTCCTTCTTCCTC |
| | | TATAGCAAGCTCACCGTGGACAAG |
| | | AGCAGGTGGCAGCAGGGGAACGTC |
| | | TTCTCATGCTCCGTGATGCATGAGG |
| | | CTCTGCACAACCACTACACGCAGA |
| | | AGAGCCTCTCCCTGTCTCCGGGCAA |
| | | ATAG |
| 1159 | Hu anti-huCCR8 LIBC318774-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGCCAGGATTACCTGTG |
| | | GGGGAAACAACATTGGAGGTAAAA |
| | | ATGTGCACTGGTACCAGCAGAAGC |
| | | CAGGCCAGGCCCCTGTGCTGGTCAT |
| | | CTATAGGGATAGCAACCGGCCCTCT |
| | | GGGATCCCTGAGCGATTCTCTGGCT |
| | | CCAAGTCGGGGAACACGGCCACCC |
| | | TGACCATCAGCAGAGCCCAAGCCG |
| | | GGGATGAGTCTGACTATTACTGTCA |
| | | GGTTTGGGACAGCAGCACTGTGGT |
| | | ATTCGGCGGAGGGACCACGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1160 | Hu anti-huCCR8 LIBC318774-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGGTGGTGGAGTCTGGGGGAGGCG |
| | | TGGTCCAGCCTGGGAGGTCCCTGA |
| | | GACTCTCCTGTGCAGCCTCTGGATT |
| | | CACCCTCAGTAGTTATGGCTTTCAC |
| | | TGGGTCCGCCAGACTCCAGGCAAG |
| | | GGGCTGGAGTGGGTGGCAGTTATA |
| | | TCATATGATGGAAGTAATAAATAct |
| | | ATGCAGACTCCGTGAAGGGCCGAT |
| | | TCACCATCTCCAGAGACAATTCCAA |
| | | GAACACGCTGTATCTCCAAATGAA |
| | | CAGCCTGAGAGGTGAGGACACGGC |
| | | GGTGTATTACTGTGCGAGAGTTTAC |
| | | TATGGTTCGGGGACTTATTATAAAA |
| | | ACCGCTACTACTACGGTATGGACGT |
| | | CTGGGGCCAAGGGACCACGGTCAC |
| | | CGTCTCCTCAGCCTCCACCAAGGGC |
| | | CCATCGGTCTTCCCCCTGGCACCCT |
| | | CCTCCAAGAGCACCTCTGGGGGCA |
| | | CAGCGGCCCTGGGCTGCCTGGTCA |
| | | AGGACTACTTCCCCGAACCGGTGA |
| | | CGGTGTCGTGGAACTCAGGCGCCCT |
| | | GACCAGCGGCGTGCACACCTTCCC |
| | | GGCTGTCCTACAGTCCTCAGGACTC |
| | | TACTCCCTCAGCAGCGTGGTGACCG |
| | | TGCCCTCCAGCAGCTTGGGCACCCA |
| | | GACCTACATCTGCAACGTGAATCAC |
| | | AAGCCCAGCAACACCAAGGTGGAC |
| | | AAGAAAGTTGAGCCCAAATCTTGT |
| | | GACAAAACTCACACATGCCCACCG |
| | | TGCCCAGCACCTGAACTCCTGGGG |
| | | GGACCGTCAGTCTTCCTCTTCCCCC |
| | | CAAAACCCAAGGACACCCTCATGA |
| | | TCTCCCGGACCCCTGAGGTCACATG |
| | | CGTGGTGGTGGACGTGAGCCACGA |
| | | AGACCCTGAGGTCAAGTTCAACTG |
| | | GTACGTGGACGGCGTGGAGGTGCA |
| | | TAATGCCAAGACAAAGCCGCGGGA |
| | | GGAGCAGTACAACAGCACGTACCG |
| | | TGTGGTCAGCGTCCTCACCGTCCTG |
| | | CACCAGGACTGGCTGAATGGCAAG |
| | | GAGTACAAGTGCAAGGTGTCCAAC |
| | | AAAGCCCTCCCAGCCCCCATCGAG |
| | | AAAACCATCTCCAAAGCCAAAGGG |
| | | CAGCCCCGAGAACCACAGGTGTAC |
| | | ACCCTGCCCCCATCCCGGGAGGAG |
| | | ATGACCAAGAACCAGGTCAGCCTG |
| | | ACCTGCCTGGTCAAAGGCTTCTATC |
| | | CCAGCGACATCGCCGTGGAGTGGG |
| | | AGAGCAATGGGCAGCCGGAGAACA |
| | | ACTACAAGACCACGCCTCCCGTGCT |
| | | GGACTCCGACGGCTCCTTCTTCCTC |
| | | TATAGCAAGCTCACCGTGGACAAG |
| | | AGCAGGTGGCAGCAGGGGAACGTC |
| | | TTCTCATGCTCCGTGATGCATGAGG |
| | | CTCTGCACAACCACTACACGCAGA |
| | | AGAGCCTCTCCCTGTCTCCGGGCAA |
| | | ATAG |
| 1161 | Hu anti-huCCR8 LIBC319840-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGAGGCCCTGG |
| | | GACAGACGGCCAGGATTACCTGTG |
| | | GGGGAAACAACATTGGAAGTAAAA |
| | | ATGTGCACTGGTACCAGCAGAAGC |
| | | CAGGCCAGGCCCCTGTACTGGTCAT |
| | | CTATAGGGATAGCAACCGGCCCTCT |
| | | GGGATCCCTGAGCGATTCTCTGGCT |
| | | CCAAGTCGGGGAACACGGCCACCC |
| | | TGACCATCAGCAGAGCCCAAGCCG |
| | | GGGATGAGGCTGACTATTACTGTCA |
| | | GGTGTGGGACAGCAGCACTGTGGT |
| | | TTTCGGCGGAGGGACCAAGGTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1162 | Hu anti-huCCR8 LIBC319840-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGCTGGTGGAGTCTGGGGGAGGCG |
| | | TGGTCCAGCCTGGGAGGTCCCTGA |
| | | GACTCTCCTGTGTAGTCTCTGGATT |
| | | CAACTTCATTAACAATGGCATGCAC |
| | | TGGGTCCGCCAGGCTCCAGGCAAG |
| | | GGGCTGGACTGGGTGGCAGTTATA |
| | | TCAAATGATGGAAGTAATAAATAC |
| | | TATCCAGACTCCGTGAAGGGCCGA |
| | | TTCACCATCTCCAGAGACAATTCCA |
| | | AGAACACGCTGTATCTGCAAATGA |
| | | ACAGCCTGAGAGCTGAGGACTCGG |
| | | CTGTGTATTACTGTGCGAAAGTTTA |
| | | CTATGGTTCGGGAAATTATTATAAA |
| | | AACAACTACTACTACGGTATGGAC |
| | | GTCTGGGGCCAAGGGACCACGGTC |
| | | ACCGTCTCCTCAGCCTCCACCAAGG |
| | | GCCCATCGGTCTTCCCCCTGGCACC |
| | | CTCCTCCAAGAGCACCTCTGGGGGC |
| | | ACAGCGGCCCTGGGCTGCCTGGTC |
| | | AAGGACTACTTCCCCGAACCGGTG |
| | | ACGGTGTCGTGGAACTCAGGCGCC |
| | | CTGACCAGCGGCGTGCACACCTTCC |
| | | CGGCTGTCCTACAGTCCTCAGGACT |
| | | CTACTCCCTCAGCAGCGTGGTGACC |
| | | GTGCCCTCCAGCAGCTTGGGCACCC |
| | | AGACCTACATCTGCAACGTGAATC |
| | | ACAAGCCCAGCAACACCAAGGTGG |
| | | ACAAGAAAGTTGAGCCCAAATCTT |
| | | GTGACAAAACTCACACATGCCCAC |
| | | CGTGCCCAGCACCTGAACTCCTGGG |
| | | GGGACCGTCAGTCTTCCTCTTCCCC |
| | | CCAAAACCCAAGGACACCCTCATG |
| | | ATCTCCCGGACCCCTGAGGTCACAT |
| | | GCGTGGTGGTGGACGTGAGCCACG |
| | | AAGACCCTGAGGTCAAGTTCAACT |
| | | GGTACGTGGACGGCGTGGAGGTGC |
| | | ATAATGCCAAGACAAAGCCGCGGG |
| | | AGGAGCAGTACAACAGCACGTACC |
| | | GTGTGGTCAGCGTCCTCACCGTCCT |
| | | GCACCAGGACTGGCTGAATGGCAA |
| | | GGAGTACAAGTGCAAGGTGTCCAA |
| | | CAAAGCCCTCCCAGCCCCCATCGA |
| | | GAAAACCATCTCCAAAGCCAAAGG |
| | | GCAGCCCCGAGAACCACAGGTGTA |
| | | CACCCTGCCCCCATCCCGGGAGGA |
| | | GATGACCAAGAACCAGGTCAGCCT |
| | | GACCTGCCTGGTCAAAGGCTTCTAT |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTATAGCAAGCTCACCGTGGACA |
| | | AGAGCAGGTGGCAGCAGGGGAACG |
| | | TCTTCTCATGCTCCGTGATGCATGA |
| | | GGCTCTGCACAACCACTACACGCA |
| | | GAAGAGCCTCTCCCTGTCTCCGGGC |
| | | AAATAG |
| 1163 | Hu anti-huCCR8 LIBC320212-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGCCAGGATTACCTGTG |
| | | AGGGAAACAACATTGGAAGTCAAA |
| | | ATGTGCACTGGTACCAGCAGAAGC |
| | | CAGGCCAGGCCCCTGTGCTGGTCAT |
| | | GTATAGGGATAGCAACCGGCCCTC |
| | | TGGGATCCCTGAACGATTCTCTGGC |
| | | TCCAAGTCGGGGAACACGGCCACC |
| | | CTGGCCATCAGCAGAGCCCAAGCC |
| | | GGGGATGAGTCTGACTATTACTGTC |
| | | AGGTGTGGGACGGCAGTGCCGTGG |
| | | TATTCGGCGGAGGGACCACGCTGA |
| | | CCGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1164 | Hu anti-huCCR8 LIBC320212-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGATGC |
| | | AGGTGGTGGAGTCTGGGGGAGGCG |
| | | TGGTCCAGCCTGGGAGGTCCCTGA |
| | | GACTCTCCTGTGCAGCCTCTGGATT |
| | | CACCTTCAGTAGCTCTGGCATGCAC |
| | | TGGGTCCGCCAGGCTCCAGGCAAG |
| | | GGCCTGGAGTGGGTGGCAGTTATA |
| | | TCACATGATGGAAGTAATAAATAC |
| | | TATGCAGACTCCGTGAAGGGCCGA |
| | | TTCACCATCTCCAGAGACAATTCCA |
| | | AGAACACGCTGTATCTGCAAATGA |
| | | ATAGCCTGGGAGGTGAGGACACGG |
| | | CGGTGTATTACTGTGCGAAAGTTTA |
| | | CTATGGTTCGGGGATTTATTATAAA |
| | | AACCGCTATTACTACGGTATGGACG |
| | | TCTGGGGCCAAGGGACCACGGTCA |
| | | TCGTCTCGTCAGCCTCCACCAAGGG |
| | | CCCATCGGTCTTCCCCCTGGCACCC |
| | | TCCTCCAAGAGCACCTCTGGGGGC |
| | | ACAGCGGCCCTGGGCTGCCTGGTC |
| | | AAGGACTACTTCCCCGAACCGGTG |
| | | ACGGTGTCGTGGAACTCAGGCGCC |
| | | CTGACCAGCGGCGTGCACACCTTCC |
| | | CGGCTGTCCTACAGTCCTCAGGACT |
| | | CTACTCCCTCAGCAGCGTGGTGACC |
| | | GTGCCCTCCAGCAGCTTGGGCACCC |
| | | AGACCTACATCTGCAACGTGAATC |
| | | ACAAGCCCAGCAACACCAAGGTGG |
| | | ACAAGAAAGTTGAGCCCAAATCTT |
| | | GTGACAAAACTCACACATGCCCAC |
| | | CGTGCCCAGCACCTGAACTCCTGGG |
| | | GGGACCGTCAGTCTTCCTCTTCCCC |
| | | CCAAAACCCAAGGACACCCTCATG |
| | | ATCTCCCGGACCCCTGAGGTCACAT |
| | | GCGTGGTGGTGGACGTGAGCCACG |
| | | AAGACCCTGAGGTCAAGTTCAACT |
| | | GGTACGTGGACGGCGTGGAGGTGC |
| | | ATAATGCCAAGACAAAGCCGCGGG |
| | | AGGAGCAGTACAACAGCACGTACC |
| | | GTGTGGTCAGCGTCCTCACCGTCCT |
| | | GCACCAGGACTGGCTGAATGGCAA |
| | | GGAGTACAAGTGCAAGGTGTCCAA |
| | | CAAAGCCCTCCCAGCCCCCATCGA |
| | | GAAAACCATCTCCAAAGCCAAAGG |
| | | GCAGCCCCGAGAACCACAGGTGTA |
| | | CACCCTGCCCCCATCCCGGGAGGA |
| | | GATGACCAAGAACCAGGTCAGCCT |
| | | GACCTGCCTGGTCAAAGGCTTCTAT |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTATAGCAAGCTCACCGTGGACA |
| | | AGAGCAGGTGGCAGCAGGGGAACG |
| | | TCTTCTCATGCTCCGTGATGCATGA |
| | | GGCTCTGCACAACCACTACACGCA |
| | | GAAGAGCCTCTCCCTGTCTCCGGGC |
| | | AAATAG |
| 1165 | Hu anti-huCCR8 LIBC320384-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGCCAGGATTACCTGTG |
| | | GGGGACACAACATTGGAAGTAAAG |
| | | GTGTGCACTGGTACCAGCAGAAGC |
| | | CAGGCCAGGCCCCTGTGCTGGTCAT |
| | | CTATAGAAATAGCAACCGGCCCTCT |
| | | GGGATCCCTGAGCGATTCTCTGGCT |
| | | CCAACTCGGGGAACACGGCCACCC |
| | | TGACCATCAGCAGAGCCCAAGCCG |
| | | GGGATGAGGCTGACTATTACTGTCA |
| | | GGTGTGGGACAGCAGCACTGTGGT |
| | | TTTCGGCGGAGGGACCGAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1166 | Hu anti-huCCR8 LIBC320384-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGctGGTGGAGtctGGGGGAGGCGTG |
| | | GCCCAGCCTGGGAGGTCCCTGAGA |
| | | CTCTCCTGTGCAGCCTCTGGATTCA |
| | | ACTTCAGTGattGTGGCATGCACTGG |
| | | GTCCGCCaggCTCCAGGCAAGGGGC |
| | | TGGAGTGGGTGGCAGTTATATCATA |
| | | TGATGGAGGTAATAAATATTATGC |
| | | GGACTCCGTGAAGGGCCGGTTCAC |
| | | CATCTCCAGAGacgATTCCAAGAAC |
| | | ACACTGTAtcTGCAAacggacAGCCTG |
| | | AGAACTGAGGACACGGCTGTGTAT |
| | | TACTGTGCGAAAGTTTACTATGGTT |
| | | CGGGTATTTATTATAAAAACAGGTA |
| | | CTACTACGGGATGGACGTctggggCC |
| | | AAGGGACCACGGTcaccgTCTCCTCA |
| | | GCCTCCACCAAGGGCCCATCGGTCT |
| | | TCCCCCTGGCACCCTCCTCCAAGAG |
| | | CACCTCTGGGGGCACAGCGGCCCT |
| | | GGGCTGCCTGGTCAAGGACTACTTC |
| | | CCCGAACCGGTGACGGTGTCGTGG |
| | | AACTCAGGCGCCCTGACCAGCGGC |
| | | GTGCACACCTTCCCGGCTGTCCTAC |
| | | AGTCCTCAGGACTCTACTCCCTCAG |
| | | CAGCGTGGTGACCGTGCCCTCCAGC |
| | | AGCTTGGGCACCCAGACCTACATCT |
| | | GCAACGTGAATCACAAGCCCAGCA |
| | | ACACCAAGGTGGACAAGAAAGTTG |
| | | AGCCCAAATCTTGTGACAAAACTC |
| | | ACACATGCCCACCGTGCCCAGCAC |
| | | CTGAACTCCTGGGGGGACCGTCAG |
| | | TCTTCCTCTTCCCCCCAAAACCCAA |
| | | GGACACCCTCATGATCTCCCGGACC |
| | | CCTGAGGTCACATGCGTGGTGGTG |
| | | GACGTGAGCCACGAAGACCCTGAG |
| | | GTCAAGTTCAACTGGTACGTGGAC |
| | | GGCGTGGAGGTGCATAATGCCAAG |
| | | ACAAAGCCGCGGGAGGAGCAGTAC |
| | | AACAGCACGTACCGTGTGGTCAGC |
| | | GTCCTCACCGTCCTGCACCAGGACT |
| | | GGCTGAATGGCAAGGAGTACAAGT |
| | | GCAAGGTGTCCAACAAAGCCCTCC |
| | | CAGCCCCCATCGAGAAAACCATCT |
| | | CCAAAGCCAAAGGGCAGCCCCGAG |
| | | AACCACAGGTGTACACCCTGCCCCC |
| | | ATCCCGGGAGGAGATGACCAAGAA |
| | | CCAGGTCAGCCTGACCTGCCTGGTC |
| | | AAAGGCTTCTATCCCAGCGACATCG |
| | | CCGTGGAGTGGGAGAGCAATGGGC |
| | | AGCCGGAGAACAACTACAAGACCA |
| | | CGCCTCCCGTGCTGGACTCCGACGG |
| | | CTCCTTCTTCCTCTATAGCAAGCTC |
| | | ACCGTGGACAAGAGCAGGTGGCAG |
| | | CAGGGGAACGTCTTCTCATGCTCCG |
| | | TGATGCATGAGGCTCTGCACAACC |
| | | ACTACACGCAGAAGAGCCTCTCCCT |
| | | GTCTCCGGGCAAATAG |
| 1167 | Hu anti-huCCR8 LIBC320689-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGGCAGGATTACCTGTG |
| | | GGGGAAACAACATTGGAAGTAAAA |
| | | ATGTGCACTGGTACCAGCAGAAGC |
| | | CAGGCCAGGCCCCTGTGCTGGTCAT |
| | | CTATAGGAGTAGCAACCGGCCCTCT |
| | | GGGATCCCTGAGCGATTCTCTGGCT |
| | | CCAACTCGGGGAACACGGCCACCC |
| | | TGACCATCAGCAGAGCCCAAGCCG |
| | | GGGATGAGTCTGACTATTACTGTCA |
| | | AATATGGGACAGCAGCACTGTGGT |
| | | ATTCGGCGGAGGGACCAAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1168 | Hu anti-huCCR8 LIBC320689-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGGTGGTGGAGTCTGGGGGAGGCG |
| | | TGGTCCAGCCTGGGAGGTCCCTGA |
| | | GACTCTCCTGTGCAGCCTCTGGATT |
| | | CACCTTCAGTAGCTATGGCATGCAC |
| | | TGGGTCCGCCAGGCTCCAGGCAAG |
| | | GGGCTGGAGTGGGTGGCAGTTATA |
| | | TCATTTGATGGAAATAATAAATACT |
| | | ATGCAGACTCCGTGAAGGGCCGAT |
| | | TCACCATCTCCAGAGACAATTCCAA |
| | | GAACACGCTATATCTGCAAATGAA |
| | | CAGCCTGAGAGGTGAGGACACGGC |
| | | GGTGTATTACTGTGCGAGAGTTTAT |
| | | TATGGTTCGGGGAGTTATTATAAAA |
| | | ACCGCTACTACTACGGTATGGACGT |
| | | CTGGGGCCAAGGGACCACGGTCAC |
| | | CGTCTCCACAGCCTCCACCAAGGGC |
| | | CCATCGGTCTTCCCCCTGGCACCCT |
| | | CCTCCAAGAGCACCTCTGGGGGCA |
| | | CAGCGGCCCTGGGCTGCCTGGTCA |
| | | AGGACTACTTCCCCGAACCGGTGA |
| | | CGGTGTCGTGGAACTCAGGCGCCCT |
| | | GACCAGCGGCGTGCACACCTTCCC |
| | | GGCTGTCCTACAGTCCTCAGGACTC |
| | | TACTCCCTCAGCAGCGTGGTGACCG |
| | | TGCCCTCCAGCAGCTTGGGCACCCA |
| | | GACCTACATCTGCAACGTGAATCAC |
| | | AAGCCCAGCAACACCAAGGTGGAC |
| | | AAGAAAGTTGAGCCCAAATCTTGT |
| | | GACAAAACTCACACATGCCCACCG |
| | | TGCCCAGCACCTGAACTCCTGGGG |
| | | GGACCGTCAGTCTTCCTCTTCCCCC |
| | | CAAAACCCAAGGACACCCTCATGA |
| | | TCTCCCGGACCCCTGAGGTCACATG |
| | | CGTGGTGGTGGACGTGAGCCACGA |
| | | AGACCCTGAGGTCAAGTTCAACTG |
| | | GTACGTGGACGGCGTGGAGGTGCA |
| | | TAATGCCAAGACAAAGCCGCGGGA |
| | | GGAGCAGTACAACAGCACGTACCG |
| | | TGTGGTCAGCGTCCTCACCGTCCTG |
| | | CACCAGGACTGGCTGAATGGCAAG |
| | | GAGTACAAGTGCAAGGTGTCCAAC |
| | | AAAGCCCTCCCAGCCCCCATCGAG |
| | | AAAACCATCTCCAAAGCCAAAGGG |
| | | CAGCCCCGAGAACCACAGGTGTAC |
| | | ACCCTGCCCCCATCCCGGGAGGAG |
| | | ATGACCAAGAACCAGGTCAGCCTG |
| | | ACCTGCCTGGTCAAAGGCTTCTATC |
| | | CCAGCGACATCGCCGTGGAGTGGG |
| | | AGAGCAATGGGCAGCCGGAGAACA |
| | | ACTACAAGACCACGCCTCCCGTGCT |
| | | GGACTCCGACGGCTCCTTCTTCCTC |
| | | TATAGCAAGCTCACCGTGGACAAG |
| | | AGCAGGTGGCAGCAGGGGAACGTC |
| | | TTCTCATGCTCCGTGATGCATGAGG |
| | | CTCTGCACAACCACTACACGCAGA |
| | | AGAGCCTCTCCCTGTCTCCGGGCAA |
| | | ATAG |
| 1169 | Hu anti-huCCR8 LIBC321408-1 HuIgG1z | ATGGCCTGGGCTCTGCTGCTCCTCA |
| | mAb_LC | CCCTCCTCACTCAGGGCACAGGGTC |
| | | CTGGGCCTCCTATGAACTGACTCAG |
| | | CCACTCTCAGTGTCAGTGGCCCTGG |
| | | GACAGACGGCCAGGATTACCTGTG |
| | | GGGGAAACAACATTGGAAGTAAAA |
| | | ATGTACACTGGTACCAGCAGAGGC |
| | | CAGGCCAGGCCCCTGTGTTGGTCAT |
| | | CTACAGGGATAGCAACCGGCCCTC |
| | | TGGGATCCCTGAGCGATTATCTGGC |
| | | TCCAAAGCGGGGAACACGGCCACC |
| | | CTGACCATCAGCAGAGCCCACGCC |
| | | GGGGATGAGGCTGACTATTACTGTC |
| | | AGGTGTGGGACAGCAGCACTGTGG |
| | | TTTTCGGCGGAGGGACCGAGCTGA |
| | | CCGTCCAAGGTCAGCCCAAGGCTG |
| | | CACCCTCGGTCACTCTGTTCCCGCC |
| | | CTCCTCTGAGGAGCTTCAAGCCAAC |
| | | AAGGCCACACTGGTGTGTCTCATCA |
| | | GTGACTTCTACCCGGGAGCCGTGAC |
| | | AGTGGCCTGGAAGGCAGATAGCAG |
| | | CCCCGTCAAGGCGGGAGTGGAAAC |
| | | CACCACACCCTCCAAACAAAGCAA |
| | | CAACAAGTACGCGGCCAGCAGCTA |
| | | TCTGAGCCTGACGCCTGAGCAGTG |
| | | GAAGTCCCACAGAAGCTACAGCTG |
| | | CCAGGTCACGCATGAAGGGAGCAC |
| | | CGTGGAGAAGACAGTGGCCCCTAC |
| | | AGAATGTTCATAG |
| 1170 | Hu anti-huCCR8 LIBC321408-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AATTGGTGGAGTCTGGGGGAGGCG |
| | | TGGTCCAGCCTGGGAGGTCTCTGAG |
| | | ACTCTCCTGTGCAGTCTCTGGATTC |
| | | ACGTTCAGTAGCAATGGCATGCACT |
| | | GGGTCCGCCAGGCTCCAGGCAAGG |
| | | GGCTGGAGTGGGTGGCAGTTATAT |
| | | CAAATGATGGAAGTAATAAATATT |
| | | ATGGAGACTCCGTGAAGGGCCGAT |
| | | TCACCATCTCCAGAGACAATTCCAA |
| | | GAACACGCTGTATCTGCAAATGAA |
| | | CAGCCTGAGAGCTGAGGACACGGC |
| | | TGTGTATTACTGTGCGAAAGTTTAC |
| | | TATGGTTCGGGAATTTATTACAGAA |
| | | ACAACTACTACTACGGTATGGACGT |
| | | CTGGGGCCAAGGGACCACGGTCAC |
| | | CGTCTCCTCAGCCTCCACCAAGGGC |
| | | CCATCGGTCTTCCCCCTGGCACCCT |
| | | CCTCCAAGAGCACCTCTGGGGGCA |
| | | CAGCGGCCCTGGGCTGCCTGGTCA |
| | | AGGACTACTTCCCCGAACCGGTGA |
| | | CGGTGTCGTGGAACTCAGGCGCCCT |
| | | GACCAGCGGCGTGCACACCTTCCC |
| | | GGCTGTCCTACAGTCCTCAGGACTC |
| | | TACTCCCTCAGCAGCGTGGTGACCG |
| | | TGCCCTCCAGCAGCTTGGGCACCCA |
| | | GACCTACATCTGCAACGTGAATCAC |
| | | AAGCCCAGCAACACCAAGGTGGAC |
| | | AAGAAAGTTGAGCCCAAATCTTGT |
| | | GACAAAACTCACACATGCCCACCG |
| | | TGCCCAGCACCTGAACTCCTGGGG |
| | | GGACCGTCAGTCTTCCTCTTCCCCC |
| | | CAAAACCCAAGGACACCCTCATGA |
| | | TCTCCCGGACCCCTGAGGTCACATG |
| | | CGTGGTGGTGGACGTGAGCCACGA |
| | | AGACCCTGAGGTCAAGTTCAACTG |
| | | GTACGTGGACGGCGTGGAGGTGCA |
| | | TAATGCCAAGACAAAGCCGCGGGA |
| | | GGAGCAGTACAACAGCACGTACCG |
| | | TGTGGTCAGCGTCCTCACCGTCCTG |
| | | CACCAGGACTGGCTGAATGGCAAG |
| | | GAGTACAAGTGCAAGGTGTCCAAC |
| | | AAAGCCCTCCCAGCCCCCATCGAG |
| | | AAAACCATCTCCAAAGCCAAAGGG |
| | | CAGCCCCGAGAACCACAGGTGTAC |
| | | ACCCTGCCCCCATCCCGGGAGGAG |
| | | ATGACCAAGAACCAGGTCAGCCTG |
| | | ACCTGCCTGGTCAAAGGCTTCTATC |
| | | CCAGCGACATCGCCGTGGAGTGGG |
| | | AGAGCAATGGGCAGCCGGAGAACA |
| | | ACTACAAGACCACGCCTCCCGTGCT |
| | | GGACTCCGACGGCTCCTTCTTCCTC |
| | | TATAGCAAGCTCACCGTGGACAAG |
| | | AGCAGGTGGCAGCAGGGGAACGTC |
| | | TTCTCATGCTCCGTGATGCATGAGG |
| | | CTCTGCACAACCACTACACGCAGA |
| | | AGAGCCTCTCCCTGTCTCCGGGCAA |
| | | ATAG |
| 1171 | Hu anti-huCCR8 LIBC321824-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGCCAGGATTACCTGTG |
| | | GGGGAAACAACATTGGAAGTAAAA |
| | | ATGTGCACTGGTACCAGCAGAAGC |
| | | CAGGCCAGGCCCCTATACTGGTCAT |
| | | CTATAGGAATACCAACCGGCCCTCT |
| | | GGGATCCCTGAGCGATTCTCTGGCT |
| | | CCAACTCGGGGAACACGGCCACCC |
| | | TGACCATCAGCAGAGCCCAAGTCG |
| | | GGGATGAGTCTGACTATTTCTGTCA |
| | | GGTGTGGGACAGCAGCACTGTGGT |
| | | ATTCGGCGGAGGGACCAAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1172 | Hu anti-huCCR8 LIBC321824-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGGTGGTGGAGtctGGGGGAGGCGT |
| | | GGTCCAGccTGGGAGGTCcCTGAGA |
| | | CTCTCCTGTGGAGCCTCTGGATTCA |
| | | CcttCAGtggcTATGGCATgcACTGGGT |
| | | CcgcCAggcTCCAGGCAAGGGGCTGG |
| | | AGTGGGTGGCAGTTATATCATATGA |
| | | TGGAAGTAATAAATACTATGCAGA |
| | | CTCCGTgAAGGGCCGATTCCCCATC |
| | | TCAAGAgaCAATTCCAAGAACACGC |
| | | TGTATCTGCAAATGAACAGcCTGAG |
| | | AGGTGAGGACACGGCGGTGTATTA |
| | | CTGTgcGAGAGTTTATTATGGTTCGG |
| | | GGATTTATTATAAAAACCGCTacTaC |
| | | TACGGTAtgGACGtctGGGGCCAAGG |
| | | GACCACGGTcgcCGTCTCCTCAGCCT |
| | | CCACCAAGGGCCCATCGGTCTTCCC |
| | | CCTGGCACCCTCCTCCAAGAGCACC |
| | | TCTGGGGGCACAGCGGCCCTGGGC |
| | | TGCCTGGTCAAGGACTACTTCCCCG |
| | | AACCGGTGACGGTGTCGTGGAACT |
| | | CAGGCGCCCTGACCAGCGGCGTGC |
| | | ACACCTTCCCGGCTGTCCTACAGTC |
| | | CTCAGGACTCTACTCCCTCAGCAGC |
| | | GTGGTGACCGTGCCCTCCAGCAGCT |
| | | TGGGCACCCAGACCTACATCTGCA |
| | | ACGTGAATCACAAGCCCAGCAACA |
| | | CCAAGGTGGACAAGAAAGTTGAGC |
| | | CCAAATCTTGTGACAAAACTCACAC |
| | | ATGCCCACCGTGCCCAGCACCTGA |
| | | ACTCCTGGGGGGACCGTCAGTCTTC |
| | | CTCTTCCCCCCAAAACCCAAGGACA |
| | | CCCTCATGATCTCCCGGACCCCTGA |
| | | GGTCACATGCGTGGTGGTGGACGT |
| | | GAGCCACGAAGACCCTGAGGTCAA |
| | | GTTCAACTGGTACGTGGACGGCGT |
| | | GGAGGTGCATAATGCCAAGACAAA |
| | | GCCGCGGGAGGAGCAGTACAACAG |
| | | CACGTACCGTGTGGTCAGCGTCCTC |
| | | ACCGTCCTGCACCAGGACTGGCTG |
| | | AATGGCAAGGAGTACAAGTGCAAG |
| | | GTGTCCAACAAAGCCCTCCCAGCCC |
| | | CCATCGAGAAAACCATCTCCAAAG |
| | | CCAAAGGGCAGCCCCGAGAACCAC |
| | | AGGTGTACACCCTGCCCCCATCCCG |
| | | GGAGGAGATGACCAAGAACCAGGT |
| | | CAGCCTGACCTGCCTGGTCAAAGG |
| | | CTTCTATCCCAGCGACATCGCCGTG |
| | | GAGTGGGAGAGCAATGGGCAGCCG |
| | | GAGAACAACTACAAGACCACGCCT |
| | | CCCGTGCTGGACTCCGACGGCTCCT |
| | | TCTTCCTCTATAGCAAGCTCACCGT |
| | | GGACAAGAGCAGGTGGCAGCAGGG |
| | | GAACGTCTTCTCATGCTCCGTGATG |
| | | CATGAGGCTCTGCACAACCACTAC |
| | | ACGCAGAAGAGCCTCTCCCTGTCTC |
| | | CGGGCAAATAG |
| 1173 | Hu anti-huCCR8 LIBC321845-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGCCAGGATTACCTGTG |
| | | GGGGAAACAACATTGGAAGTAAAA |
| | | ATGTGCACTGGTACCAGCAGAAGC |
| | | CAGGCCAGGCCCCTATACTGGTCAT |
| | | CTATAGGAATACCAACCGGCCCTCT |
| | | GGGATCCCTGAGCGATTCTCTGGCT |
| | | CCAACTCGGGGAACACGGCCACCC |
| | | TGACCATCAGCAGAGCCCAAGTCG |
| | | GGGATGAGTCTGACTATTTCTGTCA |
| | | GGTGTGGGACAGCAGCACTGTGGT |
| | | ATTCGGCGGAGGGACCAAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1174 | Hu anti-huCCR8 LIBC321845-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGGTGGTGGAGTCTGGGGGAGGCG |
| | | TGGTCCAGCCTGGGAGGTCCCTGA |
| | | GACTCTCCTGTGGAGCCTCTGGATT |
| | | CACCTTCAGTGGCTATGGCATGCAC |
| | | TGGGTCCGCCAGGCTCCAGGCAAG |
| | | GGGCTGGAGTGGGTGGCAGTTATA |
| | | TCATATGATGGAAGTAATAAATACT |
| | | ATGCAGACTCCGTGAAGGGCCGAT |
| | | TCACCATCTCAAGAGACAATTCCAA |
| | | GAACACGCTGTATCTGCAAATGAA |
| | | CAGCCTGAGAGGTGAGGACACGGC |
| | | GGTGTATTACTGTGCGAGAGTTTAT |
| | | TATGGTTCGGGGATTTATTATAAAA |
| | | ACCGCTACTACTACGGTATGGACGT |
| | | CTGGGGCCAAGGGACCACGGTCGC |
| | | CGTCTCCTCAGCCTCCACCAAGGGC |
| | | CCATCGGTCTTCCCCCTGGCACCCT |
| | | CCTCCAAGAGCACCTCTGGGGGCA |
| | | CAGCGGCCCTGGGCTGCCTGGTCA |
| | | AGGACTACTTCCCCGAACCGGTGA |
| | | CGGTGTCGTGGAACTCAGGCGCCCT |
| | | GACCAGCGGCGTGCACACCTTCCC |
| | | GGCTGTCCTACAGTCCTCAGGACTC |
| | | TACTCCCTCAGCAGCGTGGTGACCG |
| | | TGCCCTCCAGCAGCTTGGGCACCCA |
| | | GACCTACATCTGCAACGTGAATCAC |
| | | AAGCCCAGCAACACCAAGGTGGAC |
| | | AAGAAAGTTGAGCCCAAATCTTGT |
| | | GACAAAACTCACACATGCCCACCG |
| | | TGCCCAGCACCTGAACTCCTGGGG |
| | | GGACCGTCAGTCTTCCTCTTCCCCC |
| | | CAAAACCCAAGGACACCCTCATGA |
| | | TCTCCCGGACCCCTGAGGTCACATG |
| | | CGTGGTGGTGGACGTGAGCCACGA |
| | | AGACCCTGAGGTCAAGTTCAACTG |
| | | GTACGTGGACGGCGTGGAGGTGCA |
| | | TAATGCCAAGACAAAGCCGCGGGA |
| | | GGAGCAGTACAACAGCACGTACCG |
| | | TGTGGTCAGCGTCCTCACCGTCCTG |
| | | CACCAGGACTGGCTGAATGGCAAG |
| | | GAGTACAAGTGCAAGGTGTCCAAC |
| | | AAAGCCCTCCCAGCCCCCATCGAG |
| | | AAAACCATCTCCAAAGCCAAAGGG |
| | | CAGCCCCGAGAACCACAGGTGTAC |
| | | ACCCTGCCCCCATCCCGGGAGGAG |
| | | ATGACCAAGAACCAGGTCAGCCTG |
| | | ACCTGCCTGGTCAAAGGCTTCTATC |
| | | CCAGCGACATCGCCGTGGAGTGGG |
| | | AGAGCAATGGGCAGCCGGAGAACA |
| | | ACTACAAGACCACGCCTCCCGTGCT |
| | | GGACTCCGACGGCTCCTTCTTCCTC |
| | | TATAGCAAGCTCACCGTGGACAAG |
| | | AGCAGGTGGCAGCAGGGGAACGTC |
| | | TTCTCATGCTCCGTGATGCATGAGG |
| | | CTCTGCACAACCACTACACGCAGA |
| | | AGAGCCTCTCCCTGTCTCCGGGCAA |
| | | ATAG |
| 1175 | Hu anti-huCCR8 LIBC322176-1 HuIgG1z | ATGGCCTGGGCTCTGCTGCTCCTCA |
| | mAb_LC | CCCTCCTCACTCAGGGCACAGGGTC |
| | | CTGGGCCTCCTATGACCTGACTCAG |
| | | CCACTCTCAGTGTCAGTGGCCCTGG |
| | | GACAGACGGCCAGGATTACCTGTG |
| | | GGGGAAACAACATTGGAGATAAAA |
| | | ATGTGCACTGGTACCAGCAGAAGC |
| | | CAGGCCAGGCCCCTGTGCTGGTCAT |
| | | CTATAGGAATAACGTCCGGCCCTCT |
| | | GGGATCCCTGAGCGATTCTCTGGCT |
| | | CCAACTCGGGGAACACGGCCACCC |
| | | TGACCATCAGCAGAGCCCAAGCCG |
| | | GGGATGAGGCTGACTATTACTGTCA |
| | | GGTGTGGGACAGCAGCACTGTGGT |
| | | TTTCGGCGGAGGGACCAAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1176 | Hu anti-huCCR8 LIBC322176-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGCTGGTGGAATCTGGGGGAGGCG |
| | | TGGTCCAGCCTGGGAGGTCCCTGA |
| | | GACTCTCCTGTGCAGCCTCTGGGCT |
| | | CAACTTCAGTAACTTTGGCATGCAC |
| | | TGGGTCCGCCAGGCTCCAGGCAAG |
| | | GGGCTGGACTGGGTGGCAGTTATA |
| | | TCATATGATGGAGGTAATAAATACT |
| | | ATGCAGACTCCGTGAAGGGCCGAT |
| | | TCACCGTCTCCAGAGACAATTCCAA |
| | | GAACACGCTCTTTCTGCAAATGAAC |
| | | AGCCTGAGAGCTGAGGACACGGCT |
| | | CTGTATTACTGTGCGAAAGTTTACT |
| | | ATGGCTCGGGCAGTTATTATAAAA |
| | | AGAGGTACTACTACGGTATGGACG |
| | | TCTGGGGCCAGGGGACCACGGTCA |
| | | CCGTCTCCTCAGCCTCCACCAAGGG |
| | | CCCATCGGTCTTCCCCCTGGCACCC |
| | | TCCTCCAAGAGCACCTCTGGGGGC |
| | | ACAGCGGCCCTGGGCTGCCTGGTC |
| | | AAGGACTACTTCCCCGAACCGGTG |
| | | ACGGTGTCGTGGAACTCAGGCGCC |
| | | CTGACCAGCGGCGTGCACACCTTCC |
| | | CGGCTGTCCTACAGTCCTCAGGACT |
| | | CTACTCCCTCAGCAGCGTGGTGACC |
| | | GTGCCCTCCAGCAGCTTGGGCACCC |
| | | AGACCTACATCTGCAACGTGAATC |
| | | ACAAGCCCAGCAACACCAAGGTGG |
| | | ACAAGAAAGTTGAGCCCAAATCTT |
| | | GTGACAAAACTCACACATGCCCAC |
| | | CGTGCCCAGCACCTGAACTCCTGGG |
| | | GGGACCGTCAGTCTTCCTCTTCCCC |
| | | CCAAAACCCAAGGACACCCTCATG |
| | | ATCTCCCGGACCCCTGAGGTCACAT |
| | | GCGTGGTGGTGGACGTGAGCCACG |
| | | AAGACCCTGAGGTCAAGTTCAACT |
| | | GGTACGTGGACGGCGTGGAGGTGC |
| | | ATAATGCCAAGACAAAGCCGCGGG |
| | | AGGAGCAGTACAACAGCACGTACC |
| | | GTGTGGTCAGCGTCCTCACCGTCCT |
| | | GCACCAGGACTGGCTGAATGGCAA |
| | | GGAGTACAAGTGCAAGGTGTCCAA |
| | | CAAAGCCCTCCCAGCCCCCATCGA |
| | | GAAAACCATCTCCAAAGCCAAAGG |
| | | GCAGCCCCGAGAACCACAGGTGTA |
| | | CACCCTGCCCCCATCCCGGGAGGA |
| | | GATGACCAAGAACCAGGTCAGCCT |
| | | GACCTGCCTGGTCAAAGGCTTCTAT |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTATAGCAAGCTCACCGTGGACA |
| | | AGAGCAGGTGGCAGCAGGGGAACG |
| | | TCTTCTCATGCTCCGTGATGCATGA |
| | | GGCTCTGCACAACCACTACACGCA |
| | | GAAGAGCCTCTCCCTGTCTCCGGGC |
| | | AAATAG |
| 1177 | Hu anti-huCCR8 LIBC323412-1 HuIgG1z | ATGGCCTGGGCTCTGCTGCTCCTCA |
| | mAb_LC | CCCTCCTCACTCAGGGCACAGGGTC |
| | | CTGGGCCTCCTATGAGCTGACTCAG |
| | | CCACTCTCAGTGTCAGTGGCCCTGG |
| | | GACAGACGGCCAGGATTACCTGTG |
| | | GGGGAAACAACATTGGAAGTAAAA |
| | | ATGTGCACTGGTACCAGCAGAAGC |
| | | CAGGCCAGGCCCCTGTGCTGGTCAT |
| | | CTATAGGGATAGCAACCGGCCCTCT |
| | | GGGATCCCTGAGCGATTCTCTGGCT |
| | | CCAAGTCGGGGAACACGGCCACCC |
| | | TGACCATCAGCAGAGCCCAAGCCG |
| | | GGGATGAGGCTGACTATTACTGTCA |
| | | GGTGTGGGACAGCAGCACTGTGGT |
| | | TTTCGGCGGAGGGGCCAAGCTGAC |
| | | CGTCCTAGGTCAGCCCAAGGCTGC |
| | | ACCCTCGGTCACTCTGTTCCCGCCC |
| | | TCCTCTGAGGAGCTTCAAGCCAACA |
| | | AGGCCACACTGGTGTGTCTCATCAG |
| | | TGACTTCTACCCGGGAGCCGTGACA |
| | | GTGGCCTGGAAGGCAGATAGCAGC |
| | | CCCGTCAAGGCGGGAGTGGAAACC |
| | | ACCACACCCTCCAAACAAAGCAAC |
| | | AACAAGTACGCGGCCAGCAGCTAT |
| | | CTGAGCCTGACGCCTGAGCAGTGG |
| | | AAGTCCCACAGAAGCTACAGCTGC |
| | | CAGGTCACGCATGAAGGGAGCACC |
| | | GTGGAGAAGACAGTGGCCCCTACA |
| | | GAATGTTCATAG |
| 1178 | Hu anti-huCCR8 LIBC323412-1 HuIgG1z | ATGGACATGAGGGTGCCCGCTCAG |
| | mAb_HC | CTCCTGGGGCTCCTGCTGCTGTGGC |
| | | TGAGAGGTGCGCGCTGTCAGGTGC |
| | | AGCTGGTGGAGTCTGGGGGAGGCG |
| | | TGGTCCAGCCTGGGAGGTCCCTGA |
| | | GACTCTCCTGTGCAGCCTCTGGATT |
| | | CAACTTCAGTAGCTGTGGCATGCAC |
| | | TGGGTCCGCCAGGCTCCAGGCAAG |
| | | GGGCTGGAGTGGGTGGCAGTTATA |
| | | TCATATGATGGAACTAATAAATACT |
| | | ATGCGGACTCCGTGAAGGGCCGAT |
| | | TCACCATCTCCAGAGACAATTCCAA |
| | | GAACACGCTGTATCTGCAAATGAA |
| | | CAGCCTGAGAGCTGAGGACACGGC |
| | | TGTGTATTACTGTGCGAAAGTTTAC |
| | | TATGGTTCGGGTATTTATTATAAAA |
| | | AGAACTACTACTACGGTATGGACG |
| | | TCTGGGGCCAAGGGACCACGGTCA |
| | | CCGTCTCCTCAGCCTCCACCAAGGG |
| | | CCCATCGGTCTTCCCCCTGGCACCC |
| | | TCCTCCAAGAGCACCTCTGGGGGC |
| | | ACAGCGGCCCTGGGCTGCCTGGTC |
| | | AAGGACTACTTCCCCGAACCGGTG |
| | | ACGGTGTCGTGGAACTCAGGCGCC |
| | | CTGACCAGCGGCGTGCACACCTTCC |
| | | CGGCTGTCCTACAGTCCTCAGGACT |
| | | CTACTCCCTCAGCAGCGTGGTGACC |
| | | GTGCCCTCCAGCAGCTTGGGCACCC |
| | | AGACCTACATCTGCAACGTGAATC |
| | | ACAAGCCCAGCAACACCAAGGTGG |
| | | ACAAGAAAGTTGAGCCCAAATCTT |
| | | GTGACAAAACTCACACATGCCCAC |
| | | CGTGCCCAGCACCTGAACTCCTGGG |
| | | GGGACCGTCAGTCTTCCTCTTCCCC |
| | | CCAAAACCCAAGGACACCCTCATG |
| | | ATCTCCCGGACCCCTGAGGTCACAT |
| | | GCGTGGTGGTGGACGTGAGCCACG |
| | | AAGACCCTGAGGTCAAGTTCAACT |
| | | GGTACGTGGACGGCGTGGAGGTGC |
| | | ATAATGCCAAGACAAAGCCGCGGG |
| | | AGGAGCAGTACAACAGCACGTACC |
| | | GTGTGGTCAGCGTCCTCACCGTCCT |
| | | GCACCAGGACTGGCTGAATGGCAA |
| | | GGAGTACAAGTGCAAGGTGTCCAA |
| | | CAAAGCCCTCCCAGCCCCCATCGA |
| | | GAAAACCATCTCCAAAGCCAAAGG |
| | | GCAGCCCCGAGAACCACAGGTGTA |
| | | CACCCTGCCCCCATCCCGGGAGGA |
| | | GATGACCAAGAACCAGGTCAGCCT |
| | | GACCTGCCTGGTCAAAGGCTTCTAT |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTATAGCAAGCTCACCGTGGACA |
| | | AGAGCAGGTGGCAGCAGGGGAACG |
| | | TCTTCTCATGCTCCGTGATGCATGA |
| | | GGCTCTGCACAACCACTACACGCA |
| | | GAAGAGCCTCTCCCTGTCTCCGGGC |
| | | AAATAG |
| 1179 | huCCR8_32360_huIgG1z mAb_HC | ATGGACATGAGGGTGCCCGCTCAG |
| | | CTCCTGGGGCTCCTGCTGCTGTGGC |
| | | TGAGAGGTGCGCGCTGTGAGGTGC |
| | | AGCTGGTGGAGTCTGGGGGAGGCT |
| | | TGGTAAAGCCTGGGGGGTCCCTGA |
| | | GACTCTCCTGTGCAGCCTCTGGATT |
| | | TACTTTCAGTAACGCCCGGATGGGC |
| | | TGGGTCCGCCAGGCTCCAGGGAAG |
| | | GGGCTGGAGTGGGTTGGCCGTATT |
| | | AAAAGCAAAACTGAAGGTGGGACA |
| | | AGAGACTACGCTGCACCCGTGAAA |
| | | GGCAGATTCACCATCTCAAGAGAT |
| | | GATTCAAAAAACACGCTGTATCTGC |
| | | AAATGAACAGCCTGAAAACCGAGG |
| | | ACACAGCCGTGTATTATTGTACCTC |
| | | GTATAGTGGGGTCTGGGGCCAAGG |
| | | GACAATGGTCACCGTCTCTTCAGCC |
| | | TCCACCAAGGGCCCATCGGTCTTCC |
| | | CCCTGGCACCCTCCTCCAAGAGCAC |
| | | CTCTGGGGGCACAGCGGCCCTGGG |
| | | CTGCCTGGTCAAGGACTACTTCCCC |
| | | GAACCGGTGACGGTGTCGTGGAAC |
| | | TCAGGCGCCCTGACCAGCGGCGTG |
| | | CACACCTTCCCGGCTGTCCTACAGT |
| | | CCTCAGGACTCTACTCCCTCAGCAG |
| | | CGTGGTGACCGTGCCCTCCAGCAGC |
| | | TTGGGCACCCAGACCTACATCTGCA |
| | | ACGTGAATCACAAGCCCAGCAACA |
| | | CCAAGGTGGACAAGAAAGTTGAGC |
| | | CCAAATCTTGTGACAAAACTCACAC |
| | | ATGCCCACCGTGCCCAGCACCTGA |
| | | ACTCCTGGGGGGACCGTCAGTCTTC |
| | | CTCTTCCCCCCAAAACCCAAGGACA |
| | | CCCTCATGATCTCCCGGACCCCTGA |
| | | GGTCACATGCGTGGTGGTGGACGT |
| | | GAGCCACGAAGACCCTGAGGTCAA |
| | | GTTCAACTGGTACGTGGACGGCGT |
| | | GGAGGTGCATAATGCCAAGACAAA |
| | | GCCGCGGGAGGAGCAGTACAACAG |
| | | CACGTACCGTGTGGTCAGCGTCCTC |
| | | ACCGTCCTGCACCAGGACTGGCTG |
| | | AATGGCAAGGAGTACAAGTGCAAG |
| | | GTGTCCAACAAAGCCCTCCCAGCCC |
| | | CCATCGAGAAAACCATCTCCAAAG |
| | | CCAAAGGGCAGCCCCGAGAACCAC |
| | | AGGTGTACACCCTGCCCCCATCCCG |
| | | GGAGGAGATGACCAAGAACCAGGT |
| | | CAGCCTGACCTGCCTGGTCAAAGG |
| | | CTTCTATCCCAGCGACATCGCCGTG |
| | | GAGTGGGAGAGCAATGGGCAGCCG |
| | | GAGAACAACTACAAGACCACGCCT |
| | | CCCGTGCTGGACTCCGACGGCTCCT |
| | | TCTTCCTCTATAGCAAGCTCACCGT |
| | | GGACAAGAGCAGGTGGCAGCAGGG |
| | | GAACGTCTTCTCATGCTCCGTGATG |
| | | CATGAGGCTCTGCACAACCACTAC |
| | | ACGCAGAAGAGCCTCTCCCTGTCTC |
| | | CGGGCAAATAG |
| 1180 | huCCR8_32360_huIgG1z mAb_LC | ATGGACATGAGGGTGCCCGCTCAG |
| | | CTCCTGGGGCTCCTGCTGCTGTGGC |
| | | TGAGAGGTGCGCGCTGTGACATCG |
| | | TGATGACCCAGTCTCCAGACTCCCT |
| | | GGCTGTGTCTCTGGGCGAGAGGGC |
| | | CACCATCAACTGCAAGTCCAGCCA |
| | | GAGTGTTTTATACAGTTCCAACAAT |
| | | AAGAACTACTTAGCTTGGTACCATC |
| | | AGAAACCAGGACAGTCTCCTAAGC |
| | | TGCTCATTTCCTGGGCATCTACCCG |
| | | GGAATCCGGGGTCCCTGACCGATTC |
| | | AGTGGCAGCGGGTCTGGGACAGAT |
| | | TTCACTCTCACCATCAACAGCCTGC |
| | | AGGCTGAAGATGTGGCAGTTTATTA |
| | | CTGTCAACAATATTATAGTATTCCG |
| | | ATCACTTTCGGCGGAGGGACCAAG |
| | | GTGGAGATCAAACGAACGGTGGCT |
| | | GCACCATCTGTCTTCATCTTCCCGC |
| | | CATCTGATGAGCAGTTGAAATCTGG |
| | | AACTGCCTCTGTTGTGTGCCTGCTG |
| | | AATAACTTCTATCCCAGAGAGGCC |
| | | AAAGTACAGTGGAAGGTGGATAAC |
| | | GCCCTCCAATCGGGTAACTCCCAGG |
| | | AGAGTGTCACAGAGCAGGACAGCA |
| | | AGGACAGCACCTACAGCCTCAGCA |
| | | GCACCCTGACGCTGAGCAAAGCAG |
| | | ACTACGAGAAACACAAAGTCTACG |
| | | CCTGCGAAGTCACCCATCAGGGCCT |
| | | GAGCTCGCCCGTCACAAAGAGCTT |
| | | CAACAGGGGAGAGTGTTAG |
| 1181 | HCDR1 Consensus | X₁X₂GX₄H |
| | | X₁ = N, S, D, G, T, or R, X₂ = C, N, Y, S, |
| | | or F, X₄ = M or F |
| 1182 | LCDR2 Consensus | RX₂X₃X₄RPS |
| | | X₂ = A, N, D, S, or Q, X₃ = S, T, N, I, F, |
| | | or A, and X₄ = N or V |

1. A T cell engager (TCE) molecule comprising (i) an scFab that binds to a tumor antigen, wherein the scFab comprises a first heavy chain variable region (scFab VH), a CHI domain, a first light chain variable region (scFab VL), and a Cκ or Cλ domain, and (ii) an scFv that binds CD3, comprising a second VL and a second VH, and wherein the TCE molecule is a single chain.

2. The TCE molecule of claim 1, wherein the scFab comprises a C-terminus portion that is connected by a linker to an N-terminal portion of the scFv.

3. The TCE molecule of claim 1, wherein the TCE molecule further comprises an scFc.

4. The TCE molecule of claim 1, wherein the TCE molecule further comprises an scFc which comprises an N-terminus portion that is connected by a linker to a C-terminal portion of the scFv.

5. The TCE molecule of claim 1, wherein the scFv binds human CD3.

6. The TCE molecule of claim 1, wherein the scFab has an orientation in the following order, from N-terminus to C-terminus, VH, CH1, VL, and either Cκ or Cλ.

7. The TCE molecule of claim 1, wherein the scFab has an orientation in the following order, from N-terminus to C-terminus, VL, either Cκ or Cλ, VH, and CH1.

8. The TCE molecule of claim 1, wherein the scFab comprises a linker that connects the CH1 and scFab VL, wherein the linker is (G4S)6, (G4S)7, (G4S)8, (G4Q)6, (G4Q)7, or (G4Q)8.

9. The TCE molecule of claim 1, wherein the TCE molecule comprises a linker that connects the scFab Cκ or Cλ and the scFab VH, wherein the linker is (G4S)6, (G4S)7, (G4S)8, (G4Q)6, (G4Q)7, or (G4Q)8.

10. The TCE molecule of claim 1, wherein CH1, Cκ and/or Cλ domains are IgG, IgM, IgA, IgD, or IgE.

11. The TCE molecule of claim 1, wherein the CH1, Cκ and/or Cλ domains are IgG.

12. The TCE molecule of claim 1, wherein the CH1, Cκ and/or Cλ domains are IgG1.

13. The TCE molecule of claim 1, wherein the scFab contains a cysteine clamp between CHI and either Cκ or Cλ.

14. The TCE molecule of claim 1, comprising an orientation from N-terminus to C-terminus of: VH-CH1-Linker-VL-Cκ or Cλ-Linker-VH-Linker-VL-Linker-Fc1 (CH2-CH3)-Linker-Fc2 (CH2-CH3).

15. The TCE molecule of claim 1, comprising an orientation from N-terminus to C-terminus of: VL-CHI-Linker-VH-Ck or Cλ-Linker-VH-Linker-VL-Linker-Fc1 (CH2-CH3)-Linker-Fc2 (CH2-CH3).

16. (canceled)

17. The TCE molecule of claim 1, wherein the scFv that binds CD3 is I2E.

18. The TCE molecule of claim 1, wherein the scFv that binds CD3 is I2C.

19-37. (canceled)

38. A method of treating cancer in a patient comprising administering an effective amount of the TCE molecule of claim 1 to the patient.

39. The method of claim 38, wherein the cancer is a solid tumor.

40. The method of claim 38, wherein the cancer is non-small cell lung cancer, gastric cancer, head and neck squamous cell carcinoma, hepatocellular carcinoma, triple-negative breast cancer, colorectal cancer, pancreatic cancer, or metastatic castrate-resistant prostate cancer.

41. The method of claim 38, wherein the method further comprises administering to the patient a PD-1 antagonist antibody or PD-L1 antagonist antibody.

42. The method of claim 38, wherein the method further comprises administering to the patient a PD-1 antagonist antibody or PD-L1 antagonist antibody which is administered prior to, concurrently with, and/or after administration of the TCE molecule.

43. The method of claim 38, wherein the method further comprises administering to the patient pembrolizumab, nivolumab, cemiplimab, or antibody 20C1.009.

44. The method of claim 38, wherein the method further comprises administering to the patient atezolizumab, avelumab, or durvalumab.

45-57. (canceled)

58. A pharmaceutical composition comprising the TCE molecule of claim 1 and one or more pharmaceutically acceptable carriers, diluents, or excipients.

59-112. (canceled)
